(19) United States
(12) Patent Application Publication
(10) Pub. No.: US 2024/0262782 A1
(43) Pub. Date: Aug. 8, 2024

(54) ORGANIC ELECTROLUMINESCENT COMPOUND AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Jin-Man KIM, Gyeonggi-do (KR); Seung-Hoon YOO, Gyeonggi-do (KR); Hong-Se OH, Gyeonggi-do (KR); Su-Hyun LEE, Gyeonggi-do (KR); So-Mi PARK, Gyeonggi-do (KR); Dong-Hyung LEE, Gyeonggi-do (KR); Hyun-Woo KANG, Gyeonggi-do (KR)

(21) Appl. No.: 18/404,138

(22) Filed: Jan. 4, 2024

(30) Foreign Application Priority Data
Jan. 10, 2023 (KR) 10-2023-0003240
Dec. 6, 2023 (KR) 10-2023-0175086

(51) Int. Cl. C07C 211/54; C07D 209/88; C07D 307/91; C07D 333/76; C07D 493/12; C09K 11/06; H10K 85/60

(52) U.S. Cl. CPC C07C 211/54; C07D 209/88; C07D 307/91; C07D 333/76; C07D 493/12; C09K 11/06; H10K 85/622; H10K 85/626; H10K 85/633; H10K 85/636; H10K 85/654; H10K 85/6574; H10K 85/658; C07C 2603/40; C09K 2211/1014

(57) ABSTRACT

The present disclosure relates to an organic electroluminescent compound and an organic electroluminescent device comprising the same. By comprising the organic electroluminescent compound according to the present disclosure as an organic electroluminescent material, an organic electroluminescent device having long lifespan characteristics can be provided.

### TECHNICAL FIELD

The present disclosure relates to an organic electroluminescent compound and an organic electroluminescent device comprising the same.

### BACKGROUND ART

Among a display device, an electroluminescent device (EL device) is a self-emitting display device that offers advantages such as a wider viewing angle, greater contrast ratio, and faster response time. An organic EL device was first developed by Eastman Kodak in 1987, by using small aromatic diamine molecules and aluminum complexes as materials for forming a light-emitting layer [Appln. Phys. Lett. 51, 913, 1987].

An organic electroluminescent device (OLED) comprises a multi-layer structure, including a hole injection layer, a hole transport layer, a light-emitting layer, an electron transport layer, and an electron injection layer, etc., aiming to enhance efficiency and stability. In this case, the selection of a compound included in the hole transport layer or the like is recognized as one of the means for improving device properties such as the hole transport efficiency to a light-emitting layer, the luminous efficiency, and the lifespan.

In this regard, as materials for a hole injection and a transport, copper phthalocyanine (CuPc), 4,4′-bis[N-(1-naphthyl)-N-phenylamino]biphenyl (NPB), N,N′-diphenyl-N,N′-bis(3-methylphenyl)-(1,1′-biphenyl)-4,4′-diamine (TPD), 4,4′,4″-tris(3-methylphenylphenylamino)triphenylamine (MTDATA), etc., were used in an OLED. However, an OLED prepared using these materials have problems of reduction in quantum efficiency and lifespan. This is due to the circumstance when an OLED is driven under high current, thermal stress occurs between an anode and a hole injection layer, thereby such thermal stress significantly reduces the lifespan of the device. Further, since the organic material used in the hole injection layer has very high hole mobility, there have been problems in that the hole-electron charge balance is broken and the quantum efficiency (cd/A) is lowered.

Therefore, the development of a material for a hole transport layer for improving the performance of an OLED is still required.

KR 2012-0029446 A, WO 2013/065589 A1, and WO 2007/119800 A1 disclose an amine derivative having benzofluorene-based substituents as compounds for organic electroluminescent devices. However, said references do not disclose a deuterated amine derivative specified herein as organic electroluminescent materials.

### DISCLOSURE OF THE INVENTION

### Technical Problem

The object of the present disclosure is firstly, to provide an organic electroluminescent compound which can be prepared for an organic electroluminescent device having long lifespan characteristics, and an organic electroluminescent material comprising the same, and secondly, to provide an organic electroluminescent device comprising the organic electroluminescent compound and the organic electroluminescent material.

### Solution to Problem

As a result of intensive studies to solve the technical problem above, the present inventors found that the aforementioned objective can be achieved by an organic electroluminescent compound represented by the following Formula 1, so that the present invention was completed.

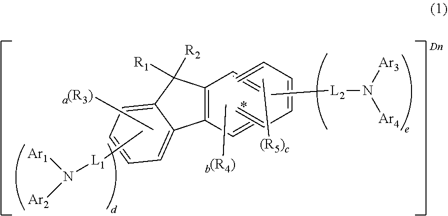

In Formula 1,
R₁ and R₂ each independently represent, a substituted or unsubstituted (C1-C30)alkyl or a substituted or unsubstituted (C6-C30)aryl;
L₁ and L₂ each independently represent, a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
R₃ to R₅ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, or —N—(Ar₅)(Ar₆);
Ar₁ to Ar₄ each independently represent, a substituted or unsubstituted (C6-C30)aryl or a substituted or unsubstituted (3- to 30-membered)heteroaryl;
Ar₅ and Ar₆ each independently represent, hydrogen, a substituted or unsubstituted (C1-C30)alkyl, a substituted or unsubstituted (C2-C30)alkenyl, a substituted or unsubstituted (C6-C30)aryl, or a substituted or unsubstituted (5- to 30-membered)heteroaryl;
a and c each independently represent, an integer of 1 to 4, b is an integer of 1 or 2, when a to c are 2 or more, each of R₃ to R₅ may be the same or different;
d and e each independently represent, 0 or 1, with at least one of d and e being 1; and
Dn signifies that n hydrogens are replaced with deuterium, where n represents an integer of 1 or more.

### Advantageous Effects of Invention

An organic electroluminescent device with long lifespan characteristics can be manufactured by using an organic electroluminescent compound and an organic electroluminescent material comprising the same according to the present disclosure.

### MODE FOR THE INVENTION

Hereinafter, the present disclosure will be described in detail. However, the following description is intended to explain the invention and is not meant in any way to restrict the scope of the invention.

The present disclosure relates to an organic electroluminescent compound represented by Formula 1 above, an organic electroluminescent material comprising the organic electroluminescent compound, and an organic electroluminescent device comprising the organic electroluminescent material.

The term “organic electroluminescent compound” in the present disclosure means a compound that may be used in an organic electroluminescent device, and may be comprised in any material layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including host and dopant materials), an electron buffer material, a hole blocking material, an electron transport material, or an electron injection material, etc.

The term “deuterated” in the present disclosure means that one or more hydrogen atoms of a compound or functional group have been replaced by deuterium, encompassing the partial or complete substitution of hydrogen atoms with deuterium.

Herein, “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. Herein, the term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. Herein, the term “(3- to 7-membered)heterocycloalkyl” is meant to be a cycloalkyl having 3 to 7 ring backbone atoms including at least one, preferably 1 to 4 heteroatoms selected from the group consisting of B, N, O, S, Si, and P, preferably selected from the group consisting of O, S, and N in which the number of the ring backbone carbon atoms is preferably 5 to 7. The above heterocycloalkyl may be a tetrahydrofuran, pyrrolidine, thiolane, and tetrahydropyran, etc. Herein, “(C6-C30)aryl(ene)” is a monocyclic or fused ring radical derived from an aromatic hydrocarbon having 6 to 30 ring backbone carbon atoms, in which the number of the ring backbone carbon atoms is preferably 6 to 20, more preferably 6 to 15, may be partially saturated, and may include a spiro structure. Examples of the aryl specifically may be phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluoren-fluoren]yl, spiro[fluoren-benzofluoren]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. Herein, “(3- to 30-membered)heteroaryl(ene)” is an aryl having 3 to 30 ring backbone atoms including at least one, preferably 1 to 4 heteroatoms selected from the group consisting of B, N, O, S, Si, P, Se, and Ge, in which the number of the ring backbone carbon atoms is preferably 5 to 24. The above heteroaryl(ene) may be a monocyclic ring, or a fused ring condensed with at least one benzene ring; and may be partially saturated. Also, the above heteroaryl or heteroarylene herein may be one formed by linking at least one heteroaryl or aryl group to a heteroaryl group via a single bond(s). Examples of the heteroaryl specifically may be a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthiridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzopyrimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Additionally, “heteroaryl(ene)” can be classified into heteroaryl(ene) with electronic properties and heteroaryl(ene) with hole properties. A heteroaryl(ene) with electronic properties is a substituent with relatively abundant electrons in the parent nucleus. For example, it may be a substituted or unsubstituted pyridinyl, a substituted or unsubstituted pyrimidinyl, a substituted or unsubstituted triazinyl, a substituted or unsubstituted quina It may be zolinyl, a substituted or unsubstituted quinoxalinil, a substituted or unsubstituted quinolyl, etc. Heteroaryl(ene), which has hole characteristics, is a substituent with a relative lack of electrons in the parent nucleus, and for example, it may be a substituted or unsubstituted carbazolyl, a substituted or unsubstituted dibenzofuranyl, or a substituted or unsubstituted dibenzothiophenyl. Herein, the term “a fused ring of (C3-C30) aliphatic ring and (C6-C30) aromatic ring” means a ring formed by fusing at least one aliphatic ring having 3 to 30 ring backbone carbon atoms in which the carbon atoms number is preferably 3 to 25, more preferably 3 to 18, and at least one aromatic ring having 6 to 30 ring backbone carbon atoms in which the carbon atoms number is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of (C3-C30) aliphatic ring and (C6-C30) aromatic ring may be replaced with at least one heteroatom selected from B, N, O, S, Si, and P, preferably at least one heteroatom selected from N, O, and S. The term “Halogen” in the present disclosure includes F, Cl, Br, and I.

In addition, “ortho (o),” “meta (m),” and “para (p)” are meant to signify the substitution position of all substituents. Ortho position is a compound with substituents, which are adjacent to each other, e.g., at the 1 and 2 positions on benzene. Meta position is the next substitution position of the immediately adjacent substitution position, e.g., a compound with substituents at the 1 and 3 positions on benzene. Para position is the next substitution position of the meta position, e.g., a compound with substituents at the 1 and 4 positions on benzene.

Herein, the term “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents, preferably may be a substituted or unsubstituted (5- to 25-membered) mono- or polycyclic, alicyclic, aromatic ring, or a combination thereof. Further, the formed ring may include at least one heteroatom selected from the group consisting of B, N, O, S, Si, and P, preferably, at least one heteroatom selected from the group consisting of N, O, and S. According to one embodiment of the present disclosure, the number of atoms in the ring skeleton is 5 to 20; according to another embodiment of the present disclosure, the number of atoms in the ring skeleton is 5 to 15. In one embodiment, the fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzofluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, “substituted” in the expression “substituted or unsubstituted” means that a hydrogen atom in a certain functional group is replaced with another atom or functional group, i.e., a substituent, and substituted with a group to which two or more substituents are connected among the substituents. For example, “a substituent to which two or more substituents are connected” may be pyridine-triazine. That is, pyridine-triazine may be heteroaryl or may be interpreted as a substituent in which two heteroaryls are connected. Unless otherwise specified, the substituent may replace hydrogen at a position where the substituent can be substituted without limitation, and when two or more hydrogen atoms in a functional group are each replaced with a substituent, each substituent may be the same or different. The maximum number of substituents that can be substituted for a certain functional group may be the total number of valences that can be substituted for each atom forming the functional group. The substituted alkyl(ene), the substituted alkenyl, the substituted aryl(ene), the substituted heteroaryl(ene), and the substituted cycloalkyl(ene) in the formulas of the present disclosure, each independently are substituted by at least one selected from the group consisting of deuterium; halogen; cyano; carboxyl; nitro; hydroxy; phosphine oxide; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3- to 7-membered)heterocycloalkyl; (C6-C30)aryloxy; (C6-C30)arylthio; (3- to 30-membered)heteroaryl unsubstituted or substituted with at least one (C6-C30)aryl; (C6-C30)aryl unsubstituted or substituted with at least one of (C1-C30)alkyl, (C6-C30)aryl, and (3- to 30-membered)heteroaryl; tri(C1-C30)alkylsilyl; tri(C6-C30)arylsilyl; di(C1-C30)alkyl(C6-C30)arylsilyl; (C1-C30)alkyldi(C6-C30)arylsilyl; a fused ring of (C3-C30) aliphatic ring and (C6-C30) aromatic ring; amino; mono- or di-(C1-C30)alkylamino; mono- or di-(C2-C30)alkenylamino; (C1-C30)alkyl(C2-C30)alkenylamino; mono- or di-(C6-C30)arylamino; (C1-C30)alkyl(C6-C30)arylamino; mono- or di-(3- to 30-membered)heteroarylamino; (C1-C30)alkyl(3- to 30-membered)heteroarylamino; (C2-C30)alkenyl(C6-C30)arylamino; (C2-C30)alkenyl(3- to 30-membered)heteroarylamino; (C6-C30)aryl(3- to 30-membered)heteroarylamino; (C1-C30)alkylcarbonyl; (C1-C30) alkoxycarbonyl; (C6-C30)arylcarbonyl; (C6-C30)arylphosphinyl; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl. For example, the substituents of the above substituents may bedeuterium, methyl, phenyl, naphthyl, biphenyl, fluorenyl, dibenzofuranyl, carbazolyl, or diphenylamino, etc.

In formulas of the present disclosure, when a plurality of substituents represented by the same symbol are present, each of the substituents represented by the same symbol may be the same or different.

Hereinafter, an organic electroluminescent compound according to one embodiment will be described.

The organic electroluminescent compound according to one embodiment is represented by the following Formula 1.

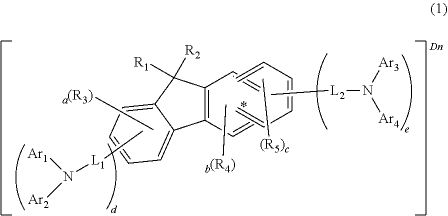

In Formula 1,
R₁ and R₂ each independently represent, a substituted or unsubstituted (C1-C30)alkyl or a substituted or unsubstituted (C6-C30)aryl;
L₁ and L₂ each independently represent, a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
R₃ to R₅ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, or —N—(Ar₅)(Ar₆);
Ar₁ to Ar₄ each independently represent, a substituted or unsubstituted (C6-C30)aryl or a substituted or unsubstituted (3- to 30-membered)heteroaryl;
Ar₅ and Ar₆ each independently represent, hydrogen, a substituted or unsubstituted (C1-C30)alkyl, a substituted or unsubstituted (C2-C30)alkenyl, a substituted or unsubstituted (C6-C30)aryl, or a substituted or unsubstituted (5- to 30-membered)heteroaryl;
a and c each independently represent, an integer of 1 to 4, b is an integer of 1 or 2, when a to c are 2 or more, each of R₃ to R₅ may be the same or different;
d and e each independently represent, 0 or 1, with at least one of d and e being 1;
Dn signifies that n hydrogens are replaced with deuterium, where n represents an integer of 1 or more.

In one embodiment, in Formula 1, d may be 0, and e may be 1.

In one embodiment, in Formula 1, d may be 1, and e may be 0.

In one embodiment, in Formula 1, both d and e may be 1.

In one embodiment, in Formula 1, n may be an integer of 1 or more, where the upper limit of n may be determined by the number of hydrogens that can be substituted in the compound represented by Formula 1.

According to one embodiment, the degree of deuterization in the deuterated organic electroluminescent compound represented by Formula 1 may be at least 15%, preferably at least 30%, more preferably at least 40%, even more preferably at least 55%, and particularly preferably at least 80%. When deuterated with a number equal to or higher than the lower limit, the bond dissociation energy according to deuteration increases, thereby increasing the stability of the compound. When such a compound is used in an organic electroluminescent device, improved lifespan characteristics may be exhibited.

In one embodiment, R₁ and R₂ each independently may be a substituted or unsubstituted (C1-C30)alkyl, preferably a substituted or unsubstituted (C1-C10)alkyl, more preferably a substituted or unsubstituted (C1-C4)alkyl. For example, R₁ and R₂ each independently may be a substituted or unsubstituted methyl.

In one embodiment, L₁ and L₂ each independently may be a single bond or a substituted or unsubstituted (C6-C30)arylene, preferably a single bond or a substituted or unsubstituted (C6-C25)arylene, more preferably a single bond or a substituted or unsubstituted (C6-C18)arylene. For example, L₁ and L₂ each independently represent, a single bond, a substituted or unsubstituted phenylene, or a substituted or unsubstituted naphthylene. The substituents in the substituted group may be for example, deuterium, phenyl, naphthyl, or diphenylamino.

In one embodiment, R₃ to R₅ each independently may be hydrogen, deuterium, halogen, cyano, or a substituted or unsubstituted (C6-C30)aryl, preferably hydrogen, deuterium, or a substituted or unsubstituted (C6-C25)aryl, more preferably hydrogen, deuterium, or a substituted or unsubstituted (C6-C18)aryl. For example, R₃ to R₅ each independently may be hydrogen, deuterium, a substituted or unsubstituted phenyl, or a substituted or unsubstituted biphenyl.

In one embodiment, Ar₁ to Ar₄ each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl. For example, Ar₁ to Ar₄ each independently may be a substituted or unsubstituted phenyl, a substituted or unsubstituted naphthyl, a substituted or unsubstituted p-biphenyl, a substituted or unsubstituted m-biphenyl, a substituted or unsubstituted o-biphenyl, a substituted or unsubstituted p-terphenyl, a substituted or unsubstituted m-terphenyl, a substituted or unsubstituted o-terphenyl, a substituted or unsubstituted o-tetraphenyl, a substituted or unsubstituted fluorenyl, a substituted or unsubstituted benzofluorenyl, a substituted or unsubstituted dibenzofuranyl, a substituted or unsubstituted dibenzothiophenyl, or a substituted or unsubstituted carbazolyl. For example, the substituents in the substituted groups may be deuterium, methyl, phenyl, biphenyl, naphthyl, fluorenyl, dibenzofuranyl, or carbazolyl.

According to one embodiment, the compound represented by Formula 1 may be represented by any one of the following formulas 1-1 to 1-3.

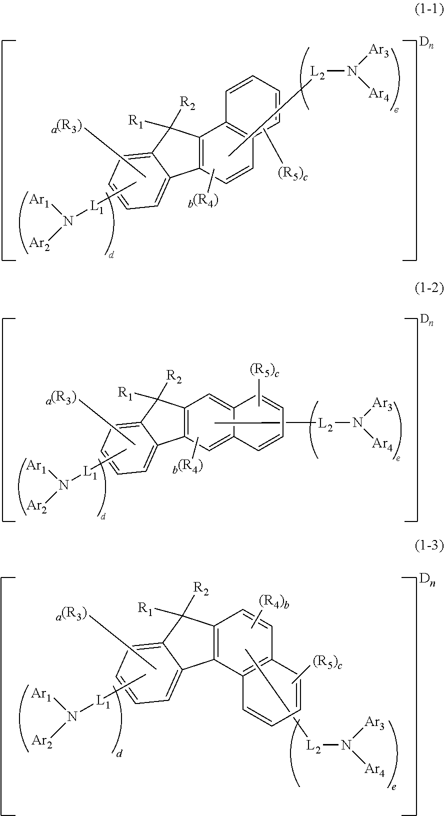

In formulas 1-1 to 1-3,
R₁ to R₅, L₁, L₂, Ar₁ to Ar₄, a to e, and Dn are as defined in Formula 1.

In one embodiment, the compound represented by Formula 1-1 may be represented by any one of the following formulas 1-1-1 to 1-1-6.

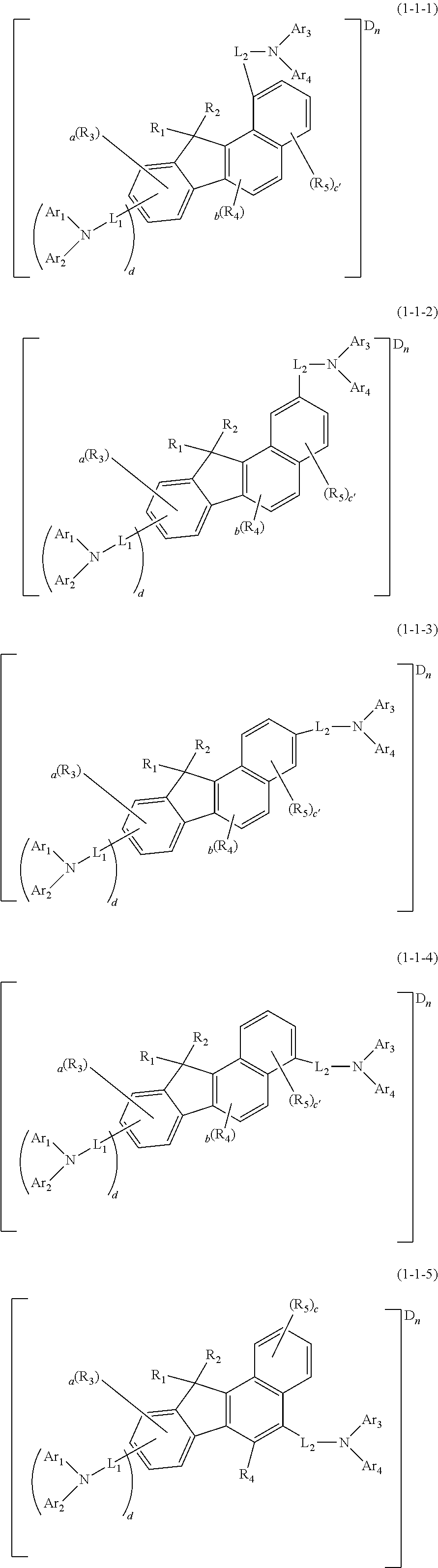

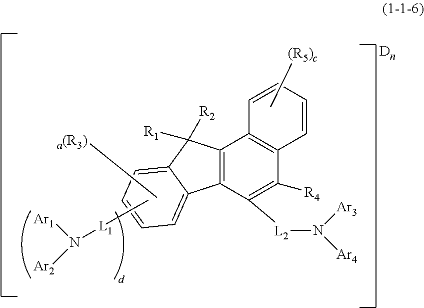

In Formula 1-1-1 to 1-1-6,
R₁ to R₅, L₁, L₂, Ar₁ to Ar₄, a to d, and Dn are as defined in Formula 1-1; and
c′ represents an integer of 1 to 3.

In one embodiment, the compound represented by Formula 1-2 may be represented by any one of the following formulas 1-2-1 to 1-2-6.

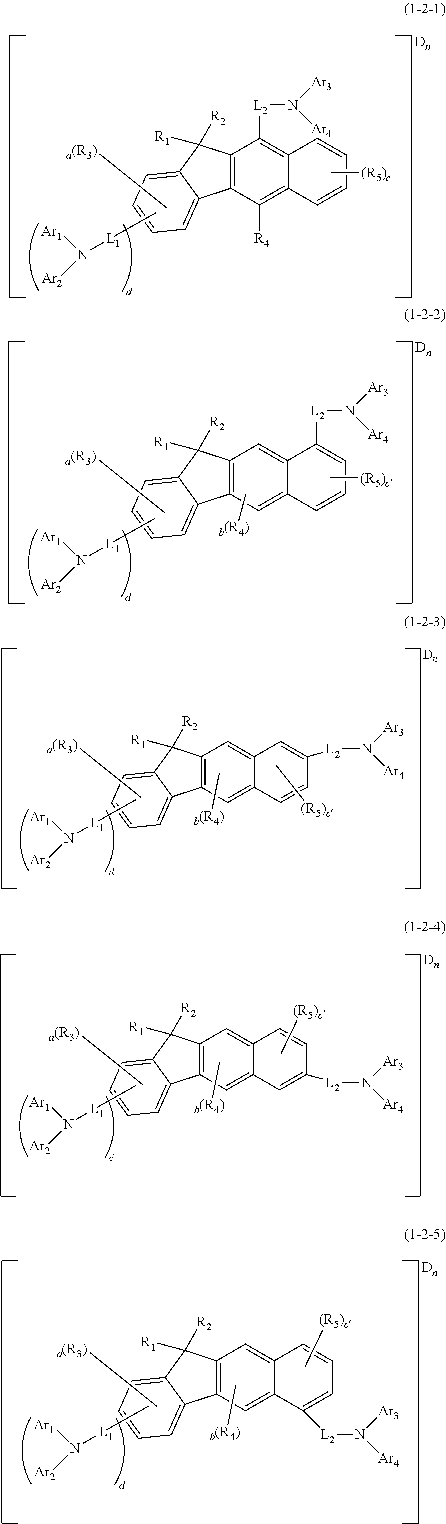

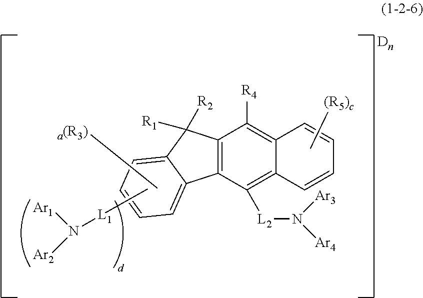

In formulas 1-2-1 to 1-2-6,
R₁ to R₅, L₁, L₂, Ar₁ to Ar₄, a to d, and Dn are as defined in Formula 1-2; and
c′ represents an integer of 1 to 3.

In one embodiment, the compound represented by Formula 1-3 may be represented by any one of the following formulas 1-3-1 to 1-3-6:

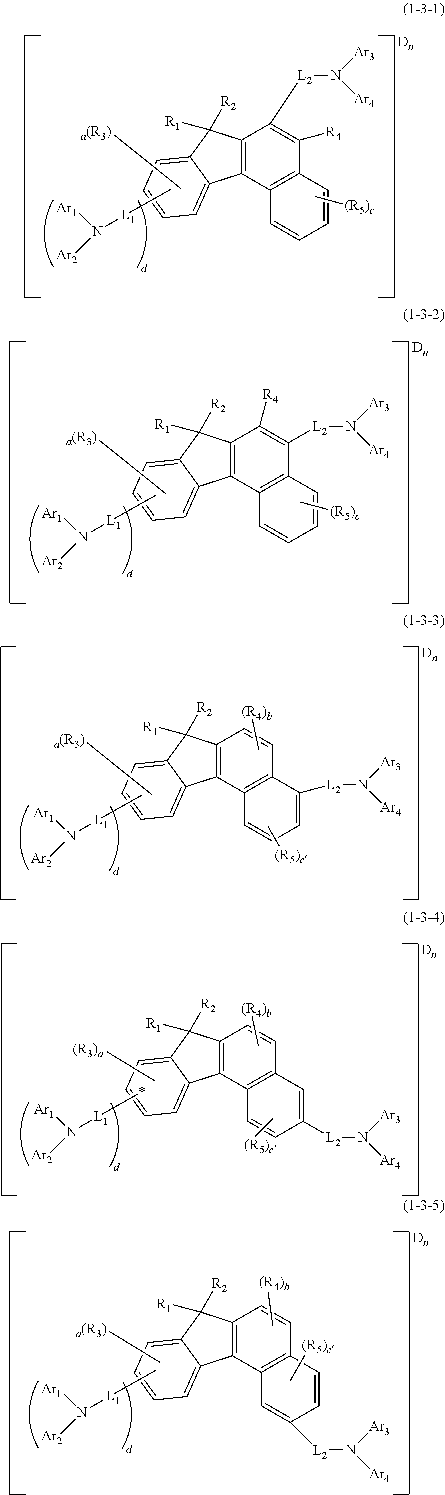

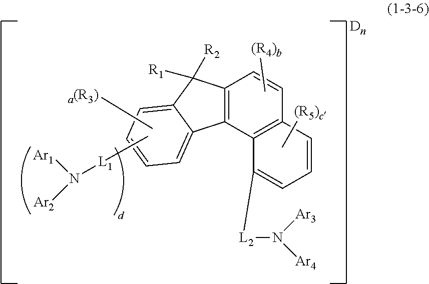

In formulas 1-3-1 to 1-3-6,
R₁ to R₅, L₁, L₂, Ar₁ to Ar₄, a to d, and Dn are as defined in Formula 1-3; and
c′ represents an integer of 1 to 3.

In one embodiment, the compound represented by Formula 1 may be represented by any one of the following formulas 1-4 to 1-7.

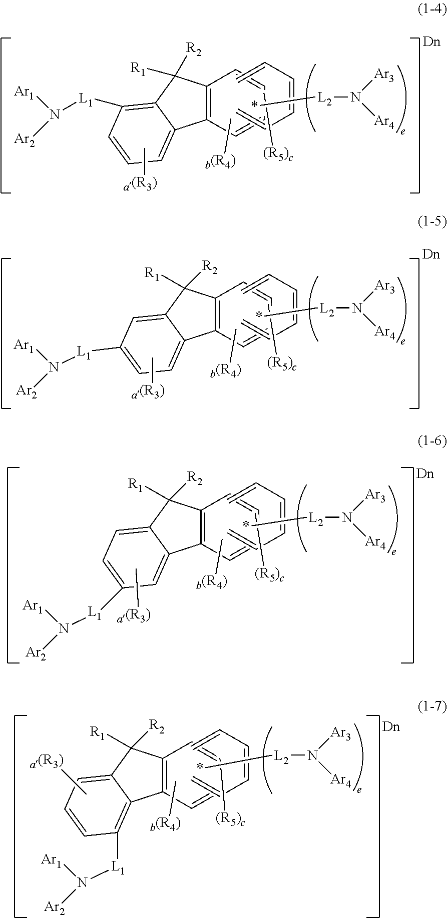

In formulas 1-4 to 1-7,
R₁ to R₅, L₁, L₂, Ar₁ to Ar₄, b, c, e, and Dn are as defined in Formula 1; and
a′ represents an integer of 1 to 3.

According to one embodiment, the organic electroluminescent compound represented by Formula 1 above may be more specifically illustrated by the following compounds, but is not limited thereto.

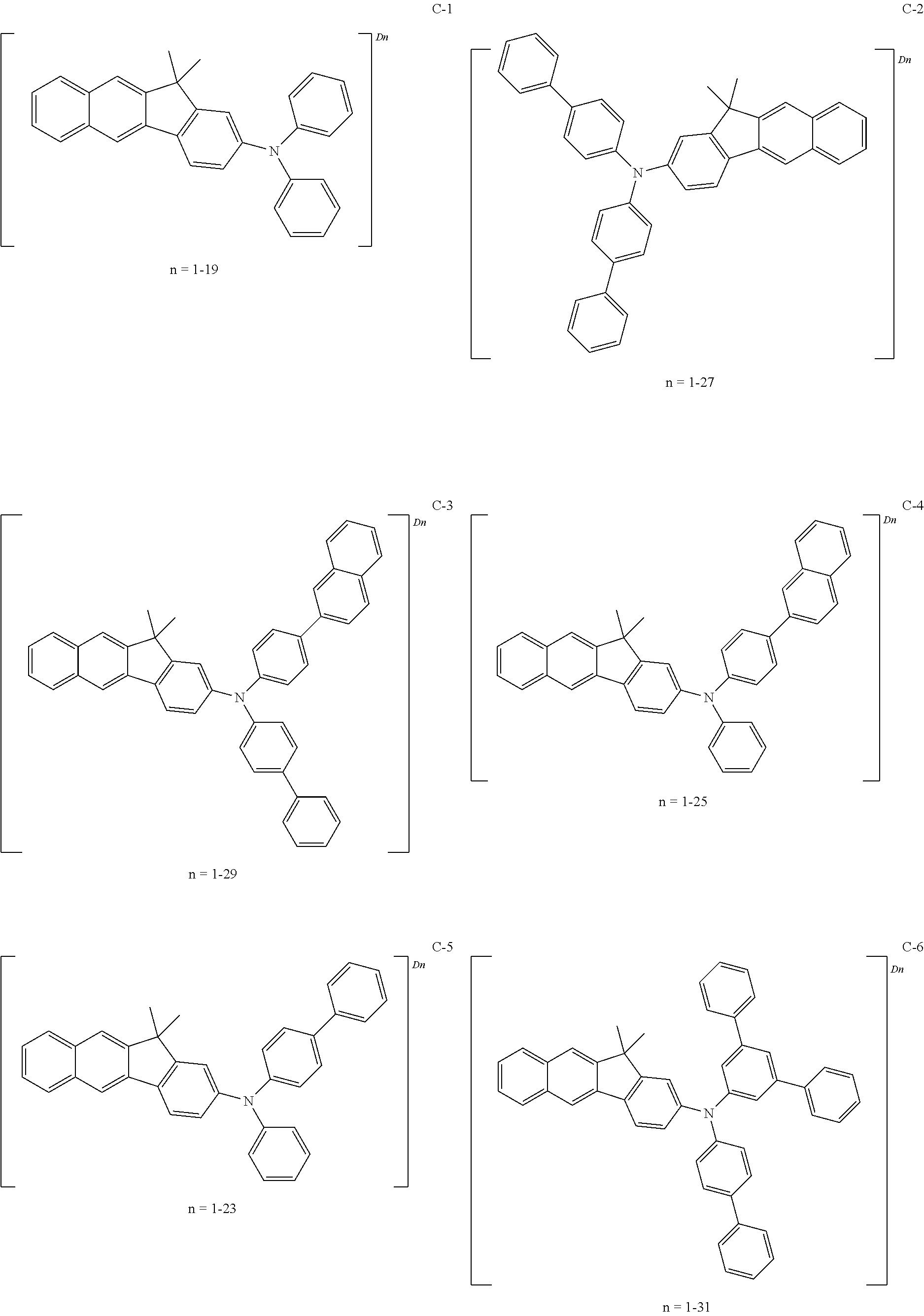

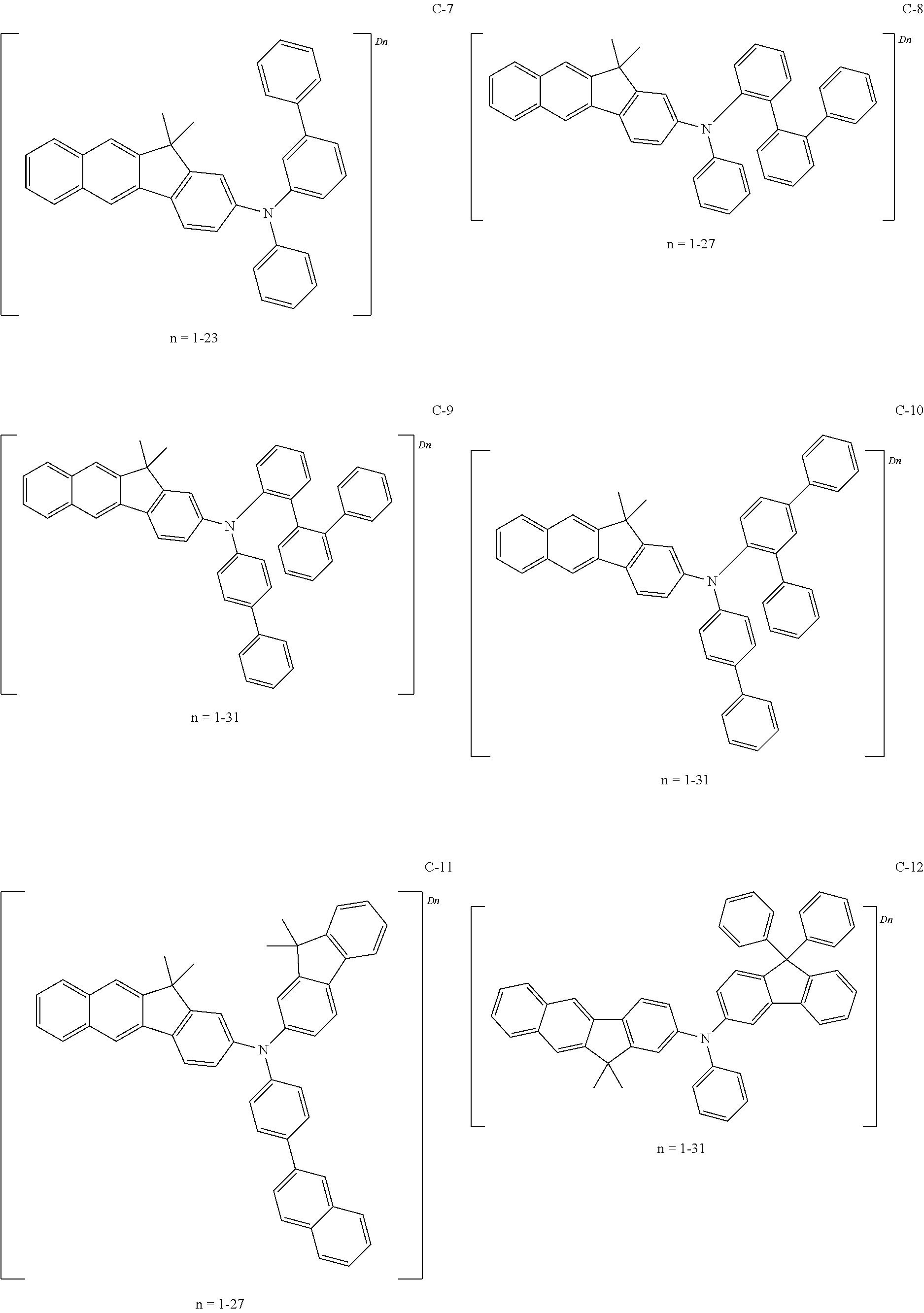

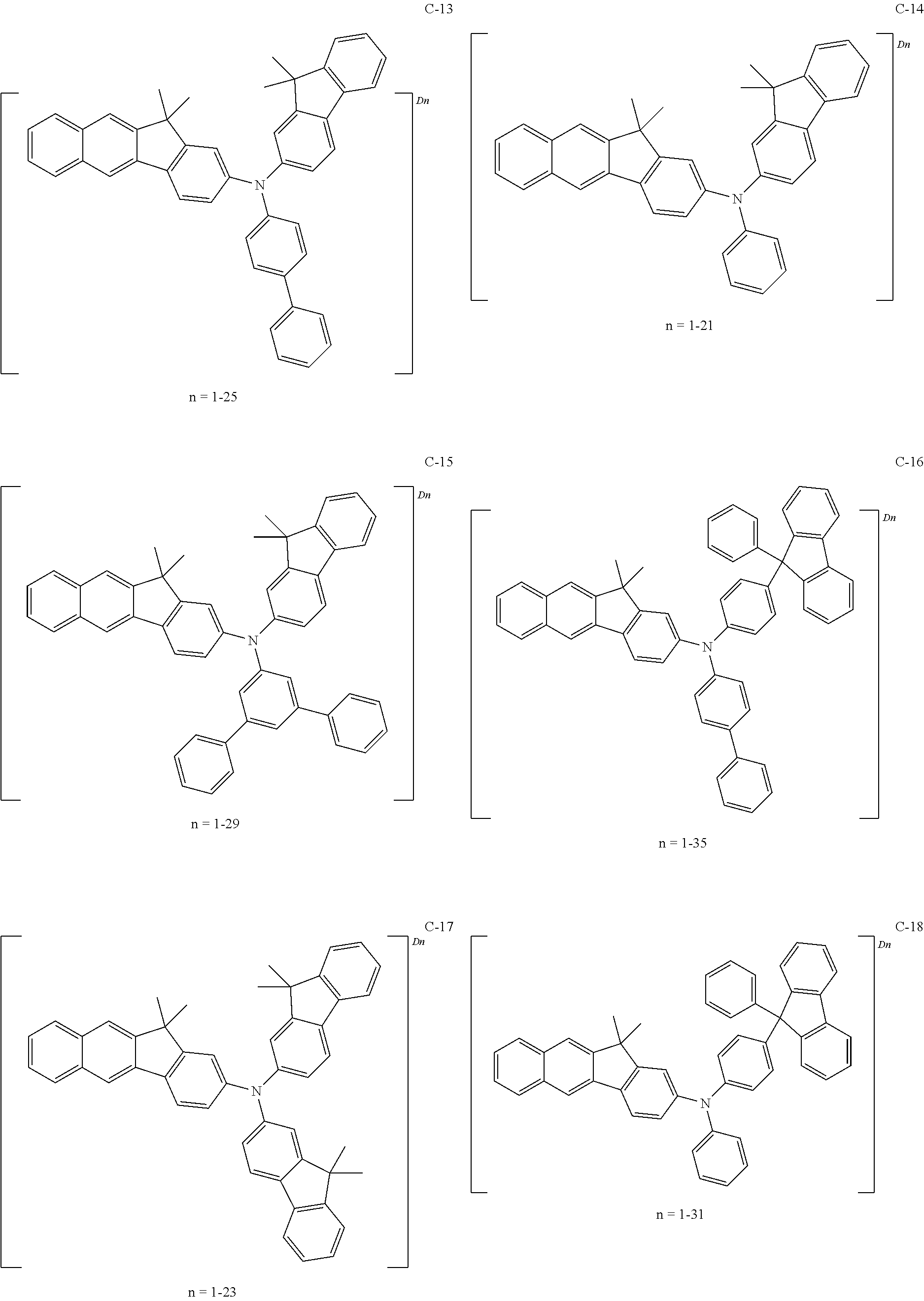

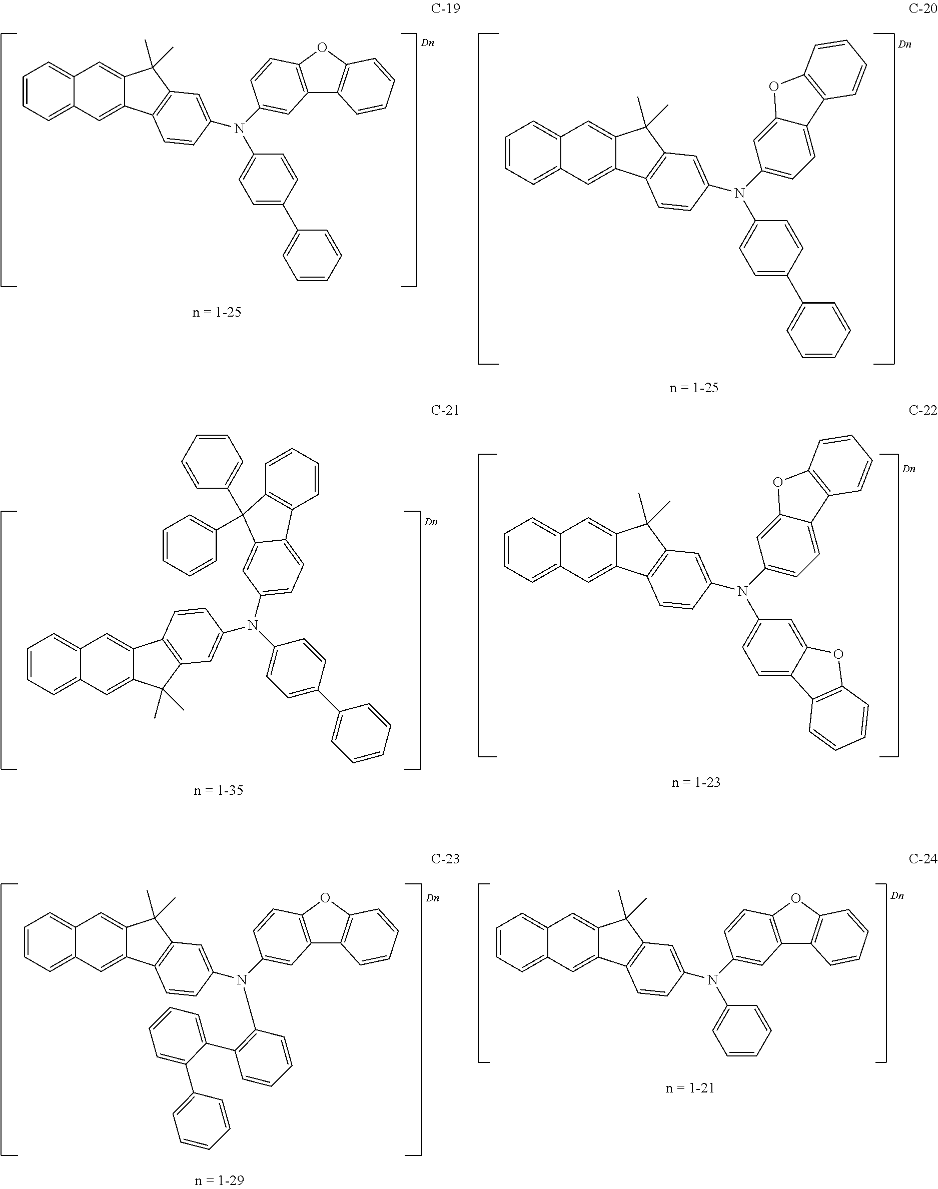

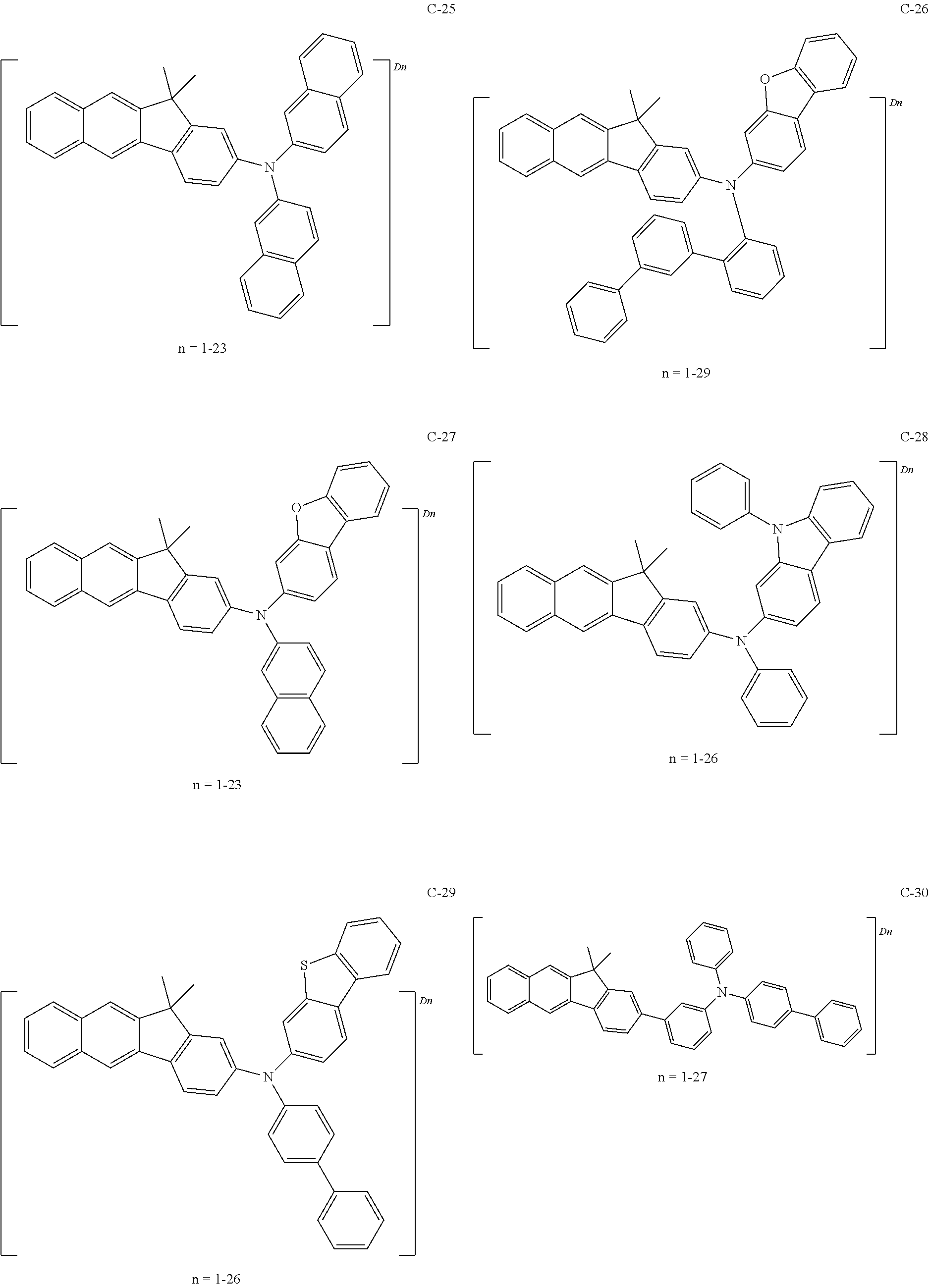

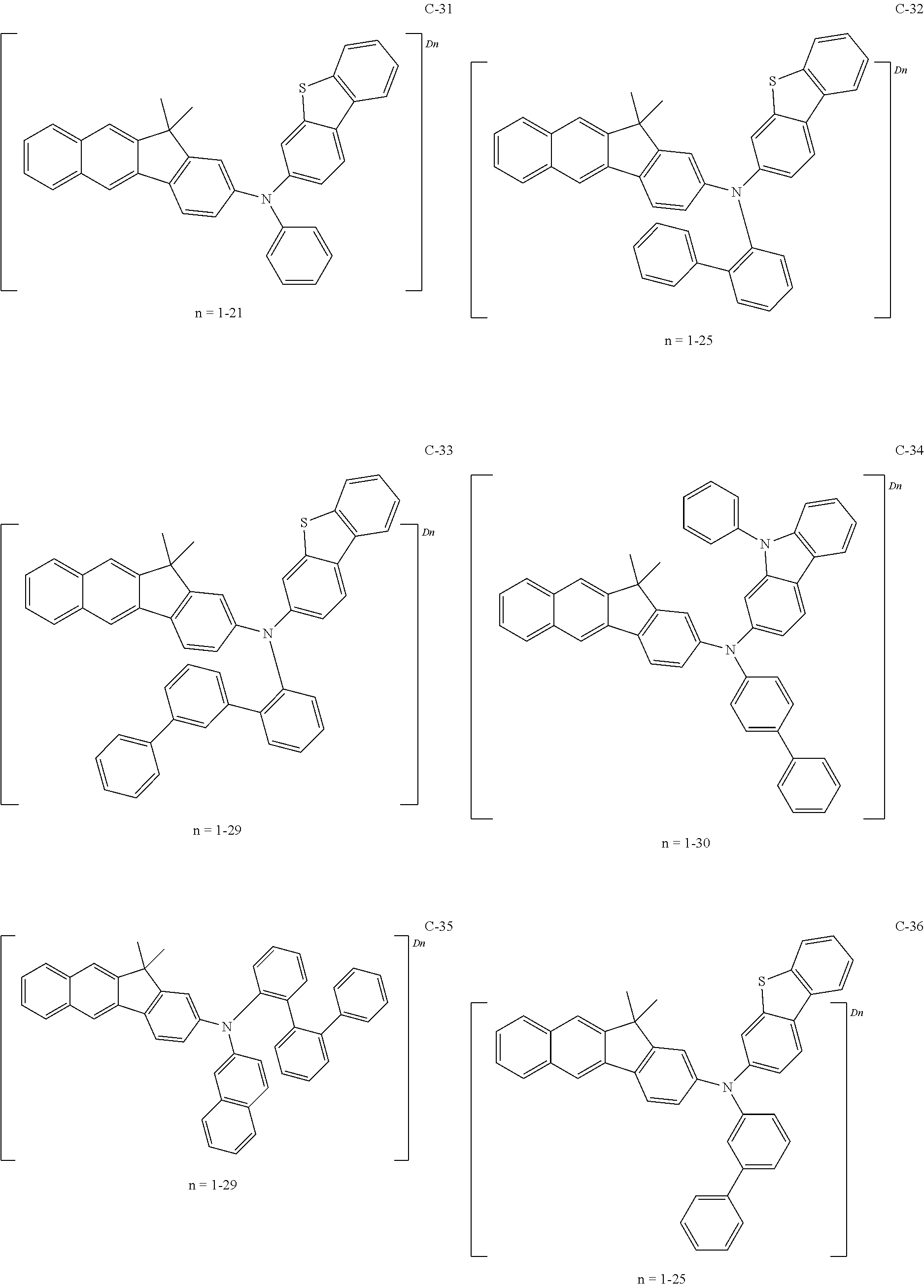

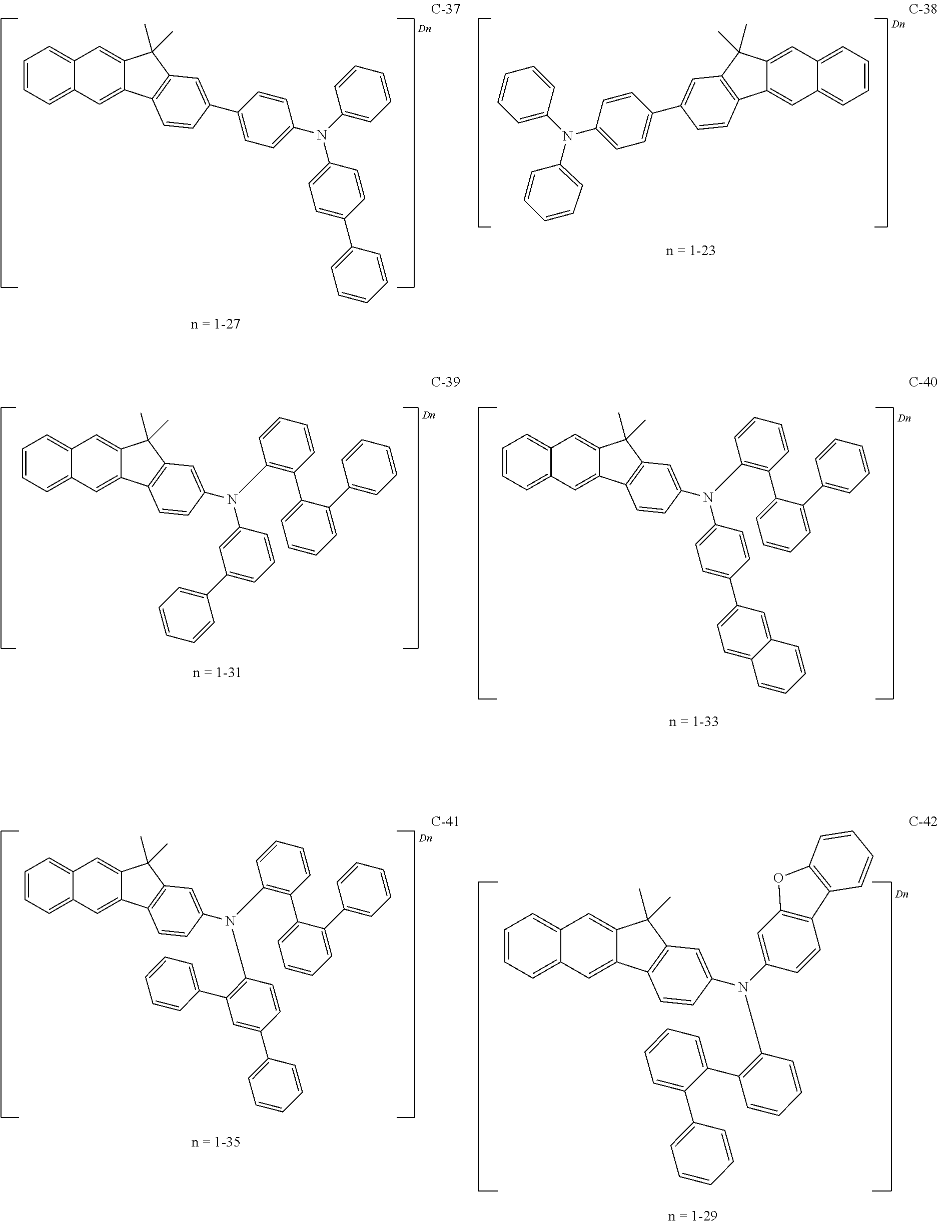

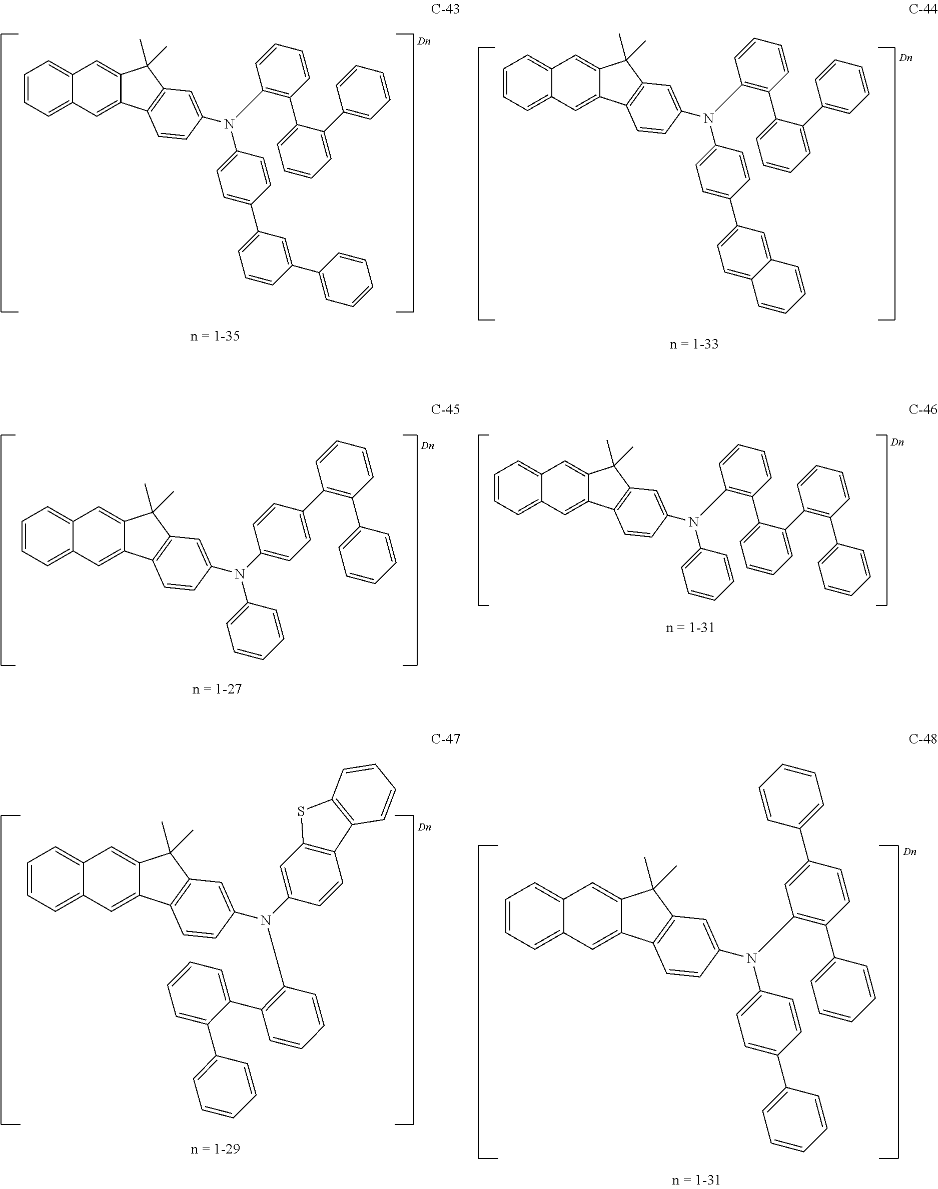

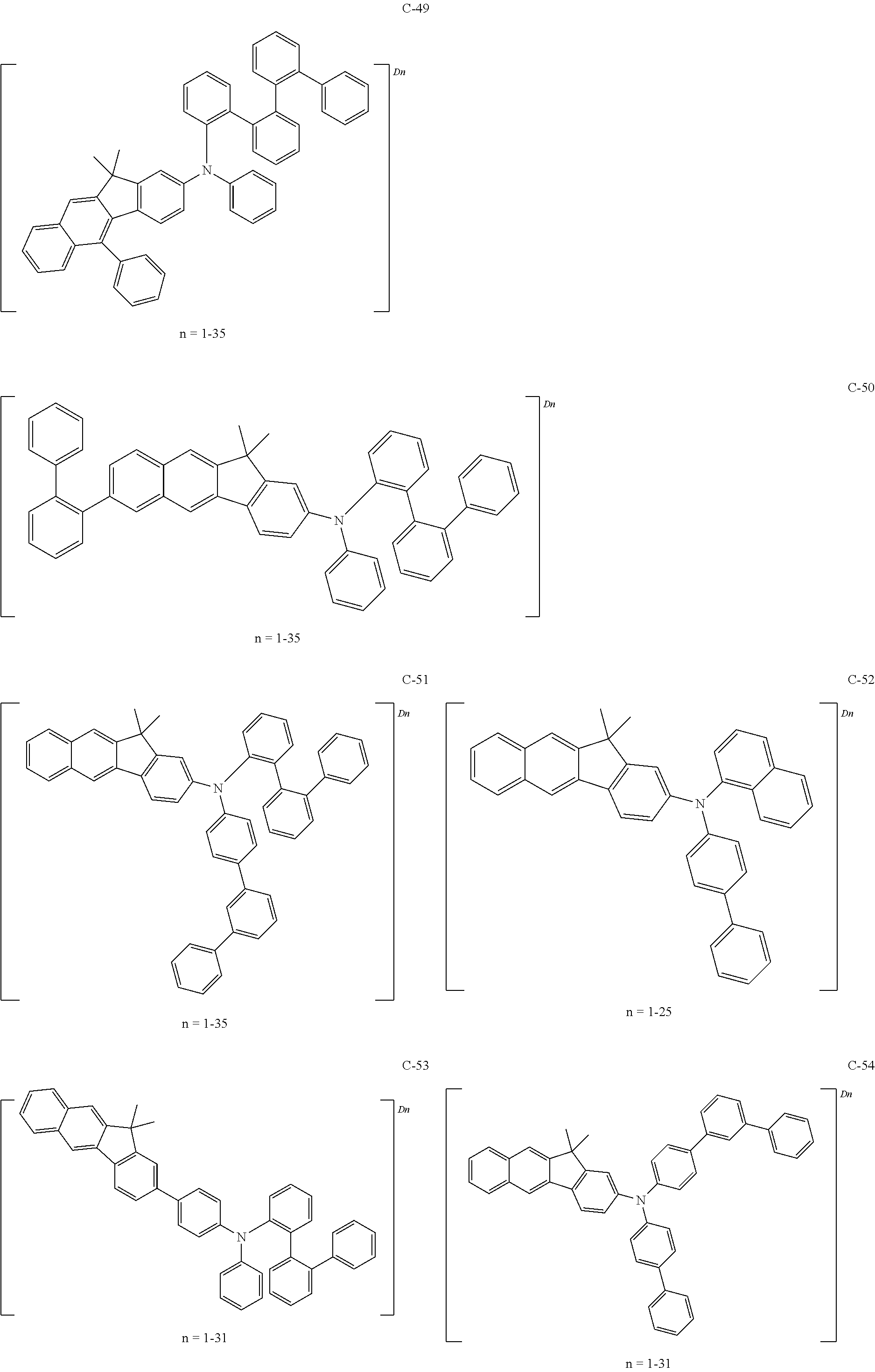

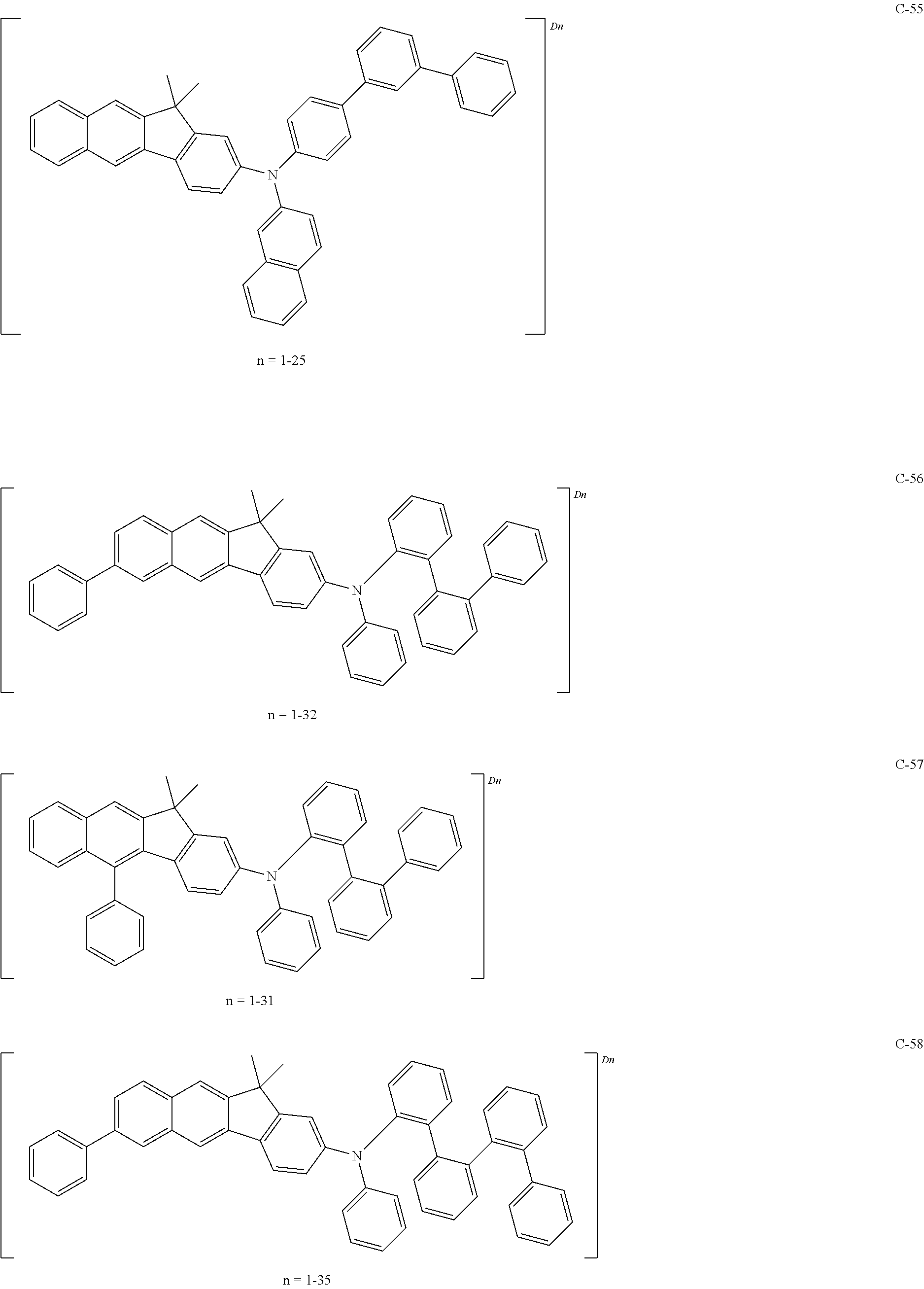

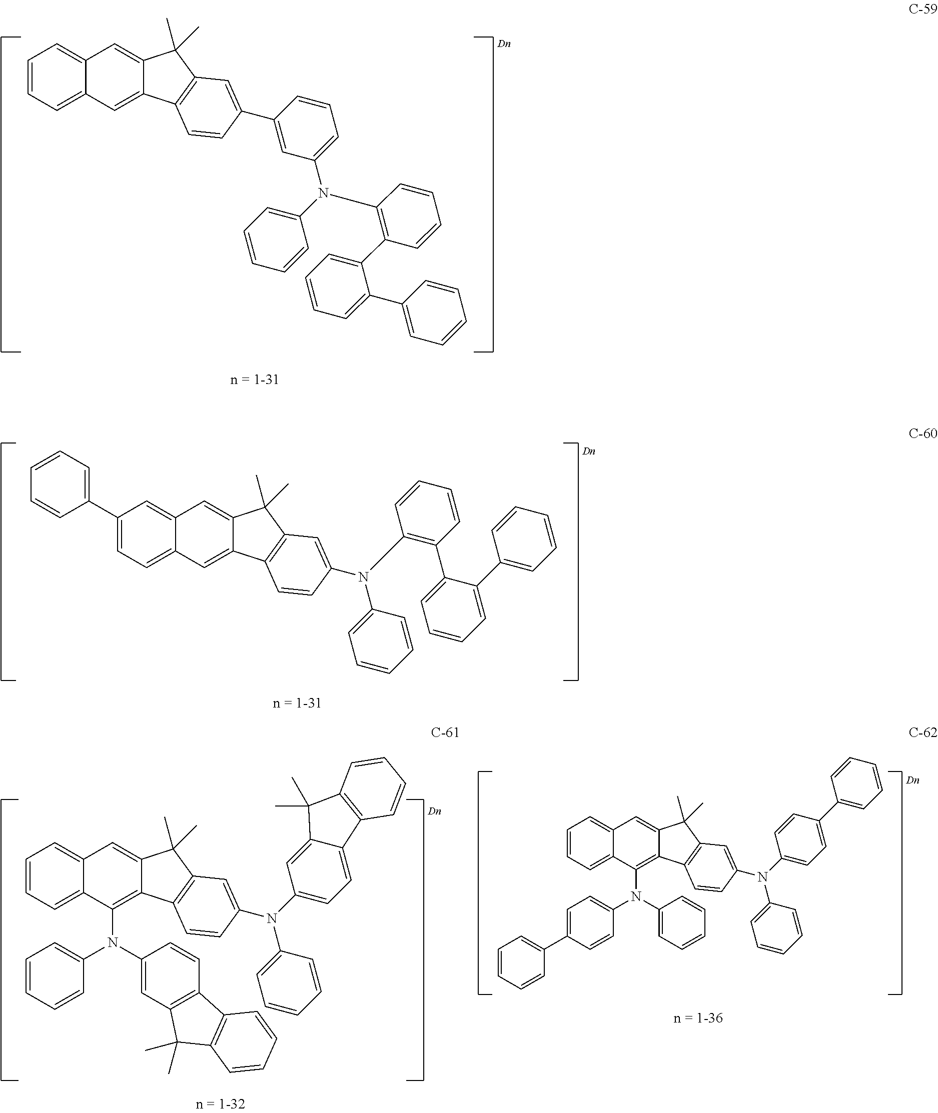

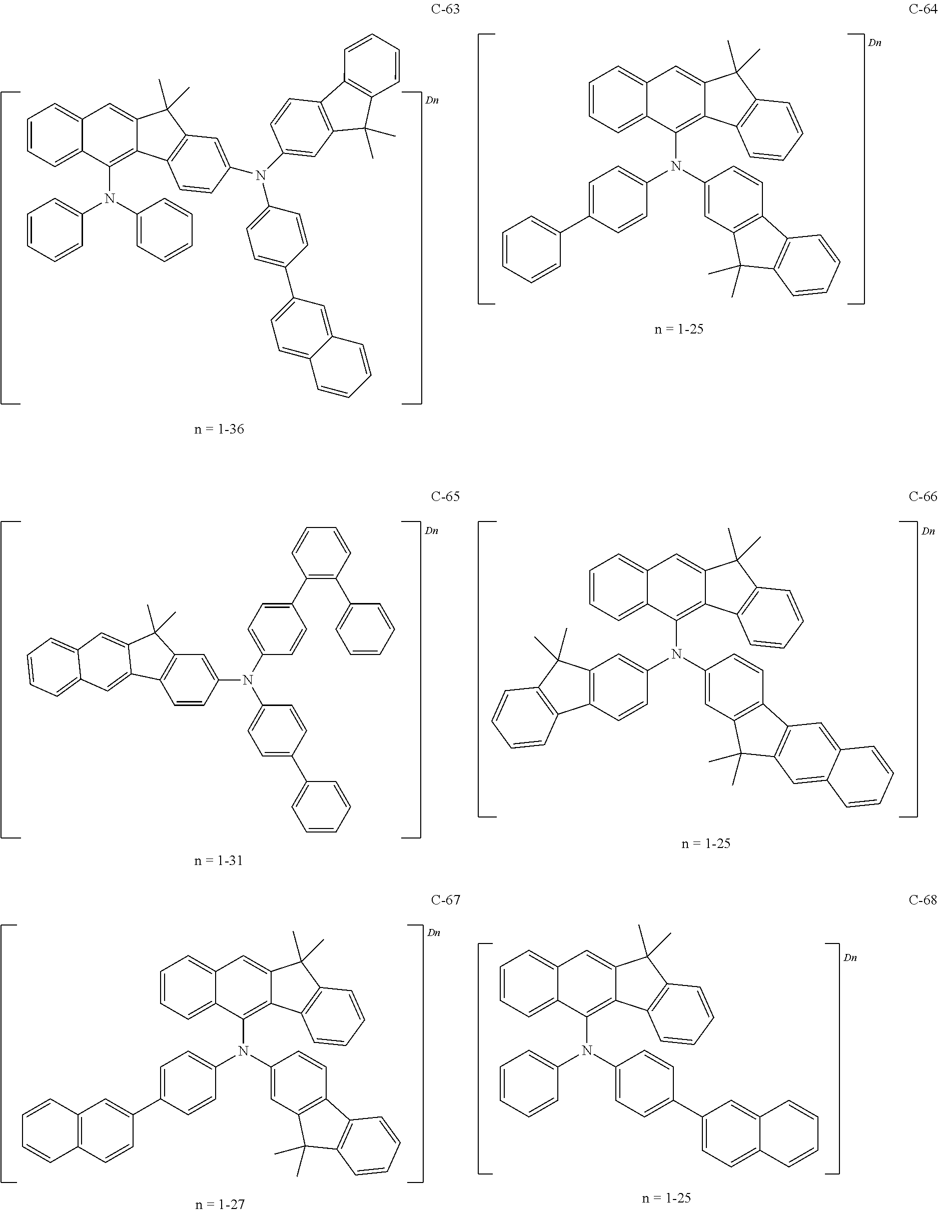

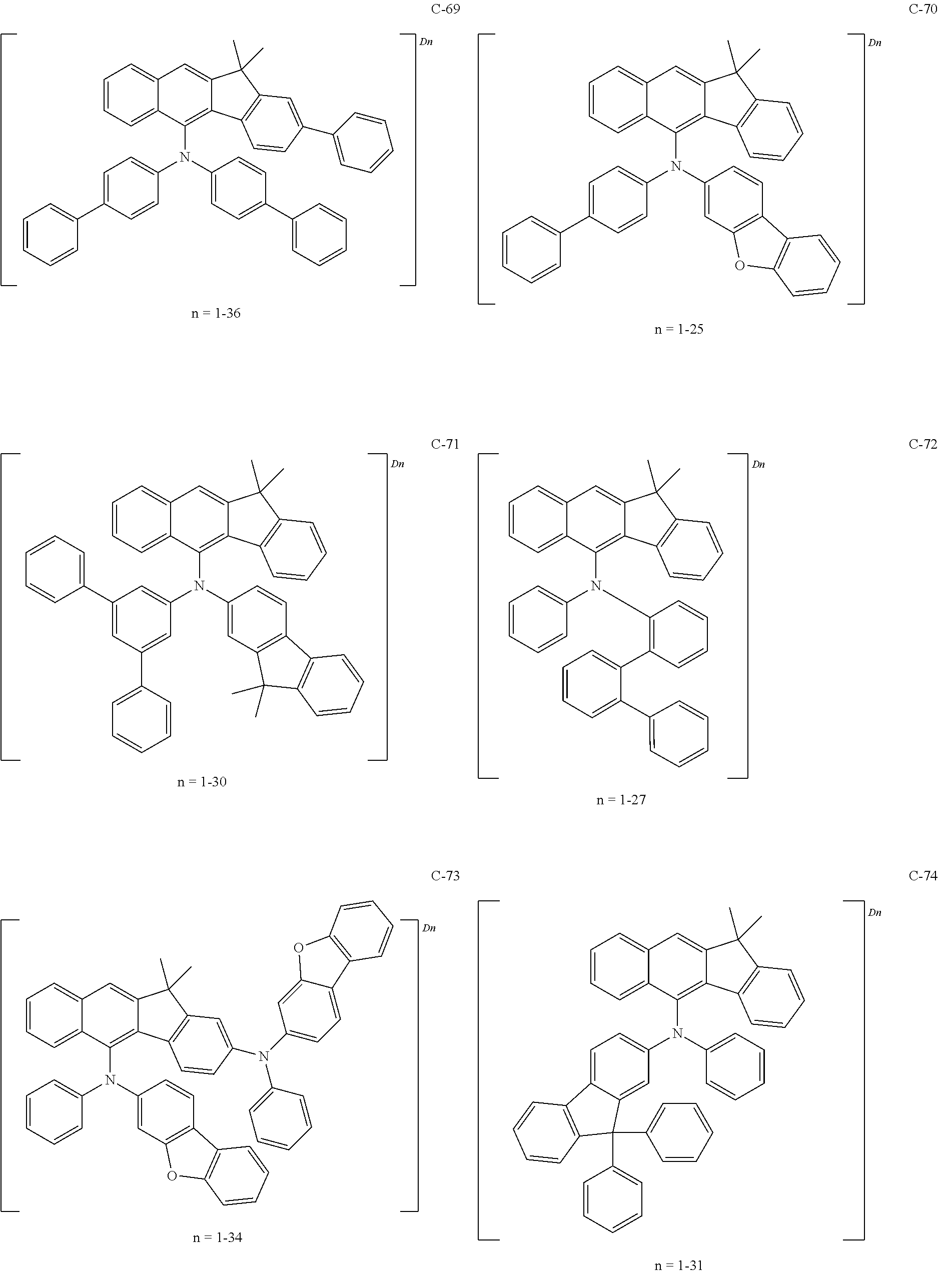

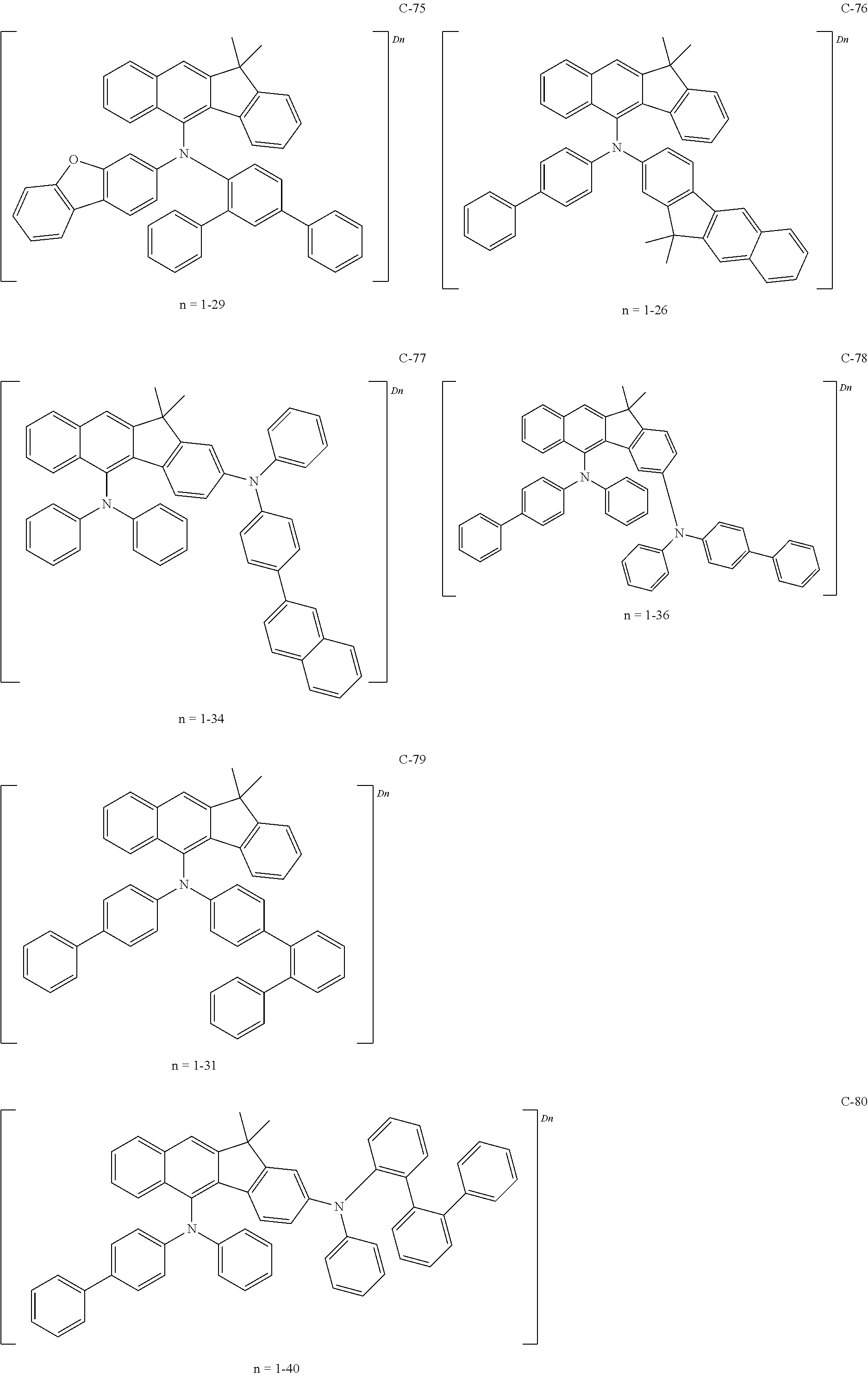

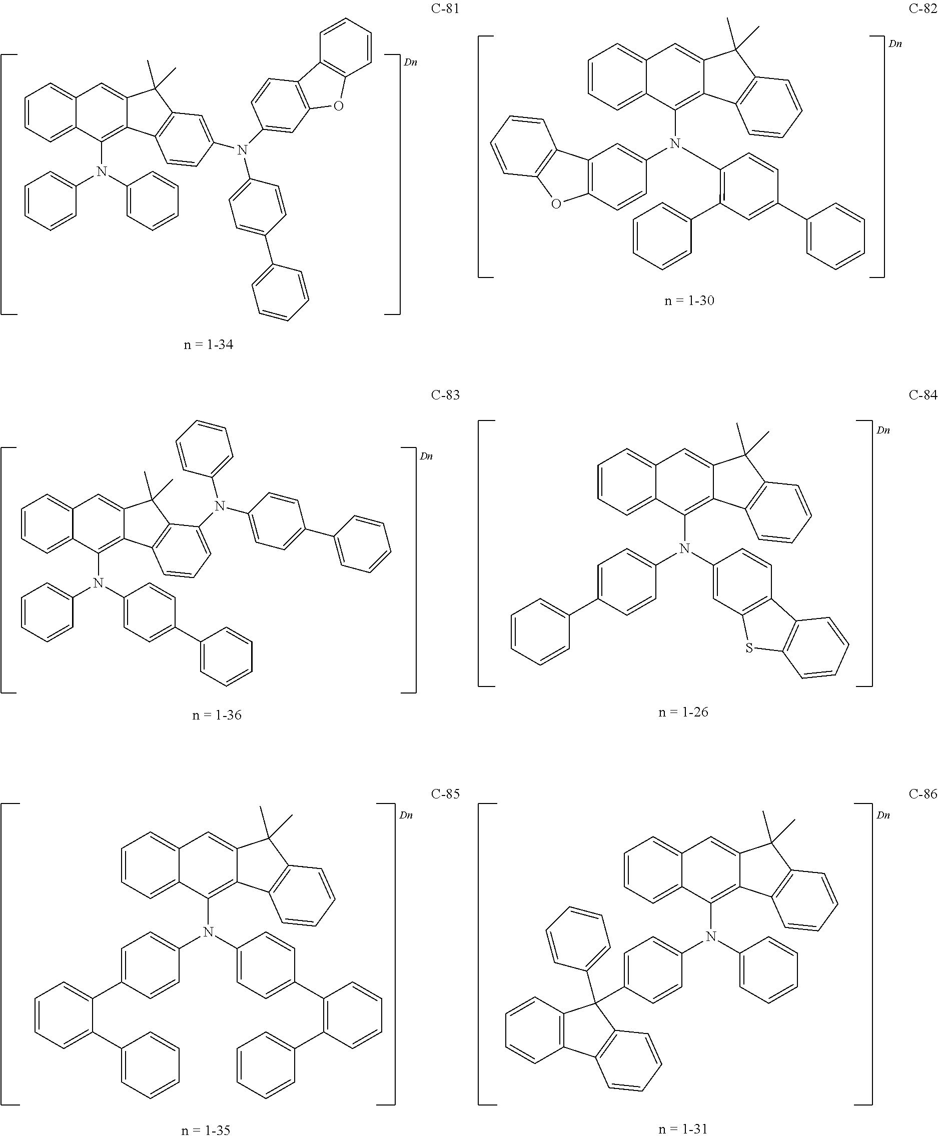

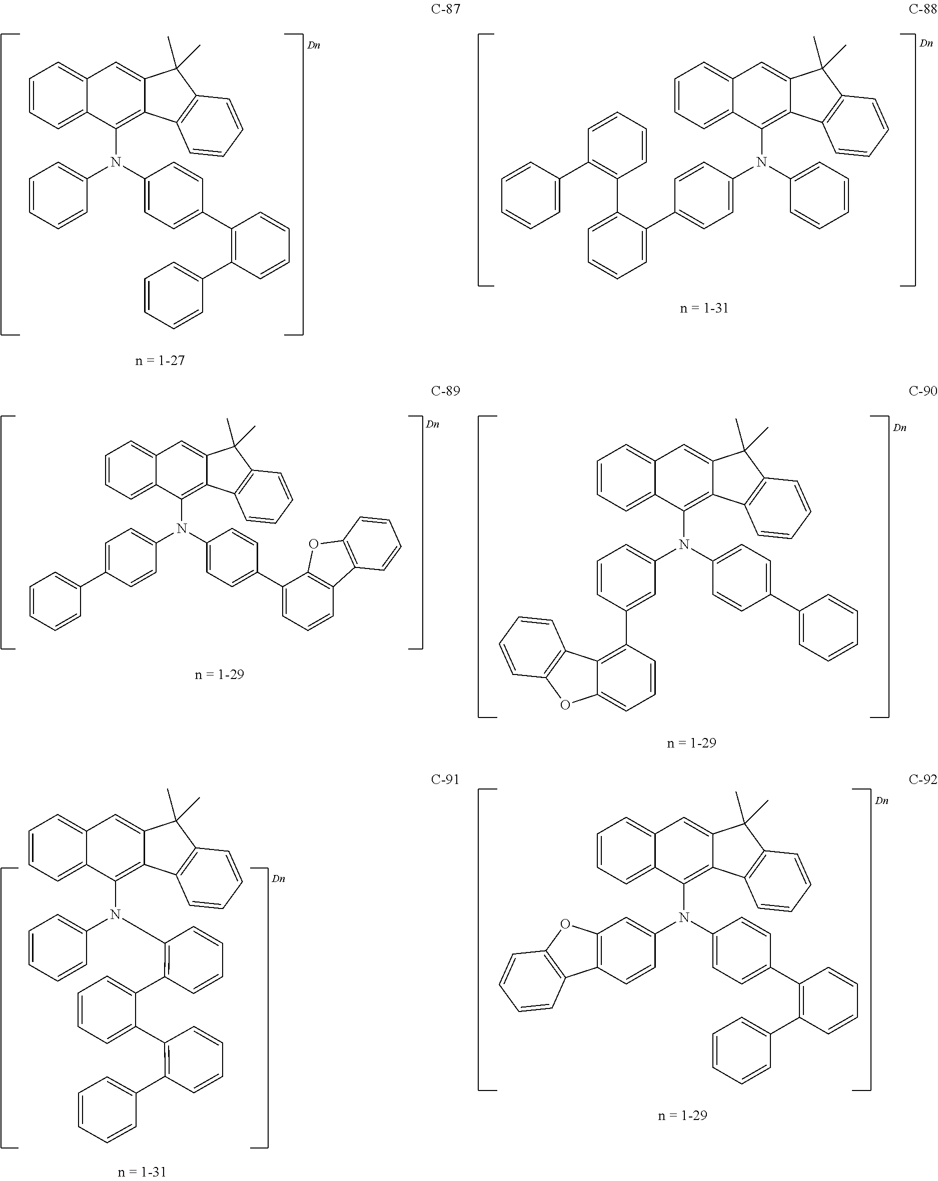

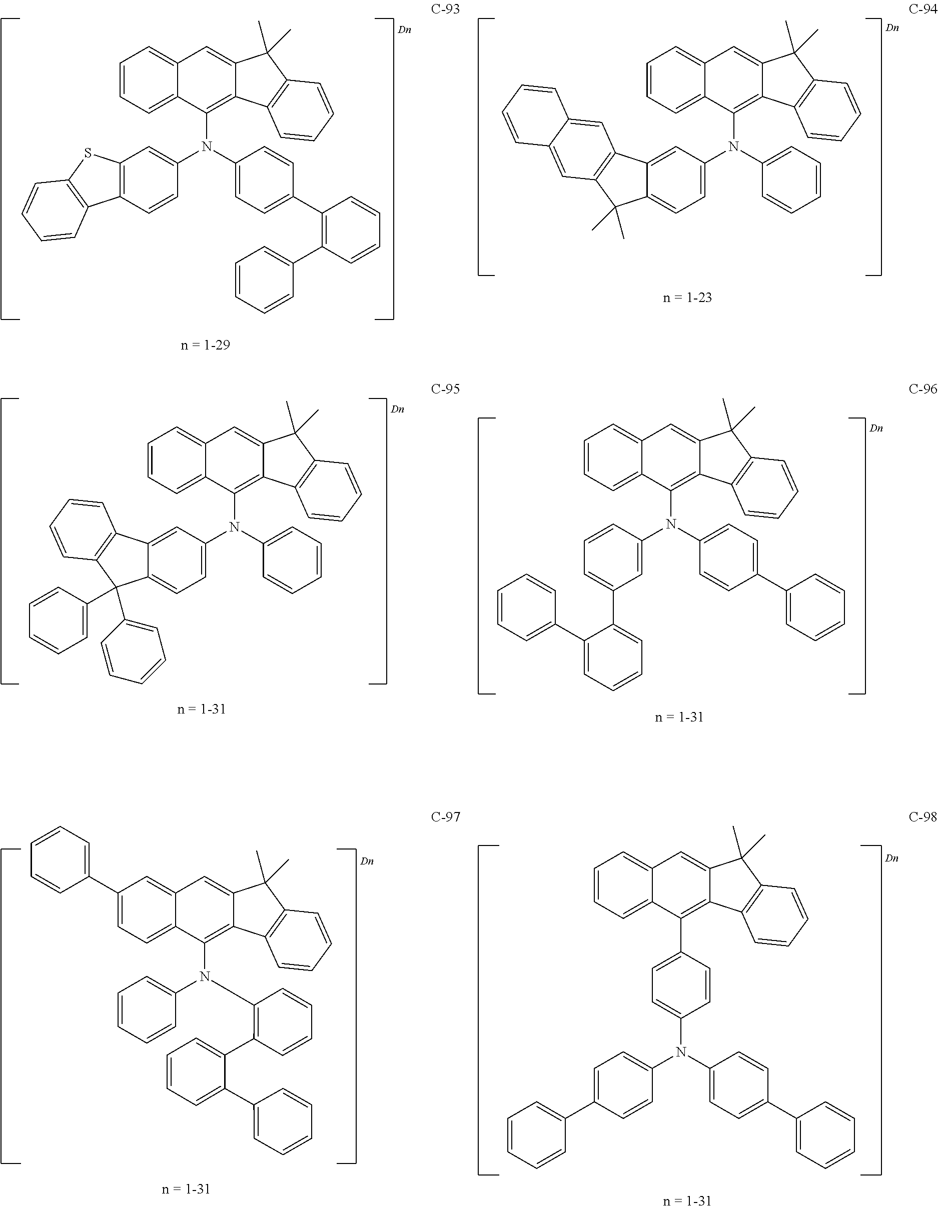

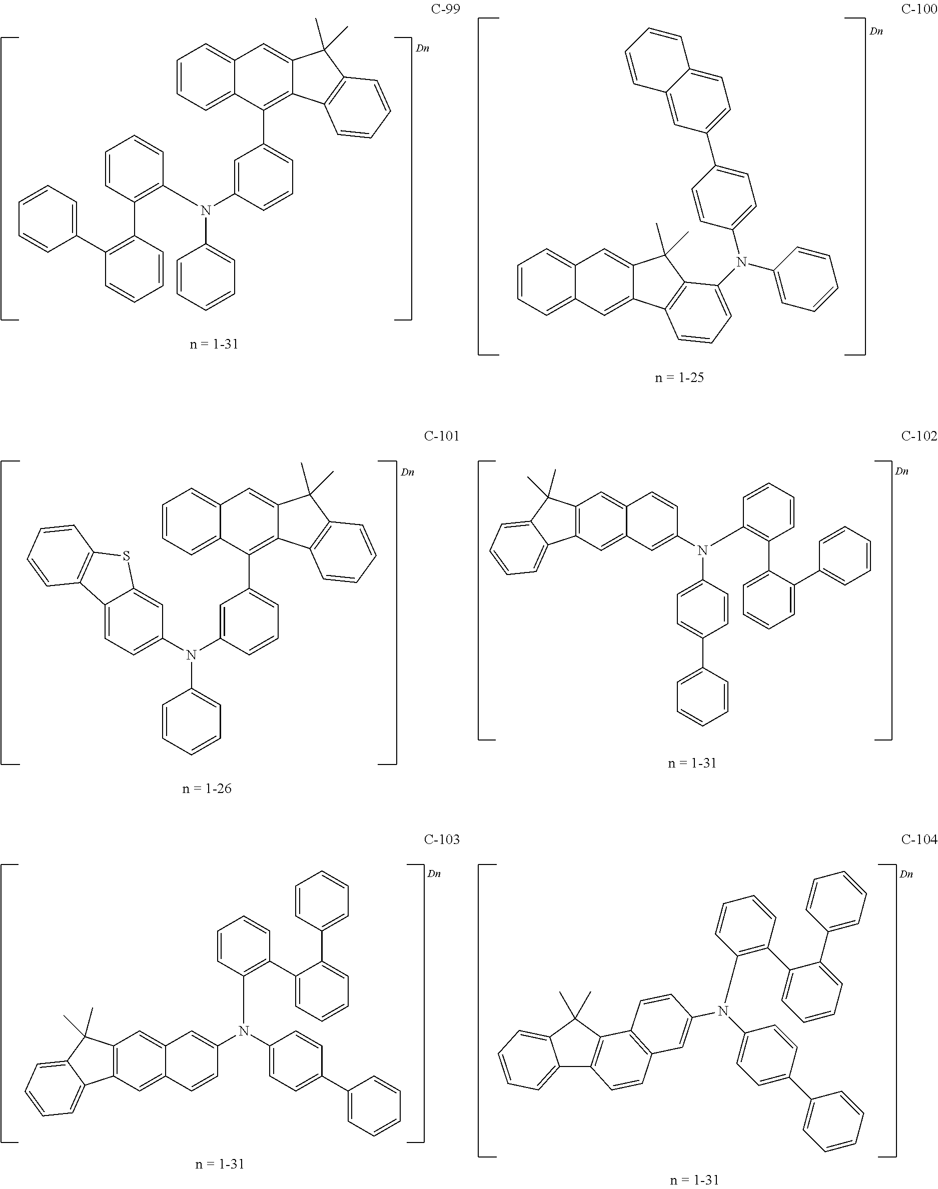

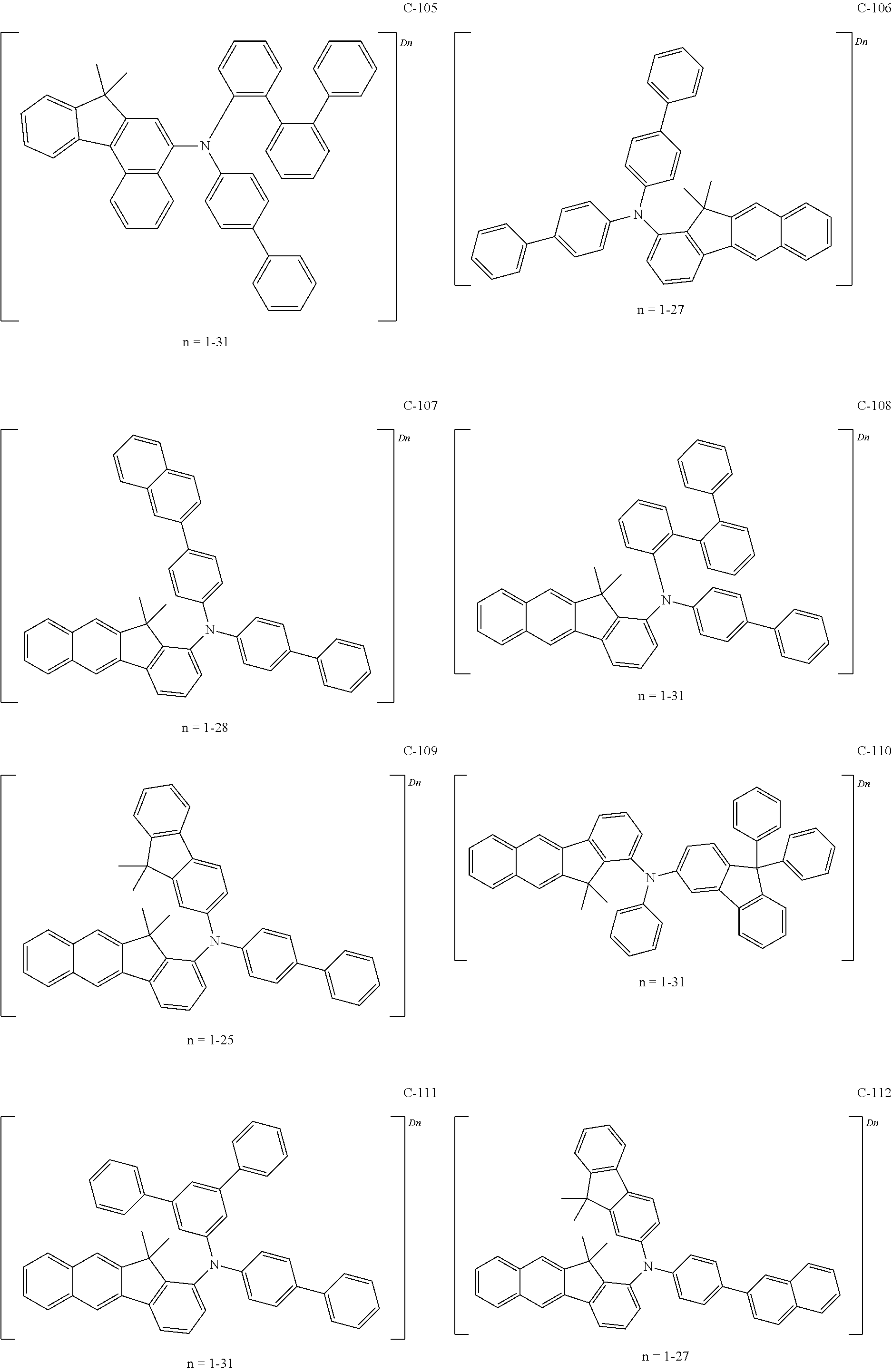

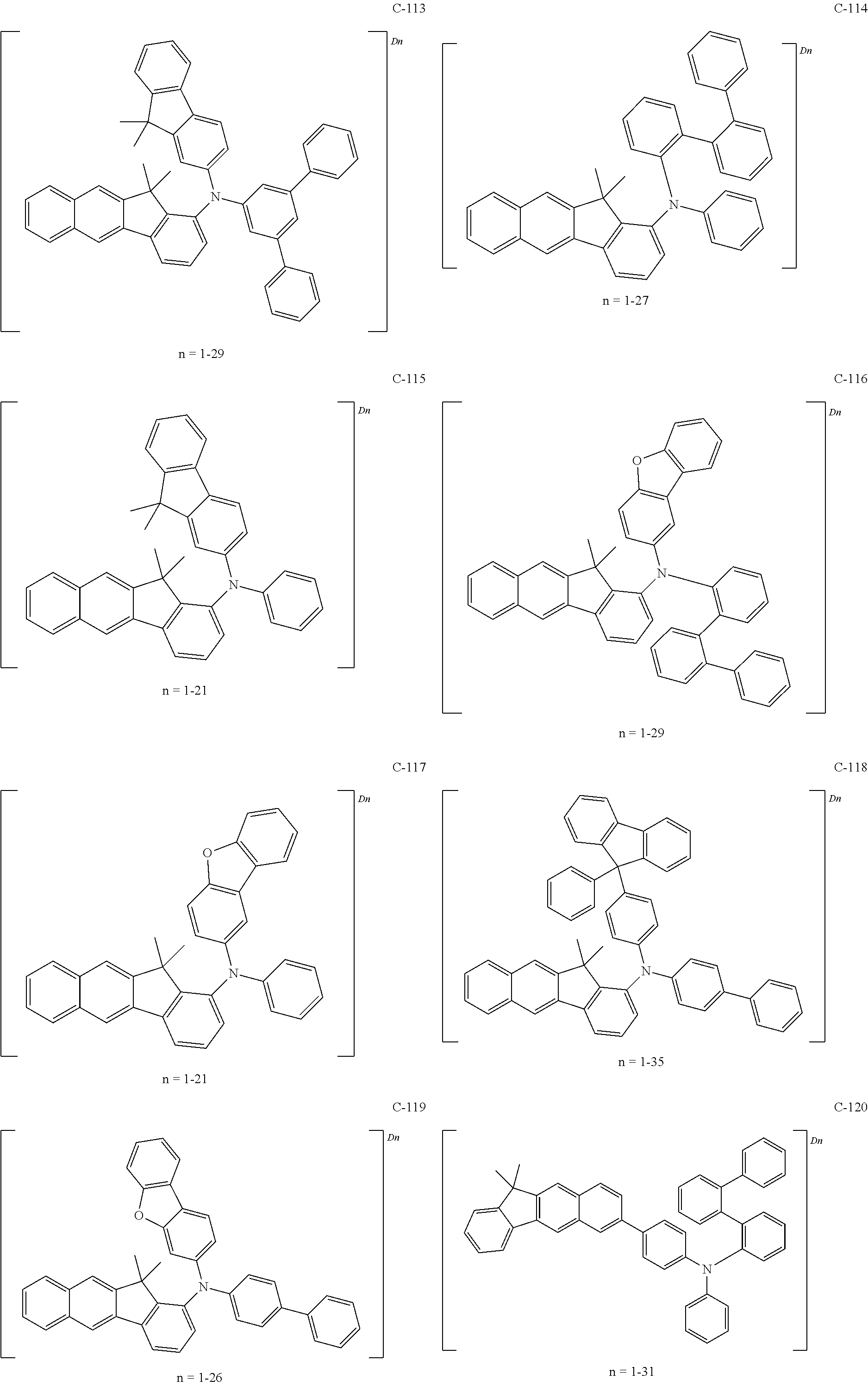

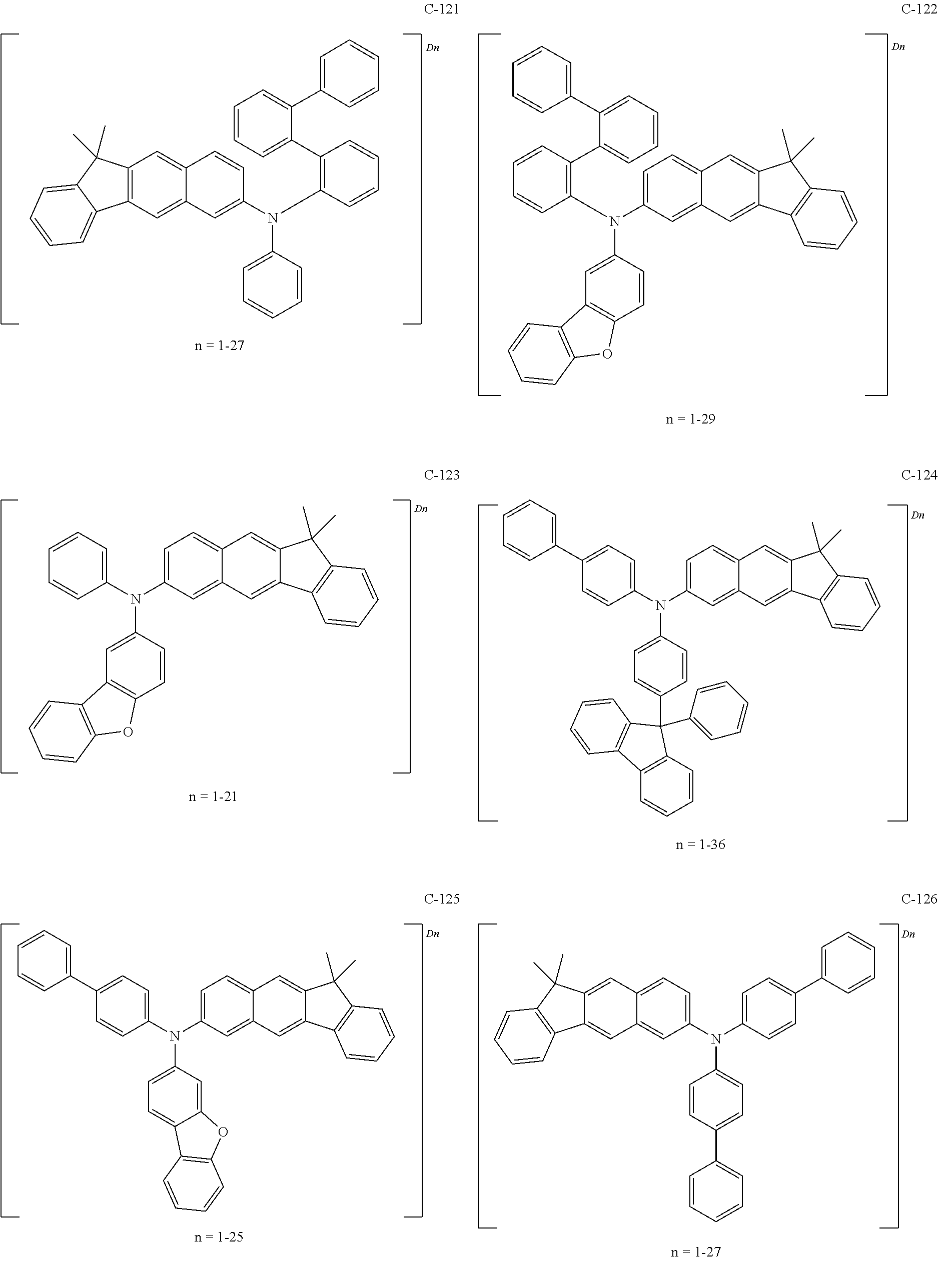

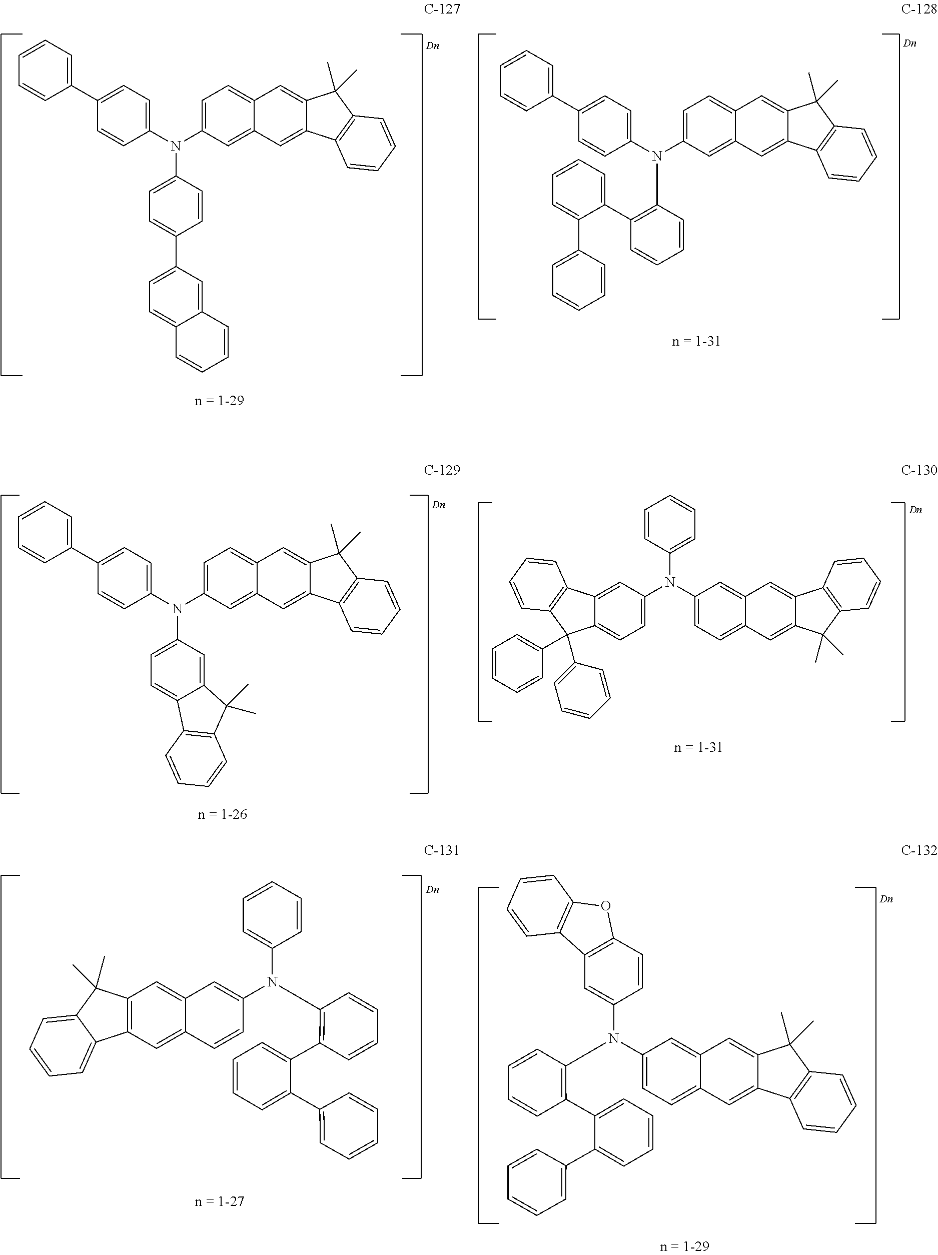

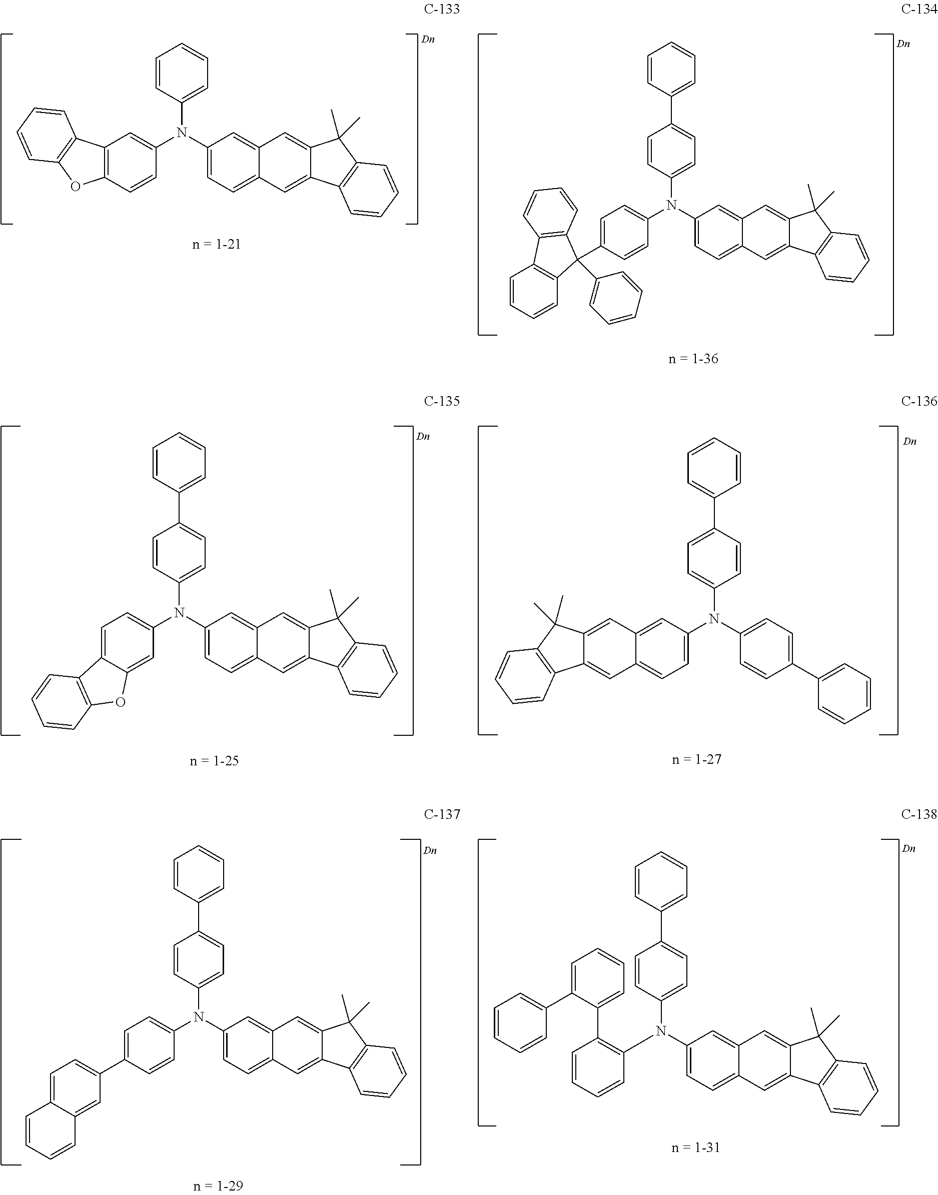

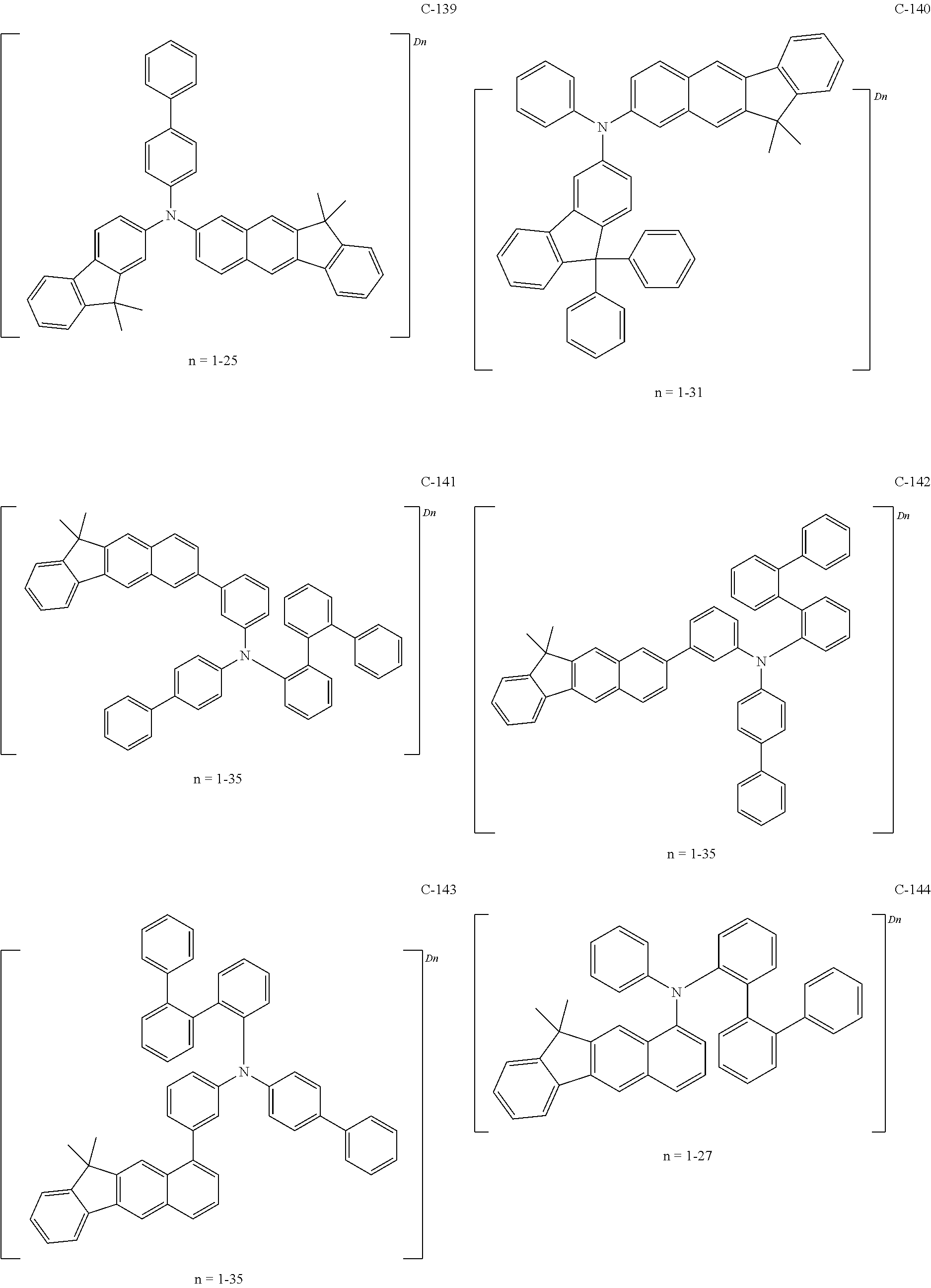

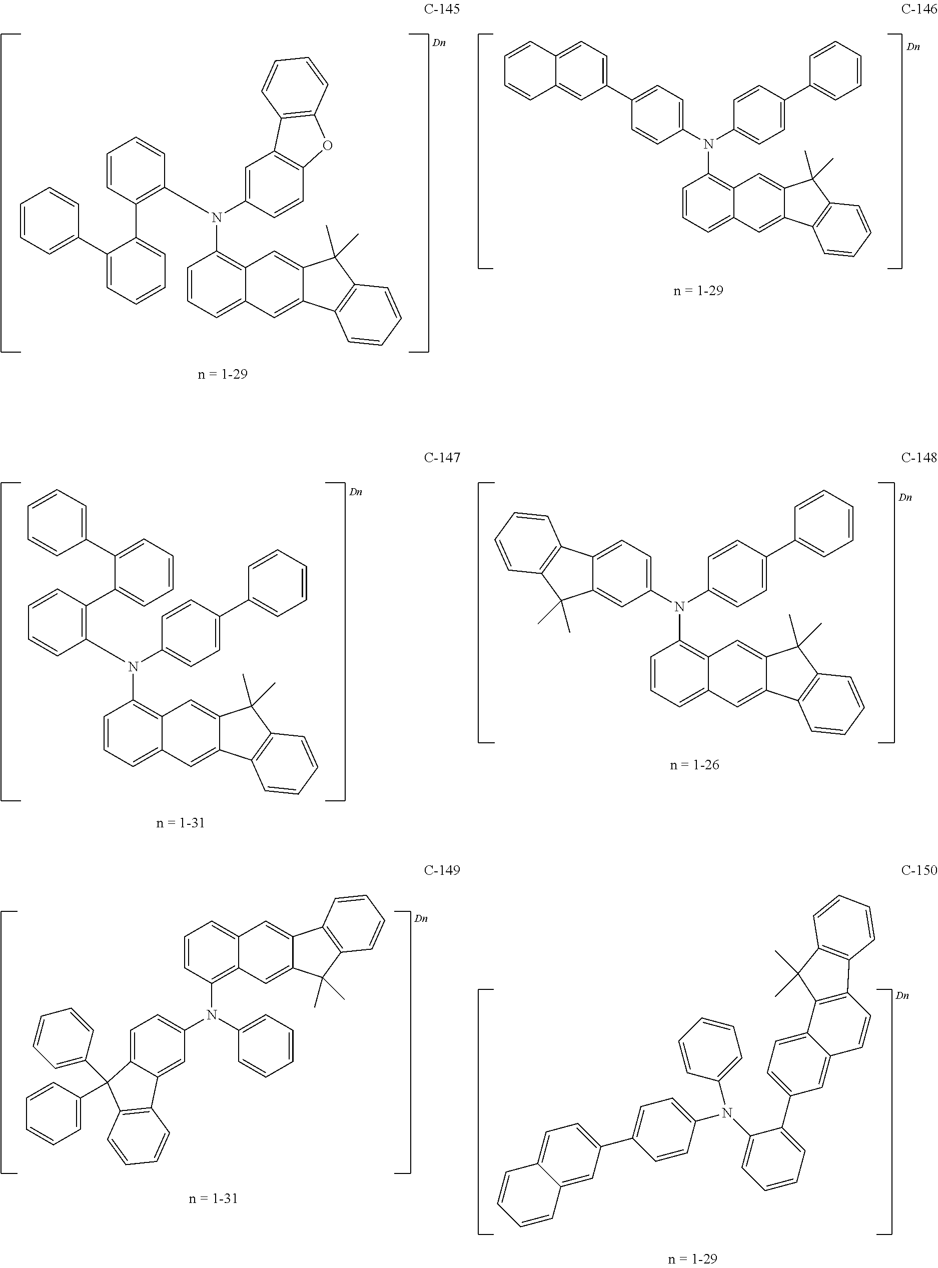

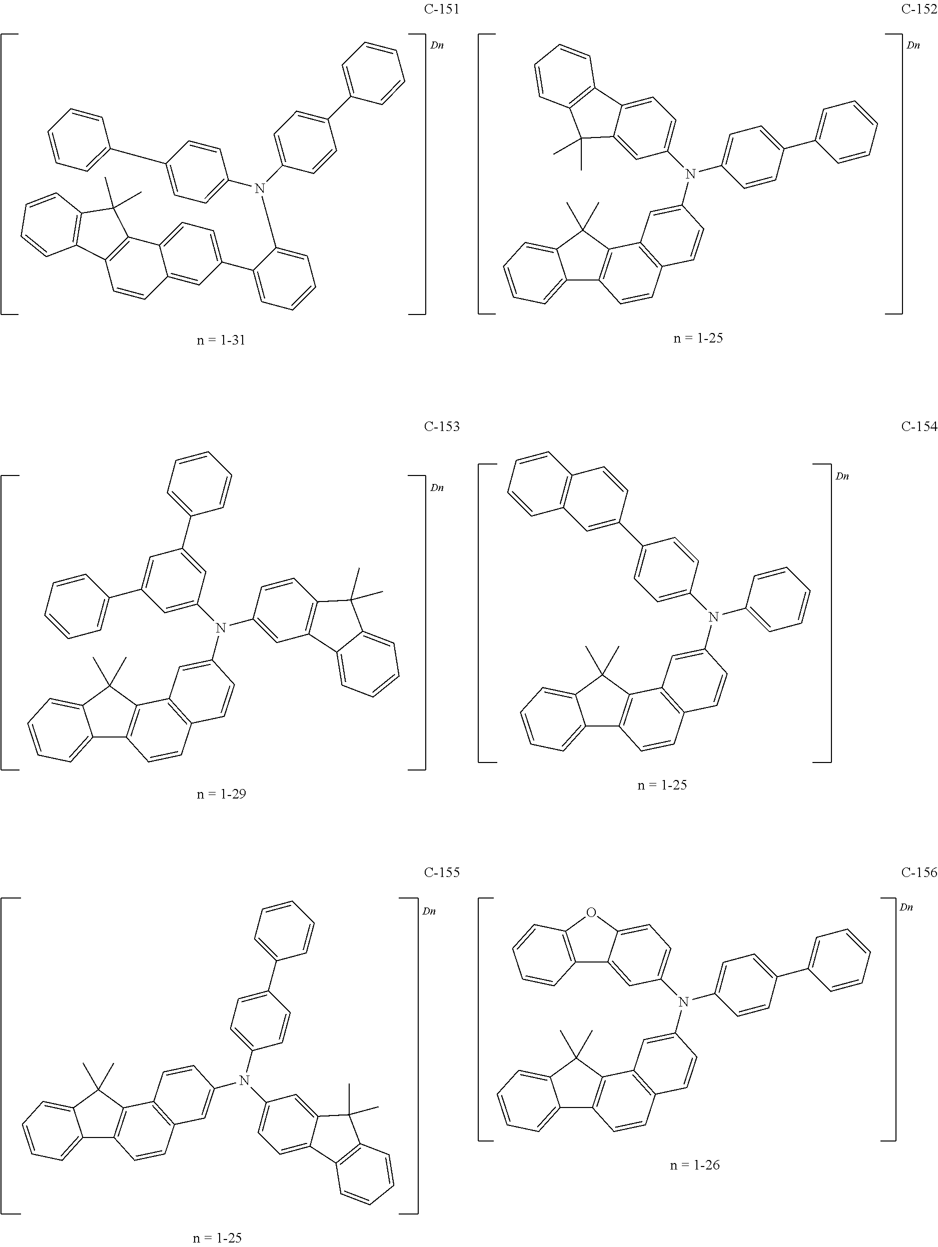

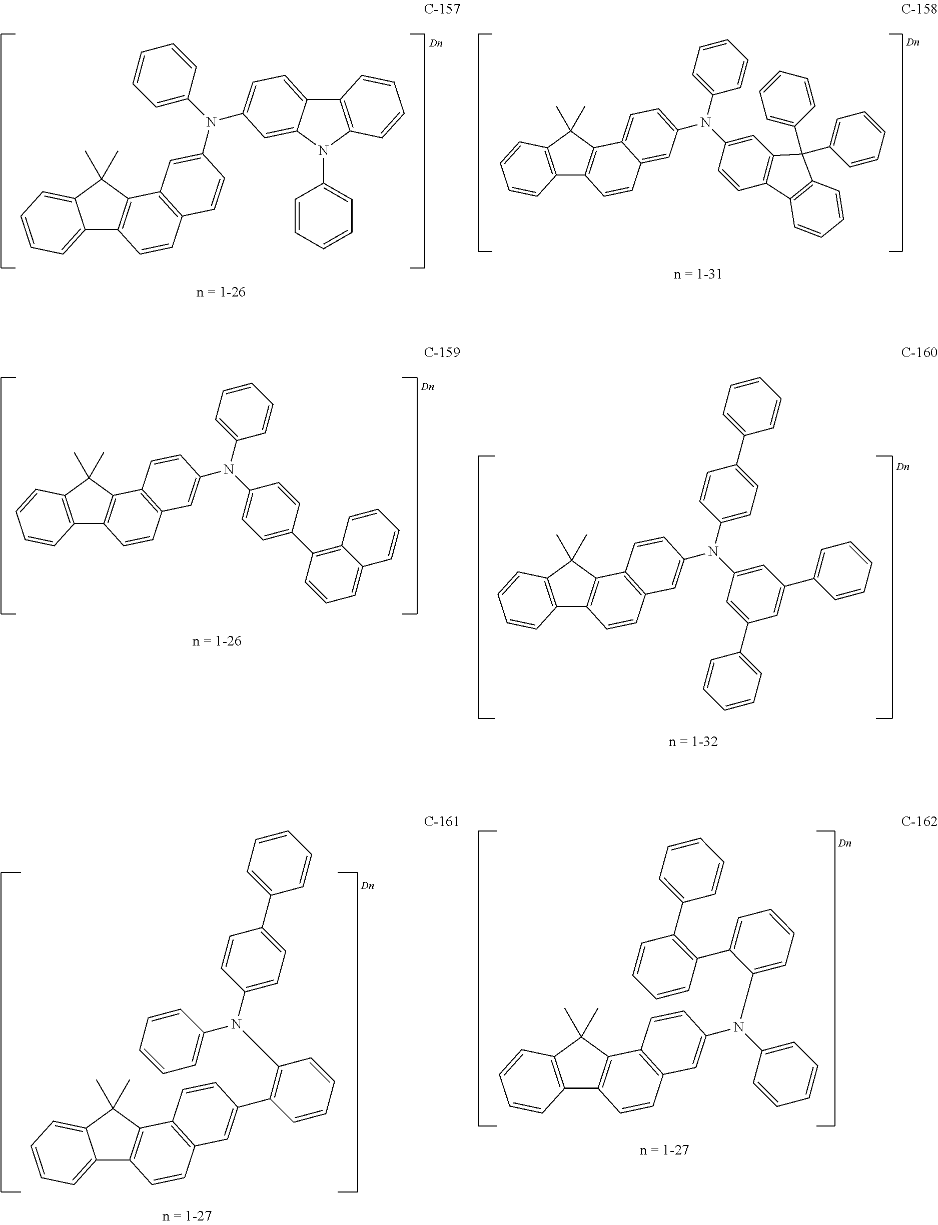

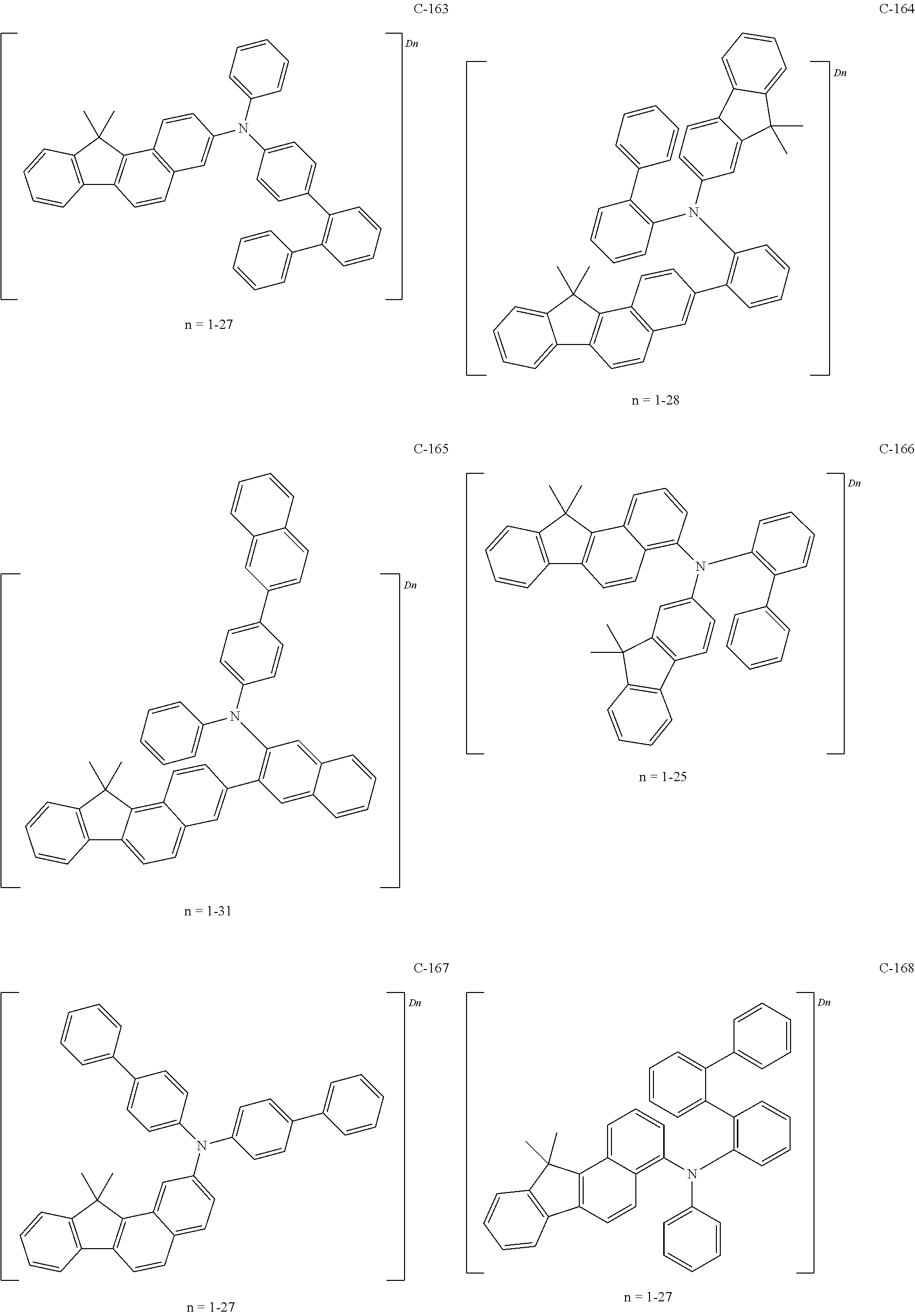

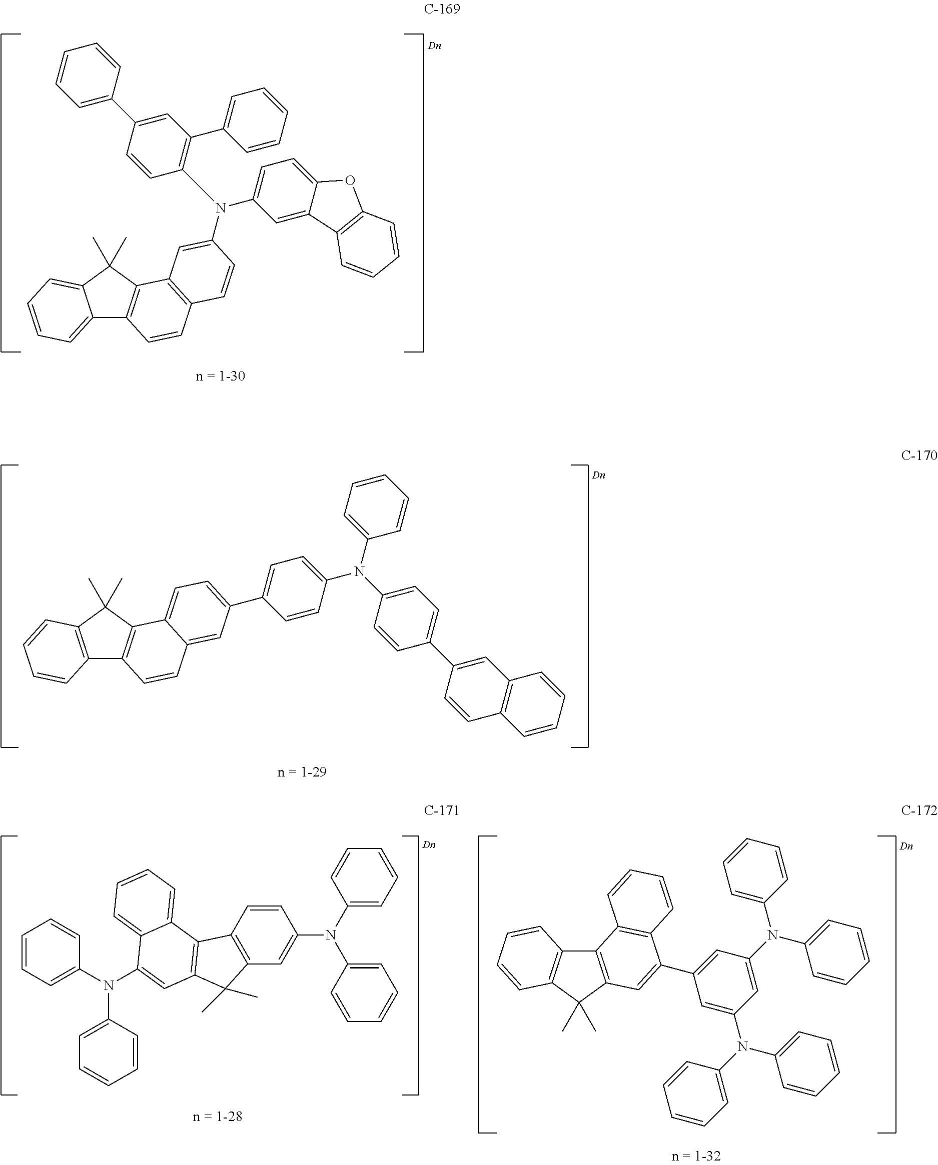

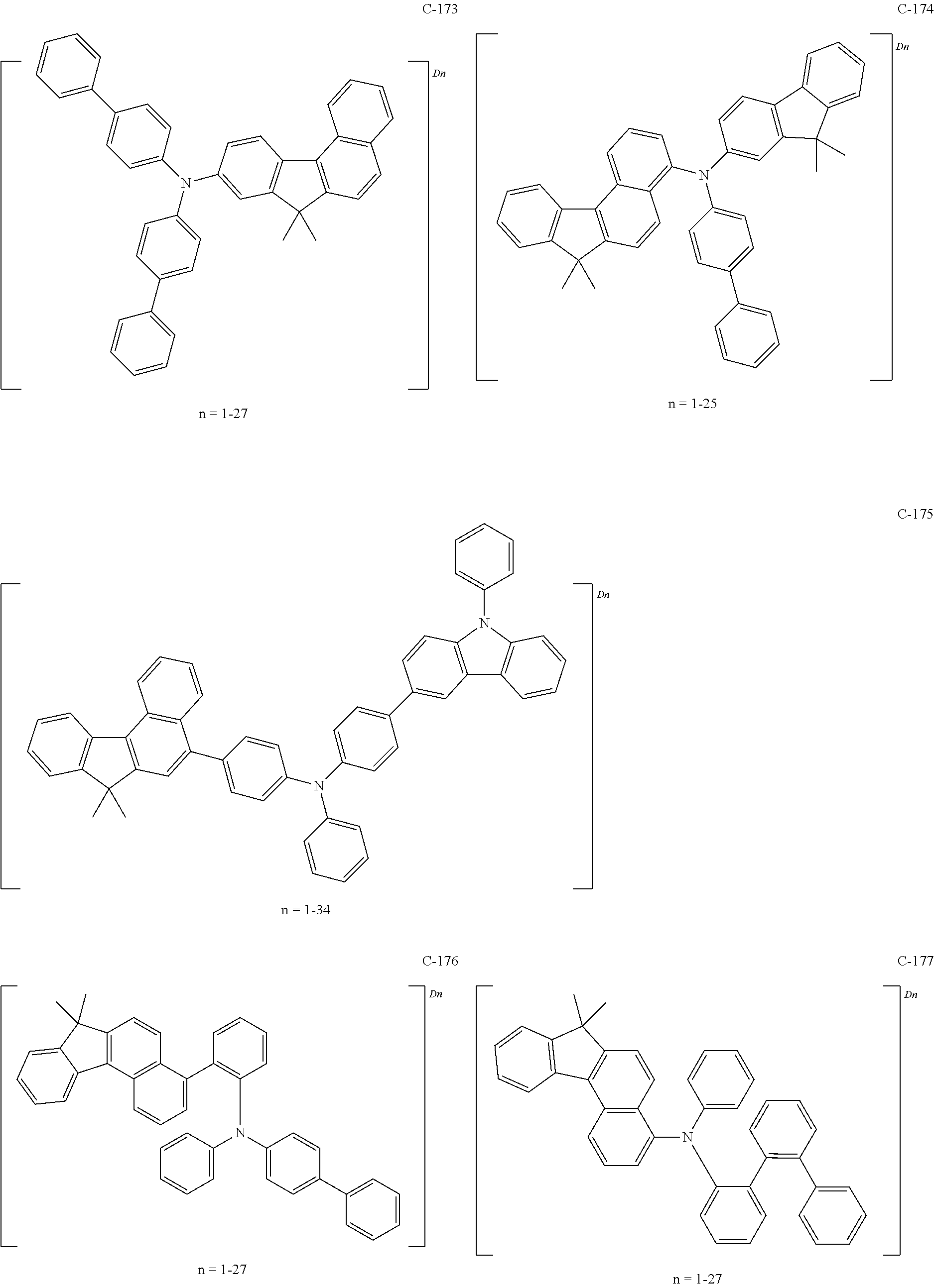

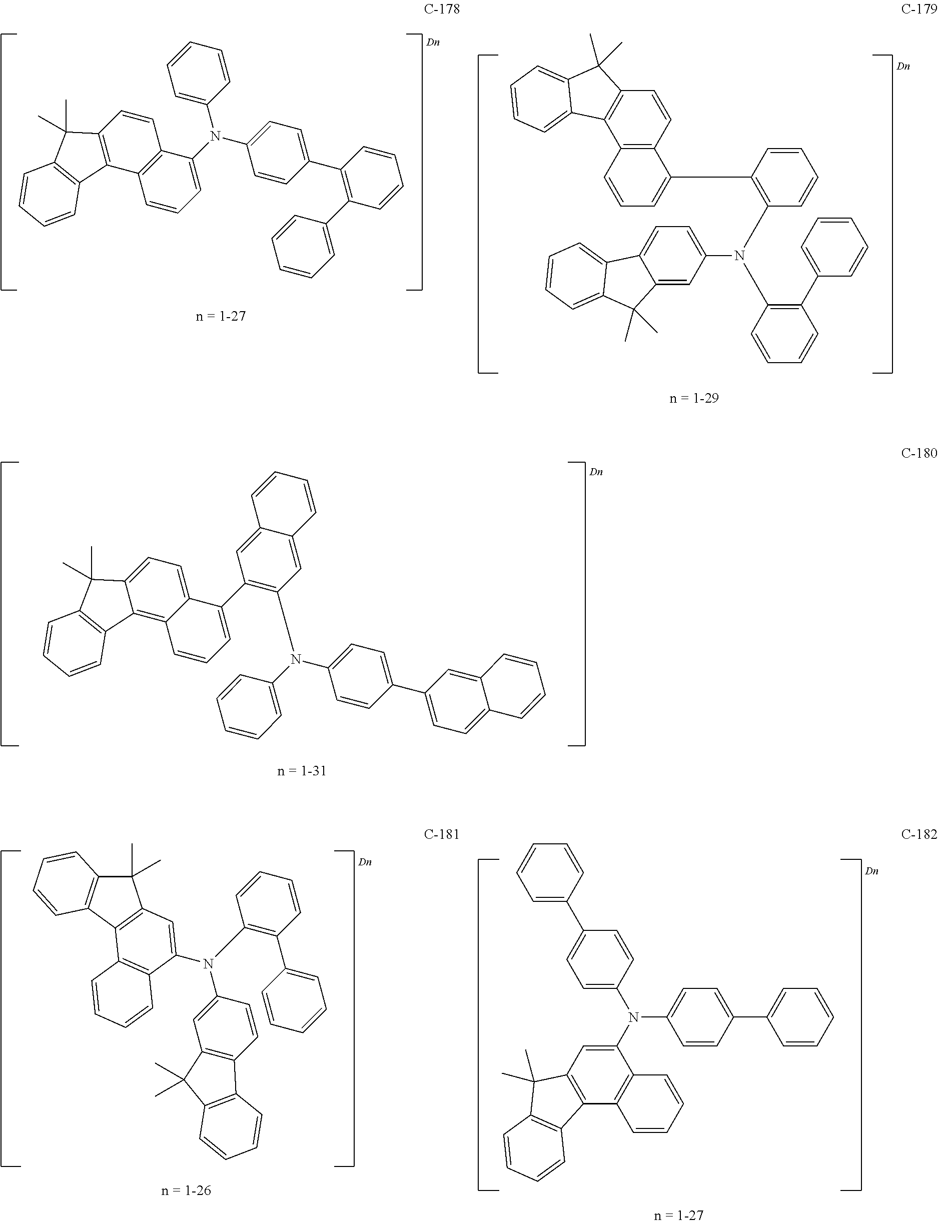

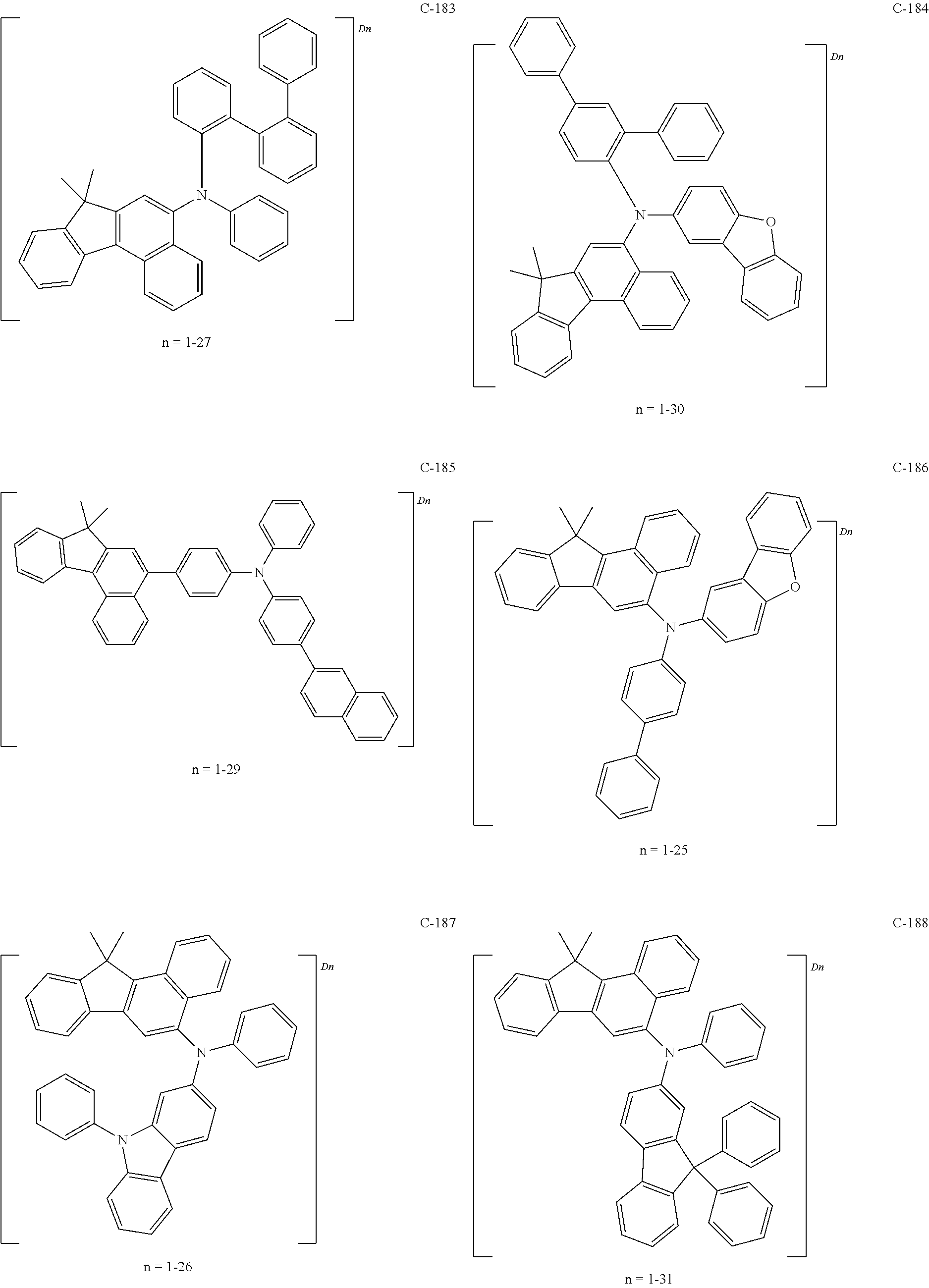

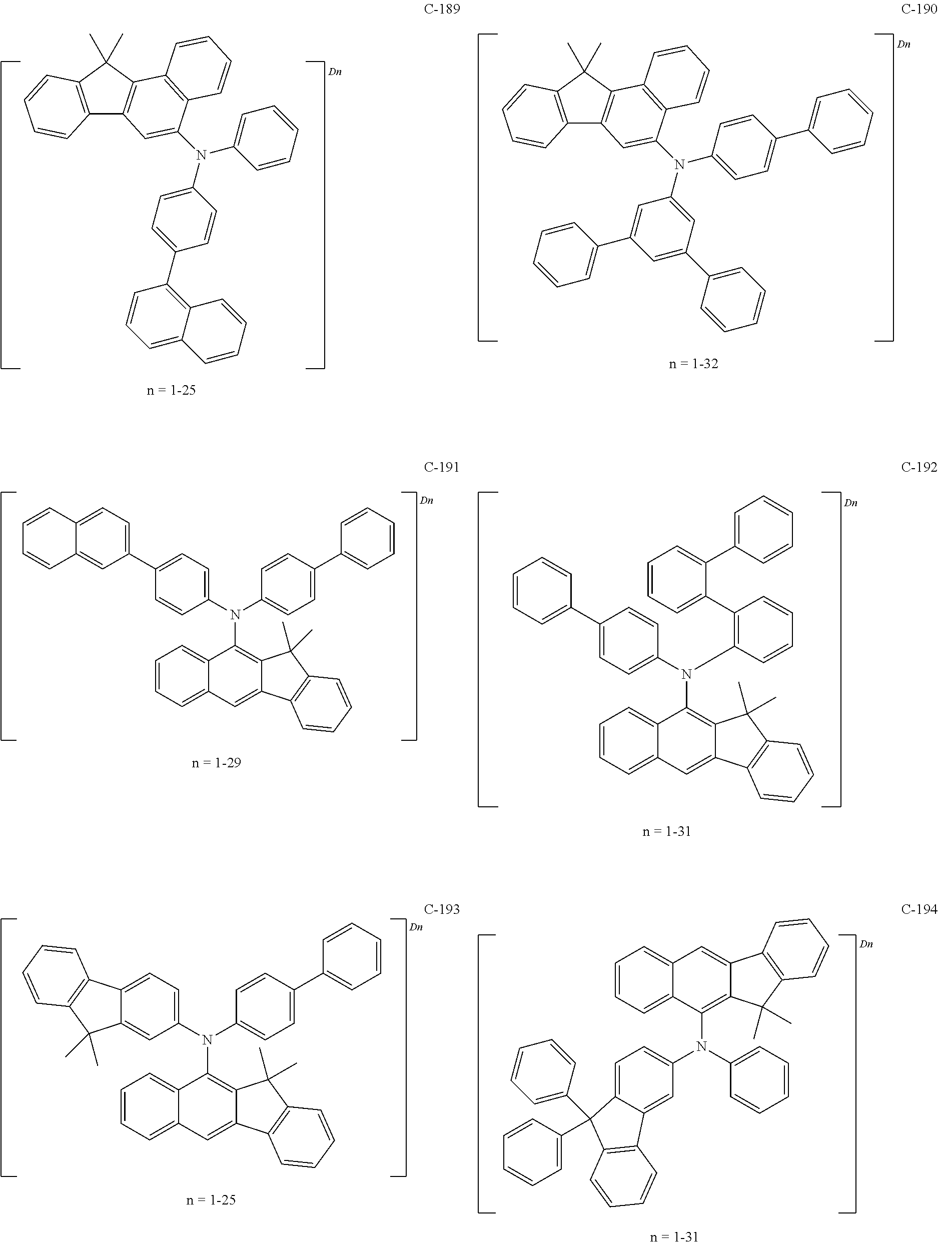

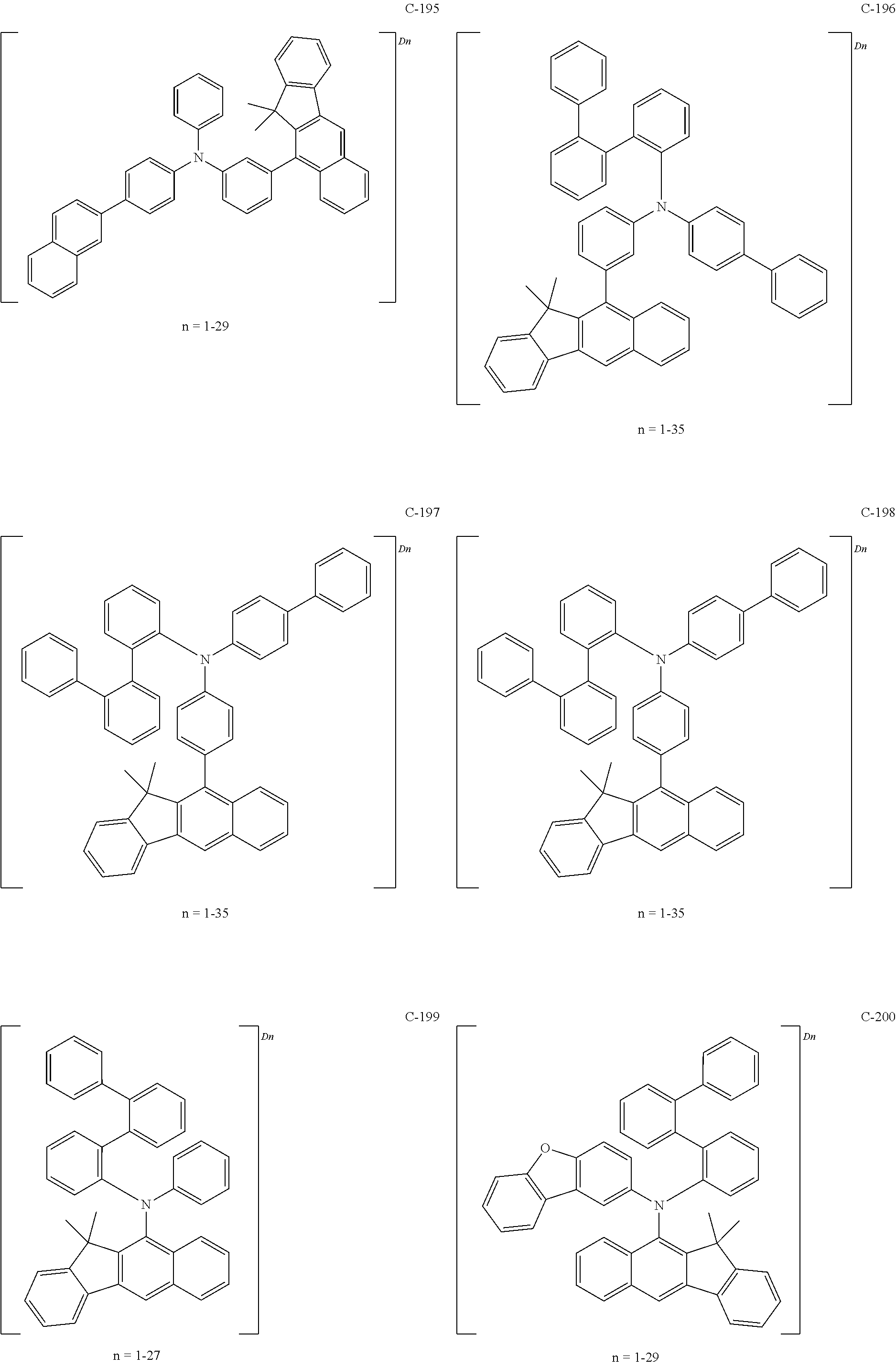

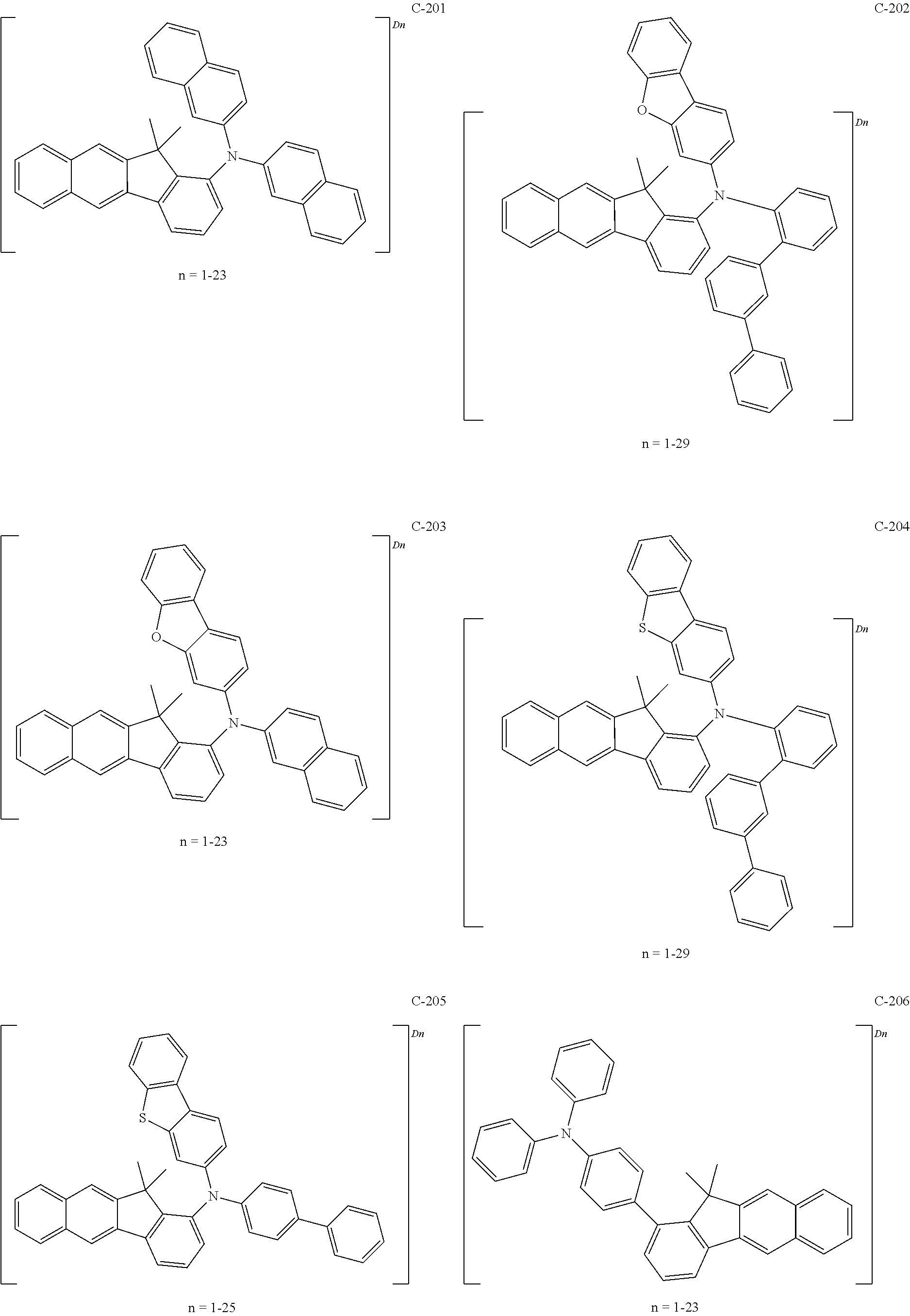

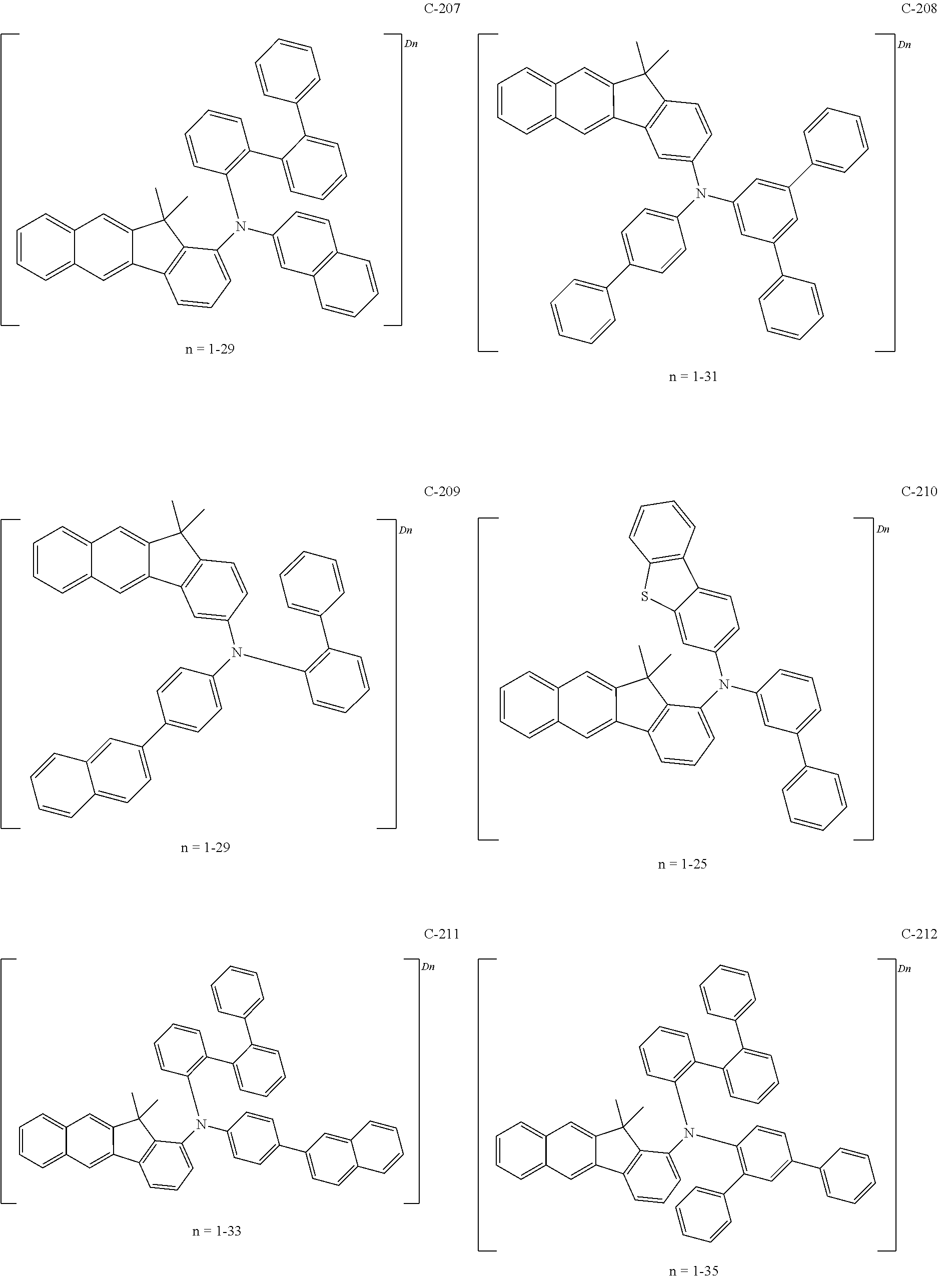

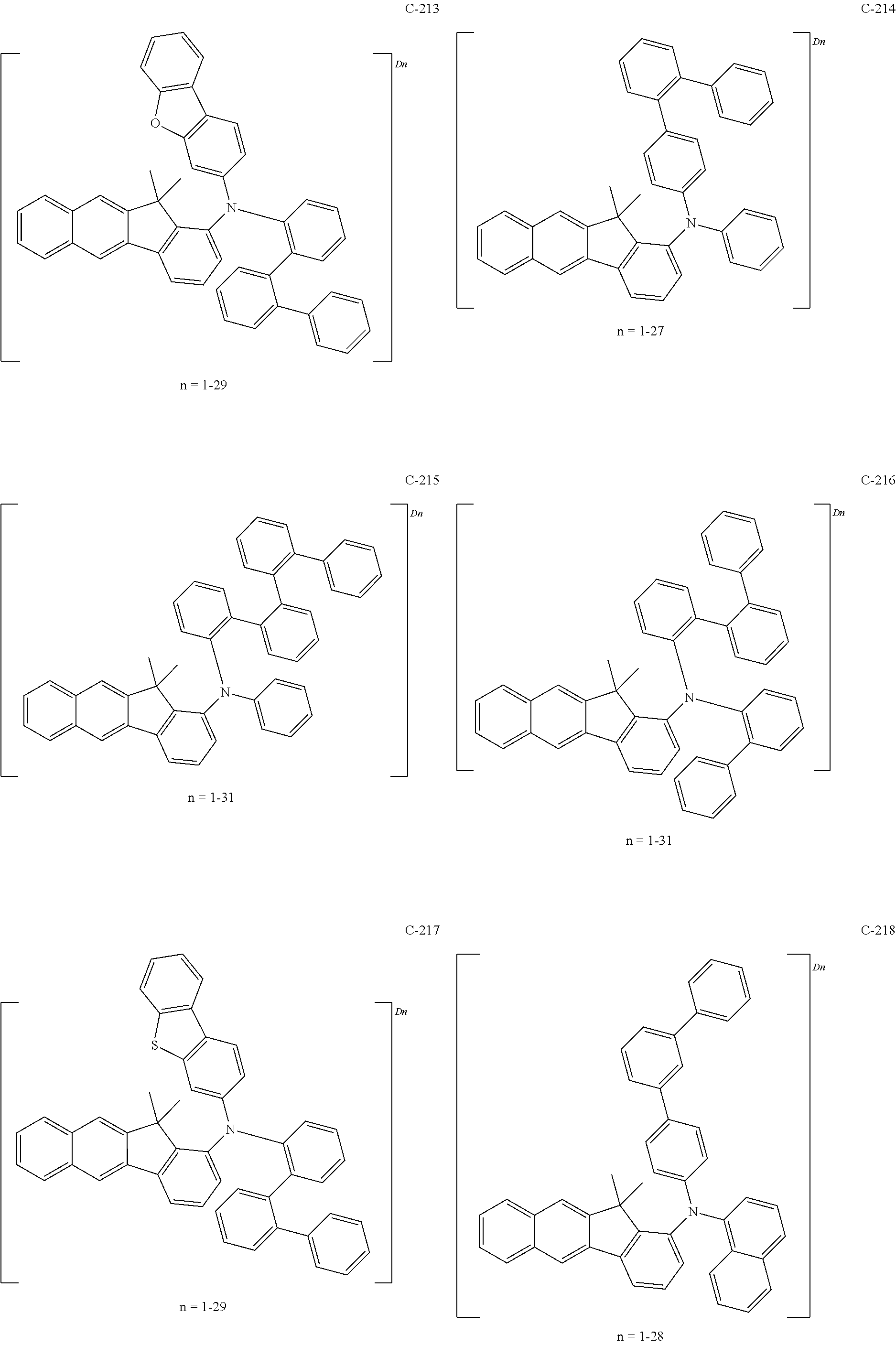

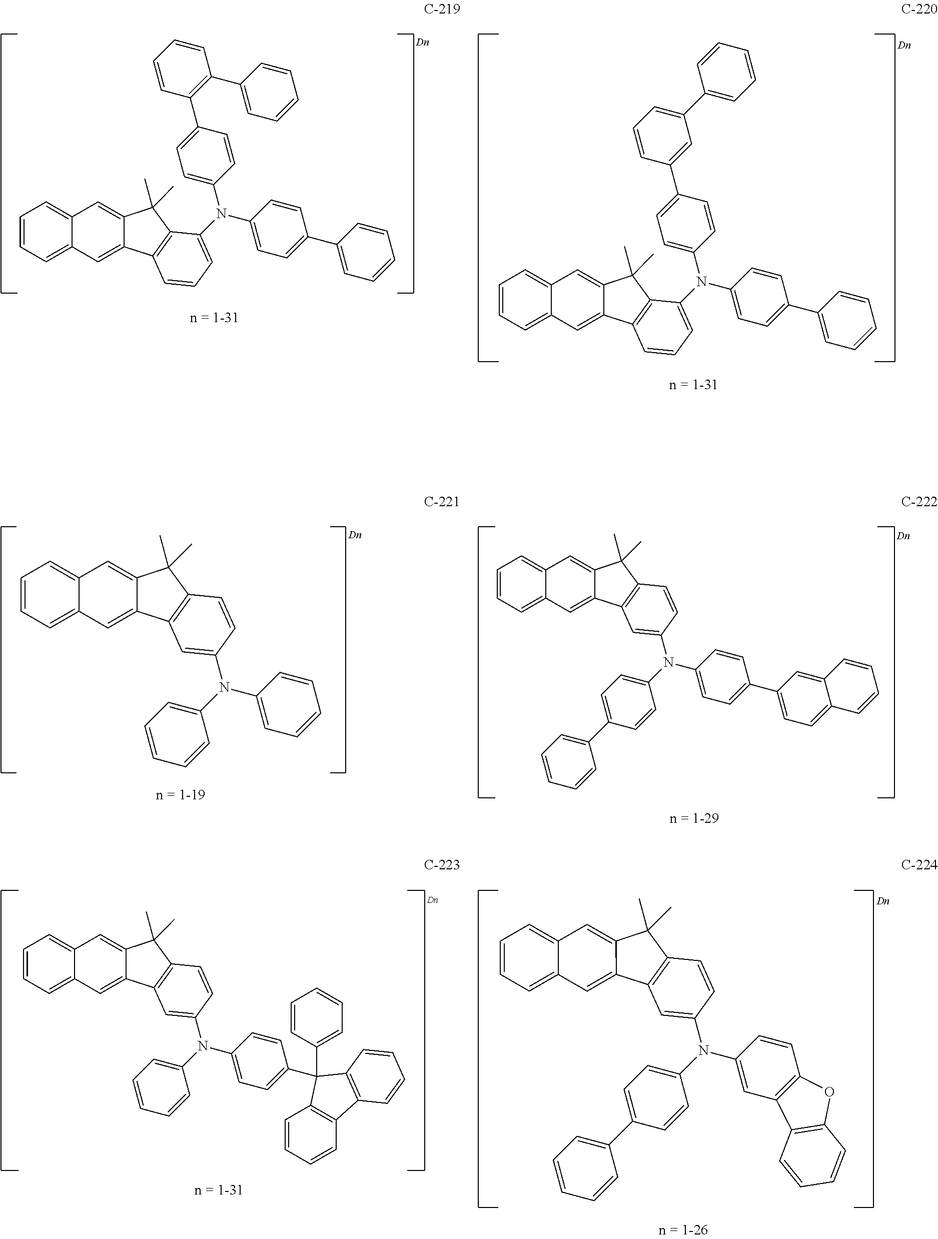

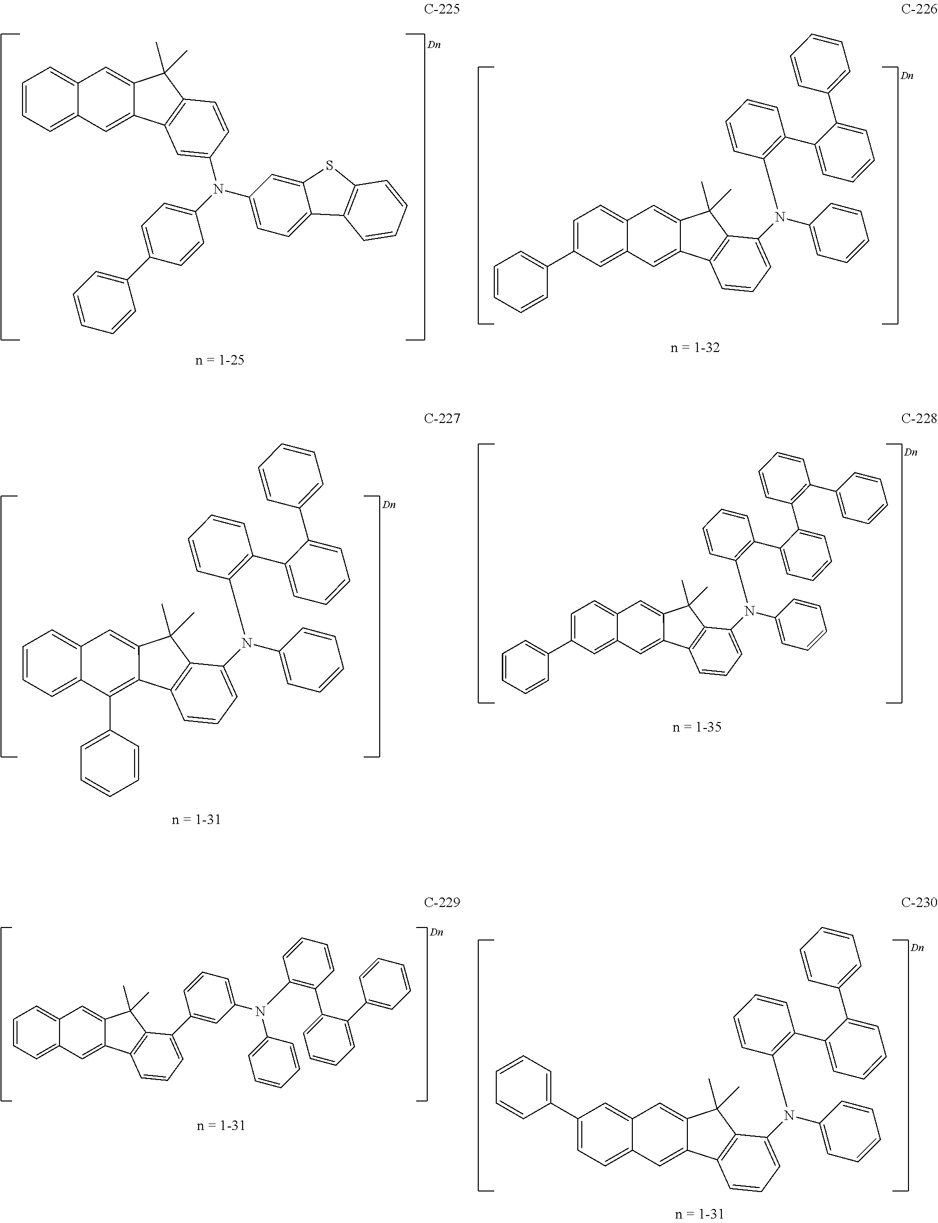

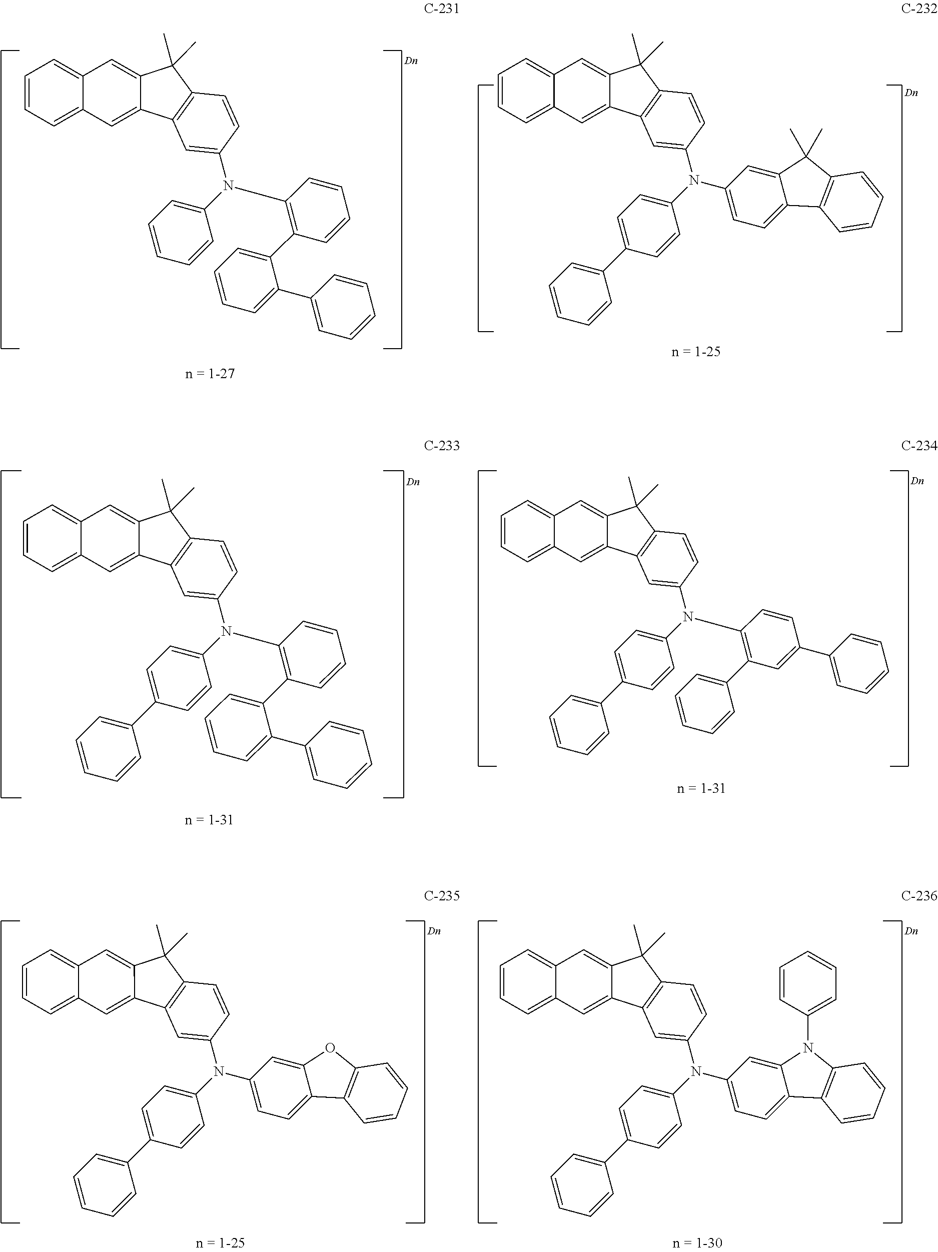

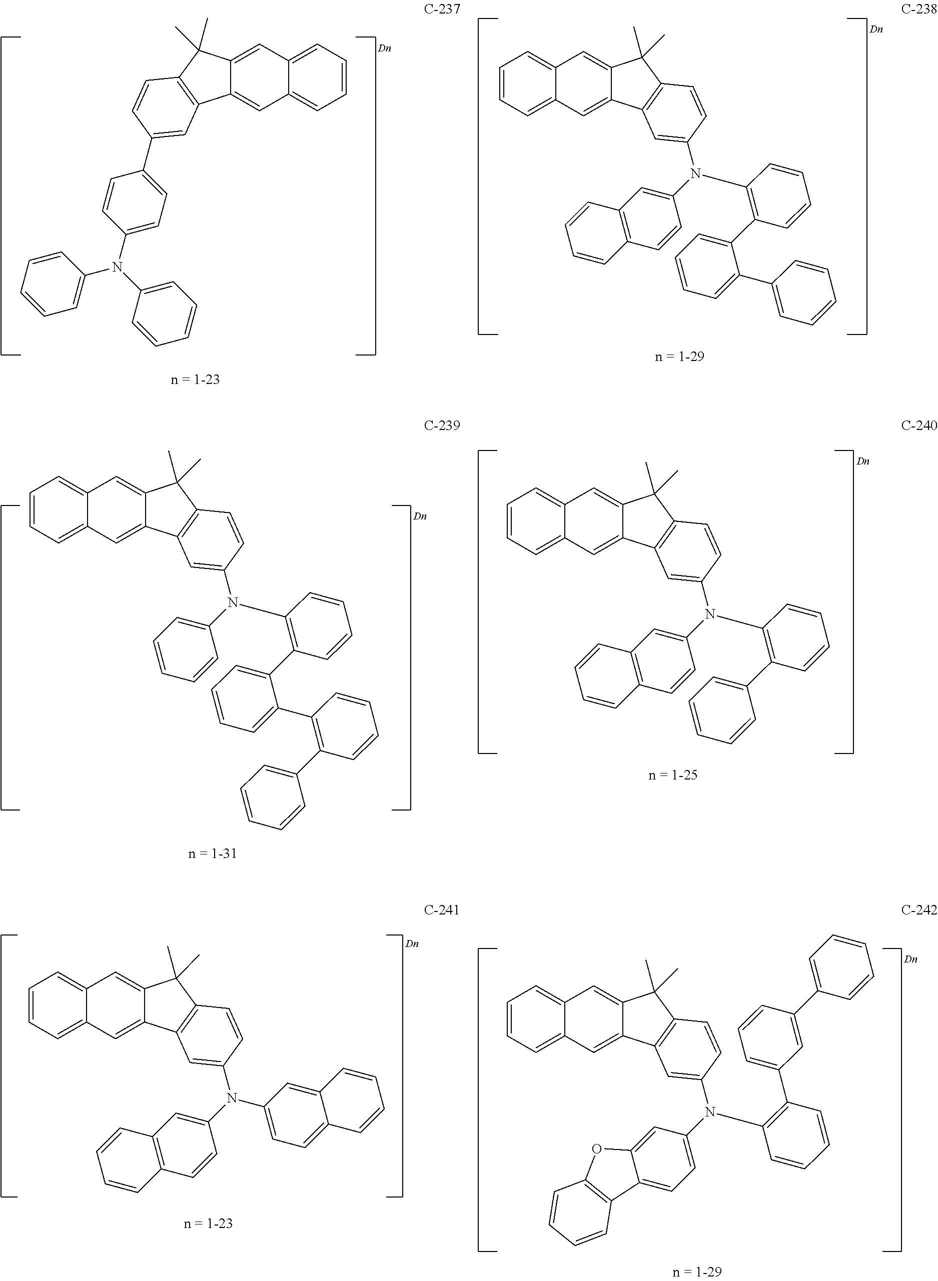

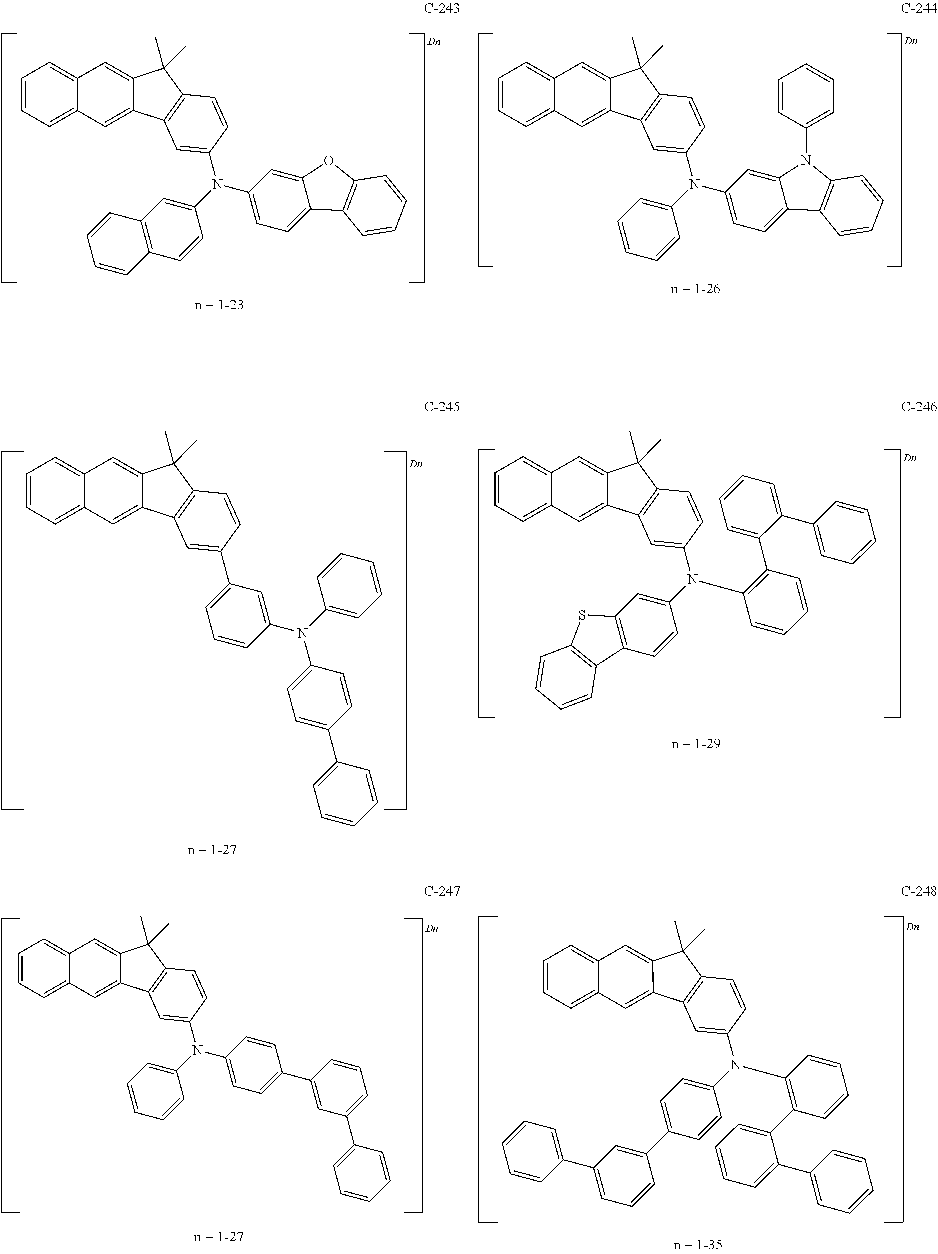

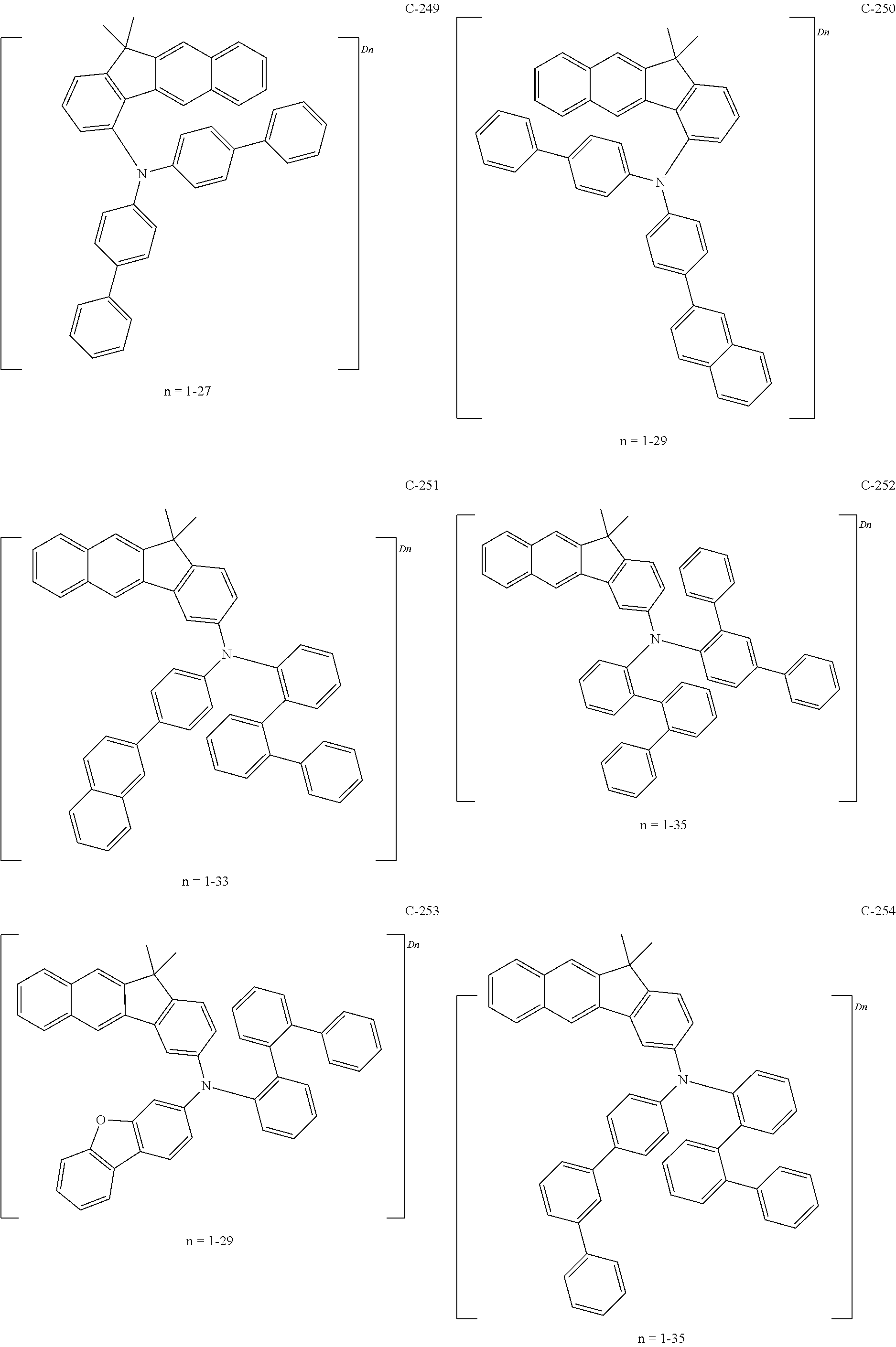

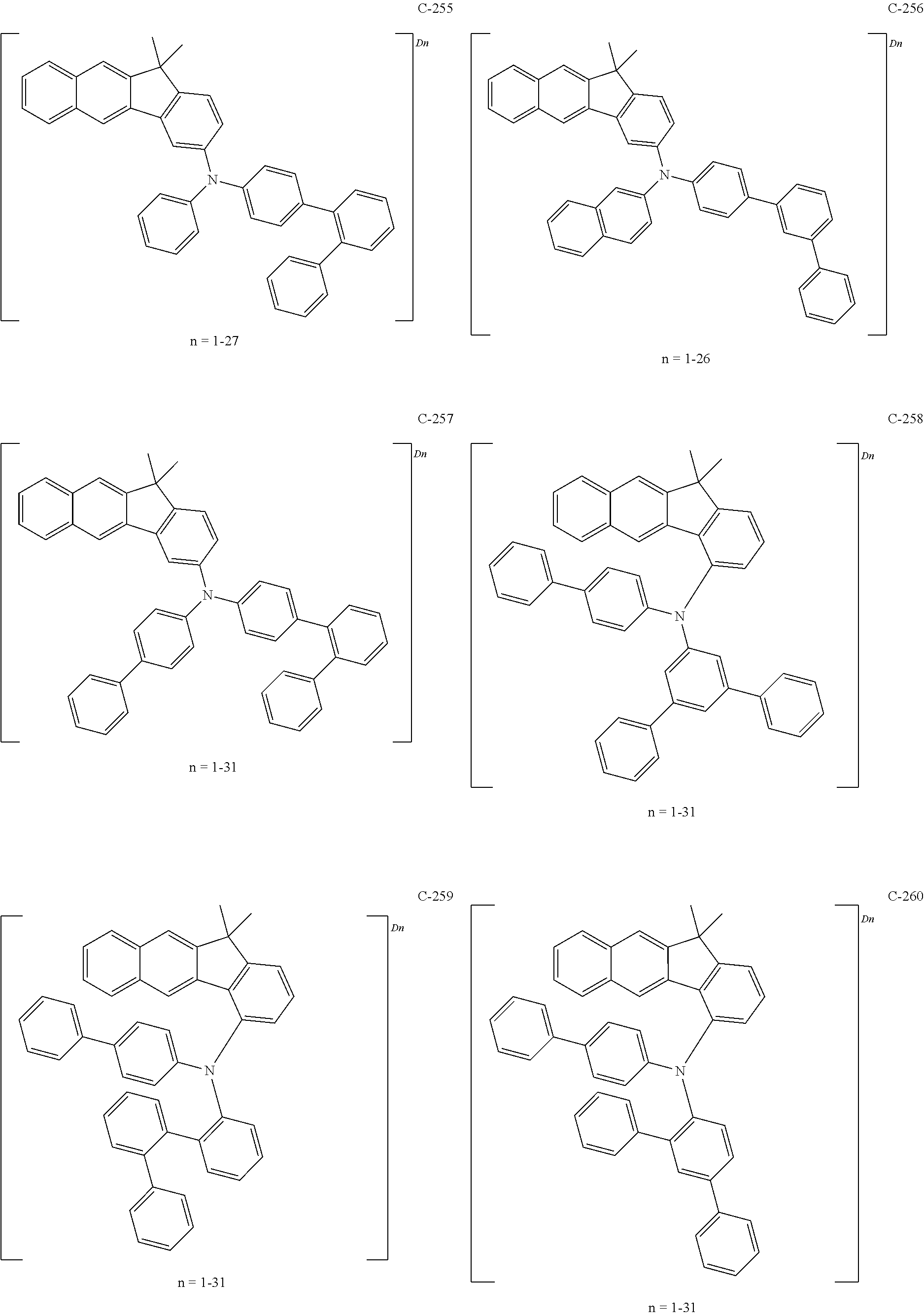

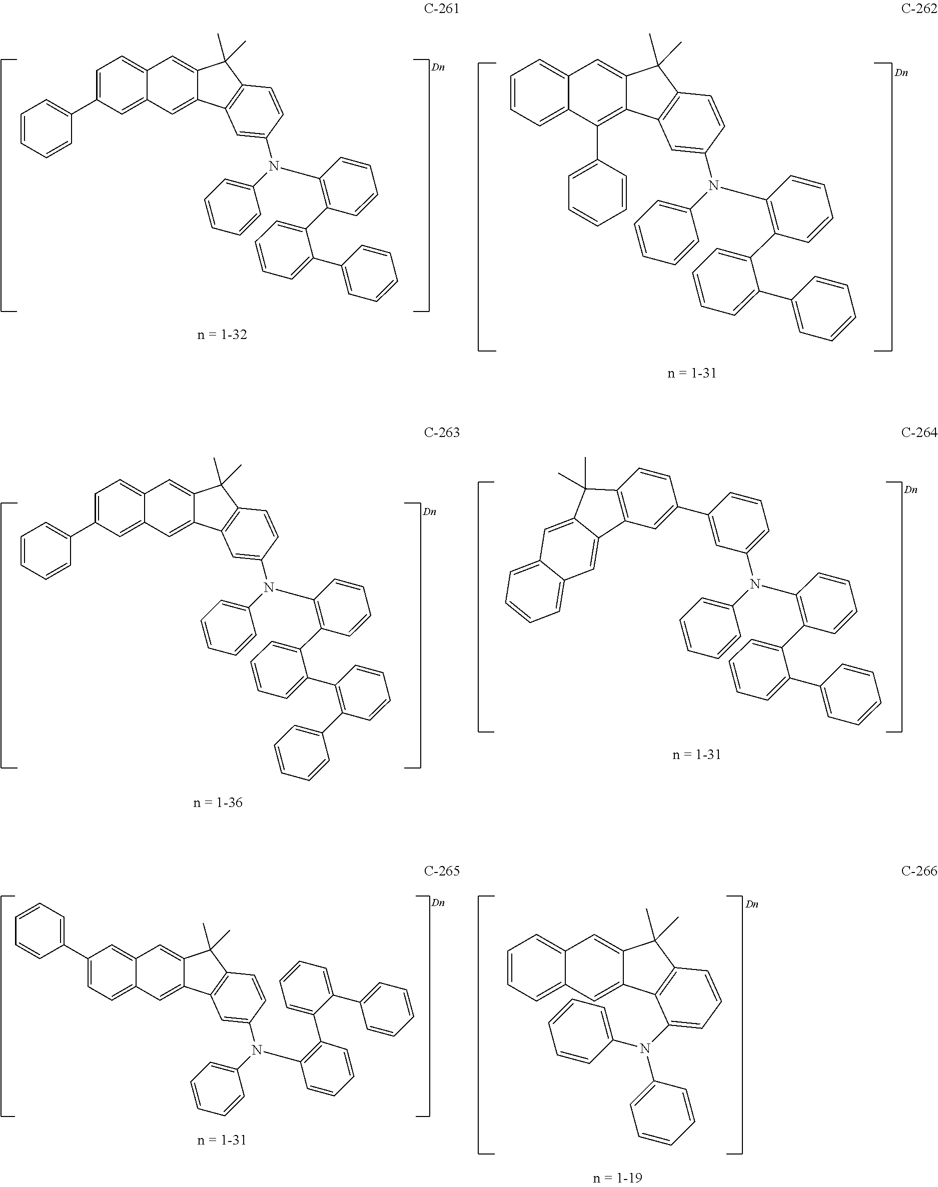

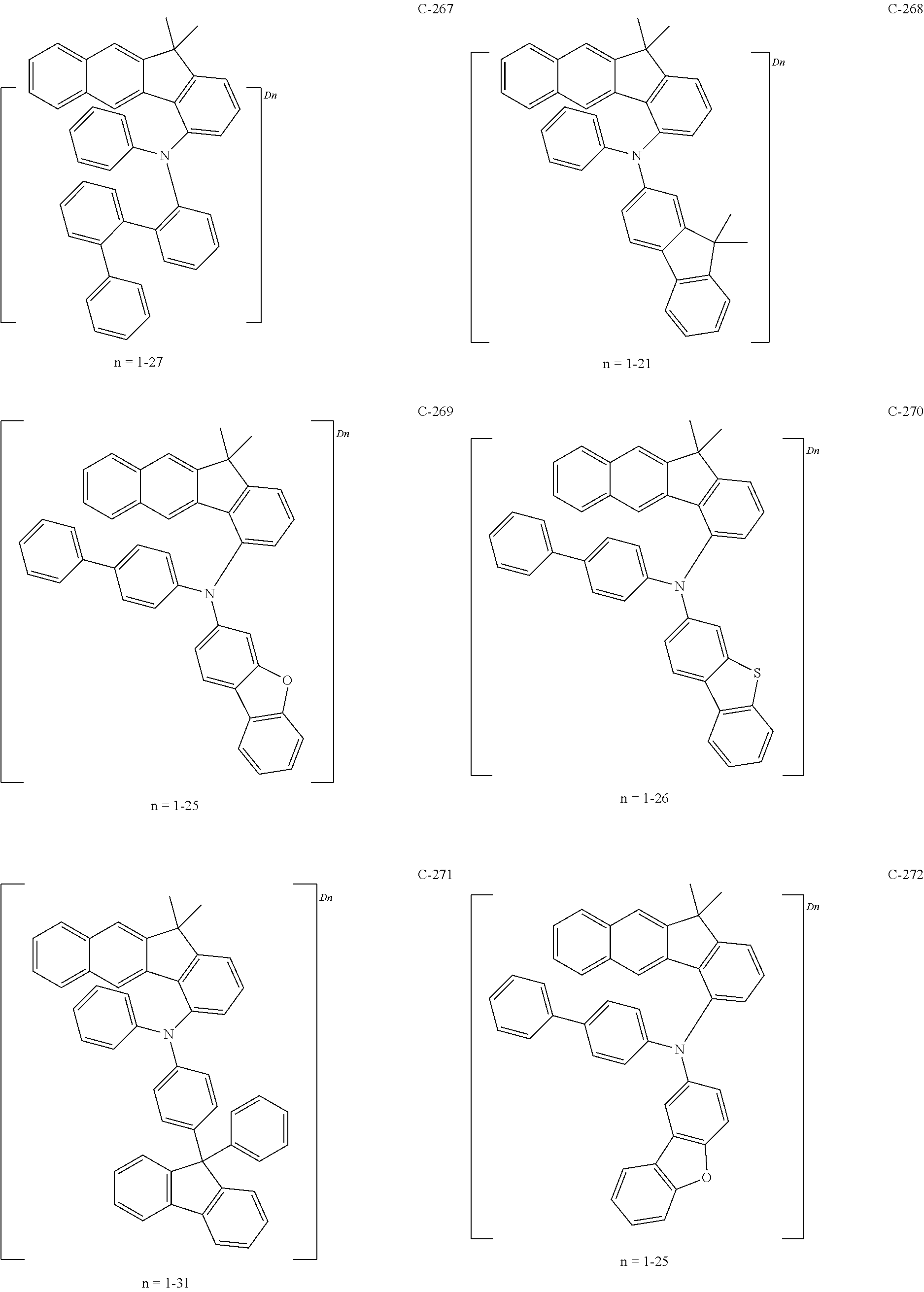

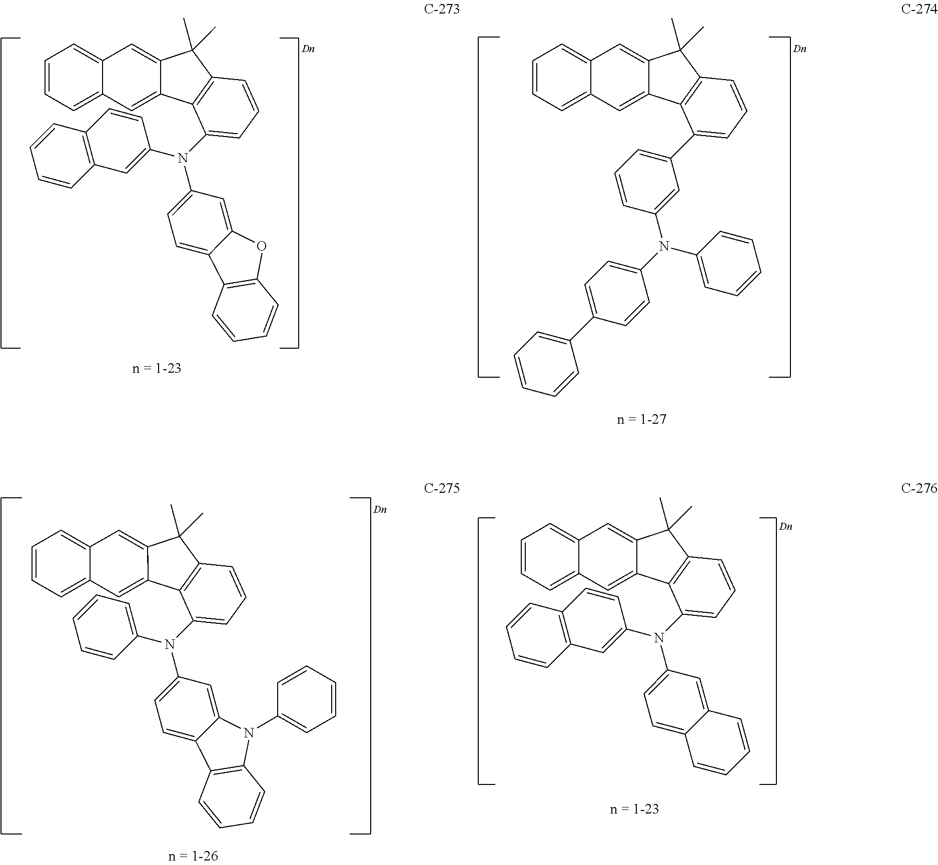

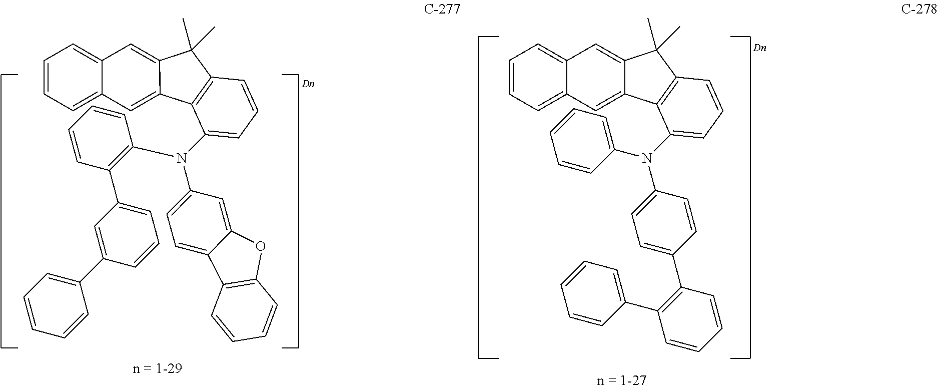

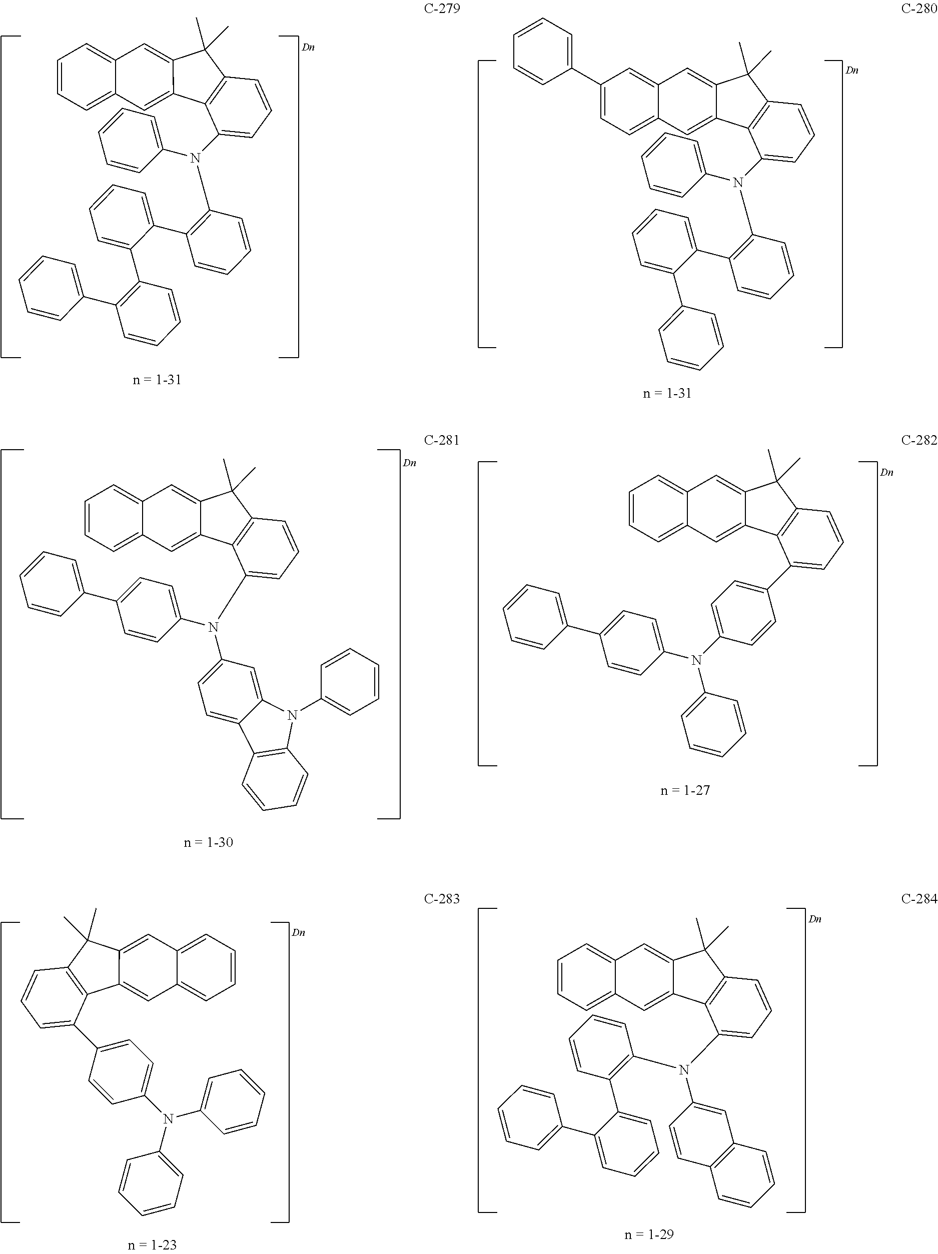

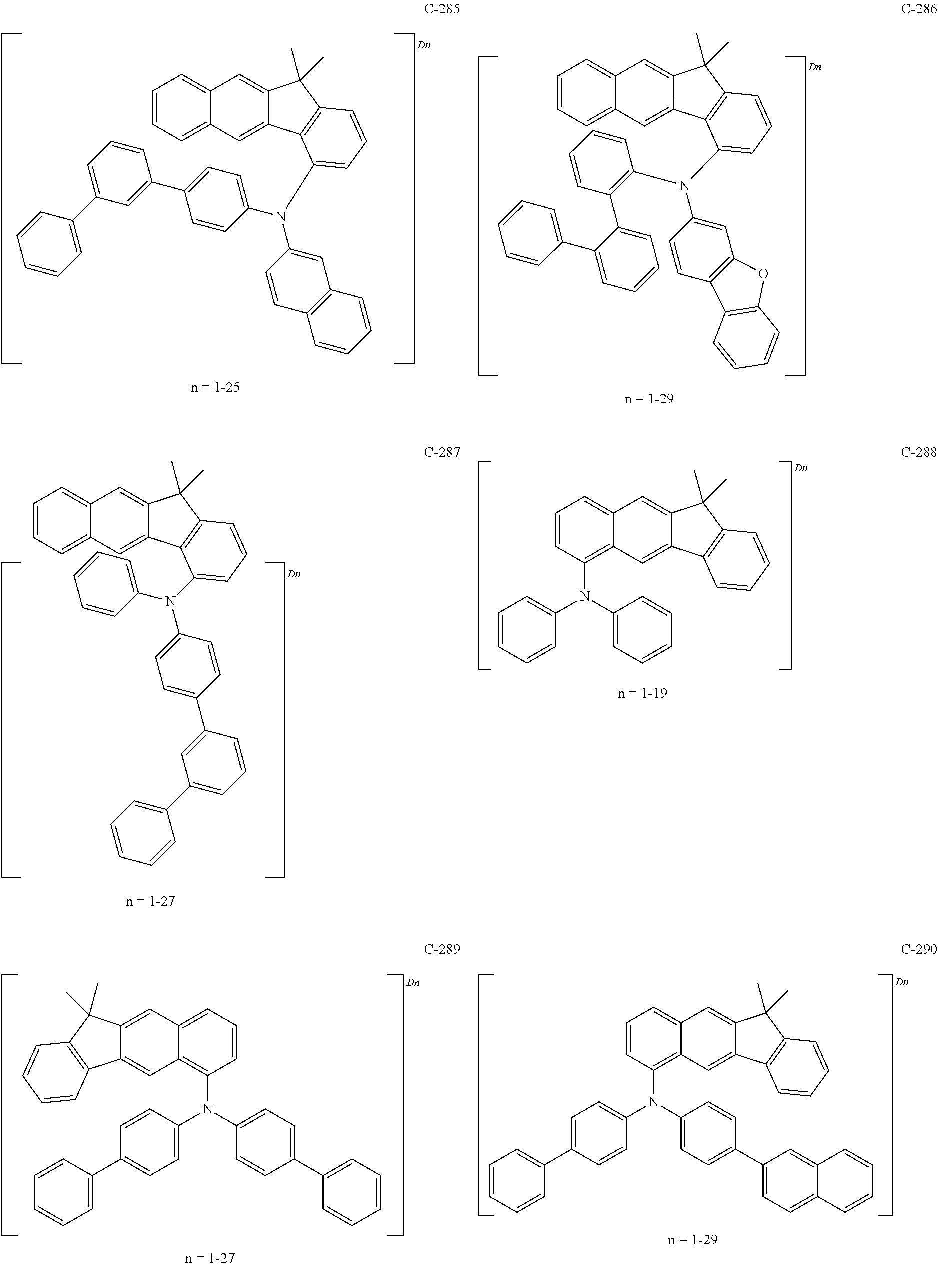

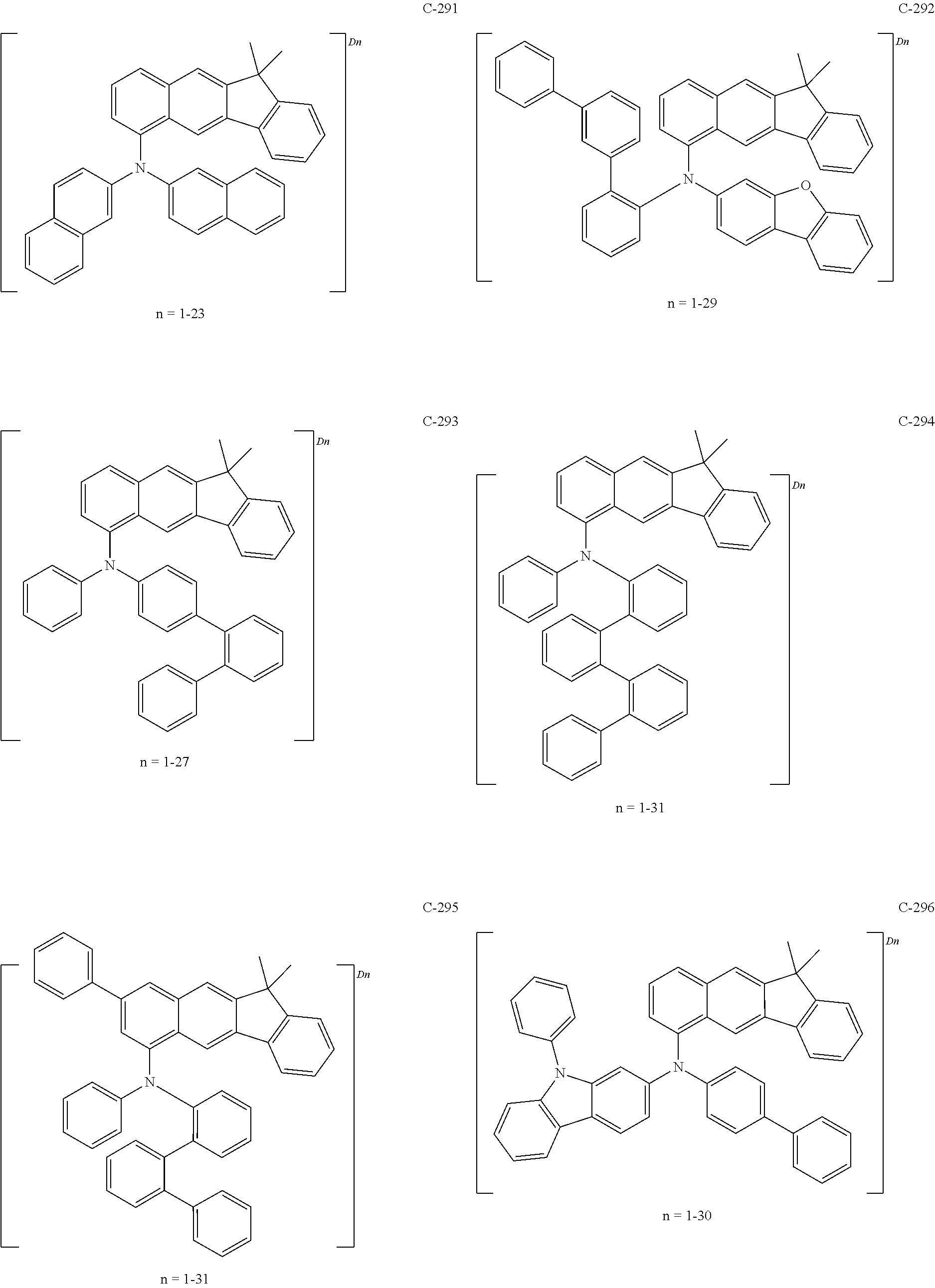

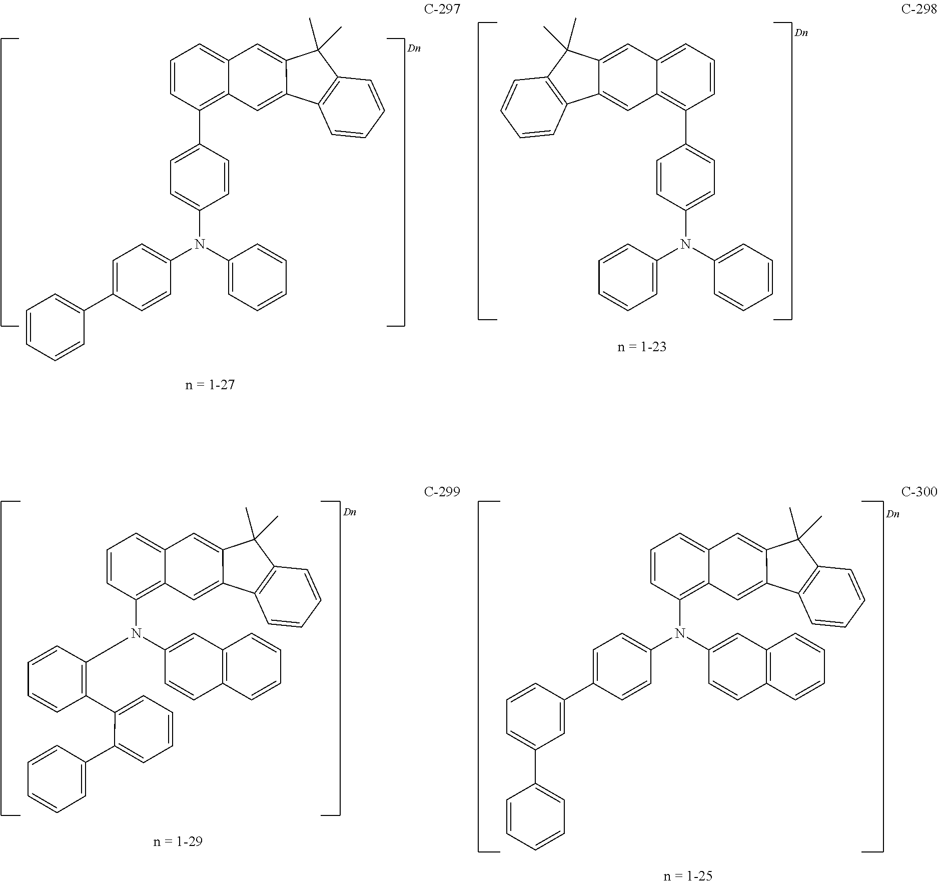

In the compounds above, Dn signifies that n hydrogens are replaced with deuterium, where n represents an integer of 1 or more, wherein the upper limit of n is determined by the number of hydrogens that can be substituted in each compound.

The organic electroluminescent compound represented by Formula 1 according to the present disclosure can be prepared by a known synthesis method. For example, the method disclosed in Korean Patent No. 10-1939552, but is not limited thereto. The deuterated compound of Formula 1 can be prepared using a deuterated precursor material in a similar manner, or more generally can be prepared by treating a non-deuterated compound with a deuterated solvent, D6-benzene in the presence of a Lewis acid H/D exchange catalyst such as aluminum trichloride or ethyl aluminum chloride. In addition, the degree of deuterization can be controlled by varying reaction conditions such as the reaction temperature. For example, the number of deuterium in Formula 1 can be adjusted by controlling the reaction temperature and time, the equivalent of acid, etc.

Hereinafter, an organic electroluminescent material to which the aforementioned organic electroluminescent compound is applied and an organic electroluminescent device comprising the organic electroluminescent material, will be described.

The organic electroluminescent device according to one embodiment includes a first electrode; a second electrode; and at least one organic layer(s) interposed between the first electrode and the second electrode. The organic layer includes a hole transport layer and a light-emitting layer.

According to another embodiment, the organic electroluminescent material of the present disclosure comprises an organic electroluminescent compound represented by Formula 1 alone or in combination of two or more. Such organic electroluminescent material may be included in an organic layer of an organic electroluminescent device, for example, a hole transport layer. In one embodiment, the hole transport layer according to the present disclosure may be comprise at least one selected from Compounds C-1 to C-300.

The light-emitting layer according to one embodiment may include a known light-emitting material. A known light-emitting material may be a known host material, and may further include one or more dopants.

In one embodiment, the host used herein may be a phosphorescent host compound or a fluorescent host compound, and these host compounds are not particularly limited.

The fluorescent host material included in the organic electroluminescent device of the present disclosure is not particularly limited, but a compound represented by the following Formula 90 can be used.

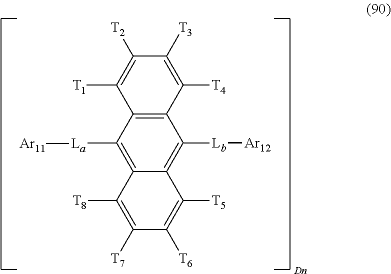

In Formula 90,
Lₐ and L_{b} each independently represent, a single bond, a substituted or unsubstituted (C6-C30)arylene, or a substituted or unsubstituted (5- to 30-membered)heteroarylene;
Ar₁₁ and Ar₁₂ each independently represent, a substituted or unsubstituted (C6-C30)aryl or a substituted or unsubstituted (5- to 30-membered)heteroaryl;
T₁ to T₈ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L_{c}-N—(Ar₁₃)(Ar₁₄);
L_{c} represents a single bond, a substituted or unsubstituted (C6-C30)arylene, or a substituted or unsubstituted (5- to 30-membered)heteroarylene;
Ar₁₃ and Ar₁₄ each independently represent, hydrogen, a substituted or unsubstituted (C1-C30)alkyl, a substituted or unsubstituted (C2-C30)alkenyl, a substituted or unsubstituted fused ring of (C3-C30) aliphatic ring and (C6-C30) aromatic ring, a substituted or unsubstituted (C6-C30)aryl, or a substituted or unsubstituted (5- to 30-membered)heteroaryl; and
Dn means the replacement of n hydrogens with deuterium, where n represents either 0 or an integer of 8 or more.

Specifically, specific examples of the fluorescent host compound are as follows, but are not limited thereto.

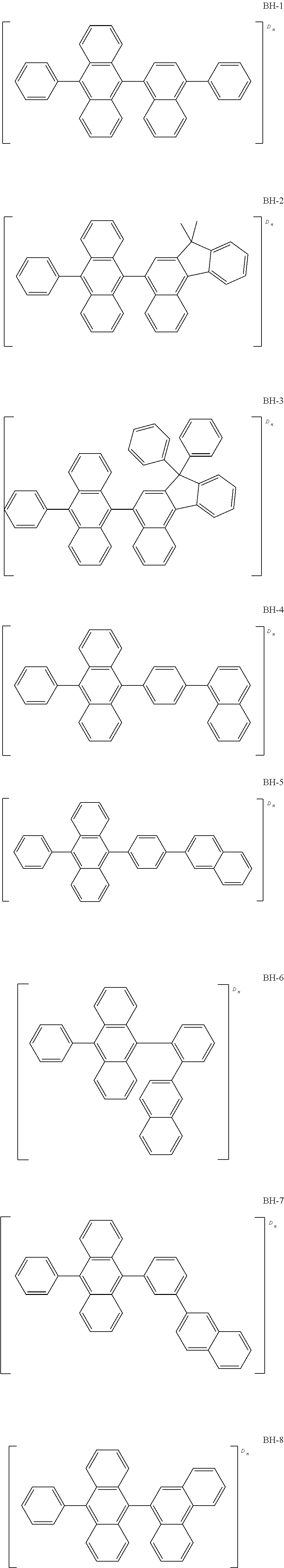

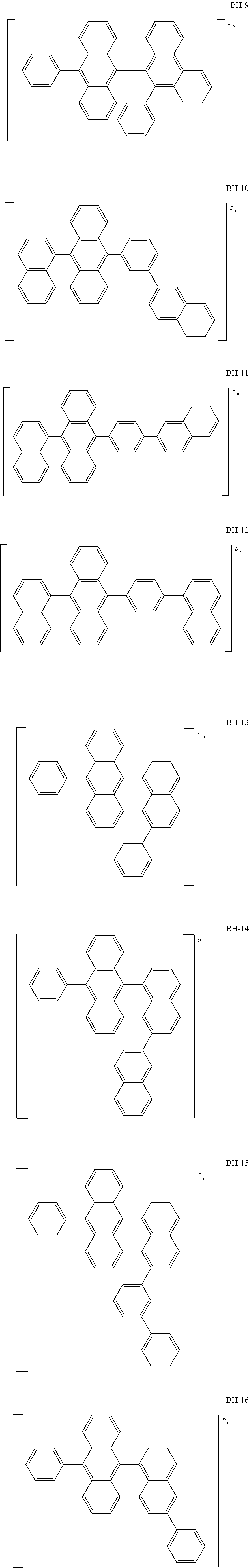

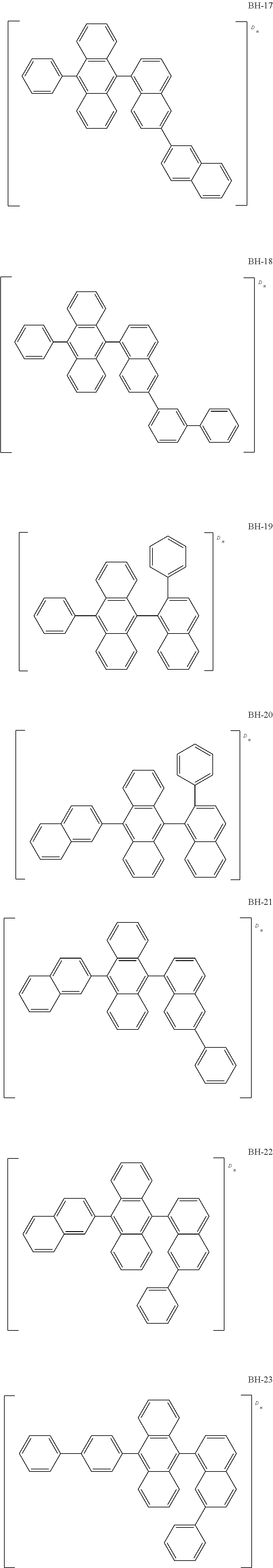

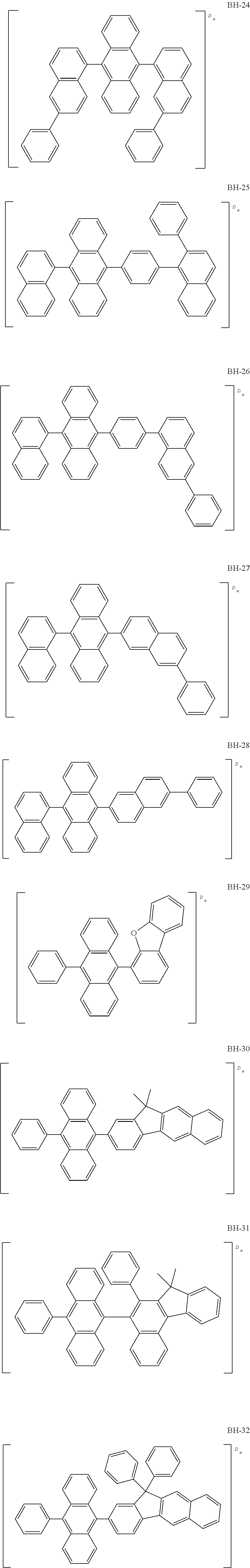

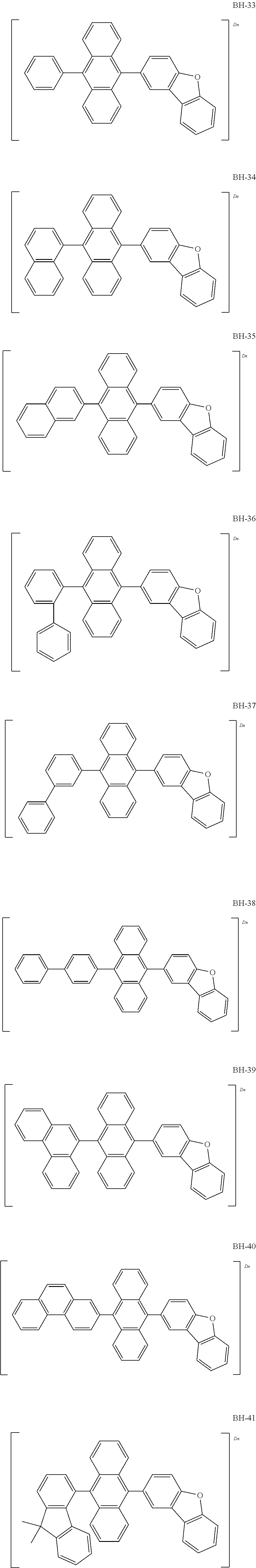

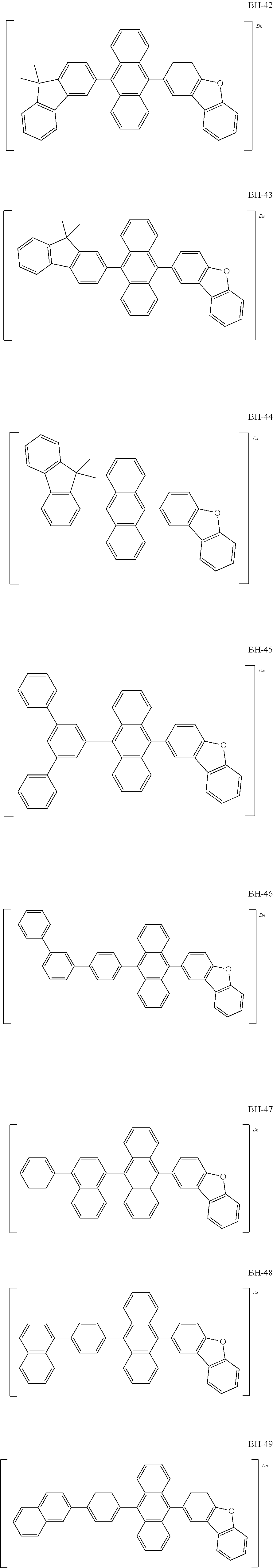

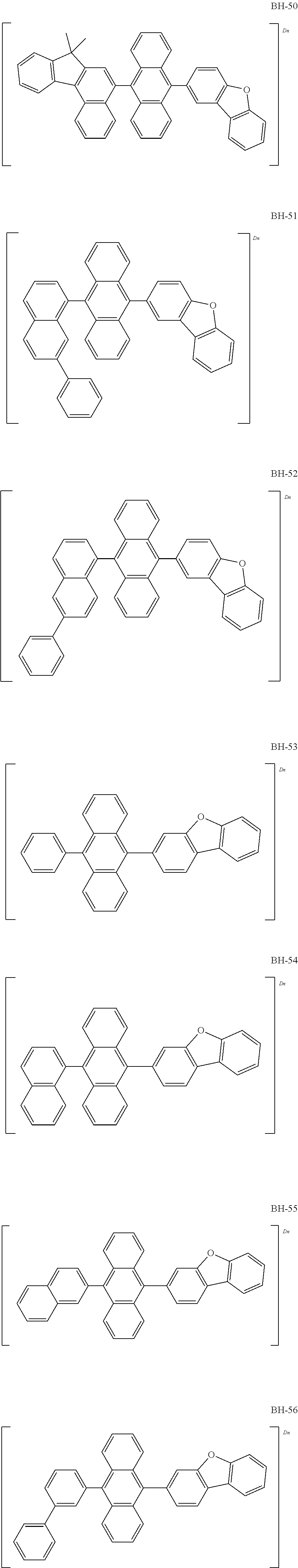

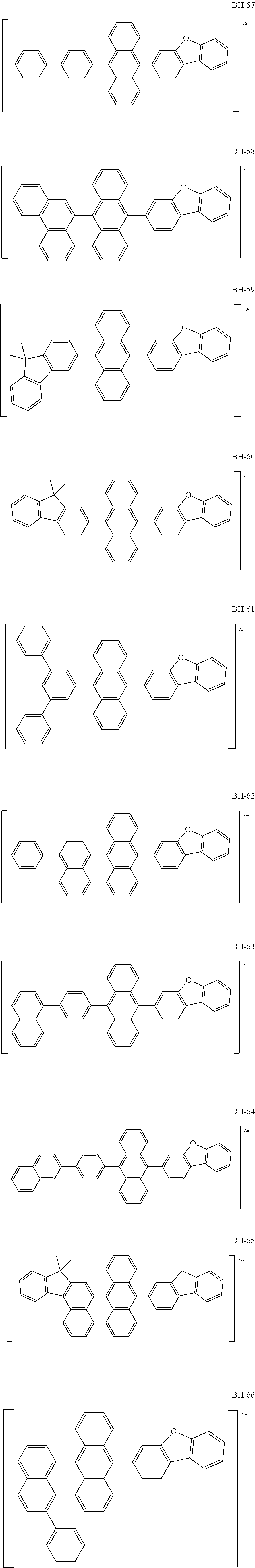

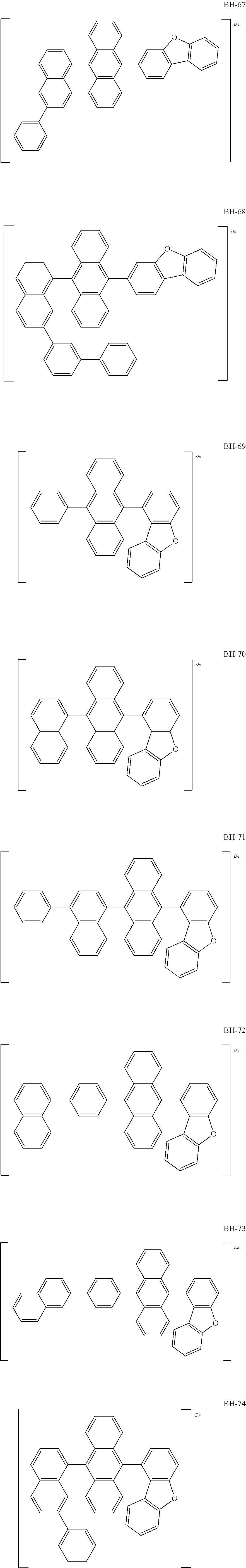

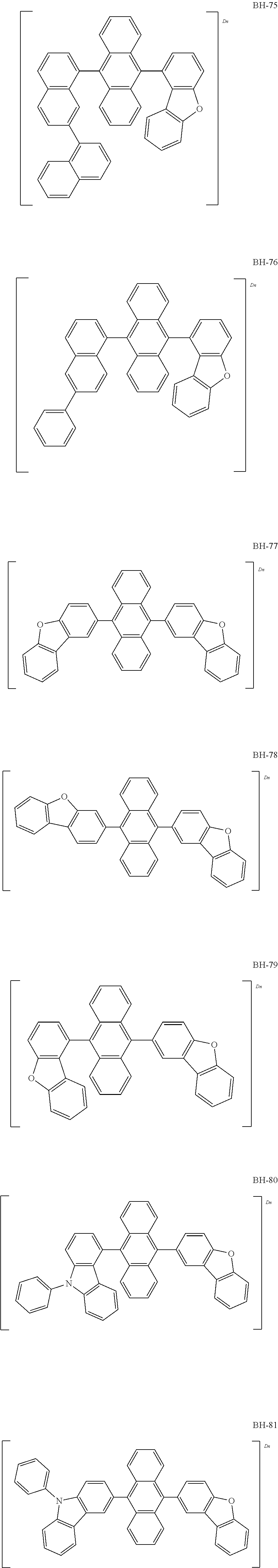

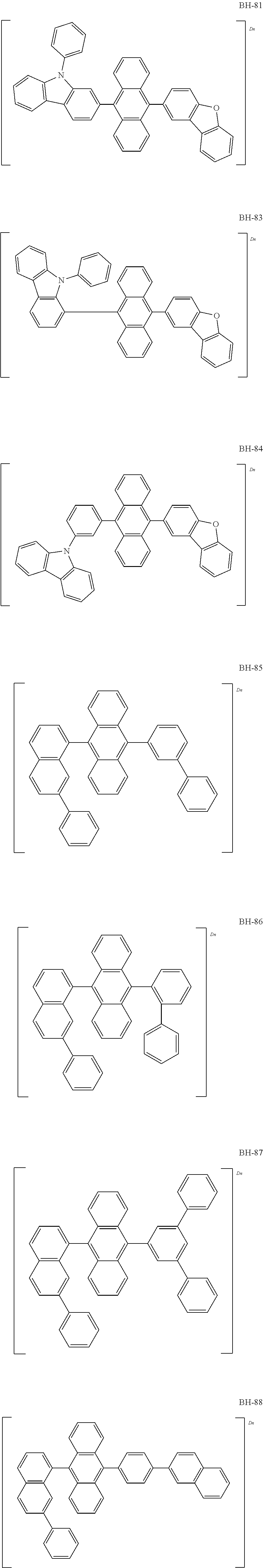

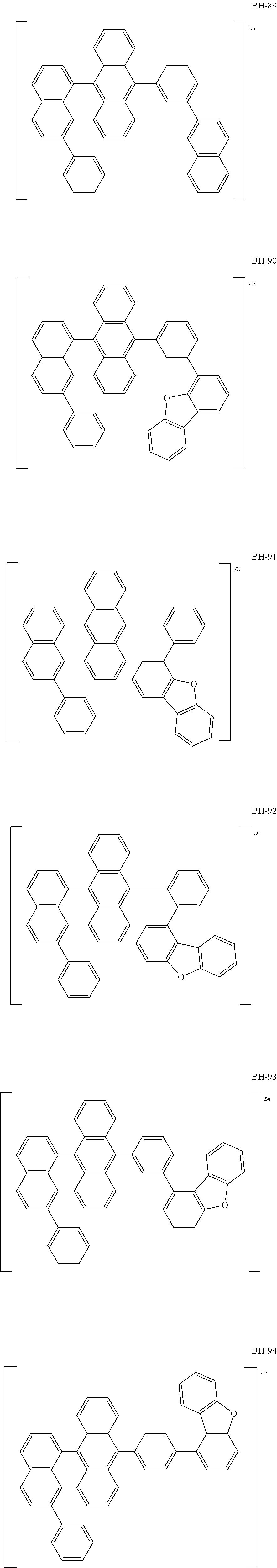

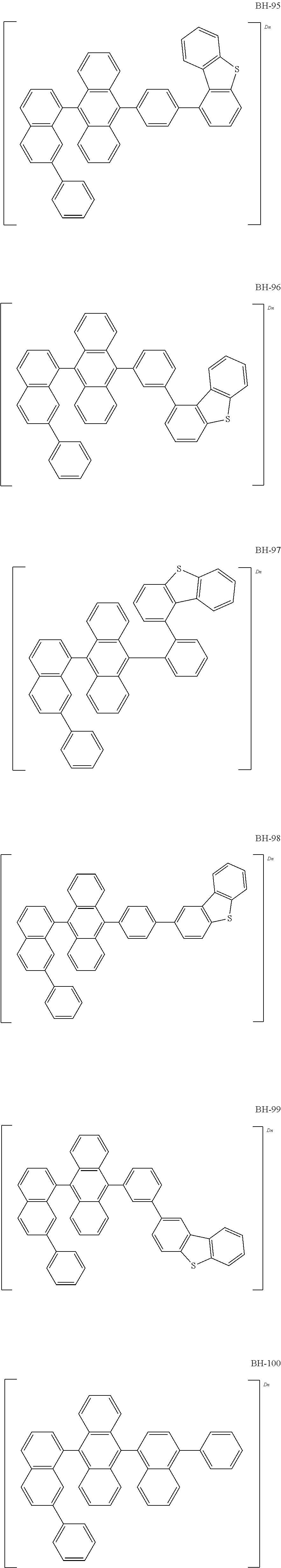

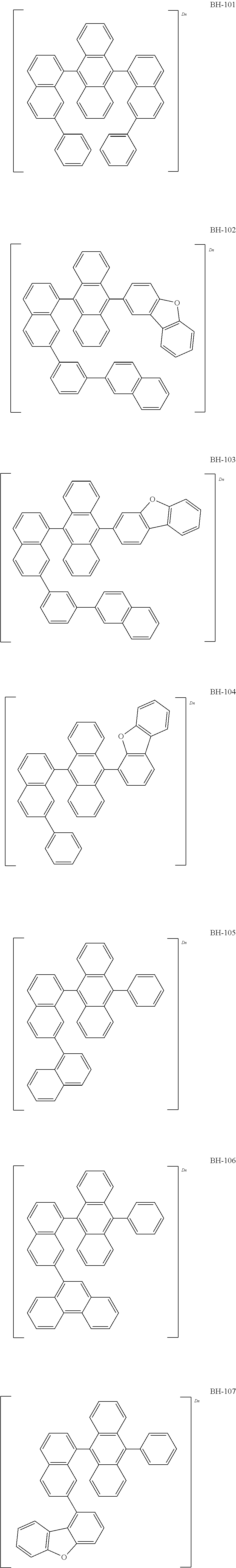

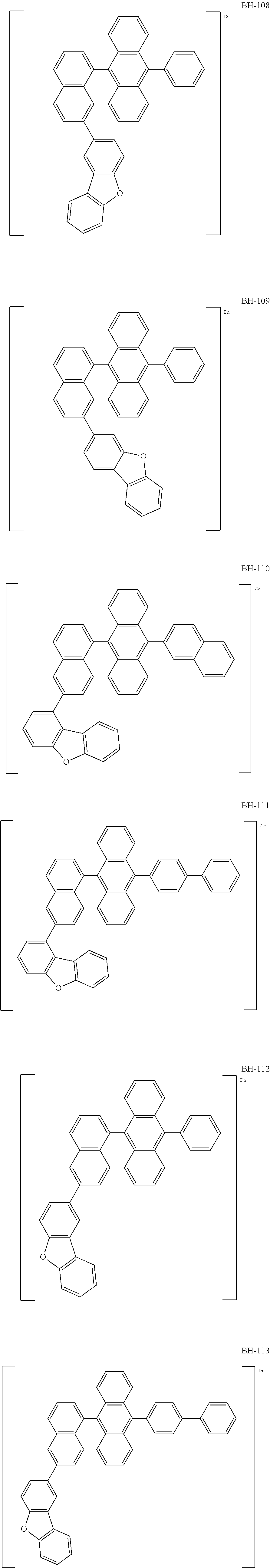

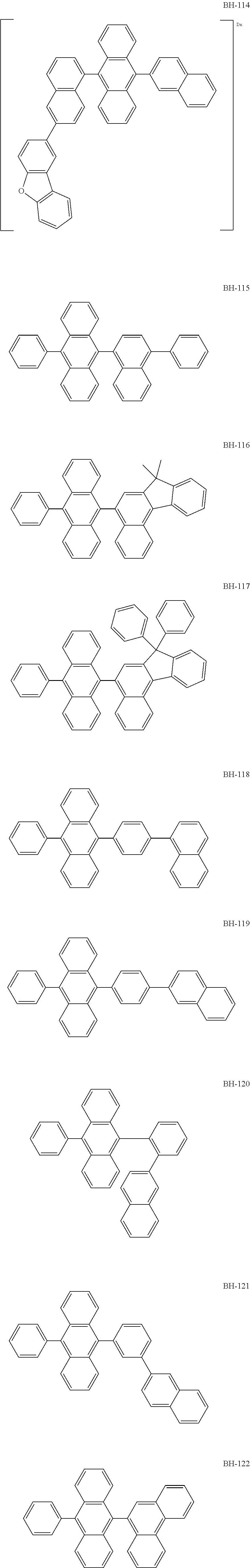

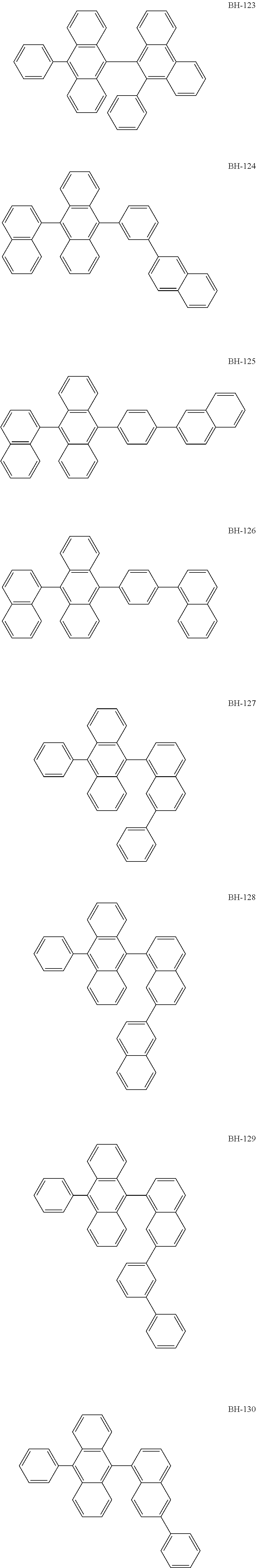

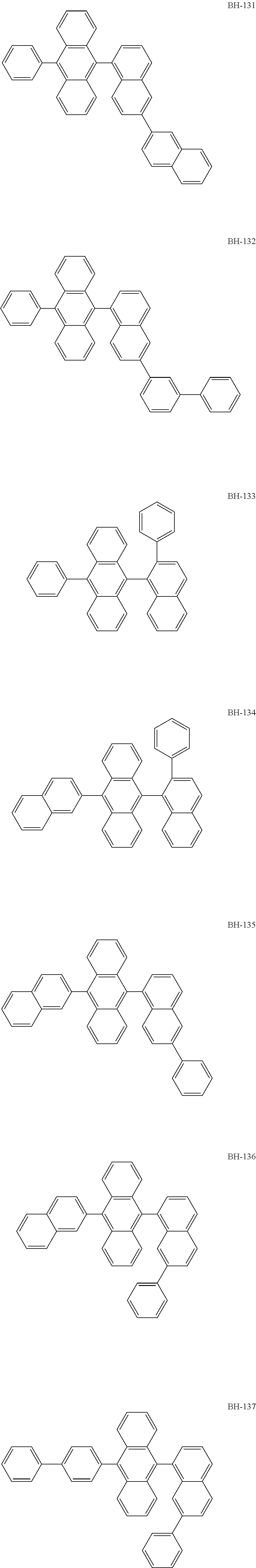

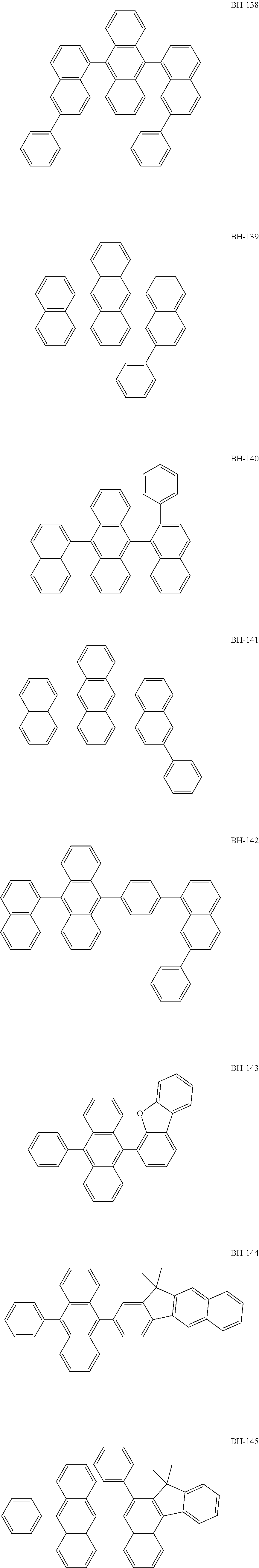

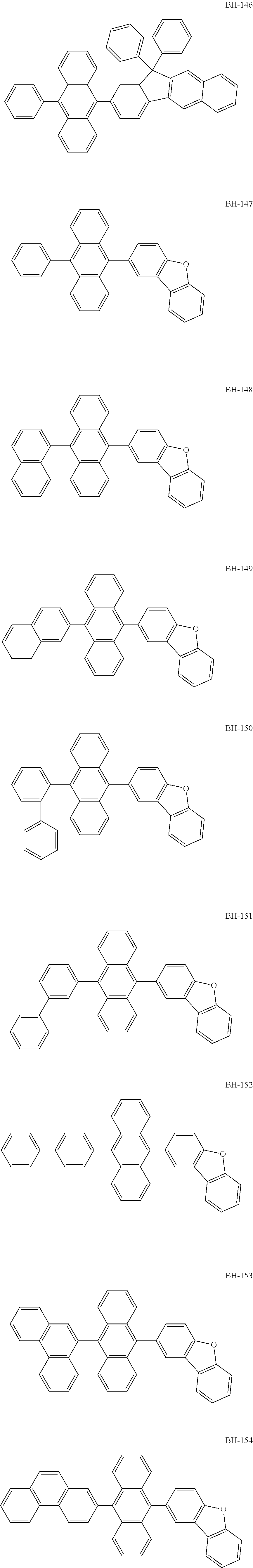

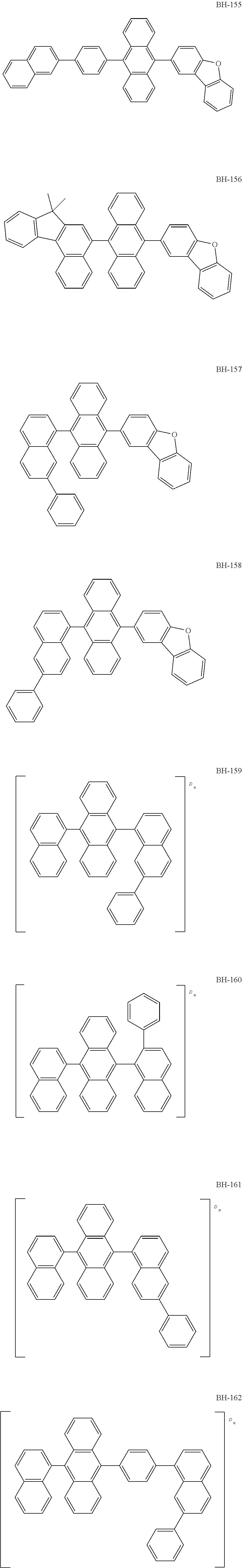

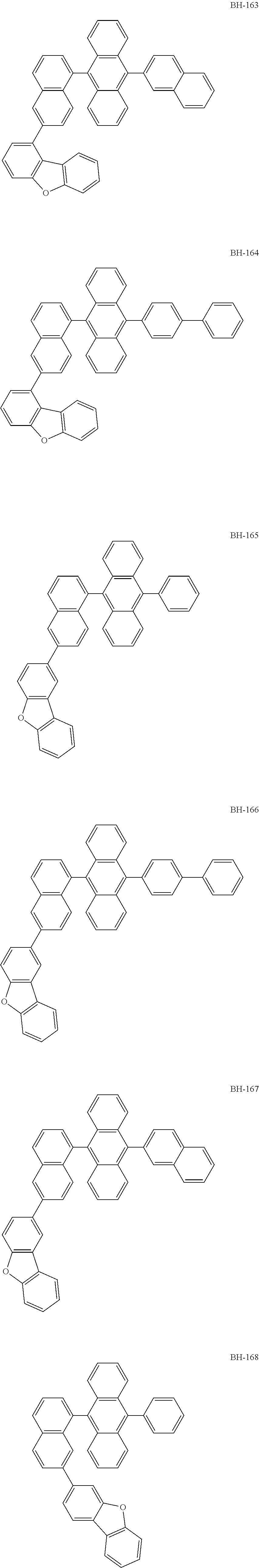 [SMILES: c1ccc2cc(-c3c4ccccc4c(-c4cccc5cc(-c6cccc7oc8ccccc8c67)ccc45)c4ccccc34)ccc2c1 ; c1ccc(-c2ccc(-c3c4ccccc4c(-c4cccc5cc(-c6cccc7oc8ccccc8c67)ccc45)c4ccccc34)cc2)cc1 ; c1ccc(-c2c3ccccc3c(-c3cccc4cc(-c5ccc6oc7ccccc7c6c5)ccc34)c3ccccc23)cc1 ; c1ccc(-c2ccc(-c3c4ccccc4c(-c4cccc5cc(-c6ccc7oc8ccccc8c7c6)ccc45)c4ccccc34)cc2)cc1 ; c1ccc2cc(-c3c4ccccc4c(-c4cccc5cc(-c6ccc7oc8ccccc8c7c6)ccc45)c4ccccc34)ccc2c1 ; c1ccc(-c2c3ccccc3c(-c3cccc4ccc(-c5ccc6c(c5)oc5ccccc56)cc34)c3ccccc23)cc1]

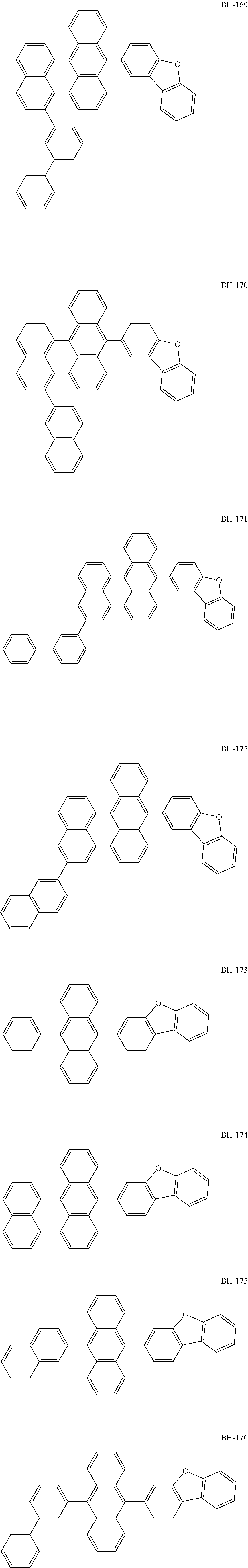

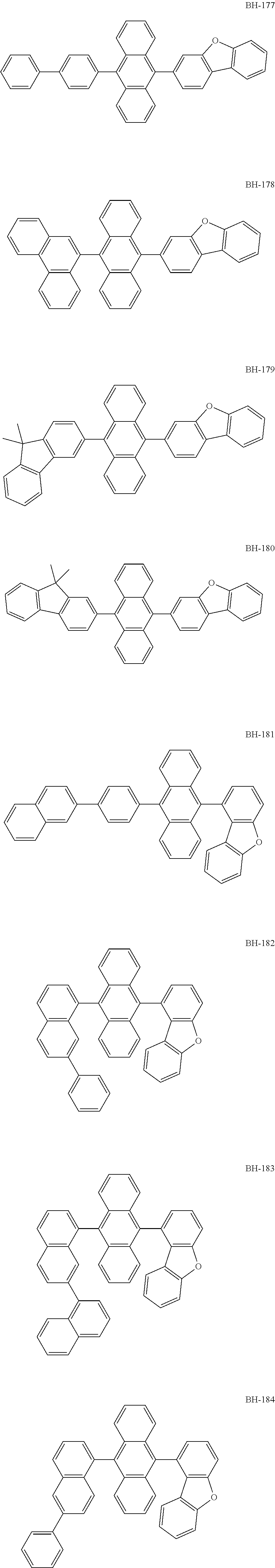

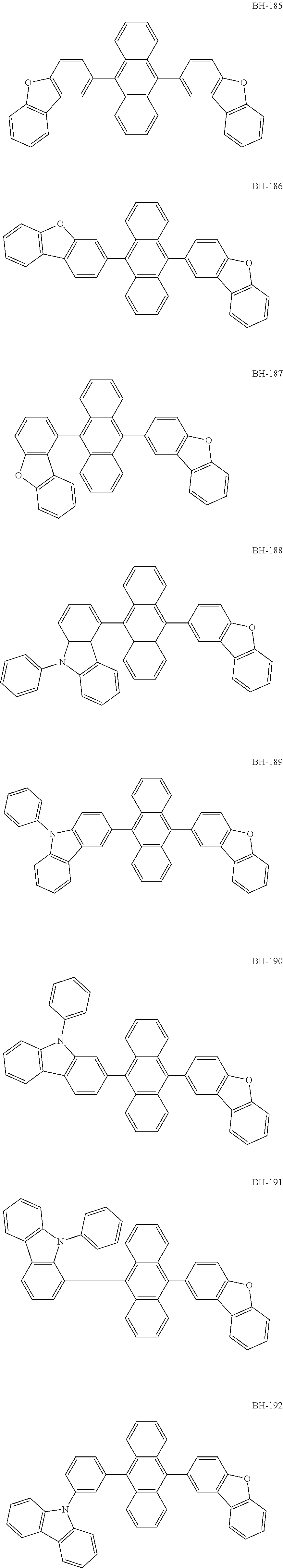

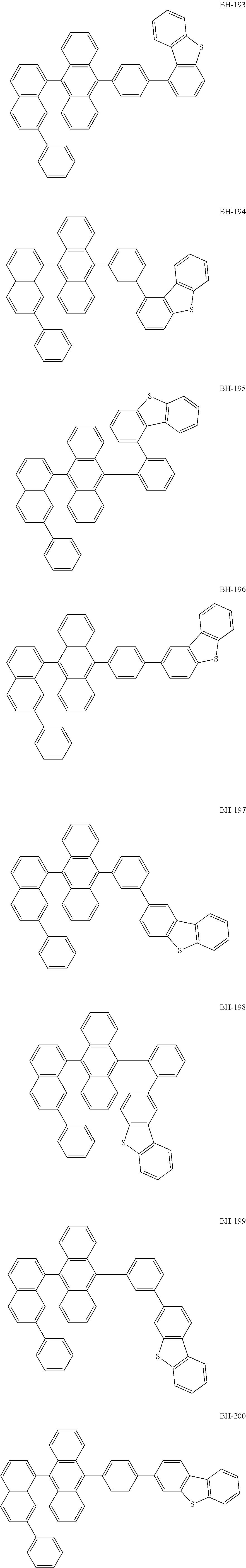

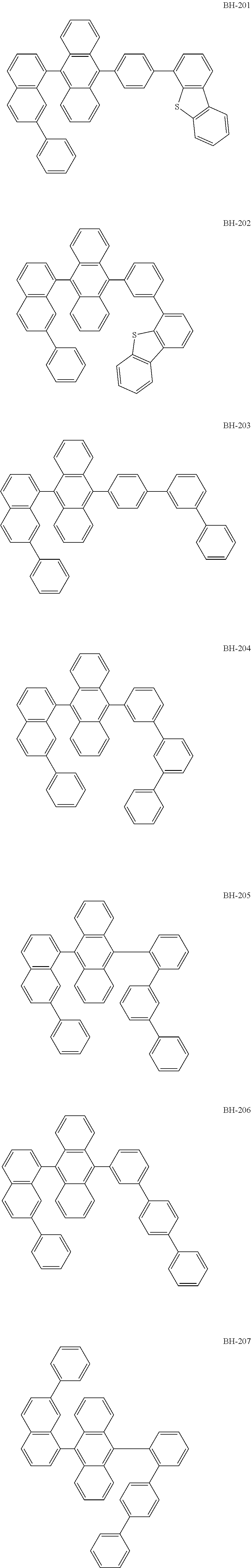

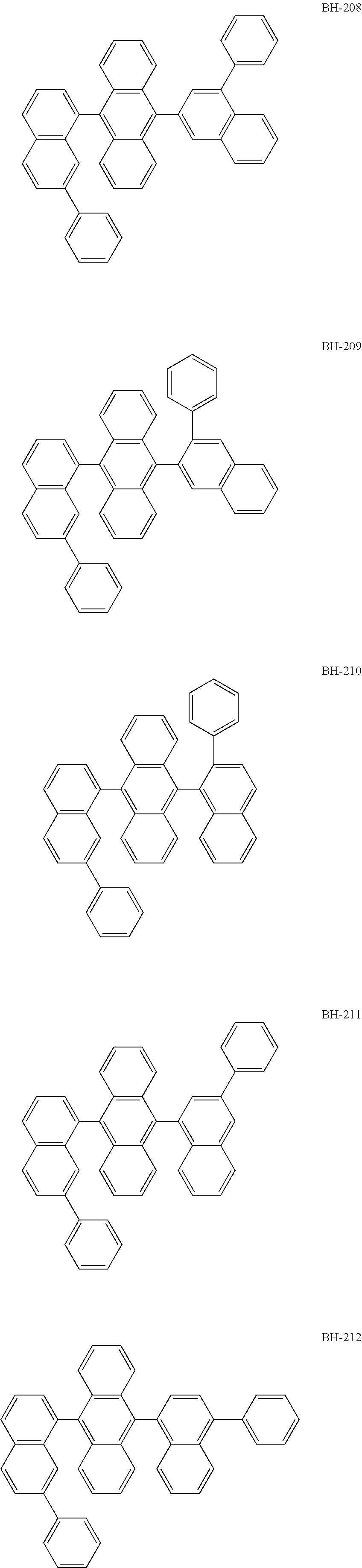

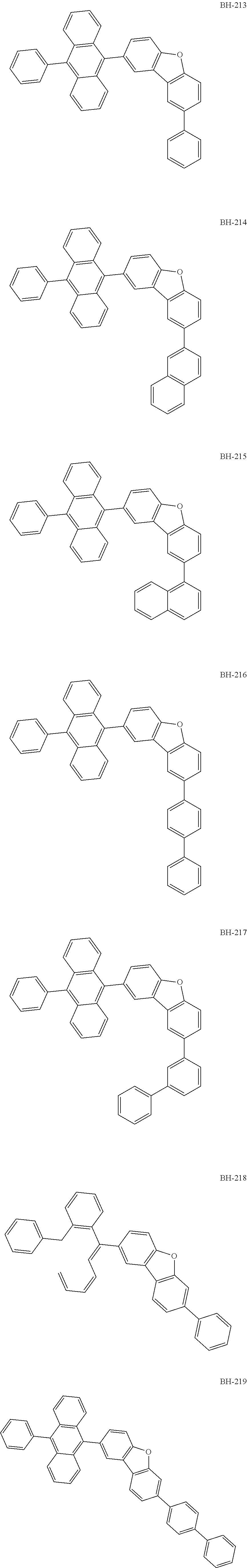

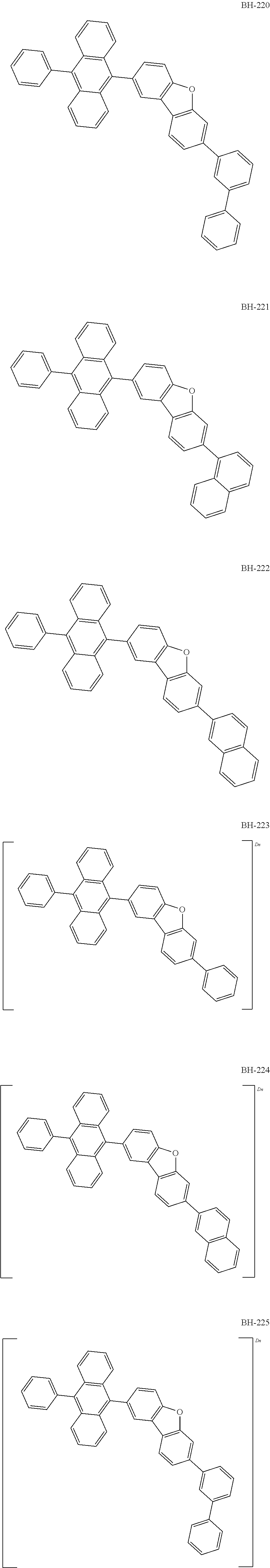

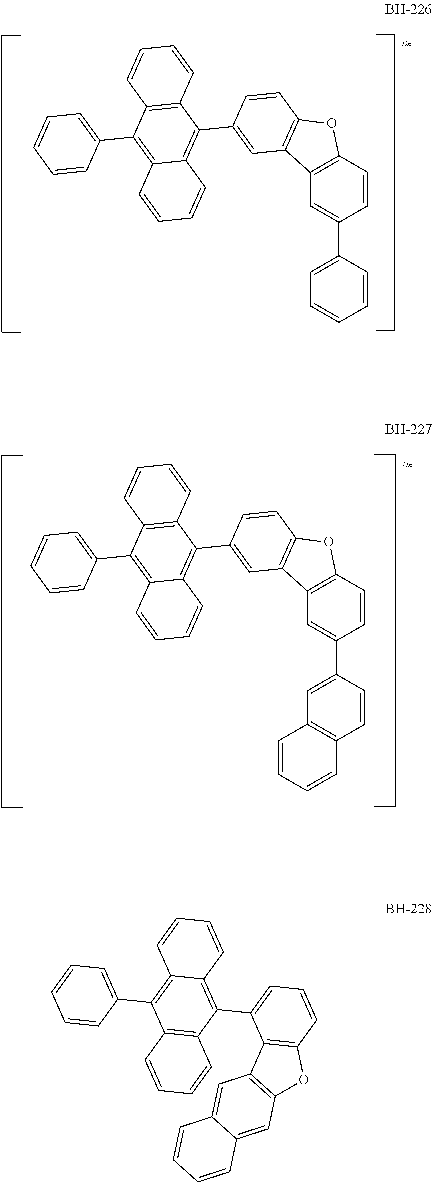

In one embodiment, one or more phosphorescent or fluorescent dopants, preferably fluorescent dopants may be used as the dopant used herein.

A compound represented by the following Formula 100 may be used as a fluorescent dopant included in the organic electroluminescent device of the present disclosure, but is not limited thereto.

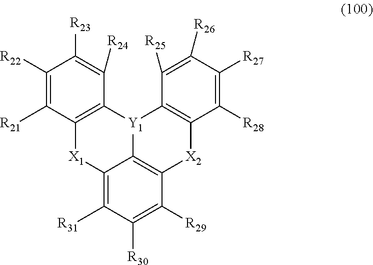

In Formula 100,
Y₁ represents B;
X₁ and X₂ each independently represent, NR or O;
R and R₂₁ to R₃₁ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₄-N—(Ar₁₅)(Ar₁₆); or may be linked to an adjacent substituent(s) to form a ring(s);
L₄ represents a single bond, a substituted or unsubstituted (C6-C30)arylene, or a substituted or unsubstituted (5- to 30-membered)heteroarylene; and
Ar₁₄ and Ar₁₅ each independently represent, hydrogen, a substituted or unsubstituted (C1-C30)alkyl, a substituted or unsubstituted (C2-C30)alkenyl, a substituted or unsubstituted fused ring of (C3-C30) aliphatic ring and (C6-C30) aromatic ring, a substituted or unsubstituted (C6-C30)aryl, or a substituted or unsubstituted (5- to 30-membered)heteroaryl.

Specifically, specific examples of the fluorescent dopant compound are as follows, but are not limited thereto.

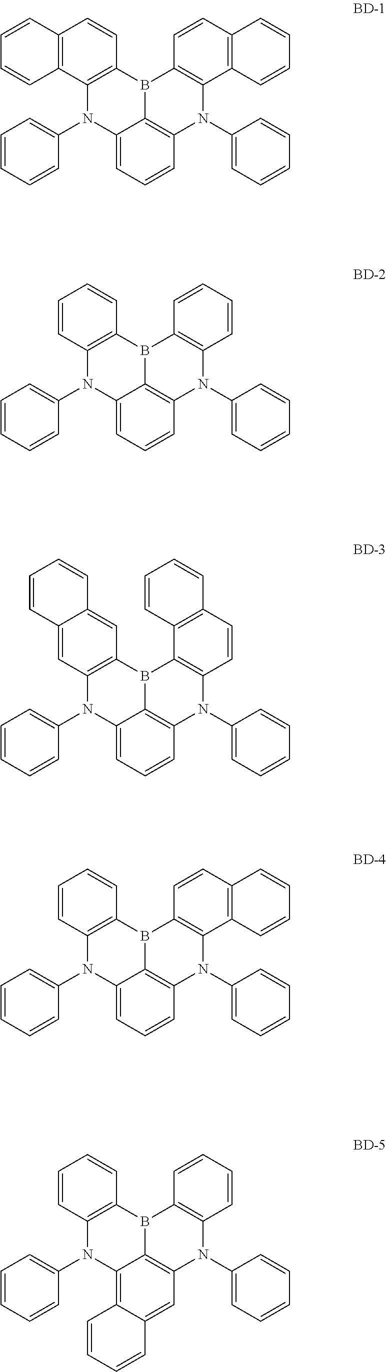 [SMILES: c1ccc(N2c3cccc4c3B(c3ccc5ccccc5c32)c2ccc3ccccc3c2N4c2ccccc2)cc1 ; c1ccc(N2c3ccccc3B3c4ccccc4N(c4ccccc4)c4cccc2c43)cc1 ; c1ccc(N2c3cc4ccccc4cc3B3c4c2cccc4N(c2ccccc2)c2ccc4ccccc4c23)cc1 ; c1ccc(N2c3ccccc3B3c4ccc5ccccc5c4N(c4ccccc4)c4cccc2c43)cc1 ; c1ccc(N2c3ccccc3B3c4ccccc4N(c4ccccc4)c4c3c2cc2ccccc42)cc1]

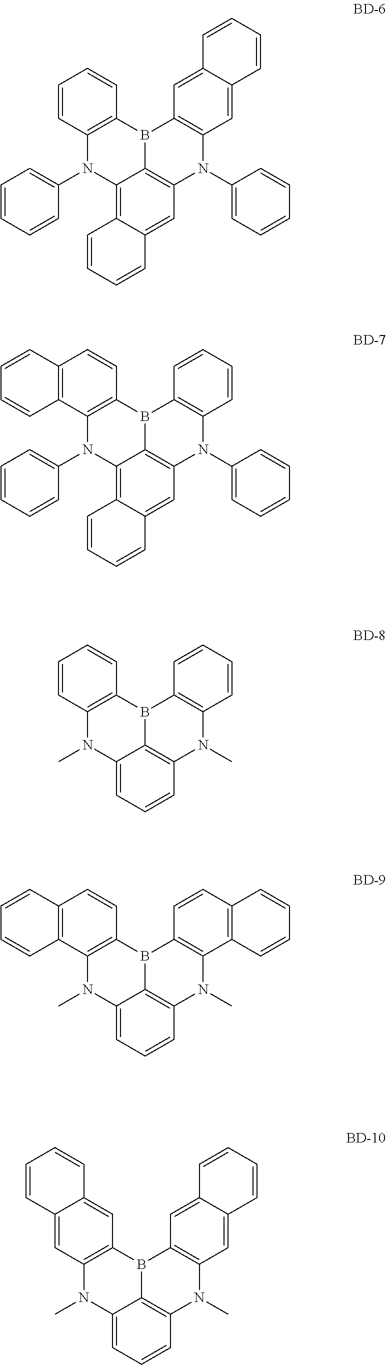 [SMILES: c1ccc(N2c3cc4ccccc4cc3B3c4ccccc4N(c4ccccc4)c4c3c2cc2ccccc42)cc1 ; c1ccc(N2c3ccccc3B3c4ccc5ccccc5c4N(c4ccccc4)c4c3c2cc2ccccc42)cc1 ; CN1c2ccccc2B2c3ccccc3N(C)c3cccc1c32 ; CN1c2cccc3c2B(c2ccc4ccccc4c21)c1ccc2ccccc2c1N3C ; CN1c2cc3ccccc3cc2B2c3cc4ccccc4cc3N(C)c3cccc1c32]

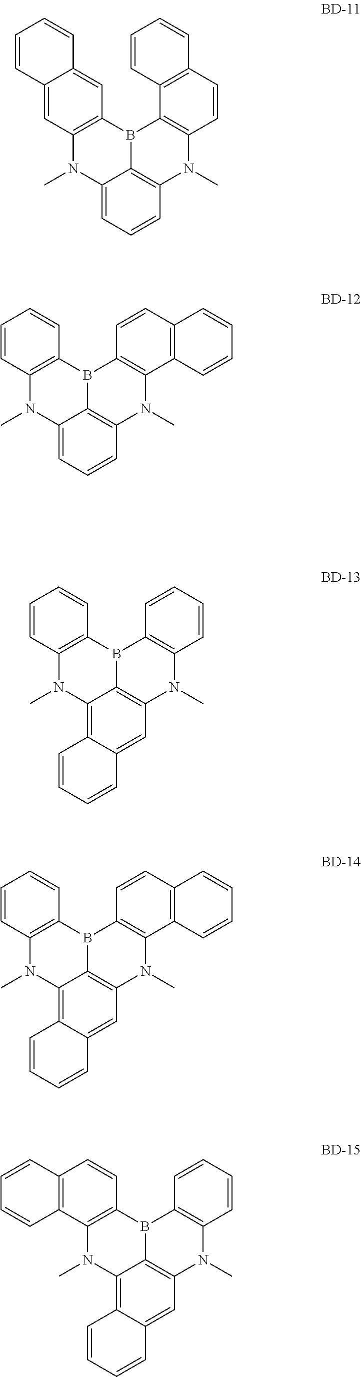 [SMILES: CN1c2cc3ccccc3cc2B2c3c1cccc3N(C)c1ccc3ccccc3c12 ; CN1c2ccccc2B2c3ccc4ccccc4c3N(C)c3cccc1c32 ; CN1c2ccccc2B2c3ccccc3N(C)c3c2c1cc1ccccc31 ; CN1c2cc3ccccc3c3c2B(c2ccccc2N3C)c2ccc3ccccc3c21 ; CN1c2ccccc2B2c3ccc4ccccc4c3N(C)c3c2c1cc1ccccc31]

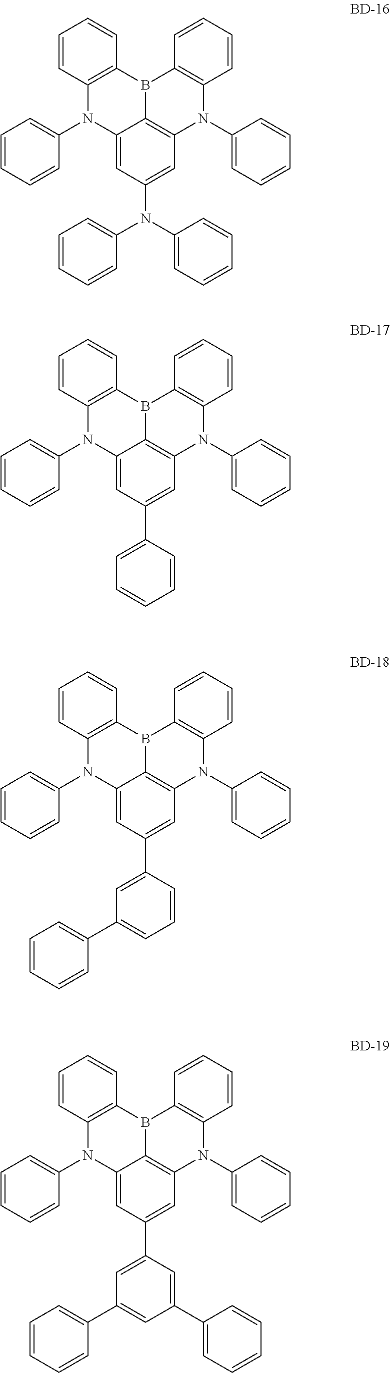 [SMILES: c1ccc(N(c2ccccc2)c2cc3c4c(c2)N(c2ccccc2)c2ccccc2B4c2ccccc2N3c2ccccc2)cc1 ; c1ccc(-c2cc3c4c(c2)N(c2ccccc2)c2ccccc2B4c2ccccc2N3c2ccccc2)cc1 ; c1ccc(-c2cccc(-c3cc4c5c(c3)N(c3ccccc3)c3ccccc3B5c3ccccc3N4c3ccccc3)c2)cc1 ; c1ccc(-c2cc(-c3ccccc3)cc(-c3cc4c5c(c3)N(c3ccccc3)c3ccccc3B5c3ccccc3N4c3ccccc3)c2)cc1]

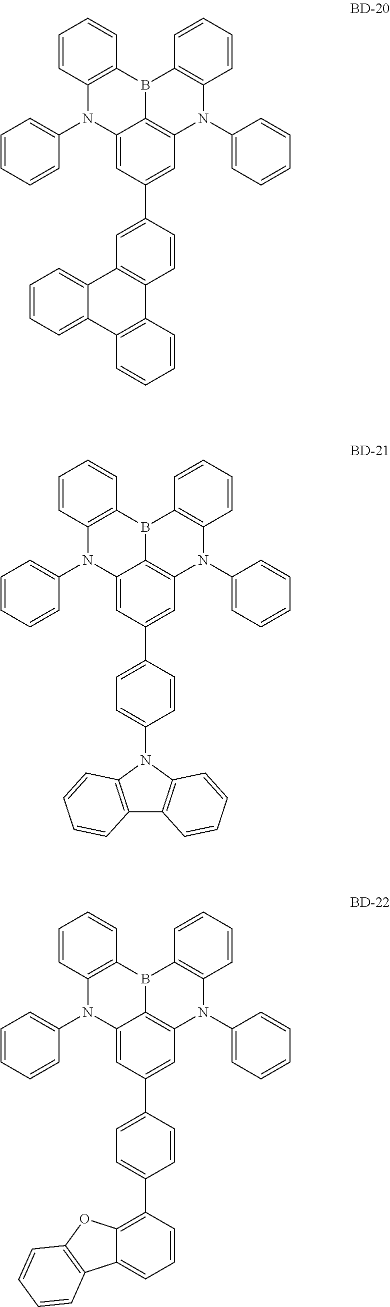 [SMILES: c1ccc(N2c3ccccc3B3c4ccccc4N(c4ccccc4)c4cc(-c5ccc6c7ccccc7c7ccccc7c6c5)cc2c43)cc1 ; c1ccc(N2c3ccccc3B3c4ccccc4N(c4ccccc4)c4cc(-c5ccc(-n6c7ccccc7c7ccccc76)cc5)cc2c43)cc1 ; c1ccc(N2c3ccccc3B3c4ccccc4N(c4ccccc4)c4cc(-c5ccc(-c6cccc7c6oc6ccccc67)cc5)cc2c43)cc1]

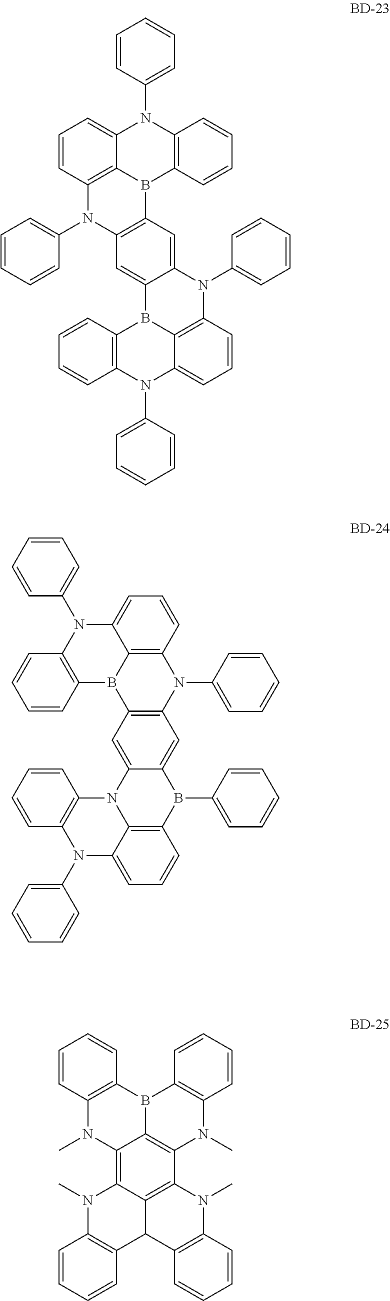 [SMILES: c1ccc(N2c3ccccc3B3c4cc5c(cc4N(c4ccccc4)c4cccc2c43)B2c3ccccc3N(c3ccccc3)c3cccc(c32)N5c2ccccc2)cc1 ; c1ccc(B2c3cc4c(cc3N3c5ccccc5N(c5ccccc5)c5cccc2c53)B2c3ccccc3N(c3ccccc3)c3cccc(c32)N4c2ccccc2)cc1 ; CN1c2ccccc2B2c3ccccc3N(C)c3c2c1c1c2c3N(C)c3ccccc3C2c2ccccc2N1C]

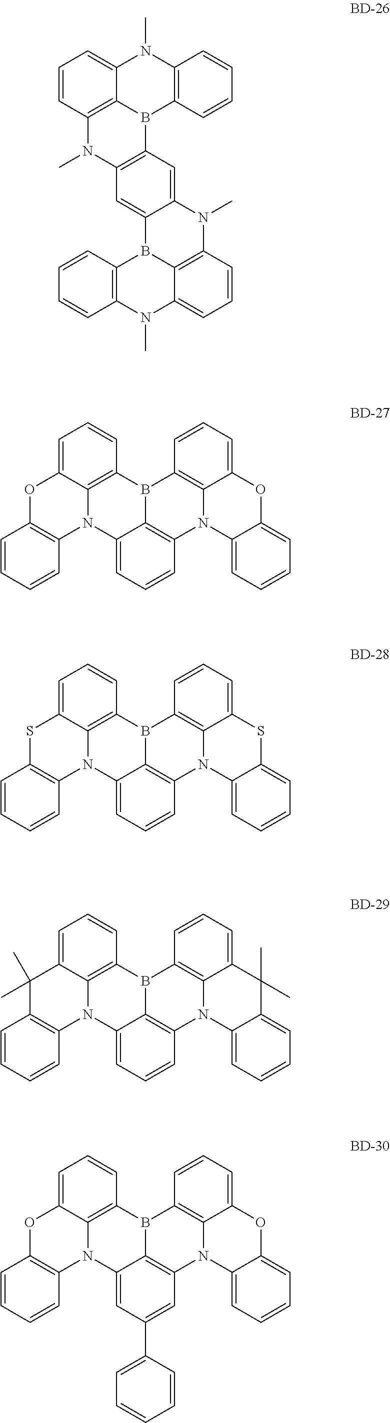 [SMILES: CN1c2ccccc2B2c3cc4c(cc3N(C)c3cccc1c32)B1c2ccccc2N(C)c2cccc(c21)N4C ; c1ccc2c(c1)Oc1cccc3c1N2c1cccc2c1B3c1cccc3c1N2c1ccccc1O3 ; c1ccc2c(c1)Sc1cccc3c1N2c1cccc2c1B3c1cccc3c1N2c1ccccc1S3 ; CC1(C)c2ccccc2N2c3cccc4c3B(c3cccc1c32)c1cccc2c1N4c1ccccc1C2(C)C ; c1ccc(-c2cc3c4c(c2)N2c5ccccc5Oc5cccc(c52)B4c2cccc4c2N3c2ccccc2O4)cc1]

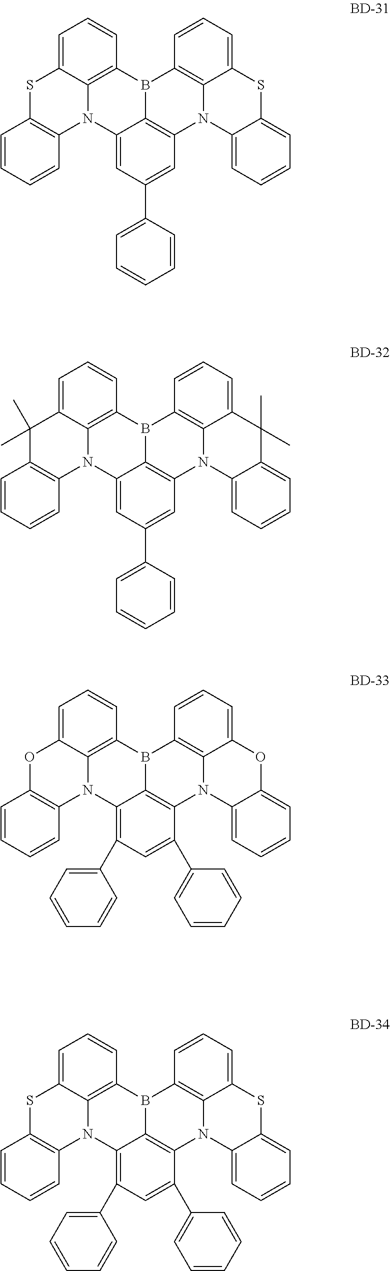 [SMILES: c1ccc(-c2cc3c4c(c2)N2c5ccccc5Sc5cccc(c52)B4c2cccc4c2N3c2ccccc2S4)cc1 ; CC1(C)c2ccccc2N2c3cc(-c4ccccc4)cc4c3B(c3cccc1c32)c1cccc2c1N4c1ccccc1C2(C)C ; c1ccc(-c2cc(-c3ccccc3)c3c4c2N2c5ccccc5Oc5cccc(c52)B4c2cccc4c2N3c2ccccc2O4)cc1 ; c1ccc(-c2cc(-c3ccccc3)c3c4c2N2c5ccccc5Sc5cccc(c52)B4c2cccc4c2N3c2ccccc2S4)cc1]

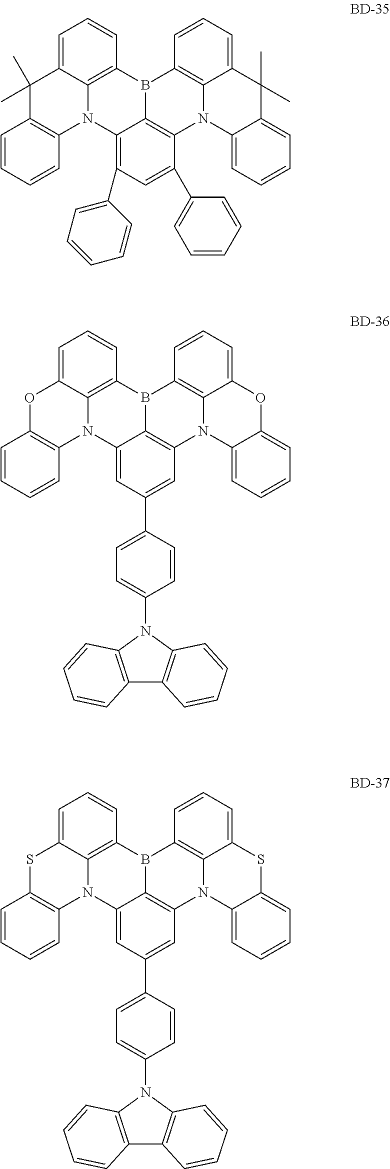 [SMILES: CC1(C)c2ccccc2N2c3c(cccc31)B1c3cccc4c3N(c3ccccc3C4(C)C)c3c(-c4ccccc4)cc(-c4ccccc4)c2c31 ; c1ccc2c(c1)Oc1cccc3c1N2c1cc(-c2ccc(-n4c5ccccc5c5ccccc54)cc2)cc2c1B3c1cccc3c1N2c1ccccc1O3 ; c1ccc2c(c1)Sc1cccc3c1N2c1cc(-c2ccc(-n4c5ccccc5c5ccccc54)cc2)cc2c1B3c1cccc3c1N2c1ccccc1S3]

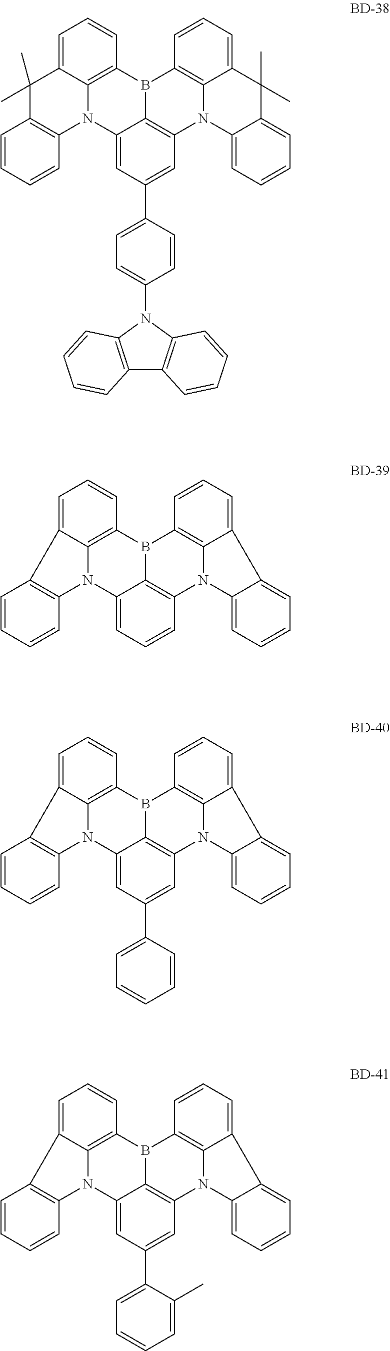 [SMILES: CC1(C)c2ccccc2N2c3cc(-c4ccc(-n5c6ccccc6c6ccccc65)cc4)cc4c3B(c3cccc1c32)c1cccc2c1N4c1ccccc1C2(C)C ; c1cc2c3c(c1)-n1c4ccccc4c4cccc(c41)B3c1cccc3c4ccccc4n-2c13 ; c1ccc(-c2cc3c4c(c2)-n2c5ccccc5c5cccc(c52)B4c2cccc4c5ccccc5n-3c24)cc1 ; Cc1ccccc1-c1cc2c3c(c1)-n1c4ccccc4c4cccc(c41)B3c1cccc3c4ccccc4n-2c13]

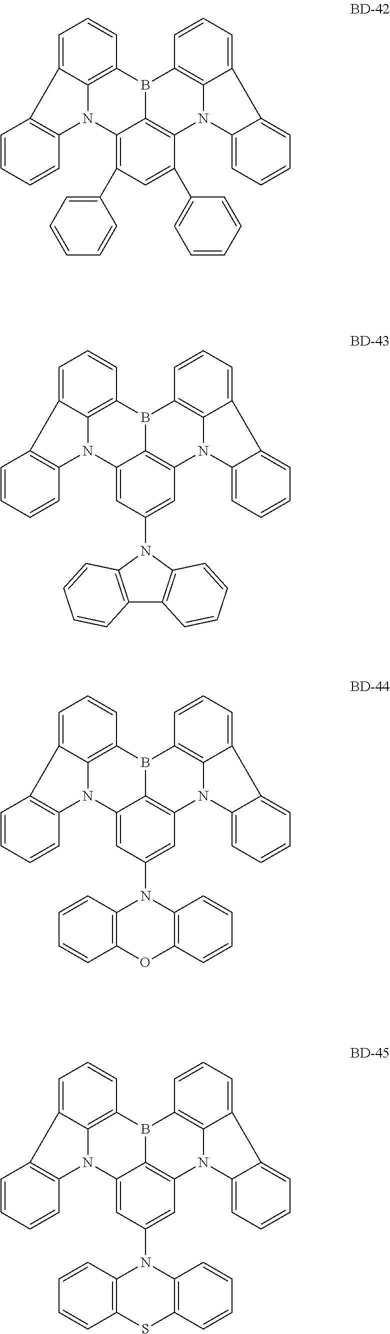 [SMILES: c1ccc(-c2cc(-c3ccccc3)c3c4c2-n2c5ccccc5c5cccc(c52)B4c2cccc4c5ccccc5n-3c24)cc1 ; c1ccc2c(c1)c1ccccc1n2-c1cc2c3c(c1)-n1c4ccccc4c4cccc(c41)B3c1cccc3c4ccccc4n-2c13 ; c1ccc2c(c1)Oc1ccccc1N2c1cc2c3c(c1)-n1c4ccccc4c4cccc(c41)B3c1cccc3c4ccccc4n-2c13 ; c1ccc2c(c1)Sc1ccccc1N2c1cc2c3c(c1)-n1c4ccccc4c4cccc(c41)B3c1cccc3c4ccccc4n-2c13]

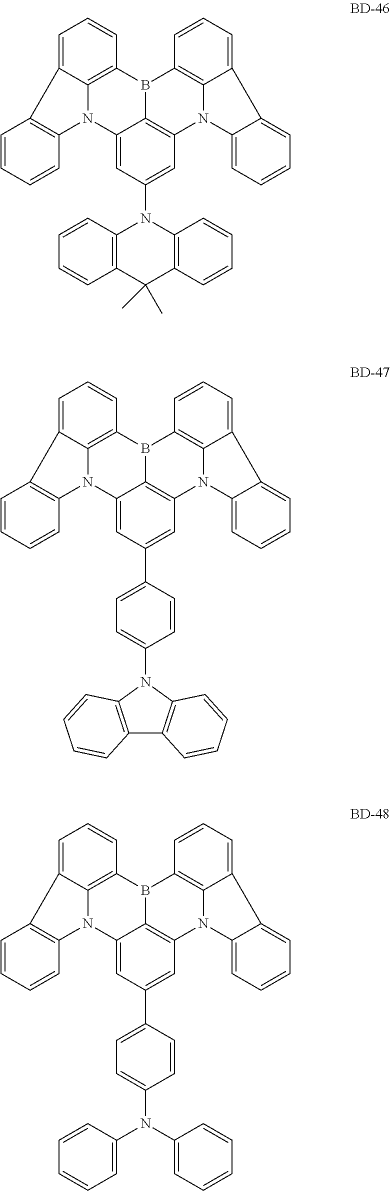 [SMILES: CC1(C)c2ccccc2N(c2cc3c4c(c2)-n2c5ccccc5c5cccc(c52)B4c2cccc4c5ccccc5n-3c24)c2ccccc21 ; c1ccc2c(c1)c1ccccc1n2-c1ccc(-c2cc3c4c(c2)-n2c5ccccc5c5cccc(c52)B4c2cccc4c5ccccc5n-3c24)cc1 ; c1ccc(N(c2ccccc2)c2ccc(-c3cc4c5c(c3)-n3c6ccccc6c6cccc(c63)B5c3cccc5c6ccccc6n-4c35)cc2)cc1]

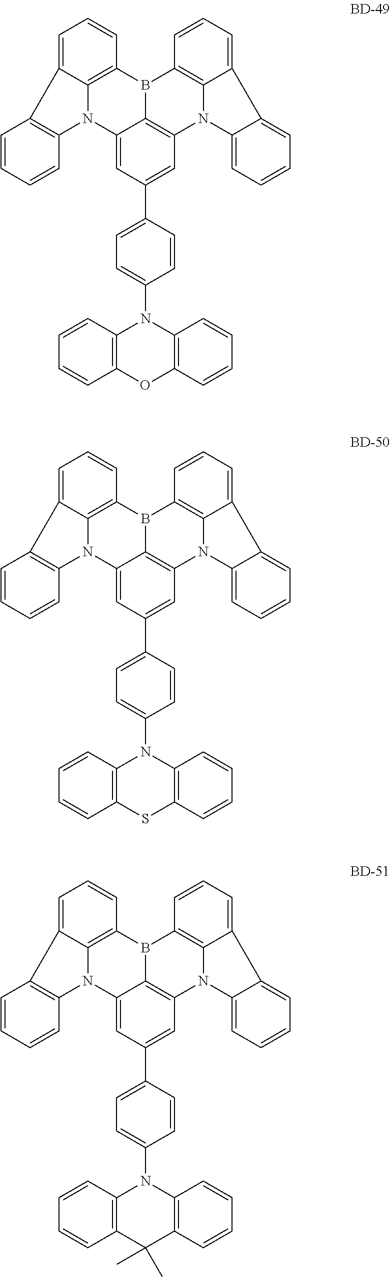 [SMILES: c1ccc2c(c1)Oc1ccccc1N2c1ccc(-c2cc3c4c(c2)-n2c5ccccc5c5cccc(c52)B4c2cccc4c5ccccc5n-3c24)cc1 ; c1ccc2c(c1)Sc1ccccc1N2c1ccc(-c2cc3c4c(c2)-n2c5ccccc5c5cccc(c52)B4c2cccc4c5ccccc5n-3c24)cc1 ; CC1(C)c2ccccc2N(c2ccc(-c3cc4c5c(c3)-n3c6ccccc6c6cccc(c63)B5c3cccc5c6ccccc6n-4c35)cc2)c2ccccc21]

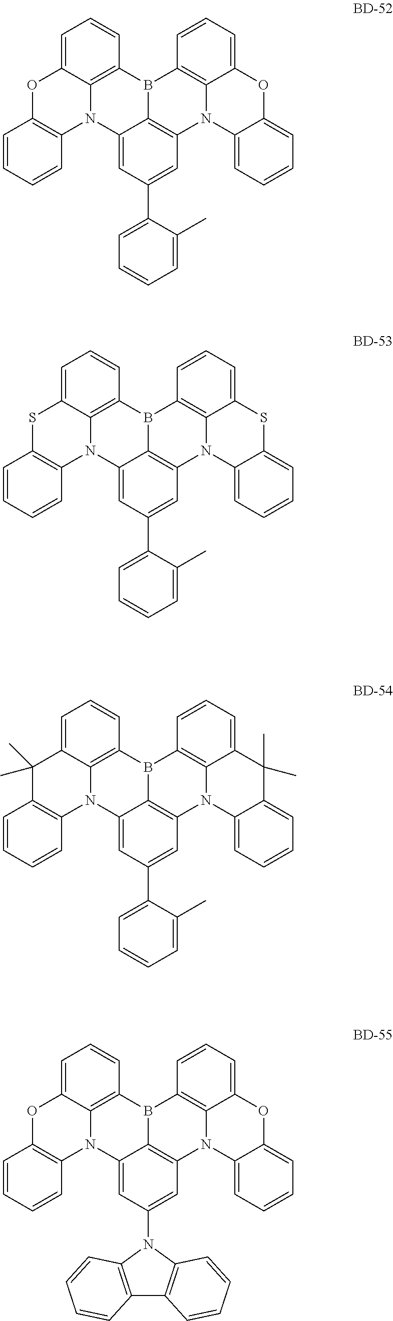

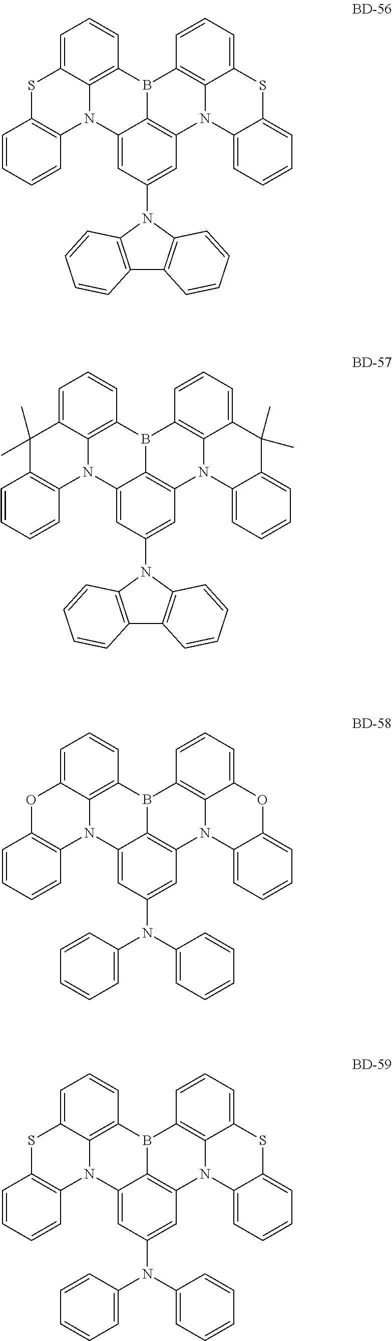 [SMILES: c1ccc2c(c1)Sc1cccc3c1N2c1cc(-n2c4ccccc4c4ccccc42)cc2c1B3c1cccc3c1N2c1ccccc1S3 ; CC1(C)c2ccccc2N2c3cc(-n4c5ccccc5c5ccccc54)cc4c3B(c3cccc1c32)c1cccc2c1N4c1ccccc1C2(C)C ; c1ccc(N(c2ccccc2)c2cc3c4c(c2)N2c5ccccc5Oc5cccc(c52)B4c2cccc4c2N3c2ccccc2O4)cc1 ; c1ccc(N(c2ccccc2)c2cc3c4c(c2)N2c5ccccc5Sc5cccc(c52)B4c2cccc4c2N3c2ccccc2S4)cc1]

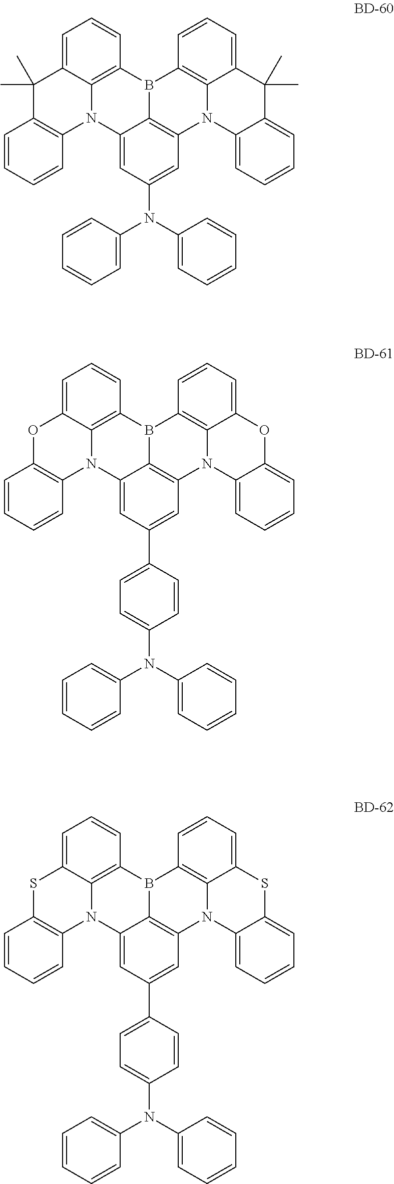

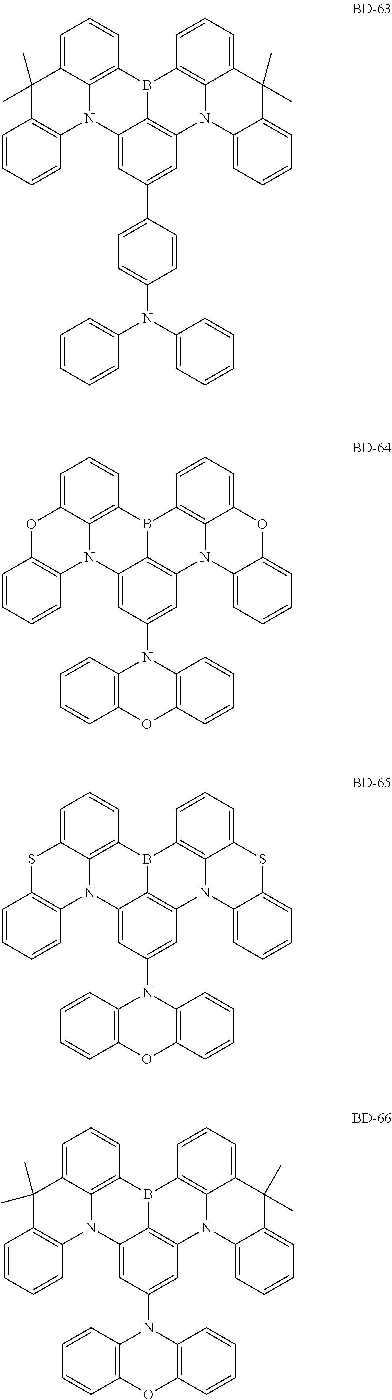

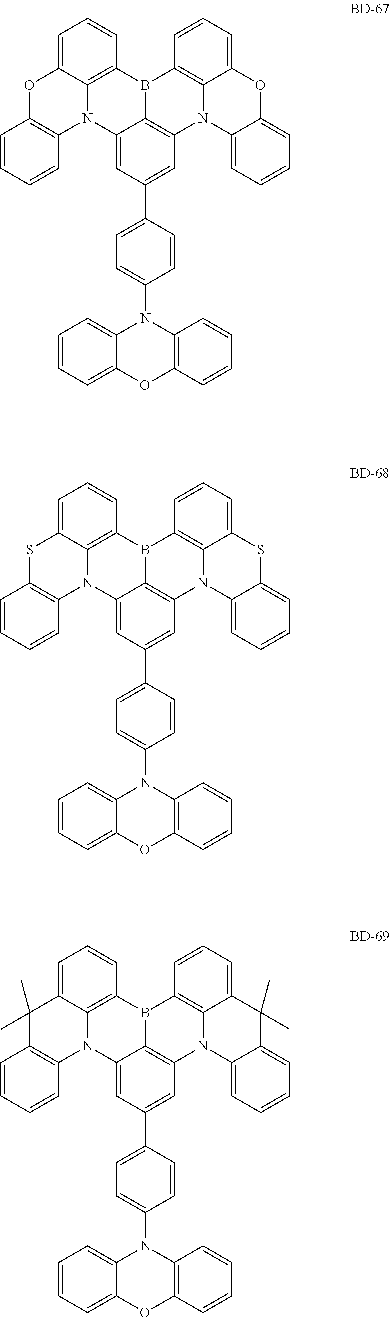 [SMILES: c1ccc2c(c1)Oc1ccccc1N2c1ccc(-c2cc3c4c(c2)N2c5ccccc5Oc5cccc(c52)B4c2cccc4c2N3c2ccccc2O4)cc1 ; c1ccc2c(c1)Oc1ccccc1N2c1ccc(-c2cc3c4c(c2)N2c5ccccc5Sc5cccc(c52)B4c2cccc4c2N3c2ccccc2S4)cc1 ; CC1(C)c2ccccc2N2c3cc(-c4ccc(N5c6ccccc6Oc6ccccc65)cc4)cc4c3B(c3cccc1c32)c1cccc2c1N4c1ccccc1C2(C)C]

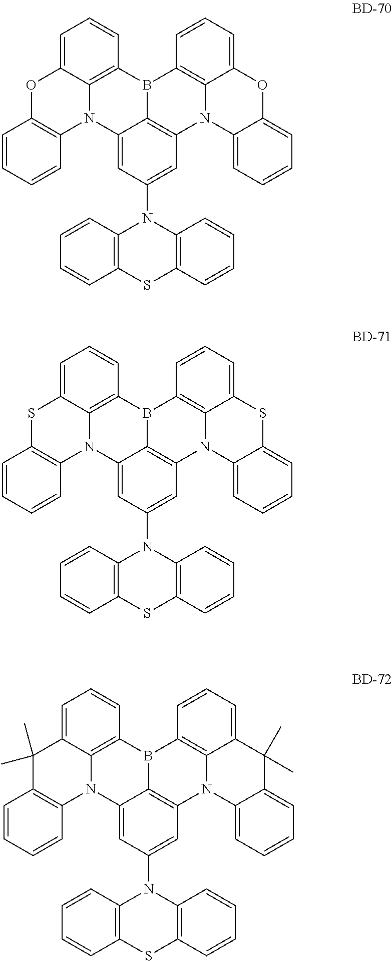

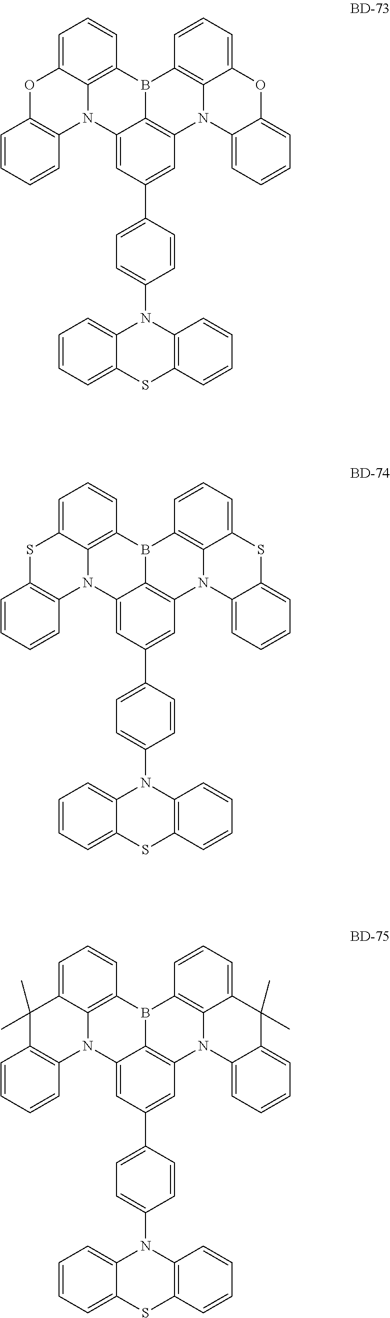 [SMILES: c1ccc2c(c1)Oc1cccc3c1N2c1cc(-c2ccc(N4c5ccccc5Sc5ccccc54)cc2)cc2c1B3c1cccc3c1N2c1ccccc1O3 ; c1ccc2c(c1)Sc1ccccc1N2c1ccc(-c2cc3c4c(c2)N2c5ccccc5Sc5cccc(c52)B4c2cccc4c2N3c2ccccc2S4)cc1 ; CC1(C)c2ccccc2N2c3cc(-c4ccc(N5c6ccccc6Sc6ccccc65)cc4)cc4c3B(c3cccc1c32)c1cccc2c1N4c1ccccc1C2(C)C]

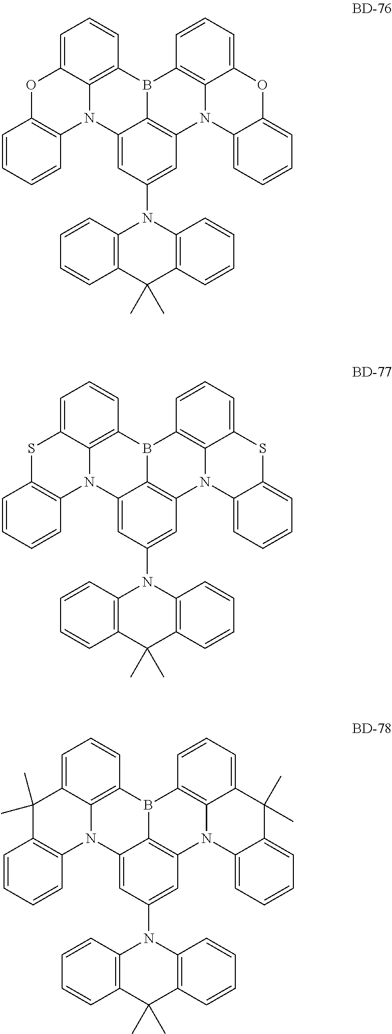

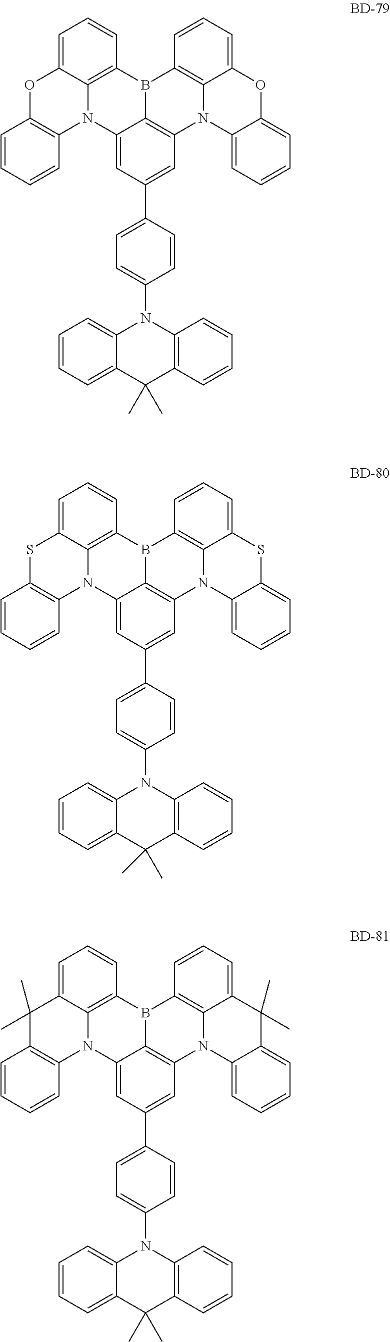 [SMILES: CC1(C)c2ccccc2N(c2ccc(-c3cc4c5c(c3)N3c6ccccc6Oc6cccc(c63)B5c3cccc5c3N4c3ccccc3O5)cc2)c2ccccc21 ; CC1(C)c2ccccc2N(c2ccc(-c3cc4c5c(c3)N3c6ccccc6Sc6cccc(c63)B5c3cccc5c3N4c3ccccc3S5)cc2)c2ccccc21 ; CC1(C)c2ccccc2N(c2ccc(-c3cc4c5c(c3)N3c6ccccc6C(C)(C)c6cccc(c63)B5c3cccc5c3N4c3ccccc3C5(C)C)cc2)c2ccccc21]

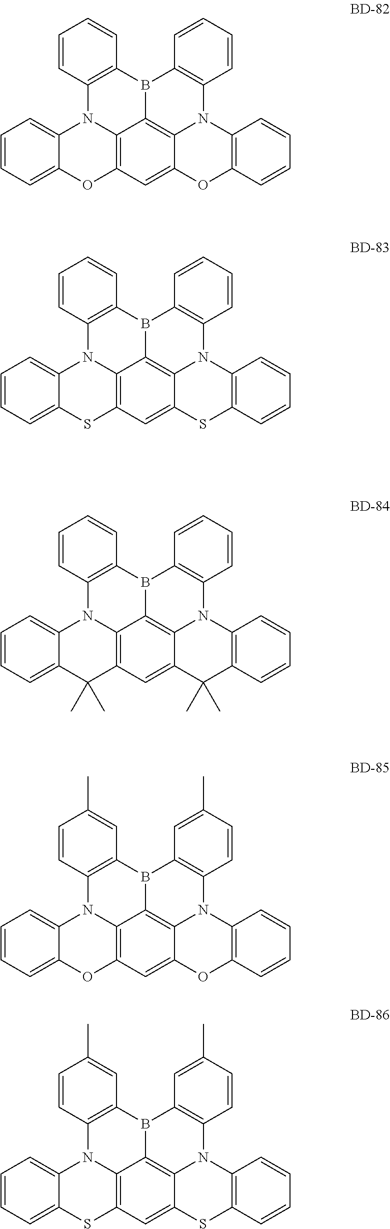 [SMILES: c1ccc2c(c1)B1c3ccccc3-n3c4ccccc4oc4cc5oc6ccccc6n-2c5c1c43 ; c1ccc2c(c1)B1c3ccccc3-n3c4ccccc4sc4cc5sc6ccccc6n-2c5c1c43 ; CC1(C)c2ccccc2N2c3ccccc3B3c4ccccc4N4c5ccccc5C(C)(C)c5cc1c2c3c54 ; Cc1ccc2c(c1)B1c3cc(C)ccc3-n3c4ccccc4oc4cc5oc6ccccc6n-2c5c1c43 ; Cc1ccc2c(c1)B1c3cc(C)ccc3-n3c4ccccc4sc4cc5sc6ccccc6n-2c5c1c43]

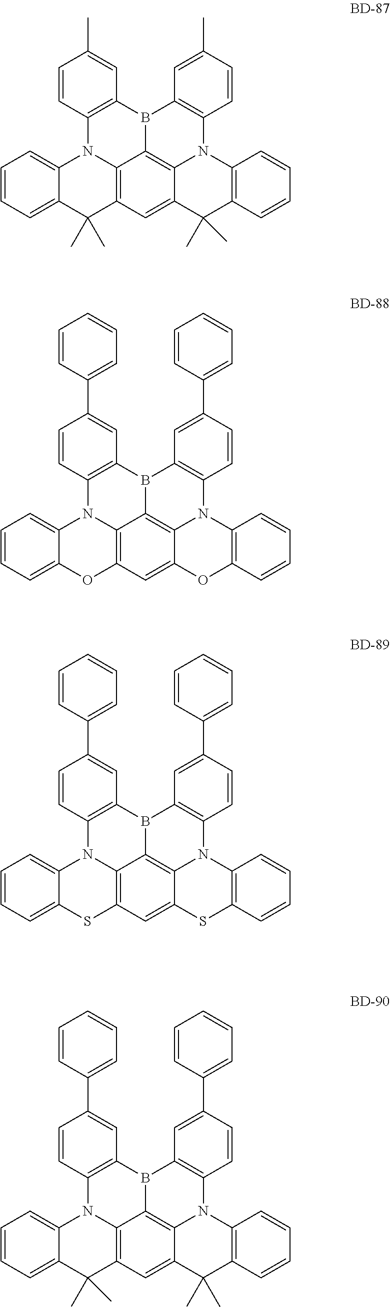 [SMILES: Cc1ccc2c(c1)B1c3cc(C)ccc3N3c4ccccc4C(C)(C)c4cc5c(c1c43)N2c1ccccc1C5(C)C ; c1ccc(-c2ccc3c(c2)B2c4cc(-c5ccccc5)ccc4-n4c5ccccc5oc5cc6oc7ccccc7n-3c6c2c54)cc1 ; c1ccc(-c2ccc3c(c2)B2c4cc(-c5ccccc5)ccc4-n4c5ccccc5sc5cc6sc7ccccc7n-3c6c2c54)cc1 ; CC1(C)c2ccccc2N2c3ccc(-c4ccccc4)cc3B3c4cc(-c5ccccc5)ccc4N4c5ccccc5C(C)(C)c5cc1c2c3c54]

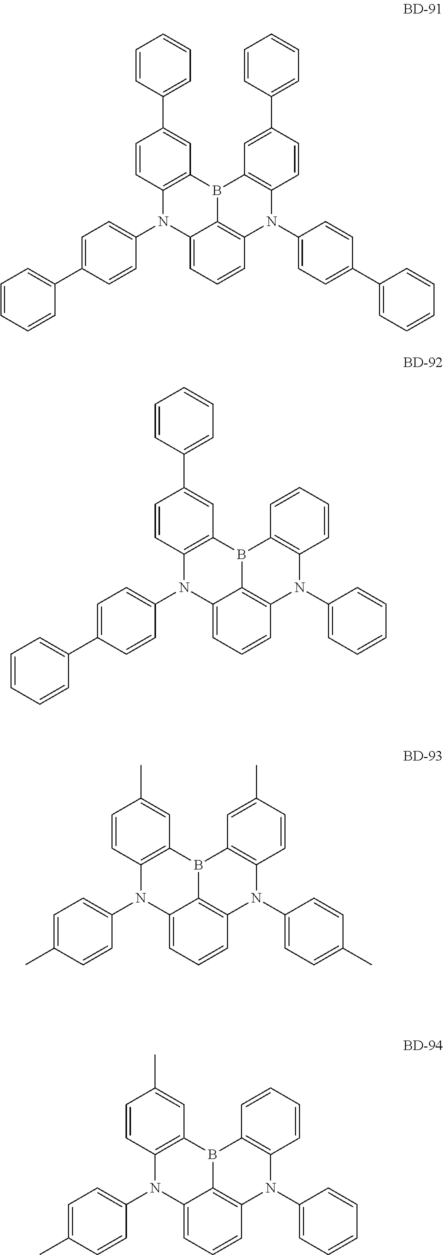

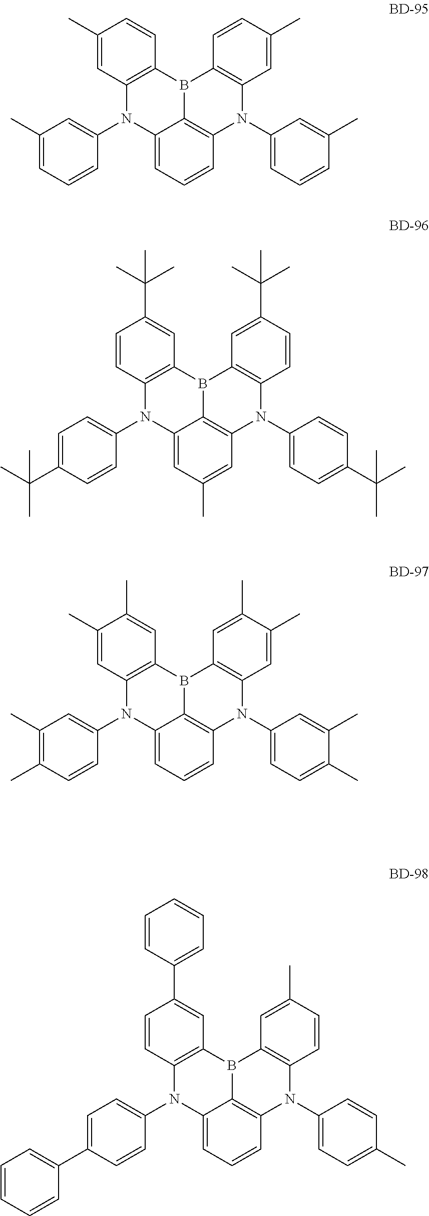

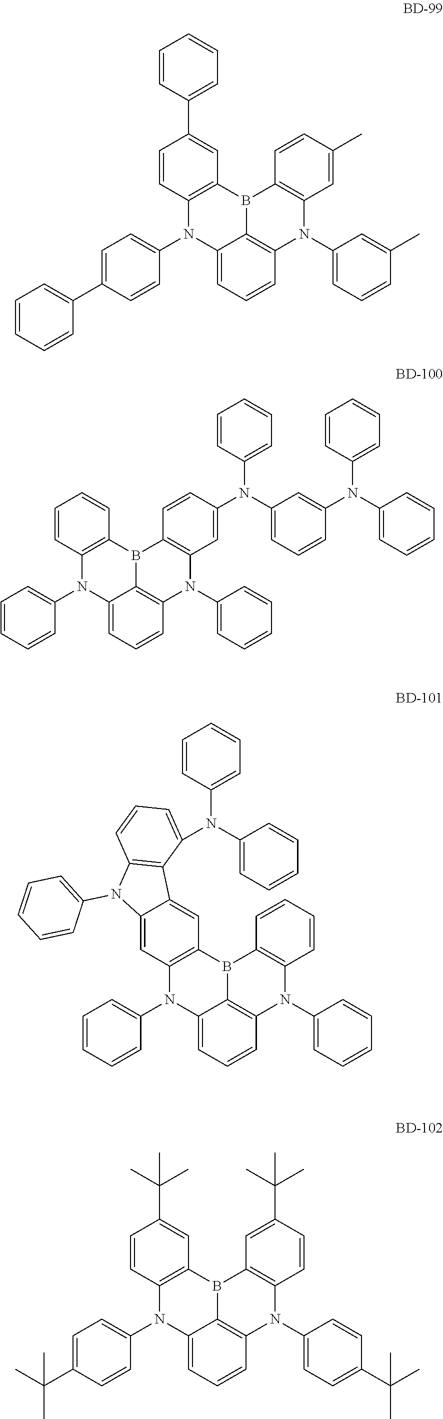

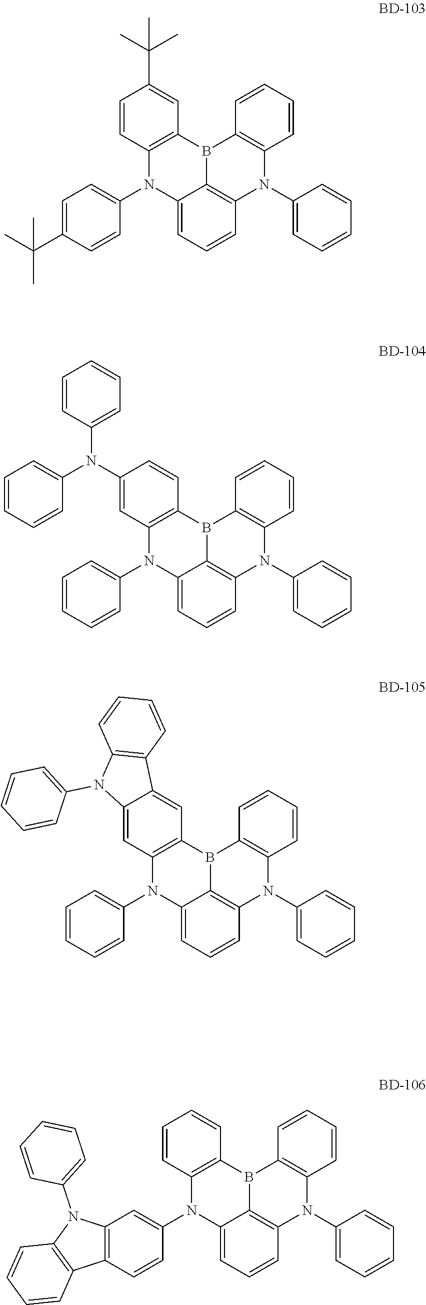 [SMILES: CC(C)(C)c1ccc(N2c3ccc(C(C)(C)C)cc3B3c4ccccc4N(c4ccccc4)c4cccc2c43)cc1 ; c1ccc(N(c2ccccc2)c2ccc3c(c2)N(c2ccccc2)c2cccc4c2B3c2ccccc2N4c2ccccc2)cc1 ; c1ccc(N2c3ccccc3B3c4cc5c6ccccc6n(-c6ccccc6)c5cc4N(c4ccccc4)c4cccc2c43)cc1 ; c1ccc(N2c3ccccc3B3c4ccccc4N(c4ccc5c6ccccc6n(-c6ccccc6)c5c4)c4cccc2c43)cc1]

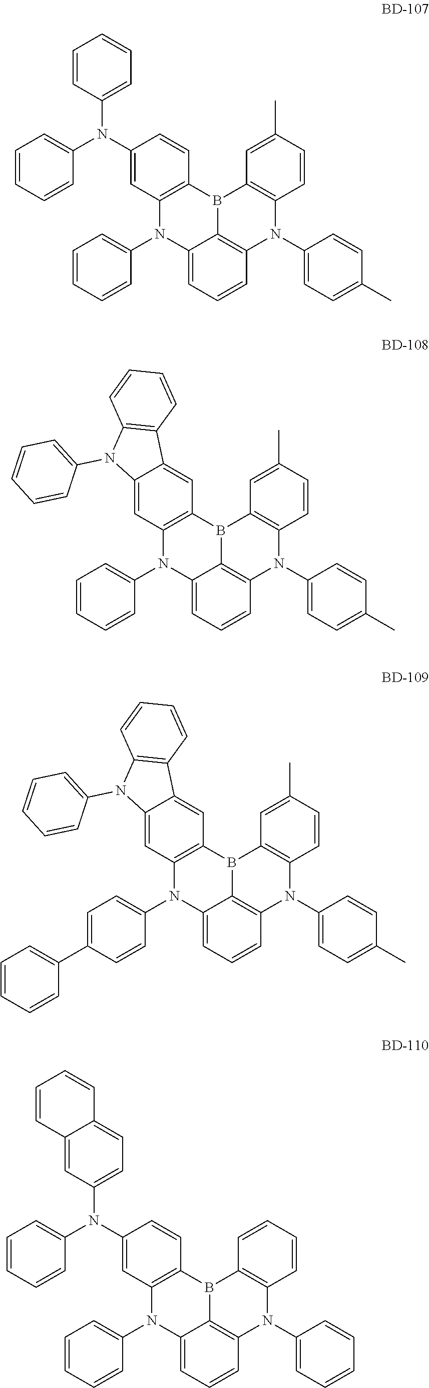 [SMILES: Cc1ccc(N2c3ccc(C)cc3B3c4ccc(N(c5ccccc5)c5ccccc5)cc4N(c4ccccc4)c4cccc2c43)cc1 ; Cc1ccc(N2c3ccc(C)cc3B3c4cc5c6ccccc6n(-c6ccccc6)c5cc4N(c4ccccc4)c4cccc2c43)cc1 ; Cc1ccc(N2c3ccc(C)cc3B3c4cc5c6ccccc6n(-c6ccccc6)c5cc4N(c4ccc(-c5ccccc5)cc4)c4cccc2c43)cc1 ; c1ccc(N(c2ccc3c(c2)N(c2ccccc2)c2cccc4c2B3c2ccccc2N4c2ccccc2)c2ccc3ccccc3c2)cc1]

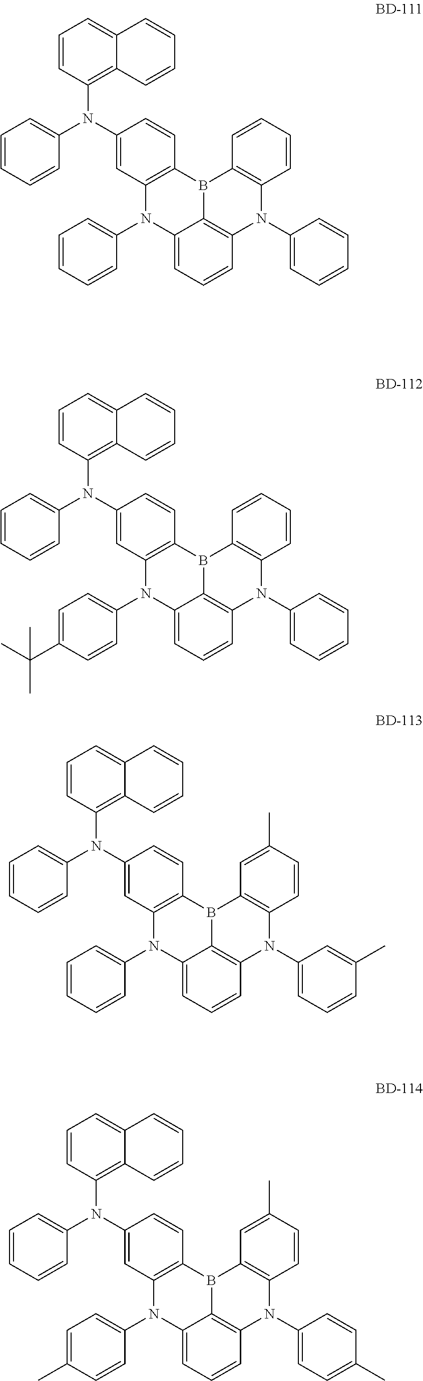 [SMILES: c1ccc(N2c3ccccc3B3c4ccc(N(c5ccccc5)c5cccc6ccccc56)cc4N(c4ccccc4)c4cccc2c43)cc1 ; CC(C)(C)c1ccc(N2c3cc(N(c4ccccc4)c4cccc5ccccc45)ccc3B3c4ccccc4N(c4ccccc4)c4cccc2c43)cc1 ; Cc1cccc(N2c3ccc(C)cc3B3c4ccc(N(c5ccccc5)c5cccc6ccccc56)cc4N(c4ccccc4)c4cccc2c43)c1 ; Cc1ccc(N2c3ccc(C)cc3B3c4ccc(N(c5ccccc5)c5cccc6ccccc56)cc4N(c4ccc(C)cc4)c4cccc2c43)cc1]

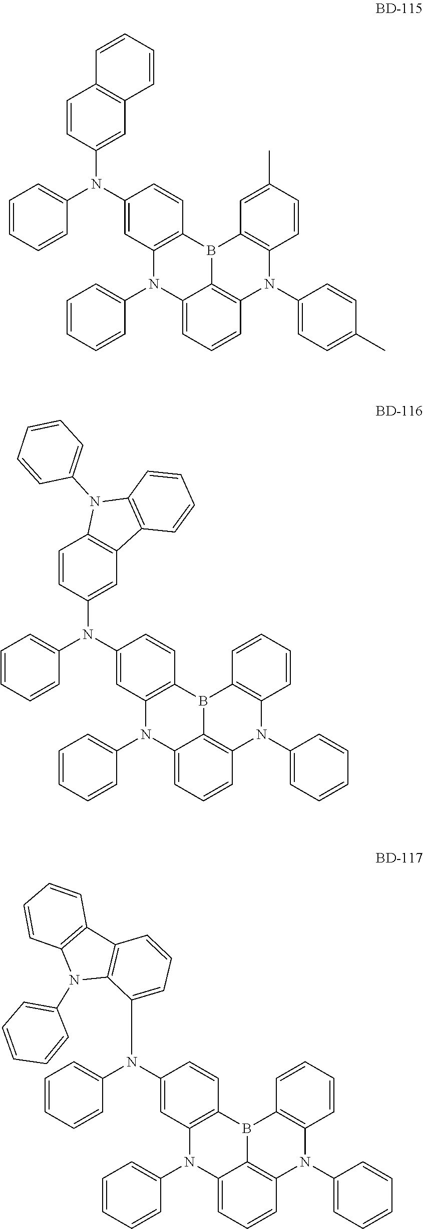 [SMILES: Cc1ccc(N2c3ccc(C)cc3B3c4ccc(N(c5ccccc5)c5ccc6ccccc6c5)cc4N(c4ccccc4)c4cccc2c43)cc1 ; c1ccc(N(c2ccc3c(c2)N(c2ccccc2)c2cccc4c2B3c2ccccc2N4c2ccccc2)c2ccc3c(c2)c2ccccc2n3-c2ccccc2)cc1 ; c1ccc(N2c3ccccc3B3c4ccc(N(c5ccccc5)c5cccc6c7ccccc7n(-c7ccccc7)c56)cc4N(c4ccccc4)c4cccc2c43)cc1]

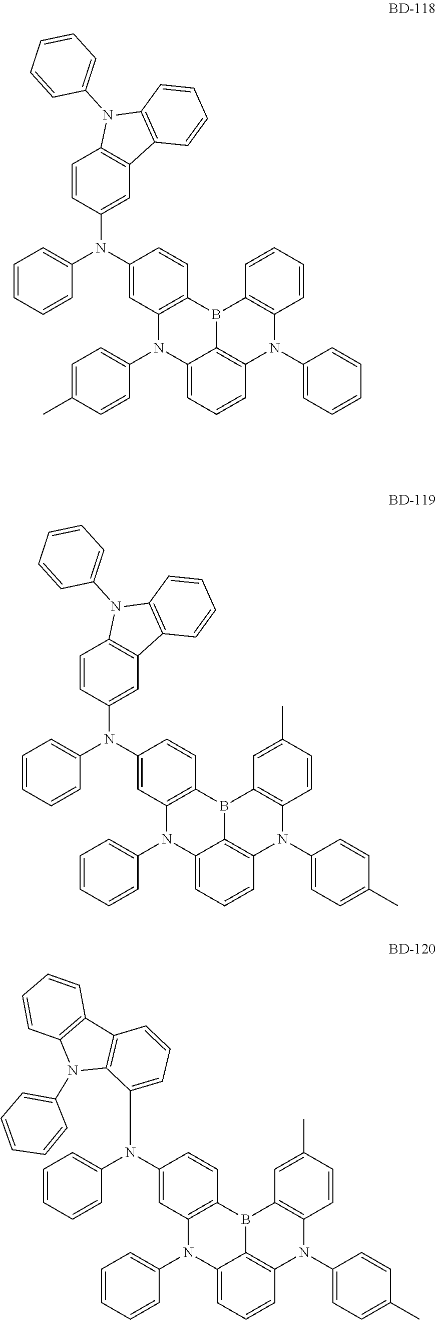 [SMILES: Cc1ccc(N2c3cc(N(c4ccccc4)c4ccc5c(c4)c4ccccc4n5-c4ccccc4)ccc3B3c4ccccc4N(c4ccccc4)c4cccc2c43)cc1 ; Cc1ccc(N2c3ccc(C)cc3B3c4ccc(N(c5ccccc5)c5ccc6c(c5)c5ccccc5n6-c5ccccc5)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccccc4)c4cccc2c43)cc1]

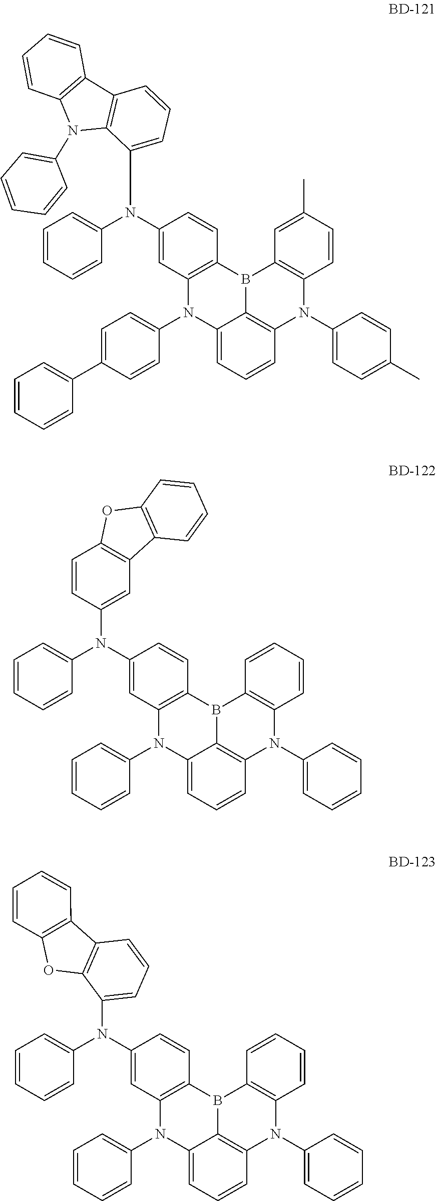 [SMILES: Cc1ccc(N2c3ccc(C)cc3B3c4ccc(N(c5ccccc5)c5cccc6c7ccccc7n(-c7ccccc7)c56)cc4N(c4ccc(-c5ccccc5)cc4)c4cccc2c43)cc1 ; c1ccc(N(c2ccc3c(c2)N(c2ccccc2)c2cccc4c2B3c2ccccc2N4c2ccccc2)c2ccc3oc4ccccc4c3c2)cc1 ; c1ccc(N2c3ccccc3B3c4ccc(N(c5ccccc5)c5cccc6c5oc5ccccc56)cc4N(c4ccccc4)c4cccc2c43)cc1]

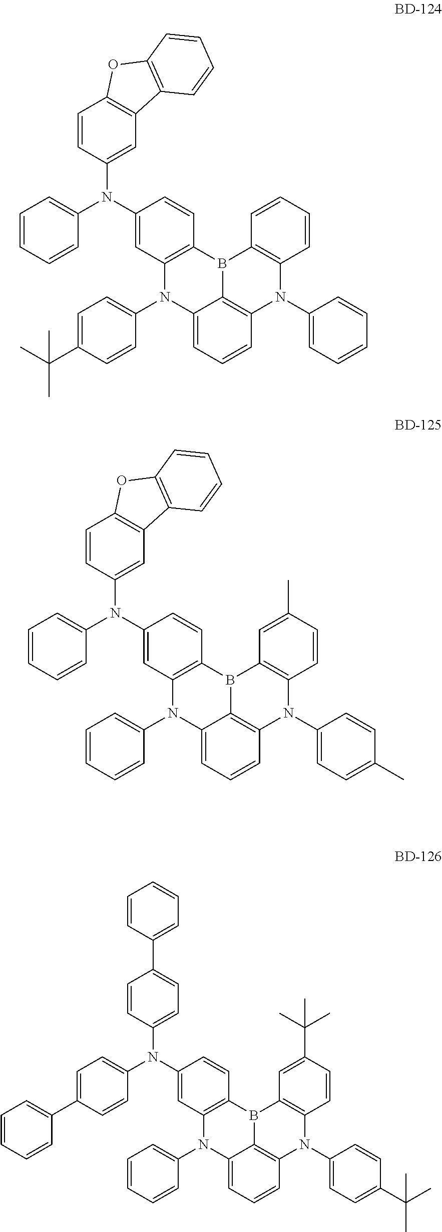 [SMILES: CC(C)(C)c1ccc(N2c3cc(N(c4ccccc4)c4ccc5oc6ccccc6c5c4)ccc3B3c4ccccc4N(c4ccccc4)c4cccc2c43)cc1 ; Cc1ccc(N2c3ccc(C)cc3B3c4ccc(N(c5ccccc5)c5ccc6oc7ccccc7c6c5)cc4N(c4ccccc4)c4cccc2c43)cc1 ; CC(C)(C)c1ccc(N2c3ccc(C(C)(C)C)cc3B3c4ccc(N(c5ccc(-c6ccccc6)cc5)c5ccc(-c6ccccc6)cc5)cc4N(c4ccccc4)c4cccc2c43)cc1]

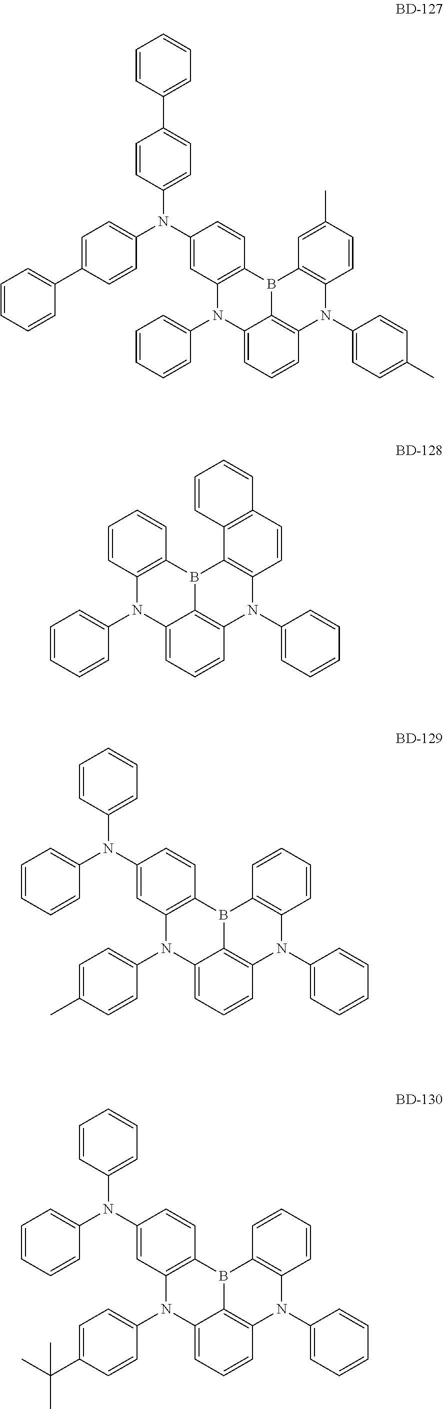 [SMILES: Cc1ccc(N2c3ccc(C)cc3B3c4ccc(N(c5ccc(-c6ccccc6)cc5)c5ccc(-c6ccccc6)cc5)cc4N(c4ccccc4)c4cccc2c43)cc1 ; c1ccc(N2c3ccccc3B3c4c2cccc4N(c2ccccc2)c2ccc4ccccc4c23)cc1 ; Cc1ccc(N2c3cc(N(c4ccccc4)c4ccccc4)ccc3B3c4ccccc4N(c4ccccc4)c4cccc2c43)cc1 ; CC(C)(C)c1ccc(N2c3cc(N(c4ccccc4)c4ccccc4)ccc3B3c4ccccc4N(c4ccccc4)c4cccc2c43)cc1]

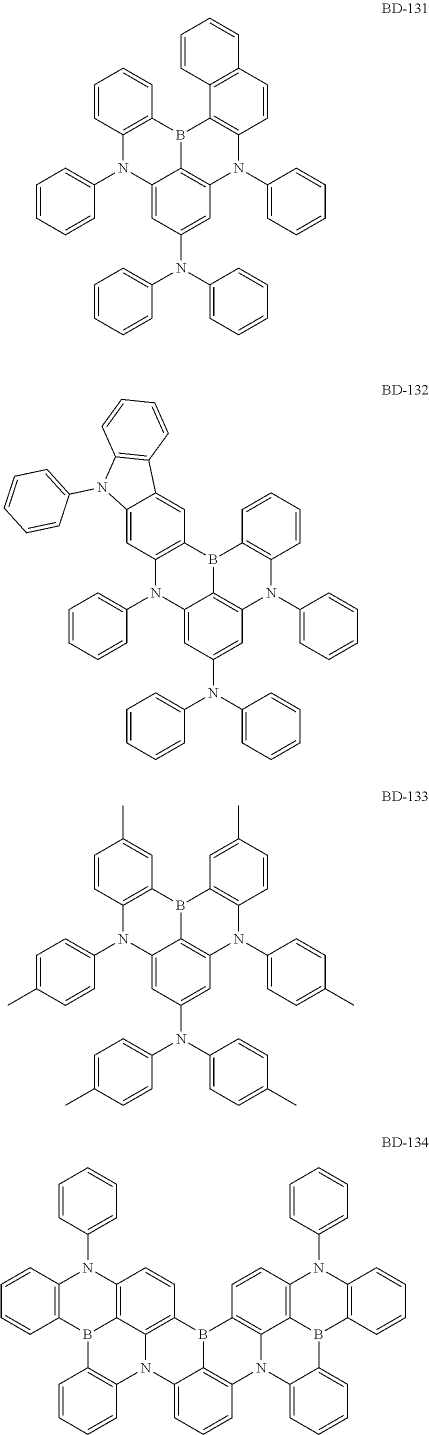

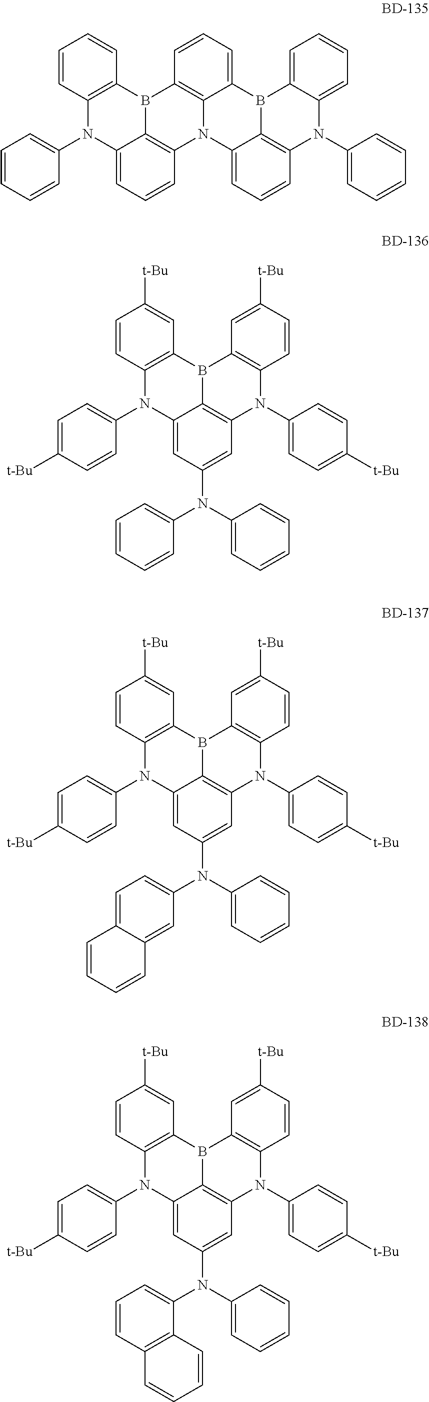 [SMILES: c1ccc(N2c3ccccc3B3c4cccc5c4N(c4cccc2c43)c2cccc3c2B5c2ccccc2N3c2ccccc2)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccc6ccccc6c5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5cccc6ccccc56)cc2c43)cc1]

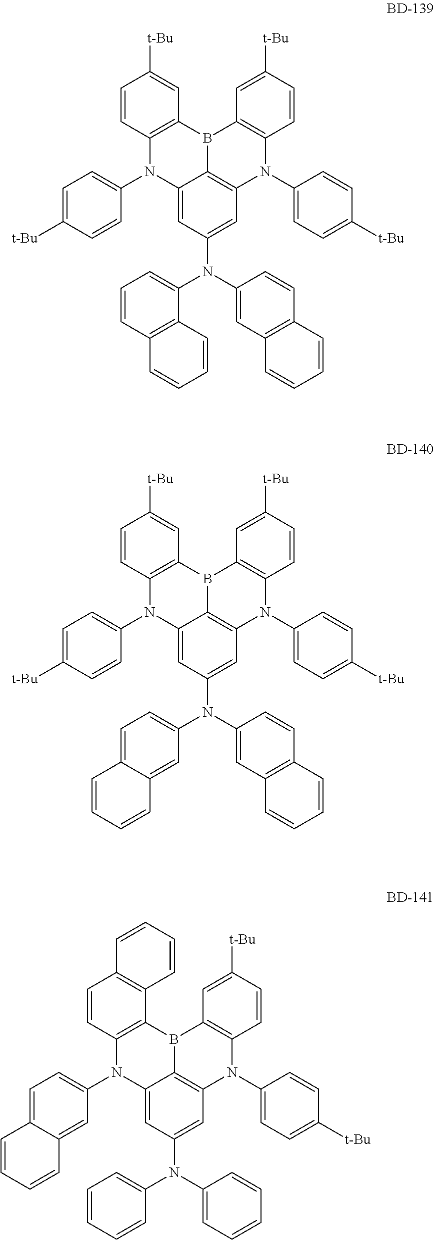 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccc6ccccc6c5)c5cccc6ccccc56)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccc6ccccc6c5)c5ccc6ccccc6c5)cc2c43)cc1 ; CC(C)(C)c1ccc(N2c3ccc(C(C)(C)C)cc3B3c4c2cc(N(c2ccccc2)c2ccccc2)cc4N(c2ccc4ccccc4c2)c2ccc4ccccc4c23)cc1]

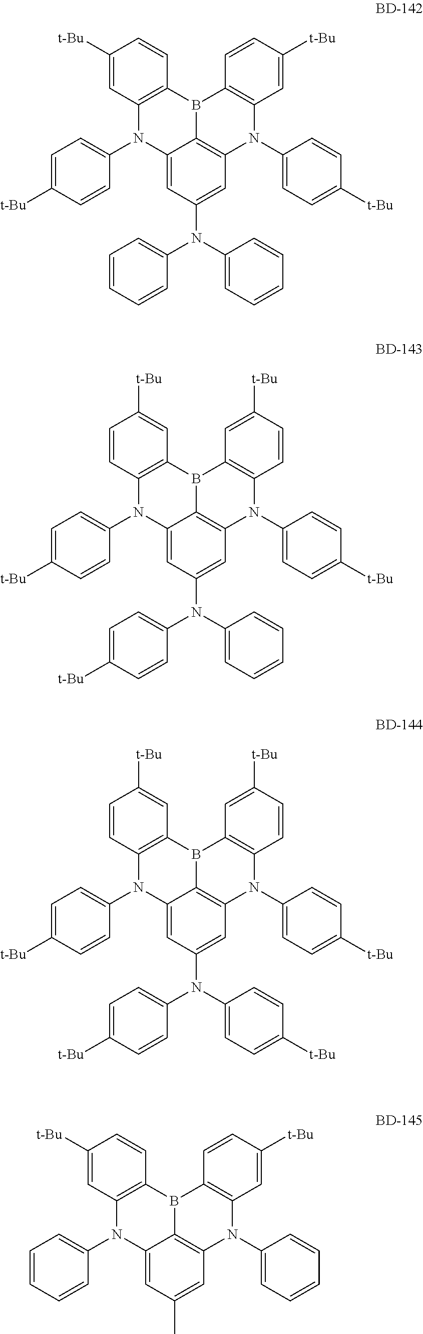 [SMILES: CC(C)(C)c1ccc(N2c3cc(C(C)(C)C)ccc3B3c4ccc(C(C)(C)C)cc4N(c4ccc(C(C)(C)C)cc4)c4cc(N(c5ccccc5)c5ccccc5)cc2c43)cc1 ; CC(C)(C)c1ccc(N(c2ccccc2)c2cc3c4c(c2)N(c2ccc(C(C)(C)C)cc2)c2ccc(C(C)(C)C)cc2B4c2cc(C(C)(C)C)ccc2N3c2ccc(C(C)(C)C)cc2)cc1 ; CC(C)(C)c1ccc(N(c2ccc(C(C)(C)C)cc2)c2cc3c4c(c2)N(c2ccc(C(C)(C)C)cc2)c2ccc(C(C)(C)C)cc2B4c2cc(C(C)(C)C)ccc2N3c2ccc(C(C)(C)C)cc2)cc1 ; Cc1cc2c3c(c1)N(c1ccccc1)c1cc(C(C)(C)C)ccc1B3c1ccc(C(C)(C)C)cc1N2c1ccccc1]

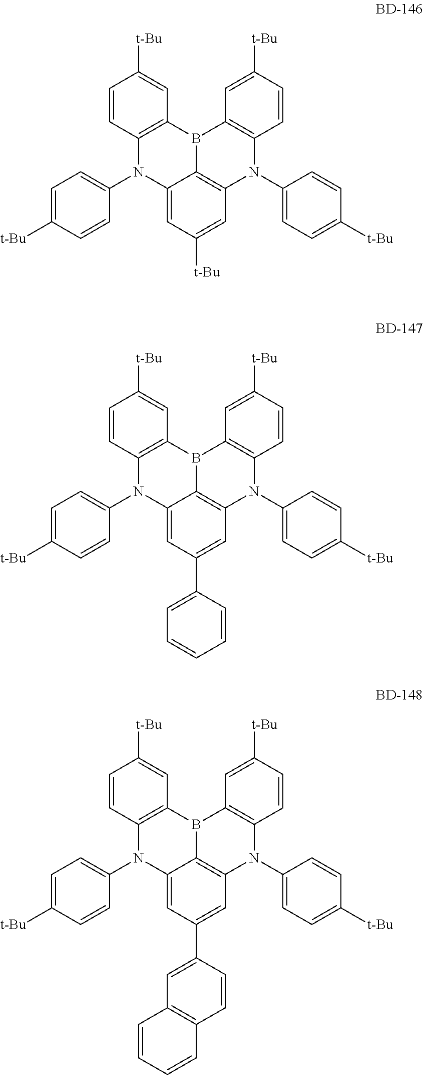 [SMILES: CC(C)(C)c1ccc(N2c3ccc(C(C)(C)C)cc3B3c4cc(C(C)(C)C)ccc4N(c4ccc(C(C)(C)C)cc4)c4cc(C(C)(C)C)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5ccccc5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5ccc6ccccc6c5)cc2c43)cc1]

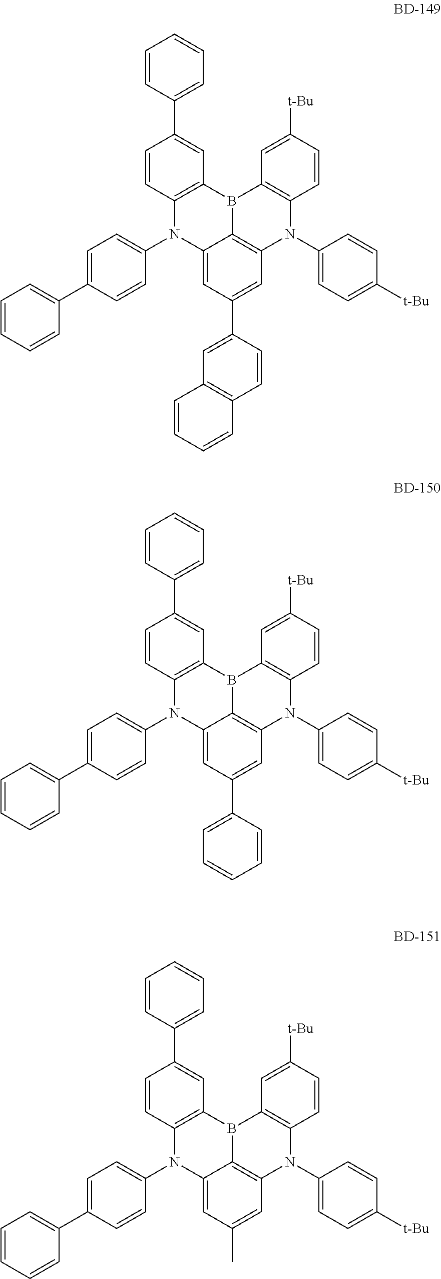 [SMILES: CC(C)(C)c1ccc(N2c3ccc(C(C)(C)C)cc3B3c4cc(-c5ccccc5)ccc4N(c4ccc(-c5ccccc5)cc4)c4cc(-c5ccc6ccccc6c5)cc2c43)cc1 ; CC(C)(C)c1ccc(N2c3ccc(C(C)(C)C)cc3B3c4cc(-c5ccccc5)ccc4N(c4ccc(-c5ccccc5)cc4)c4cc(-c5ccccc5)cc2c43)cc1 ; Cc1cc2c3c(c1)N(c1ccc(C(C)(C)C)cc1)c1ccc(C(C)(C)C)cc1B3c1cc(-c3ccccc3)ccc1N2c1ccc(-c2ccccc2)cc1]

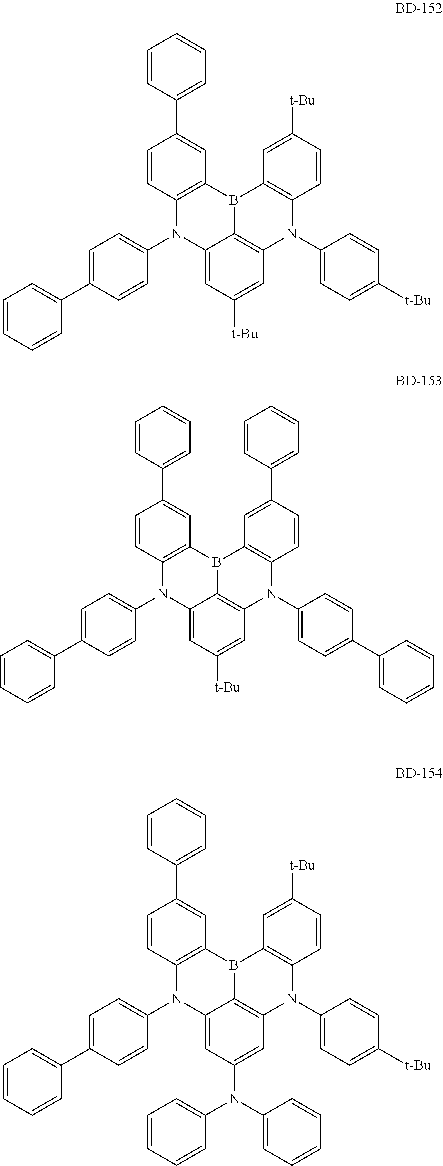 [SMILES: CC(C)(C)c1ccc(N2c3ccc(C(C)(C)C)cc3B3c4cc(-c5ccccc5)ccc4N(c4ccc(-c5ccccc5)cc4)c4cc(C(C)(C)C)cc2c43)cc1 ; CC(C)(C)c1cc2c3c(c1)N(c1ccc(-c4ccccc4)cc1)c1ccc(-c4ccccc4)cc1B3c1cc(-c3ccccc3)ccc1N2c1ccc(-c2ccccc2)cc1 ; CC(C)(C)c1ccc(N2c3ccc(C(C)(C)C)cc3B3c4cc(-c5ccccc5)ccc4N(c4ccc(-c5ccccc5)cc4)c4cc(N(c5ccccc5)c5ccccc5)cc2c43)cc1]

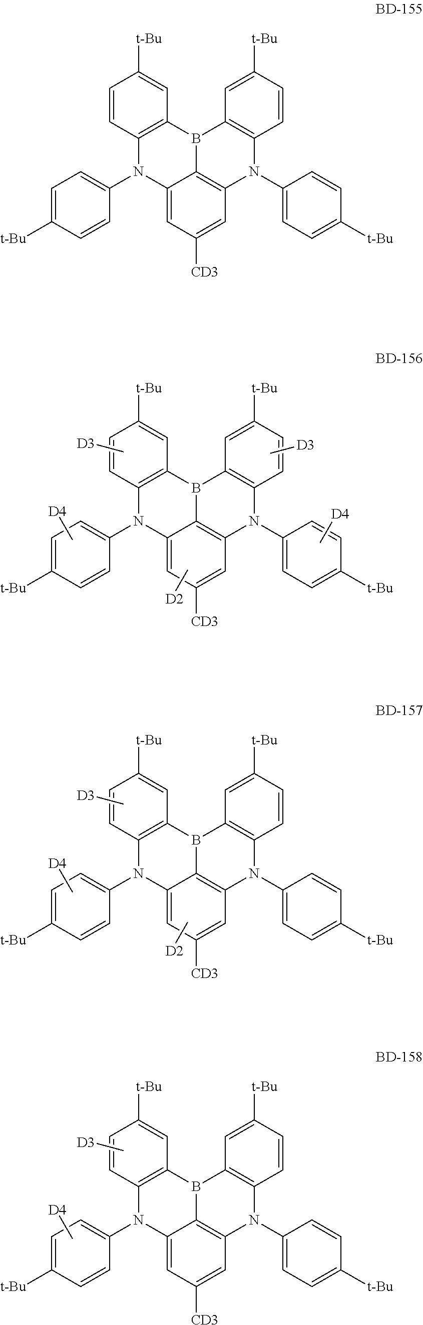

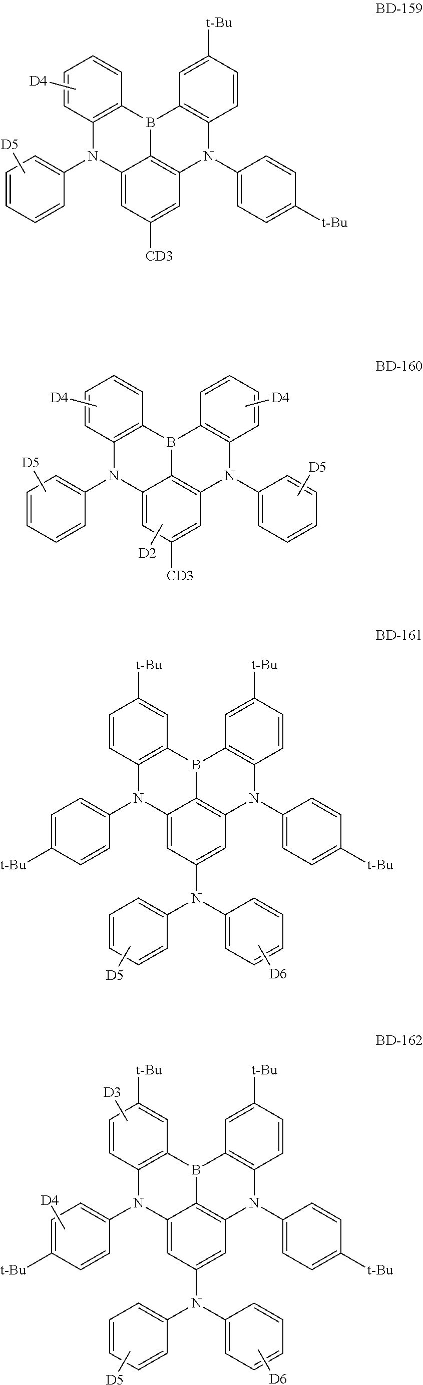

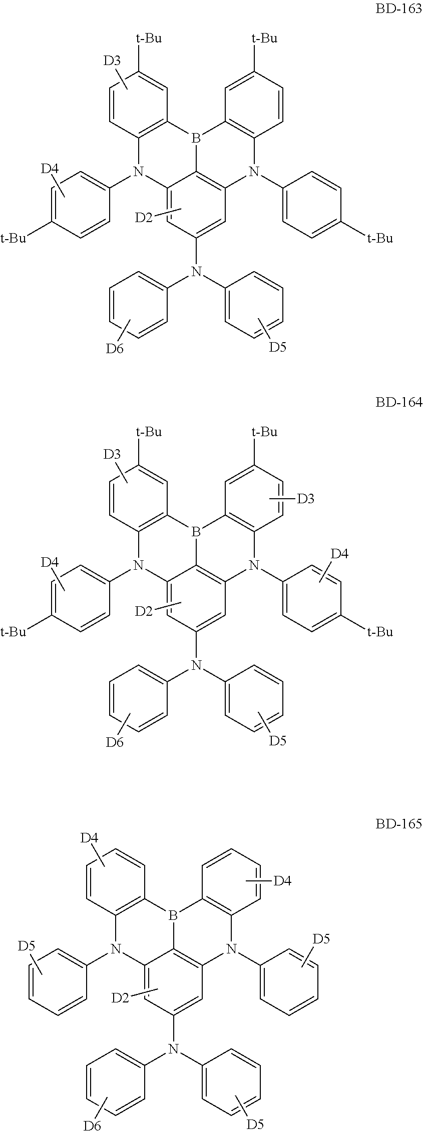

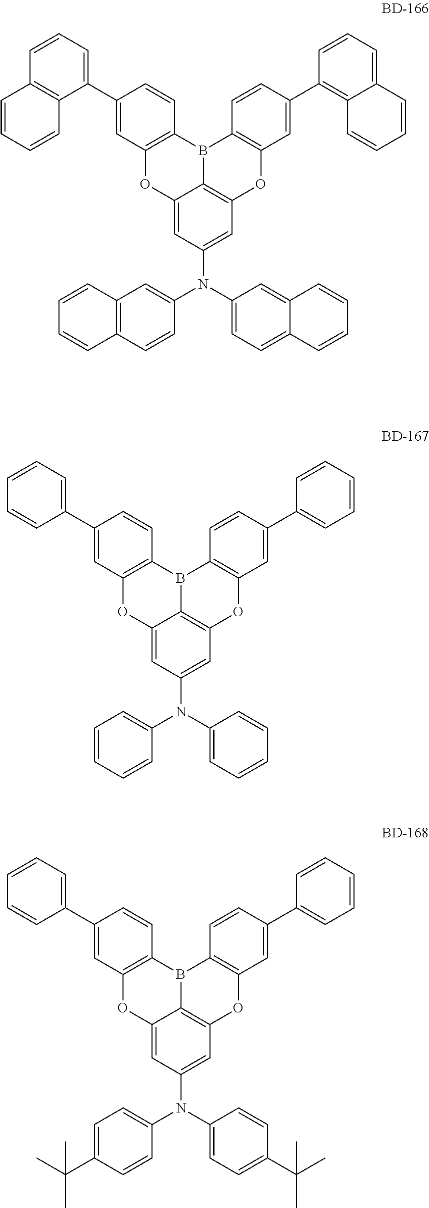 [SMILES: c1ccc2cc(N(c3cc4c5c(c3)Oc3cc(-c6cccc7ccccc67)ccc3B5c3ccc(-c5cccc6ccccc56)cc3O4)c3ccc4ccccc4c3)ccc2c1 ; c1ccc(-c2ccc3c(c2)Oc2cc(N(c4ccccc4)c4ccccc4)cc4c2B3c2ccc(-c3ccccc3)cc2O4)cc1 ; CC(C)(C)c1ccc(N(c2ccc(C(C)(C)C)cc2)c2cc3c4c(c2)Oc2cc(-c5ccccc5)ccc2B4c2ccc(-c4ccccc4)cc2O3)cc1]

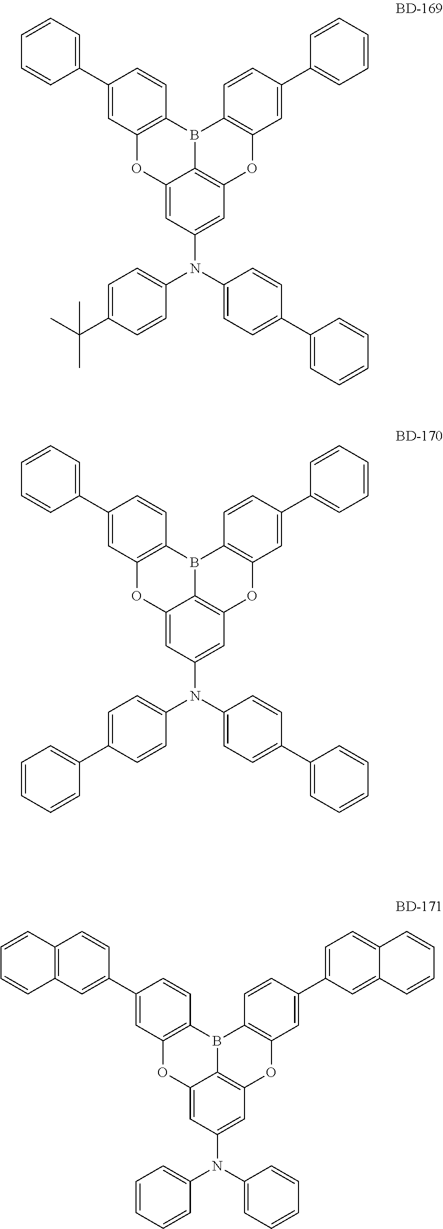 [SMILES: CC(C)(C)c1ccc(N(c2ccc(-c3ccccc3)cc2)c2cc3c4c(c2)Oc2cc(-c5ccccc5)ccc2B4c2ccc(-c4ccccc4)cc2O3)cc1 ; c1ccc(-c2ccc(N(c3ccc(-c4ccccc4)cc3)c3cc4c5c(c3)Oc3cc(-c6ccccc6)ccc3B5c3ccc(-c5ccccc5)cc3O4)cc2)cc1 ; c1ccc(N(c2ccccc2)c2cc3c4c(c2)Oc2cc(-c5ccc6ccccc6c5)ccc2B4c2ccc(-c4ccc5ccccc5c4)cc2O3)cc1]

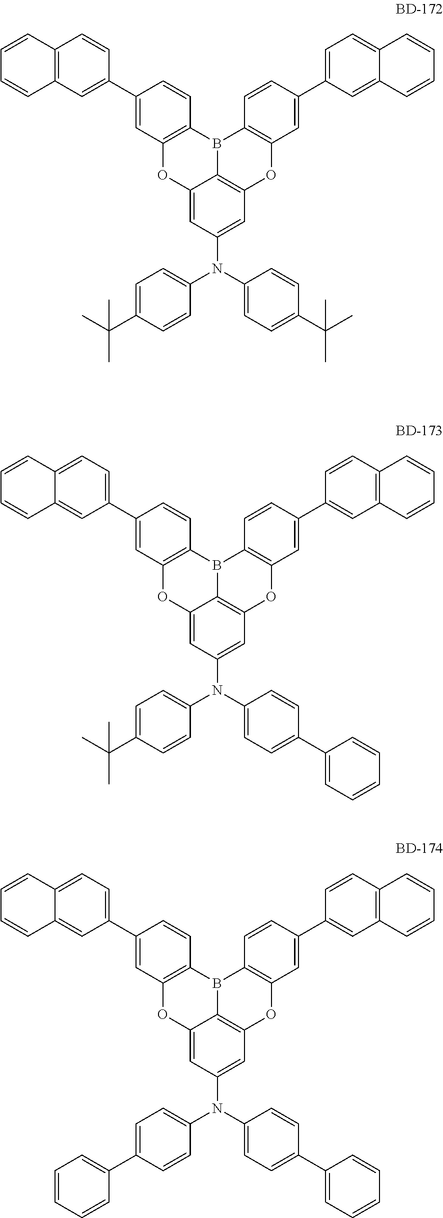 [SMILES: CC(C)(C)c1ccc(N(c2ccc(C(C)(C)C)cc2)c2cc3c4c(c2)Oc2cc(-c5ccc6ccccc6c5)ccc2B4c2ccc(-c4ccc5ccccc5c4)cc2O3)cc1 ; CC(C)(C)c1ccc(N(c2ccc(-c3ccccc3)cc2)c2cc3c4c(c2)Oc2cc(-c5ccc6ccccc6c5)ccc2B4c2ccc(-c4ccc5ccccc5c4)cc2O3)cc1 ; c1ccc(-c2ccc(N(c3ccc(-c4ccccc4)cc3)c3cc4c5c(c3)Oc3cc(-c6ccc7ccccc7c6)ccc3B5c3ccc(-c5ccc6ccccc6c5)cc3O4)cc2)cc1]

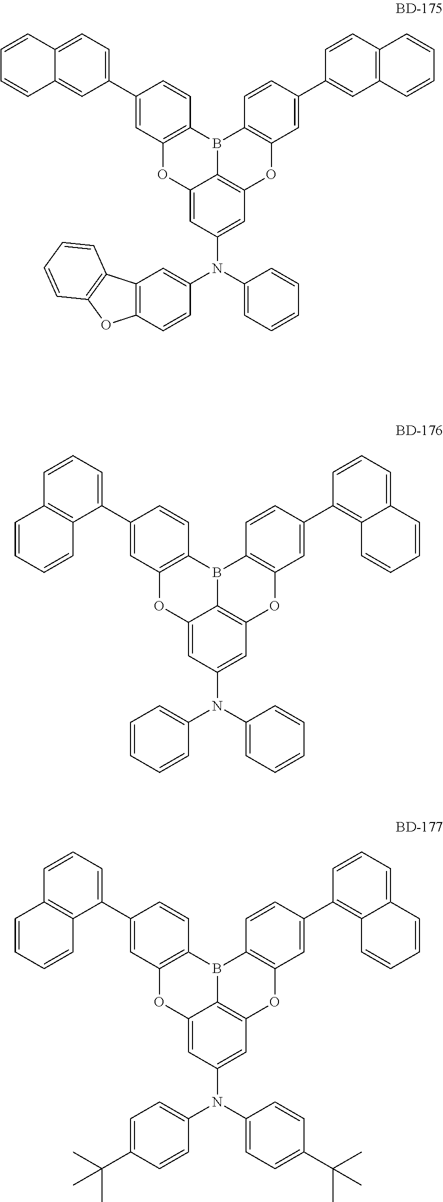 [SMILES: c1ccc(N(c2cc3c4c(c2)Oc2cc(-c5ccc6ccccc6c5)ccc2B4c2ccc(-c4ccc5ccccc5c4)cc2O3)c2ccc3oc4ccccc4c3c2)cc1 ; c1ccc(N(c2ccccc2)c2cc3c4c(c2)Oc2cc(-c5cccc6ccccc56)ccc2B4c2ccc(-c4cccc5ccccc45)cc2O3)cc1 ; CC(C)(C)c1ccc(N(c2ccc(C(C)(C)C)cc2)c2cc3c4c(c2)Oc2cc(-c5cccc6ccccc56)ccc2B4c2ccc(-c4cccc5ccccc45)cc2O3)cc1]

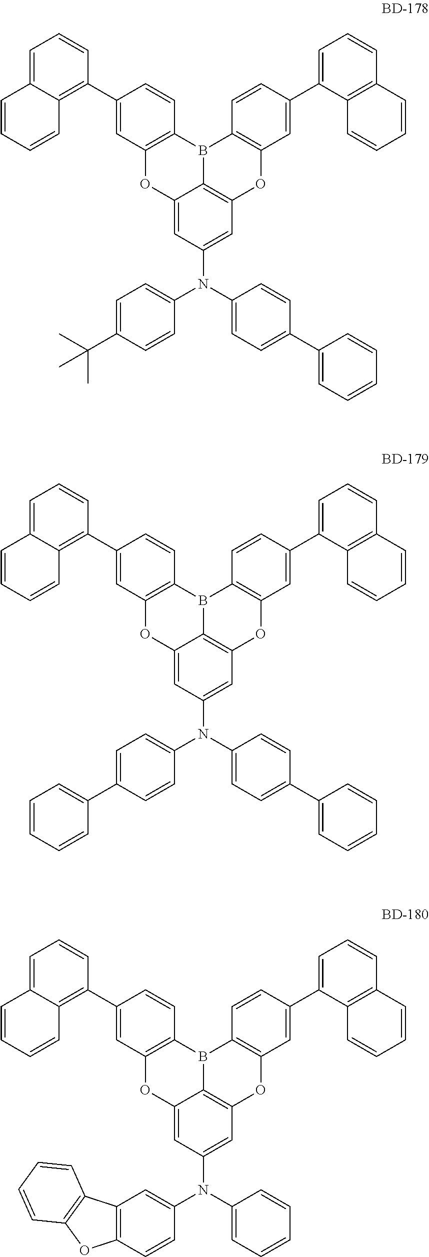 [SMILES: CC(C)(C)c1ccc(N(c2ccc(-c3ccccc3)cc2)c2cc3c4c(c2)Oc2cc(-c5cccc6ccccc56)ccc2B4c2ccc(-c4cccc5ccccc45)cc2O3)cc1 ; c1ccc(-c2ccc(N(c3ccc(-c4ccccc4)cc3)c3cc4c5c(c3)Oc3cc(-c6cccc7ccccc67)ccc3B5c3ccc(-c5cccc6ccccc56)cc3O4)cc2)cc1 ; c1ccc(N(c2cc3c4c(c2)Oc2cc(-c5cccc6ccccc56)ccc2B4c2ccc(-c4cccc5ccccc45)cc2O3)c2ccc3oc4ccccc4c3c2)cc1]

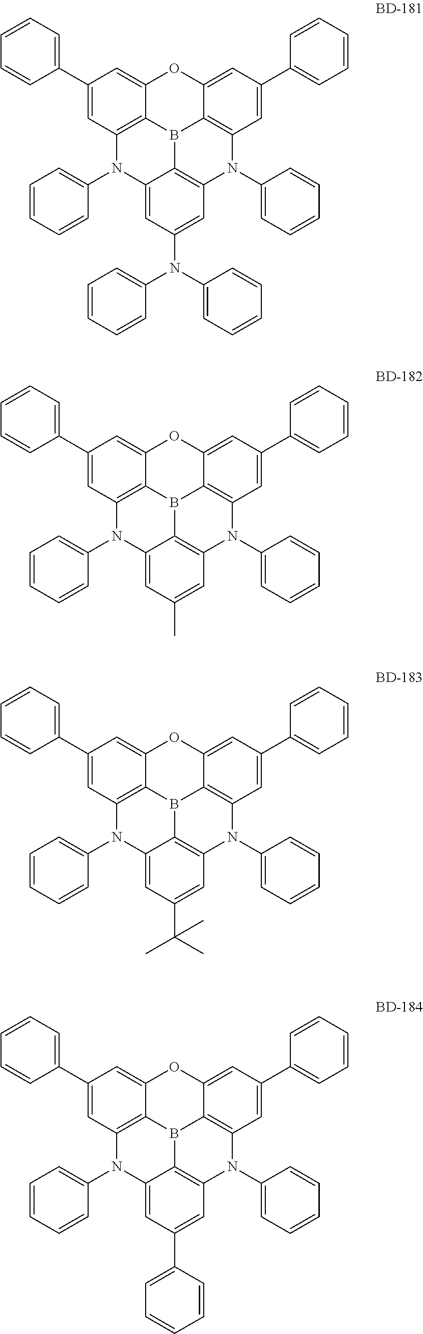 [SMILES: c1ccc(-c2cc3c4c(c2)N(c2ccccc2)c2cc(N(c5ccccc5)c5ccccc5)cc5c2B4c2c(cc(-c4ccccc4)cc2N5c2ccccc2)O3)cc1 ; Cc1cc2c3c(c1)N(c1ccccc1)c1cc(-c4ccccc4)cc4c1B3c1c(cc(-c3ccccc3)cc1N2c1ccccc1)O4 ; CC(C)(C)c1cc2c3c(c1)N(c1ccccc1)c1cc(-c4ccccc4)cc4c1B3c1c(cc(-c3ccccc3)cc1N2c1ccccc1)O4 ; c1ccc(-c2cc3c4c(c2)N(c2ccccc2)c2cc(-c5ccccc5)cc5c2B4c2c(cc(-c4ccccc4)cc2N5c2ccccc2)O3)cc1]

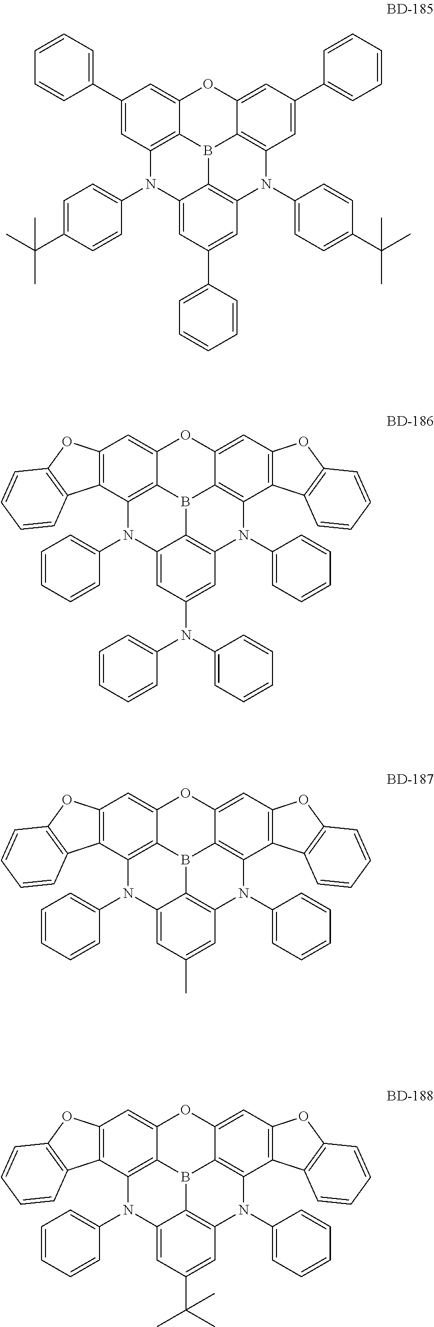 [SMILES: CC(C)(C)c1ccc(N2c3cc(-c4ccccc4)cc4c3B3c5c(cc(-c6ccccc6)cc5N(c5ccc(C(C)(C)C)cc5)c5cc(-c6ccccc6)cc2c53)O4)cc1 ; c1ccc(N(c2ccccc2)c2cc3c4c(c2)N(c2ccccc2)c2c5c(cc6oc7ccccc7c26)Oc2cc6oc7ccccc7c6c(c2B45)N3c2ccccc2)cc1 ; Cc1cc2c3c(c1)N(c1ccccc1)c1c4c(cc5oc6ccccc6c15)Oc1cc5oc6ccccc6c5c(c1B34)N2c1ccccc1 ; CC(C)(C)c1cc2c3c(c1)N(c1ccccc1)c1c4c(cc5oc6ccccc6c15)Oc1cc5oc6ccccc6c5c(c1B34)N2c1ccccc1]

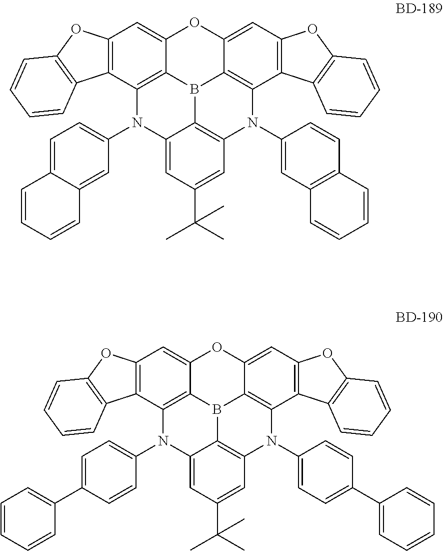 [SMILES: CC(C)(C)c1cc2c3c(c1)N(c1ccc4ccccc4c1)c1c4c(cc5oc6ccccc6c15)Oc1cc5oc6ccccc6c5c(c1B34)N2c1ccc2ccccc2c1 ; CC(C)(C)c1cc2c3c(c1)N(c1ccc(-c4ccccc4)cc1)c1c4c(cc5oc6ccccc6c15)Oc1cc5oc6ccccc6c5c(c1B34)N2c1ccc(-c2ccccc2)cc1]

The organic layer may further comprise at least one layer selected from a hole injection layer, a hole auxiliary layer, a light-emitting auxiliary layer, an electron transport layer, an electron injection layer, an interlayer, a hole blocking layer, an electron blocking layer and an electron buffer layer, in addition to the light-emitting layer and the hole transport layer. The organic layer may further comprise an amine-based compound and/or an azine-based compound other than the light-emitting material according to the present disclosure. Specifically, the hole injection layer, the hole transport layer, the hole auxiliary layer, the light-emitting layer, the light-emitting auxiliary layer, or the electron blocking layer may contain the amine-based compound, e.g., an arylamine-based compound and a styrylarylamine-based compound, etc., as a hole injection material, a hole transport material, a hole auxiliary material, a light-emitting material, a light-emitting auxiliary material, or an electron blocking material. Also, the electron transport layer, the electron injection layer, the electron buffer layer, or the hole blocking layer may contain the azine-based compound as an electron transport material, an electron injection material, an electron buffer material, or a hole blocking material. Also, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

The organic electroluminescent compound according to one embodiment may be used as light-emitting materials for a white organic light-emitting device. The white organic light-emitting device has suggested various structures such as a parallel side-by-side arrangement method, a stacking arrangement method, or CCM (color conversion material) method, etc., according to the arrangement of R (Red), G (Green), YG (yellowish green), or B (blue) light-emitting units. In addition, the plurality of host materials according to one embodiment may also be applied to the organic electroluminescent device comprising a QD (quantum dot).

One of the first electrode and the second electrode may be an anode and the other may be a cathode. Wherein, the first electrode and the second electrode may each be formed as a transmissive conductive material, a transflective conductive material, or a reflective conductive material. The organic electroluminescent device may be a top emission type, a bottom emission type, or a both-sides emission type according to the kinds of material forming the first electrode and the second electrode.

A hole injection layer, a hole transport layer, an electron blocking layer, or a combination thereof can be used between the anode and the light-emitting layer. The hole injection layer may be comprised of multi-layers in order to lower the hole injection barrier (or hole injection voltage) from the anode to the hole transport layer or the electron blocking layer, wherein each of the multi-layers may use two compounds simultaneously. Also, the hole injection layer may be doped as a p-dopant. Also, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron blocking layer may be multi-layers, and wherein each layer may use a plurality of compounds.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer may be placed between the electron transport layer (or electron injection layer) and the light-emitting layer, and blocks the arrival of holes to the cathode, thereby improving the probability of recombination of electrons and holes in the light-emitting layer. The hole blocking layer or the electron transport layer may also be multi-layers, wherein each layer may use a plurality of compounds. Also, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron blocking layer. The light-emitting auxiliary layer, the hole auxiliary layer, or the electron blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiOₓ(1≤X≤2), AlOₓ(1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

The organic electroluminescent device according to one embodiment of the present disclosure may be an organic electroluminescent device having a tandem structure. In the case of a tandem organic electroluminescent device according to one embodiment, a single light-emitting unit may be formed in a structure in which two or more units are connected by a charge generation layer. The organic electroluminescent device may include a plurality of two or more light emitting units, for example, a plurality of three or more light-emitting units, having first and second electrodes opposed to each other on a substrate and a light-emitting layer that is stacked between the first and second electrodes and emits light in a specific wavelength range. In one embodiment, the organic electroluminescent device may include a plurality of light-emitting units, and each of the light-emitting units may include a hole transport band, a light-emitting layer, and an electron transport band. The hole transport band may include a hole injection layer and a hole transport layer, and the electron transport band may include an electron transport layer and an electron injection layer. According to one embodiment, there may be three or more light-emitting layers included in the light-emitting unit. A plurality of light-emitting units may emit the same color or different colors. Additionally, one light-emitting unit may include one or more light-emitting layers, and the plurality of light-emitting layers may be light-emitting layers of the same or different colors. It may include one or more charge generation layers located between each light-emitting unit. The charge generation layer refers to the layer in which holes and electrons are generated when voltage is applied. When there are three or more light-emitting units, a charge generation layer may be located between each light-emitting unit. The plurality of charge generation layers may be the same or different from each other. By disposing the charge generation layer between light-emitting units, current efficiency is increased in each light-emitting unit and charges can be smoothly distributed. Specifically, the charge generation layer is provided between two adjacent stacks and can serve to drive a tandem organic electroluminescent device using only a pair of anodes and cathodes without a separate internal electrode located between the stacks.

The charge generation layer may be composed of an N-type charge generation layer and a P-type charge generation layer, and the N-type charge generation layer may be doped with an alkali metal, an alkaline earth metal, or a compound of an alkali metal and an alkaline earth metal, The alkali metal may include one selected from the group consisting of Li, Na, K, Rb, Cs, Fr, Yb, and combinations thereof, and the alkaline earth metal may include one selected from the group consisting of Be, Mg, Ca, Sr, Ba, Ra, and combinations thereof. The P-type charge generation layer may be made of a metal or an organic material doped with a P-type dopant. For example, the metal may be made of one or two or more alloys selected from the group consisting of Al, Cu, Fe, Pb, Zn, Au, Pt, W, In, Mo, Ni, and Ti. Additionally, commonly used materials may be used as the P-type dopant and host materials used in the P-type doped organic material.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation; thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. Also, a reductive dopant layer may be employed as a charge generating layer to prepare an organic electroluminescent device having two or more light-emitting layers and emitting white light.

In order to form each layer of the organic electroluminescent device of the present disclosure, dry film-forming methods such as vacuum evaporation, sputtering, plasma, ion plating methods, etc., or wet film-forming methods such as spin coating, dip coating, flow coating methods, etc., can be used. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by the organic electroluminescent compound according to one embodiment, the layer can be formed by the above-listed methods, and can often be formed by co-deposition or mixture-deposition. The co-deposition is a mixed deposition method in which two or more materials are put into respective individual crucible sources and a current is applied to both cells simultaneously to evaporate the materials and to perform mixed deposition; and the mixture-deposition is a mixed deposition method in which two or more materials are mixed in one crucible source before deposition, and then a current is applied to one cell to evaporate the materials.

According to one embodiment, when the organic electroluminescent materials exist in the same layer or different layers in the organic electroluminescent device, each of the layers may be separately formed.

According to one embodiment, the present disclosure can provide display devices comprising an organic electroluminescent compound represented by Formula 1. In addition, the organic electroluminescent device of the present disclosure can be used for the manufacture of display devices such as smartphones, tablets, notebooks, PCs, TVs, or display devices for vehicles, or lighting devices such as outdoor or indoor lighting.

Hereinafter, the preparation method of organic electroluminescent compounds according to the present disclosure will be explained with reference to the synthesis method of a representative compound or intermediate compound in order to understand the present disclosure in detail.

### [Example 1] Synthesis of Compound C-74-D26

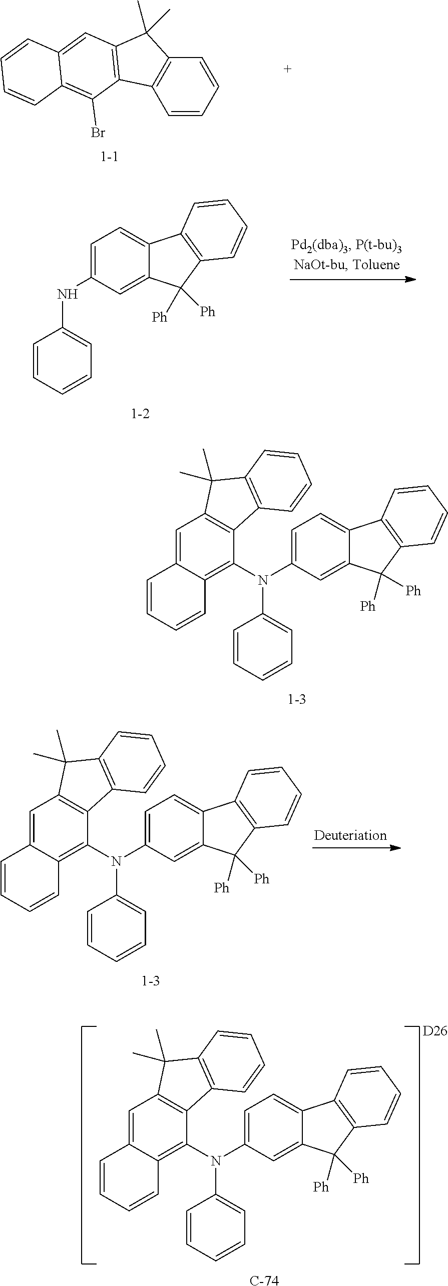

### 1) Synthesis of Compound 1-3

Compound 1-1 (10.86 g, 26.4 mmol), Compound 1-2 (9.83 g, 24 mmol), Pd₂(dba)₃ (1.10 g, 1.2 mmol), (50%) P(t-bu)₃ (1.16 mL, 2.4 mmol), NaOt-bu (6.92 g, 72.0 mmol), and 120 mL of toluene were added to a flask, and then stirred at 120° C. After completion of the reaction, the reaction mixture was cooled to room temperature, filtered through Celite filter, dissolved in methylene chloride, and separated by column chromatography. Next, methanol was added thereto, and the resulting solid was filtered under reduced pressure to obtain Compound 1-3 (7.8 g, yield: 50%, MS: [M+H]⁺=678.5).

### 2) Synthesis of Compound C-74-D26

Compound 1-3 was synthesized by selecting the deuterization methods disclosed in Korean Patent Nos. 10-2283849 and 10-1427457, etc., to obtain Compound C-74-D26 (4.5 g, yield: 49%).

### [Example 2] Synthesis of Compound C-53-D18

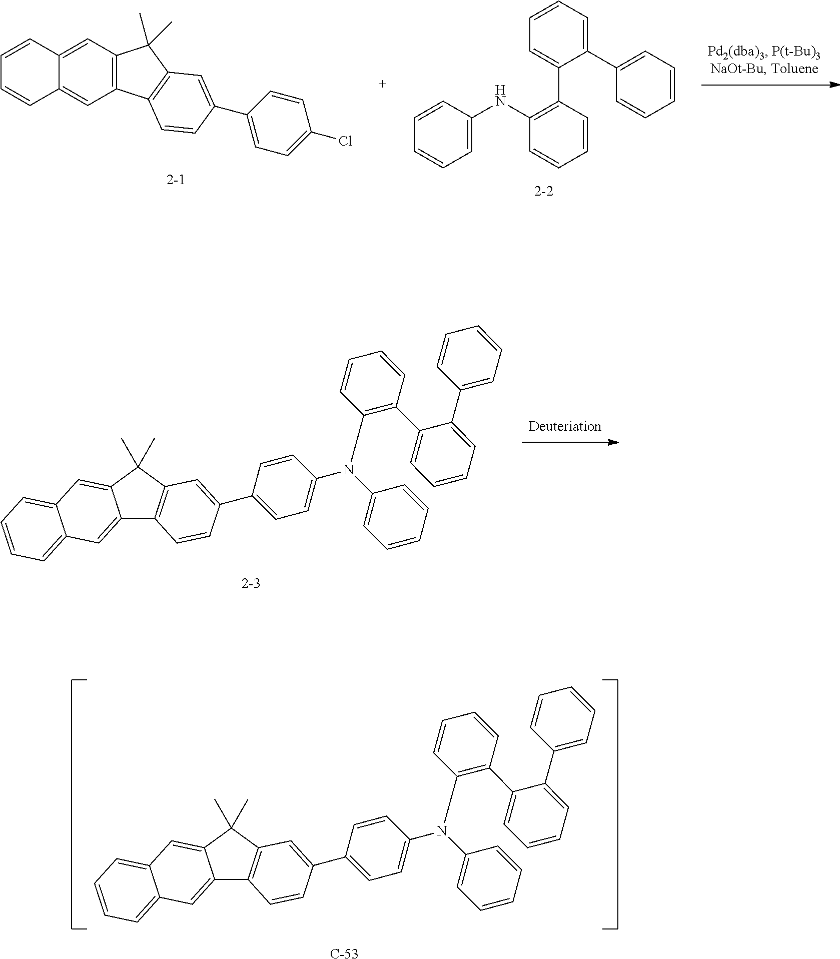

### 1) Synthesis of Compound 2-3

Compound 2-1 (47.8 g, 134.7 mmol), Compound 2-2 (43.3 g, 134.7 mmol), Pd₂(dba)₃ (6.20 g, 6.73 mmol), (50%) P(t-bu)₃ (6.6 mL, 13.47 mmol), NaOt-bu (19.4 g, 202.05 mmol), and 675 mL of toluene were added to a flask, and then stirred at 120° C. After completion of the reaction, the reaction mixture was cooled to room temperature, filtered through Celite filter, dissolved in methylene chloride, and separated by column chromatography. Next, methanol was added thereto, and the resulting solid was filtered under reduced pressure to obtain Compound 2-3 (28.0 g, yield: 32.5%).

### 2) Synthesis of Compound C-53-D18

Compound 2-3 was synthesized by selecting the deuterization methods disclosed in Korean Patent Nos. 10-2283849 and 10-1427457, etc., to obtain Compound C-53-D18 (3.8 g, yield: 45.2%, MS: [M+H]⁺=658.4).

### [Example 3] Synthesis of Compound C-102-D20

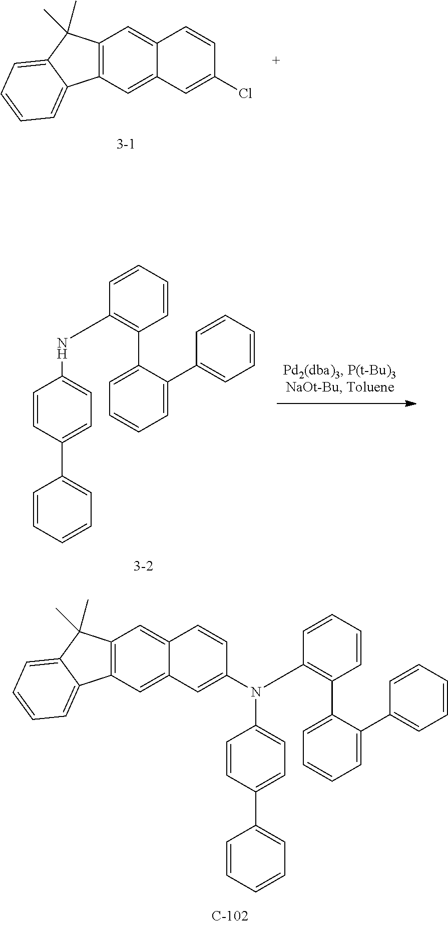 [SMILES: CC1(C)c2ccccc2-c2cc3cc(Cl)ccc3cc21 ; c1ccc(-c2ccc(Nc3ccccc3-c3ccccc3-c3ccccc3)cc2)cc1 ; CC1(C)c2ccccc2-c2cc3cc(N(c4ccc(-c5ccccc5)cc4)c4ccccc4-c4ccccc4-c4ccccc4)ccc3cc21]

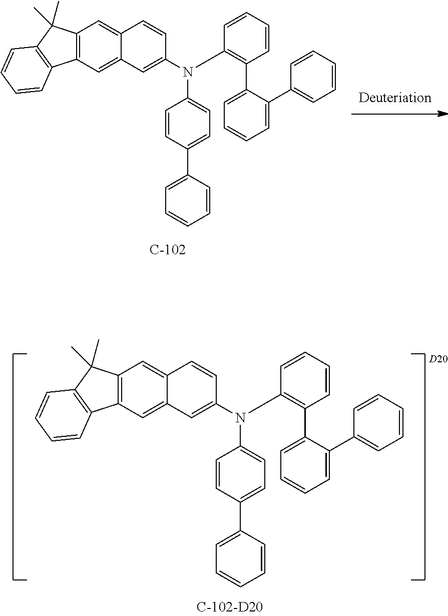

### 1) Synthesis of Compound C-102

Compound 3-1 (7 g, 25 mmol), Compound 3-2 (9 g, 23 mmol), Pd₂(dba)₃ (1.2 g, 1 mmol), (50%) P(t-Bu)₃ (1.2 mL, 25 mmol), NaOt-bu (7.2 g, 75 mmol), and 130 mL of toluene were added to a flask, and then stirred at 120° C. After completion of the reaction, the reaction mixture was cooled to room temperature, filtered through Celite filter, dissolved in methylene chloride, and separated by column chromatography. Next, methanol was added thereto, and the resulting solid was filtered under reduced pressure to obtain Compound C-102 (10.6 g, yield: 65.9%).

| | MW | M.P |
|---|---|---|
| C-102 | 639.84 | 222.9° C. |

### 2) Synthesis of Compound C-102-D20

Compound C-102 was synthesized by selecting the deuterization methods disclosed in Korean Patent Nos. 10-2283849 and 10-1427457, etc., to obtain compound C-102-D20 (7.5 g, yield: 75%).

### [Example 4] Synthesis of Compound C-103-D19

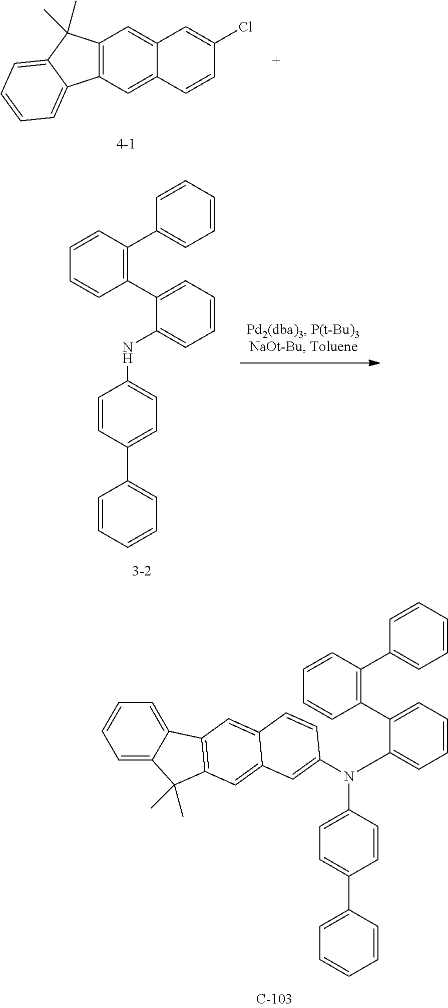 [SMILES: CC1(C)c2ccccc2-c2cc3ccc(Cl)cc3cc21 ; c1ccc(-c2ccc(Nc3ccccc3-c3ccccc3-c3ccccc3)cc2)cc1 ; CC1(C)c2ccccc2-c2cc3ccc(N(c4ccc(-c5ccccc5)cc4)c4ccccc4-c4ccccc4-c4ccccc4)cc3cc21]

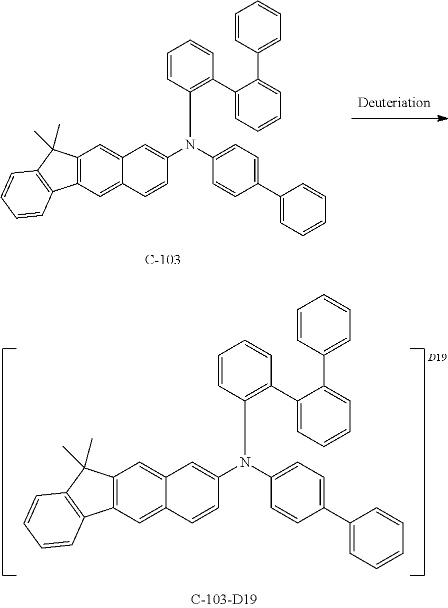

### 1) Synthesis of Compound C-103

Compound 4-1 (7 g, 25 mmol), Compound 3-2 (9 g, 23 mmol), Pd₂(dba)₃ (1.2 g, 1 mmol), (50%) P(t-Bu)₃ (1.2 mL, 25 mmol), NaOt-bu (7.2 g, 75 mmol), and 130 mL of toluene were added to a flask, and then stirred at 120° C. After completion of the reaction, the reaction mixture was cooled to room temperature, filtered through Celite filter, dissolved in methylene chloride, and separated by column chromatography. Next, methanol was added thereto, and the resulting solid was filtered under reduced pressure to obtain Compound C-103 (10.5 g, yield: 65.3%).

| | MW | M.P |
|---|---|---|
| C-103 | 639.84 | 247.1° C. |

### 2) Synthesis of Compound C-103-D19

Compound C-103 was synthesized by selecting the deuterization methods disclosed in Korean Patent Nos. 10-2283849 and 10-1427457, etc., to obtain Compound C-103-D19 (2.3 g, yield: 23%).

### [Example 5] Synthesis of Compound C-104-D19

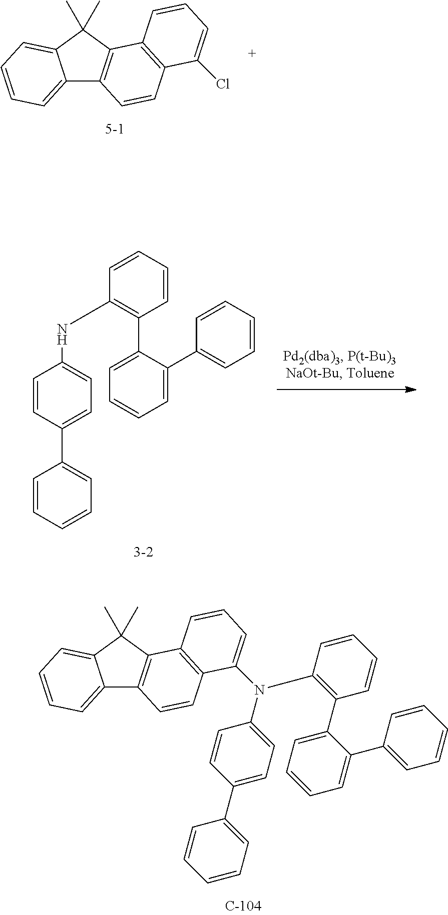 [SMILES: CC1(C)c2ccccc2-c2ccc3c(Cl)cccc3c21 ; c1ccc(-c2ccc(Nc3ccccc3-c3ccccc3-c3ccccc3)cc2)cc1 ; CC1(C)c2ccccc2-c2ccc3c(N(c4ccc(-c5ccccc5)cc4)c4ccccc4-c4ccccc4-c4ccccc4)cccc3c21]

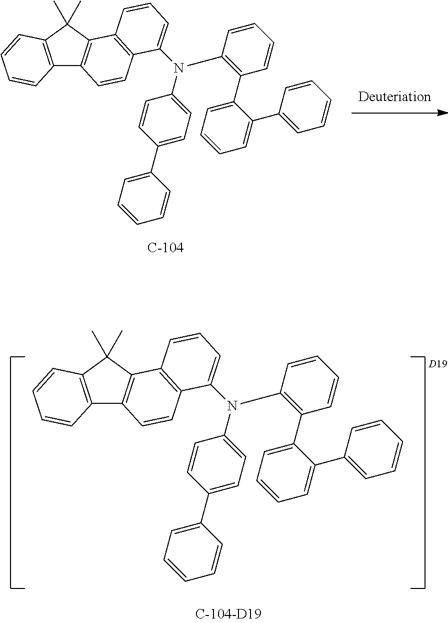

### 1) Synthesis of Compound C-104

Compound 5-1 (7 g, 25 mmol), Compound 3-2 (9 g, 23 mmol), Pd₂(dba)₃ (1.2 g, 1 mmol), (50%) P(t-Bu) (1.2 mL, 25 mmol), NaOt-bu (7.2 g, 75 mmol), and 130 mL of toluene 130 mL were added to a flask, and then stirred at 120° C. After completion of the reaction, the reaction mixture was cooled to room temperature, filtered through Celite filter, dissolved in methylene chloride, and separated by column chromatography. Next, methanol was added thereto, and the resulting solid was filtered under reduced pressure to obtain Compound C-104 (10.6 g, yield: 65.9%).

| | MW | M.P |
|---|---|---|
| C-104 | 639.84 | 250° C. |

### 2) Synthesis of Compound C-104-D19

Compound C-104 was synthesized by selecting the deuterization methods disclosed in Korean Patent Nos. 10-2283849 and 10-1427457, etc., to obtain Compound C-104-D19 (4.6 g, yield: 46%).

Hereinafter, the preparation method of an organic electroluminescent device comprising the organic electroluminescent compound according to the present disclosure and the device property thereof will be explained in order to understand the present disclosure in detail.

### [Device Examples 1 to 5] Preparation of OLEDs by Depositing the Organic Electroluminescent Compound According to the Present Disclosure as a Material for a Hole Transport Layer

OLEDs according to the present disclosure were prepared. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Then, Compound HI-1 was introduced into a cell of the vacuum vapor deposition apparatus, and Compound HT-1 was introduced into another cell. The two materials were evaporated at different rates and Compound HI-1 was deposited in a doping amount of 7 wt % based on the total amount of compounds HI-1 and HT-1 to form a hole injection layer having a thickness of 10 nm. Next, Compound HT-1 was deposited as a first hole transport layer having a thickness of 75 nm on the hole injection layer. Then, the compound described in the following Tables 1 to 5 as a second hole transport layer was then introduced into another cell of the vacuum vapor deposition apparatus and was evaporated by applying an electric current to the cell, thereby forming a second hole transport layer having a thickness of 5 nm on the first hole transport layer. After forming the hole injection layer and the hole transport layers, a light-emitting layer was formed thereon as follows: Compound BH-228 was introduced into a cell of the vacuum vapor deposition apparatus as a host and Compound BD-96 was introduced into another cell as a dopant. The dopant material was evaporated at a different rate and was deposited in a doping amount of 2 wt % based on the total amount of the host and dopant to form a light-emitting layer having a thickness of 20 nm on the second hole transport layer. Next, Compound HBL was deposited to a thickness of 5 nm as an electron buffer layer on the light-emitting layer, and Compound ET-1 and Compound EI-1 as electron transport materials was deposited at a weight ratio of 50:50 to form an electron transport layer having a thickness of 30 nm on the light-emitting layer. After depositing Compound EI-1 as an electron injection layer having a thickness of 2 nm on the electron transport layer, an Al cathode having a thickness of 80 nm was deposited on the electron injection layer by another vacuum vapor deposition apparatus. Thus, OLEDs were produced. Each compound used for all the materials were purified by vacuum sublimation under 10⁻⁶ torr.

### [Device Comparative Examples 1 to 5] Preparation of OLEDs Containing Conventional Compounds as a Material for a Hole Transport Layer

OLEDs were manufactured in the same manner as device Example 1, except that the compound shown in the following Tables 1 to 5 was used as the material for the second hole transport layer.

The driving voltage, the luminous efficiency, and the luminous color at a luminance of 1,000 nits and the time taken for luminance to decrease from 100% to 90% at a luminance of 1,890 nits (lifespan: T90) of the OLEDs of Device Examples 1 to 5 and Device Comparative Examples 1 to 5 produced as described above, are measured, and the results thereof are shown in the following Tables 1 to 5, respectively. The conversion lifetime was converted based on the lifespan of each Device Comparative Example as 100%.

TABLE 1

| | Material for | | | | |
|---|---|---|---|---|---|
| | Second Hole | Driving | Luminous | | Conversion |
| | Transport | Voltage | Efficiency | Luminous | Lifespan |
| | Layer | (V) | (cd/A) | Color | (T90, %) |
| Device | C-74-D26 | 3.2 | 6.7 | Blue | 115 |
| Example 1 | | | | | |
| Device | T-1 | 3.2 | 6.7 | Blue | 100 |
| Compar- | | | | | |
| ative | | | | | |
| Example 1 | | | | | |

TABLE 2

| | Material for | | | | |
|---|---|---|---|---|---|
| | Second Hole | Driving | Luminous | | Conversion |
| | Transport | Voltage | Efficiency | Luminous | Lifespan |
| | Layer | (V) | (cd/A) | Color | (T90, %) |
| Device | C-53-D18 | 3.2 | 6.7 | Blue | 114 |
| Example 2 | | | | | |
| Device | T-2 | 3.2 | 6.7 | Blue | 100 |
| Compar- | | | | | |
| ative | | | | | |
| Example 2 | | | | | |

TABLE 3

| | Material for | | | | |
|---|---|---|---|---|---|
| | Second Hole | Driving | Luminous | | Conversion |
| | Transport | Voltage | Efficiency | Luminous | Lifespan |
| | Layer | (V) | (cd/A) | Color | (T90, %) |
| Device | C-103-D19 | 3.2 | 6.5 | Blue | 108 |
| Example 3 | | | | | |
| Device | T3 | 3.2 | 6.5 | Blue | 100 |
| Compar- | | | | | |
| ative | | | | | |
| Example 3 | | | | | |

TABLE 4

| | Material for | | | | |
|---|---|---|---|---|---|
| | Second Hole | Driving | Luminous | | Conversion |
| | Transport | Voltage | Efficiency | Luminous | Lifespan |
| | Layer | (V) | (cd/A) | Color | (T90, %) |
| Device | C-102-D20 | 3.2 | 6.7 | Blue | 107 |
| Example 4 | | | | | |
| Device | T4 | 3.2 | 6.7 | Blue | 100 |
| Compar- | | | | | |
| ative | | | | | |
| Example 4 | | | | | |

TABLE 5

| | Material for | | | | |
|---|---|---|---|---|---|
| | Second Hole | Driving | Luminous | | Conversion |
| | Transport | Voltage | Efficiency | Luminous | Lifespan |
| | Layer | (V) | (cd/A) | Color | (T90, %) |
| Device | C-104-D19 | 3.2 | 6.6 | Blue | 111 |
| Example 5 | | | | | |
| Device | T5 | 3.2 | 6.5 | Blue | 100 |
| Compar- | | | | | |
| ative | | | | | |
| Example 5 | | | | | |

From the results of Table 1 to Table 5, it can be confirmed that an organic electroluminescent device comprising at least one deuterated organic electroluminescent compound as a hole transport layer material, according to the present disclosure, exhibits significantly improved lifespan characteristics compared to an organic electroluminescent device using non-deuterated compounds.

The compounds used in the aforementioned Device Examples and Device Comparative Examples are specifically shown in the following Table 6.

TABLE 6

| | |
|---|---|
| Hole Injection Layer/First Hole Transport Layer | 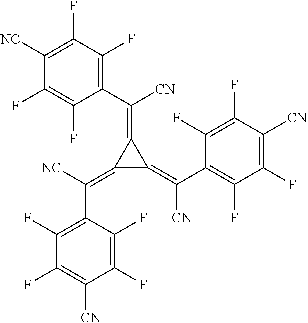 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 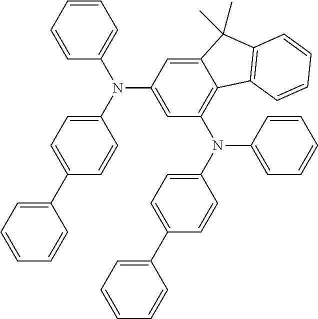 [SMILES: CC1(C)c2ccccc2-c2c(N(c3ccccc3)c3ccc(-c4ccccc4)cc3)cc(N(c3ccccc3)c3ccc(-c4ccccc4)cc3)cc21] |
| | HT-1 |
| Second Hole Transport Layer | 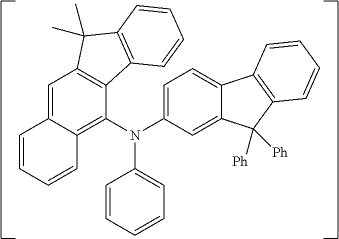 |
| | D26 |
| | C-74-D26 |
| | 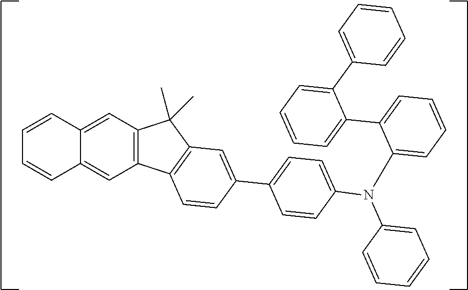 |
| | D18 |
| | C-53-D18 |
| | 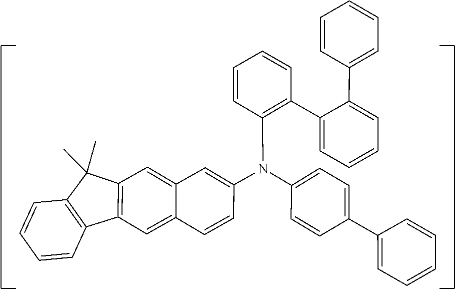 |
| | D19 |
| | C-103-D19 |
| | 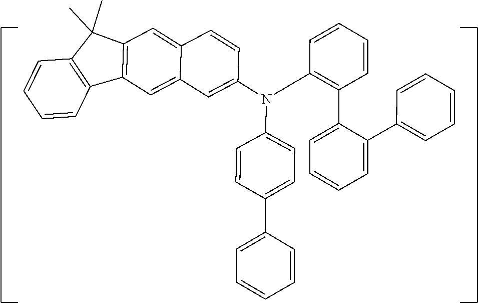 |
| | D20 |
| | C-102-D20 |
| | 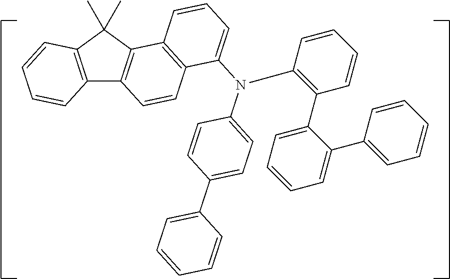 |
| | D19 |
| | C-104-D19 |
| | 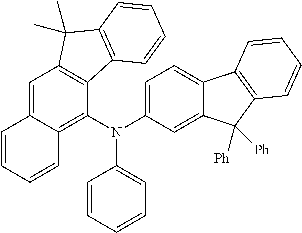 [SMILES: CC1(C)c2ccccc2-c2c1cc1ccccc1c2N(c1ccccc1)c1ccc2c(c1)C(c1ccccc1)(c1ccccc1)c1ccccc1-2] |
| | T-1 |
| | 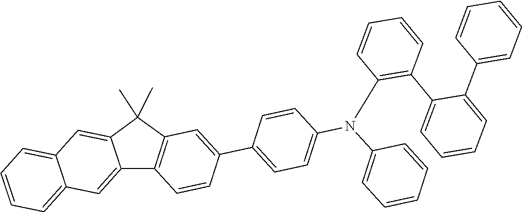 [SMILES: CC1(C)c2cc(-c3ccc(N(c4ccccc4)c4ccccc4-c4ccccc4-c4ccccc4)cc3)ccc2-c2cc3ccccc3cc21] |
| | T-2 |
| | 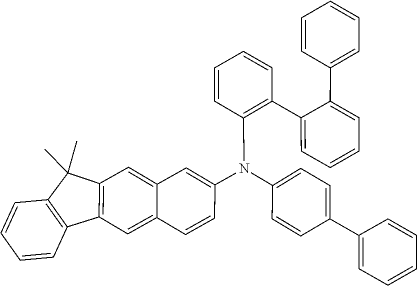 [SMILES: CC1(C)c2ccccc2-c2cc3ccc(N(c4ccc(-c5ccccc5)cc4)c4ccccc4-c4ccccc4-c4ccccc4)cc3cc21] |
| | T-3 |
| | 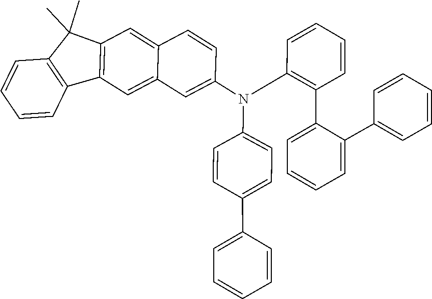 [SMILES: CC1(C)c2ccccc2-c2cc3cc(N(c4ccc(-c5ccccc5)cc4)c4ccccc4-c4ccccc4-c4ccccc4)ccc3cc21] |
| | T-4 |
| | 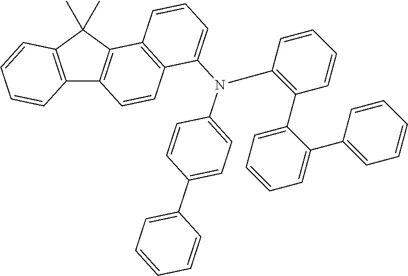 [SMILES: CC1(C)c2ccccc2-c2ccc3c(N(c4ccc(-c5ccccc5)cc4)c4ccccc4-c4ccccc4-c4ccccc4)cccc3c21] |
| | T-5 |
| Light-Emitting Layer/ Electron Buffer Layer | 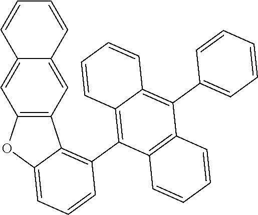 [SMILES: c1ccc(-c2c3ccccc3c(-c3cccc4oc5cc6ccccc6cc5c34)c3ccccc23)cc1] |
| | BH-228 |
| | 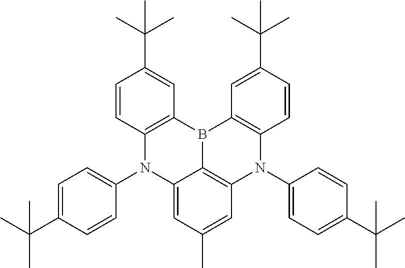 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1] |
| | BD-96 |
| | 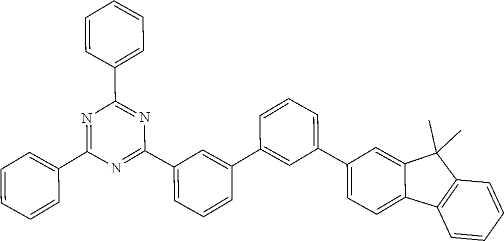 [SMILES: CC1(C)c2ccccc2-c2ccc(-c3cccc(-c4cccc(-c5nc(-c6ccccc6)nc(-c6ccccc6)n5)c4)c3)cc21] |
| | HBL |
| Electron Transport Layer/ Electron Injection Layer | 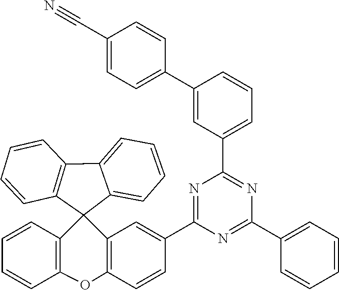 [SMILES: N#Cc1ccc(-c2cccc(-c3nc(-c4ccccc4)nc(-c4ccc5c(c4)C4(c6ccccc6O5)c5ccccc5-c5ccccc54)n3)c2)cc1] |
| | ET-1 |
| | 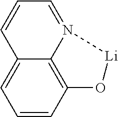 |
| | EI-1 |

1. An organic electroluminescent compound represented by the following Formula 1: 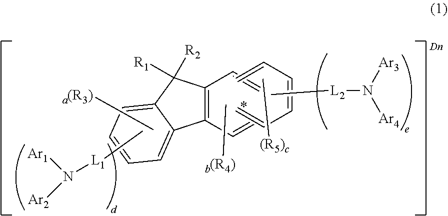
wherein,
R₁ and R₂ each independently represent, a substituted or unsubstituted (C1-C30)alkyl or a substituted or unsubstituted (C6-C30)aryl;
L₁ and L₂ each independently represent, a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
R₃ to R₅ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, or —N—(Ar₅)(Ar₆);
Ar₁ to Ar₄ each independently represent, a substituted or unsubstituted (C6-C30)aryl or a substituted or unsubstituted (3- to 30-membered)heteroaryl;
Ar₅ and Ar₆ each independently represent, hydrogen, a substituted or unsubstituted (C1-C30)alkyl, a substituted or unsubstituted (C2-C30)alkenyl, a substituted or unsubstituted (C6-C30)aryl, or a substituted or unsubstituted (5- to 30-membered)heteroaryl;
a and c each independently represent, an integer of 1 to 4, b is an integer of 1 or 2, when a to c are 2 or more, each of R₃ to R₅ may be the same or different;
d and e each independently represent, 0 or 1, with at least one of d and e being 1; and
Dn signifies that n hydrogens are replaced with deuterium, where n represents an integer of 1 or more.

2. The organic electroluminescent compound according to claim 1, wherein the formula 1 is represented by any one of the following formulas 1-1 to 1-3: 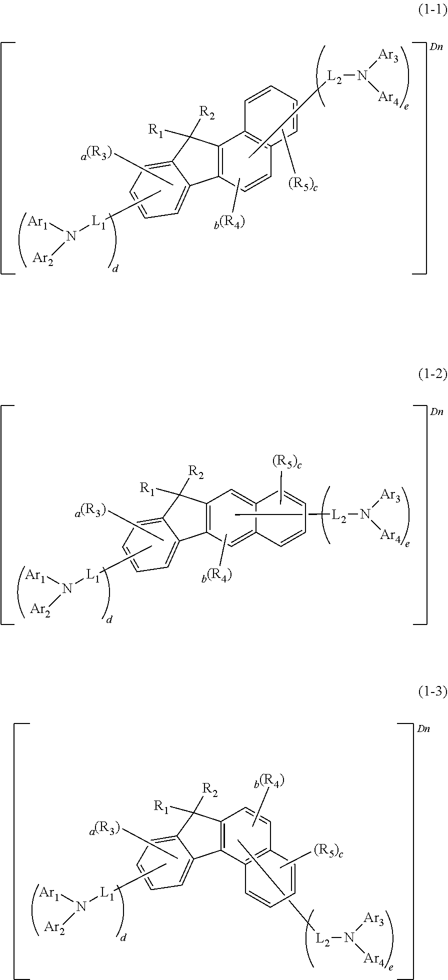
wherein,
R₁ to R₅, L₁, L₂, Ar₁ to Ar₄, a to e, and Dn are as defined in claim 1.

3. The organic electroluminescent compound according to claim 1, wherein the substituted alkyl(ene), the substituted alkenyl, the substituted aryl(ene), the substituted heteroaryl(ene), and the substituted cycloalkyl(ene) each independently are substituted with at least one selected from the group consisting of deuterium; halogen; cyano; carboxyl; nitro; hydroxy; phosphine oxide; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3- to 7-membered)heterocycloalkyl; (C6-C30)aryloxy; (C6-C30)arylthio; (3- to 30-membered)heteroaryl unsubstituted or substituted with at least one (C6-C30)aryl; (C6-C30)aryl unsubstituted or substituted with at least one of (C1-C30)alkyl, (C6-C30)aryl and (3- to 30-membered)heteroaryl; tri(C1-C30)alkylsilyl; tri(C6-C30)arylsilyl; di(C1-C30)alkyl(C6-C30)arylsilyl; (C1-C30)alkyldi(C6-C30)arylsilyl; a fused ring of (C3-C30) aliphatic ring and (C6-C30) aromatic ring; amino; mono- or di-(C1-C30)alkylamino; mono- or di-(C2-C30)alkenylamino; (C1-C30)alkyl(C2-C30)alkenylamino; mono- or di-(C6-C30)arylamino; (C1-C30)alkyl(C6-C30)arylamino; mono- or di-(3- to 30-membered)heteroarylamino; (C1-C30)alkyl(3- to 30-membered)heteroarylamino; (C2-C30)alkenyl(C6-C30)arylamino; (C2-C30)alkenyl(3- to 30-membered)heteroarylamino; (C6-C30)aryl(3- to 30-membered)heteroarylamino; (C1-C30)alkylcarbonyl; (C1-C30)alkoxycarbonyl; (C6-C30)arylcarbonyl; (C6-C30)arylphosphinyl; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl.

4. The organic electroluminescent compound according to claim 1, wherein the compound represented by the Formula 1 is selected from the following compounds:

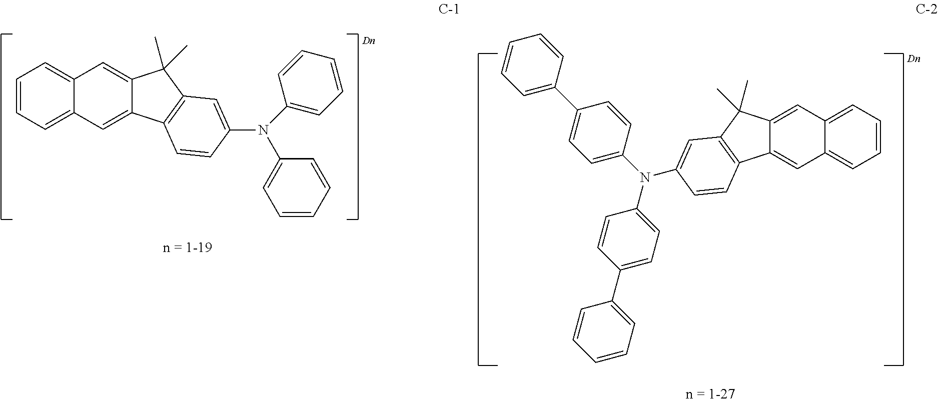

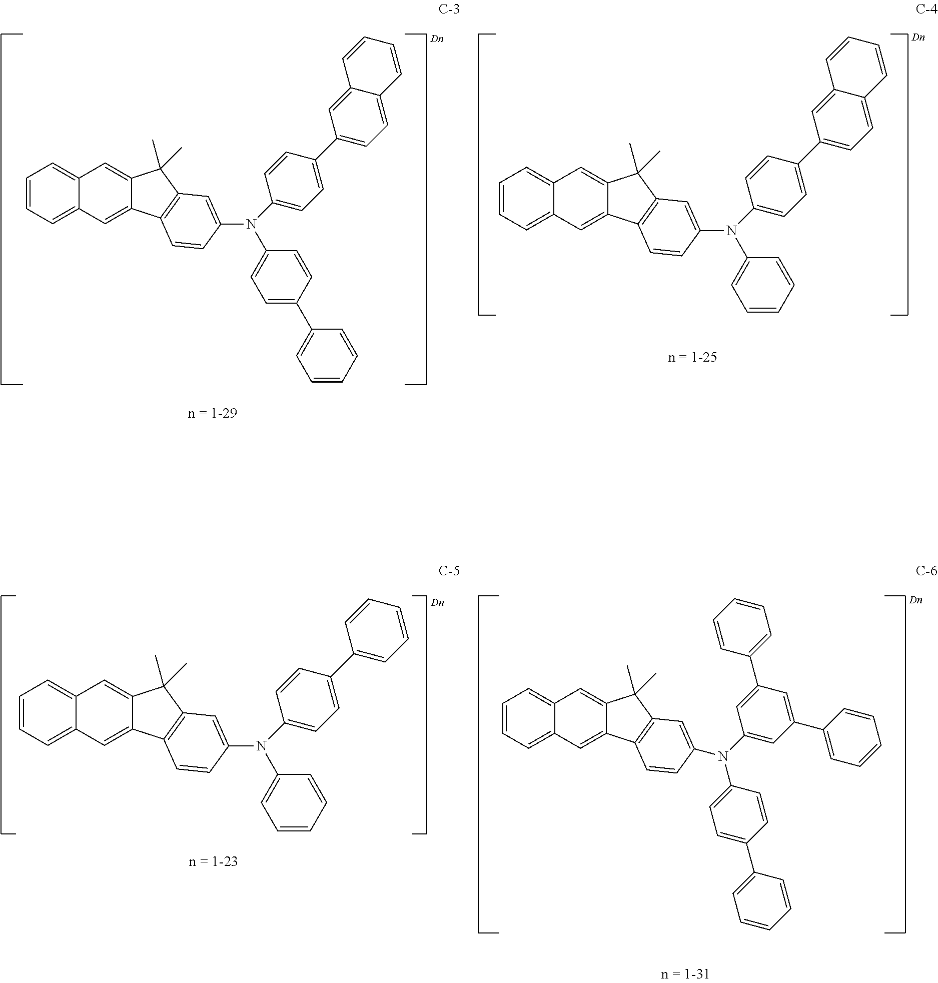

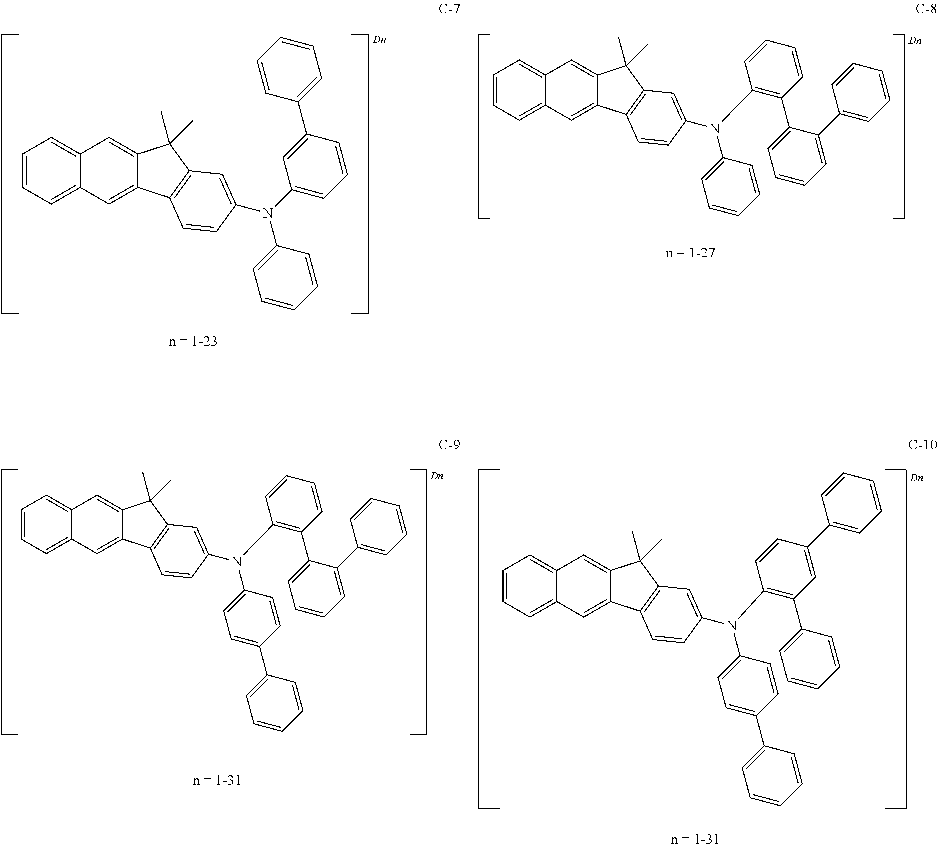

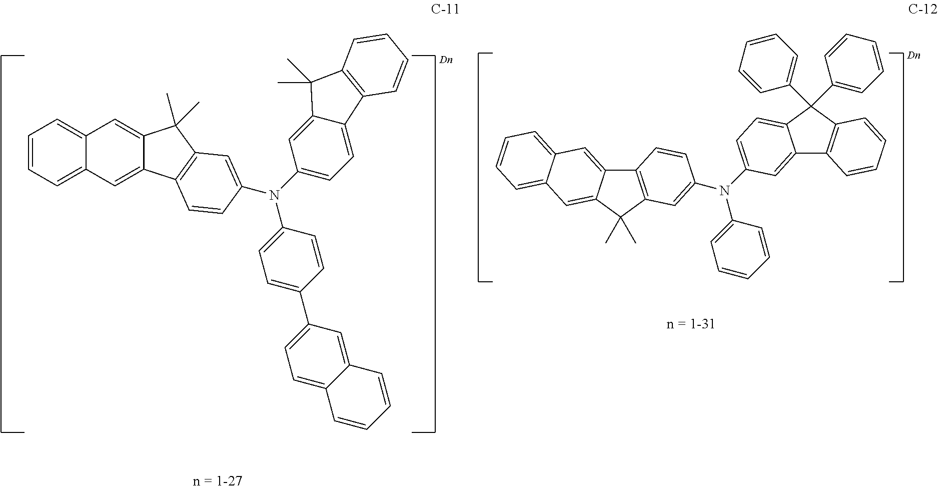

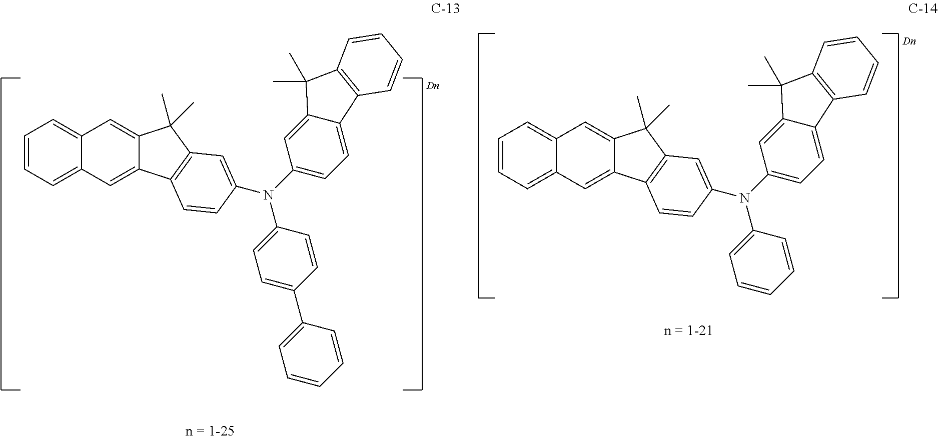

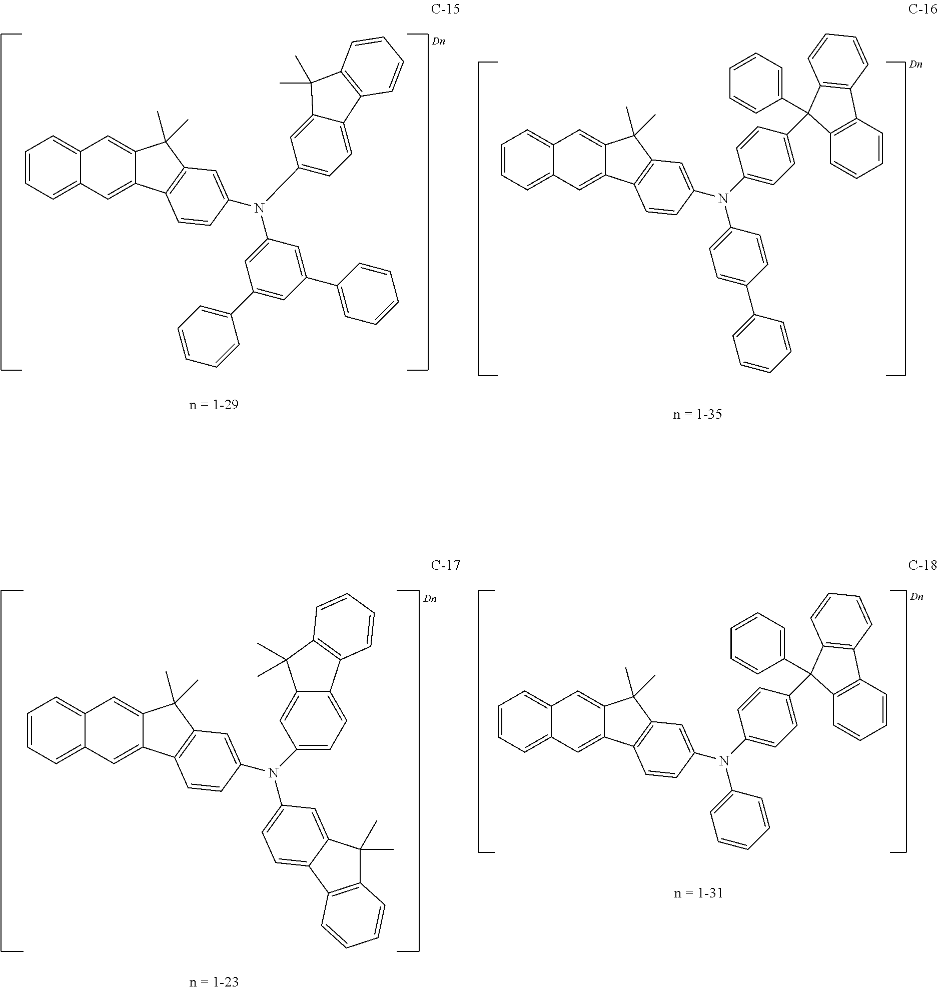

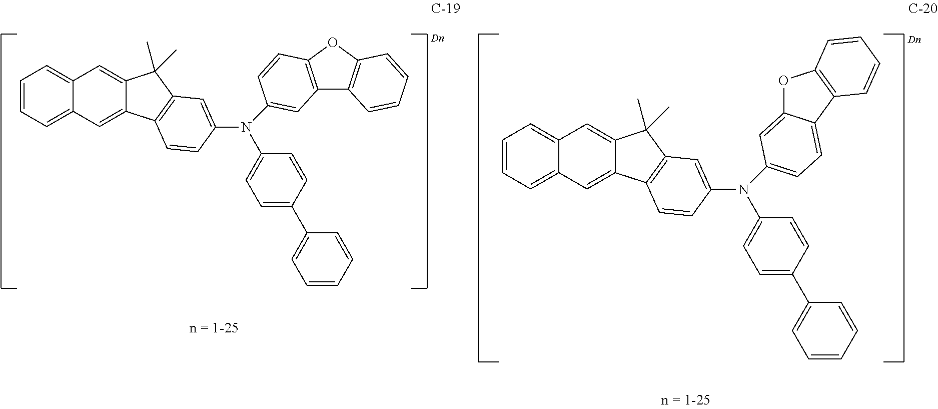

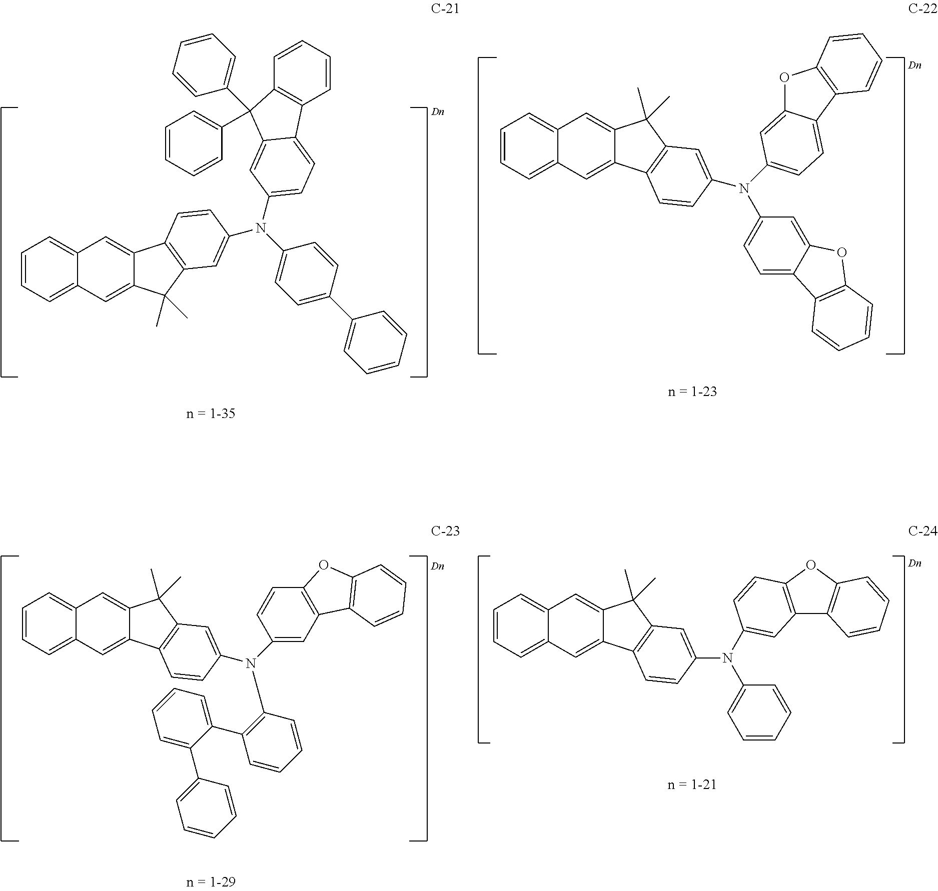

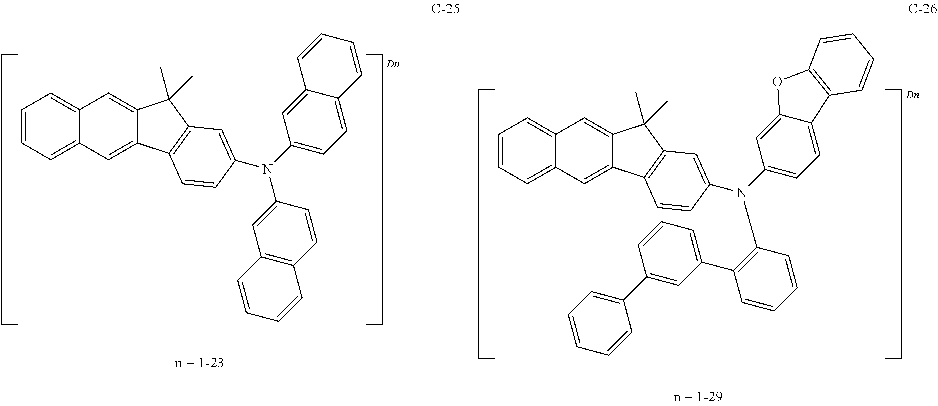

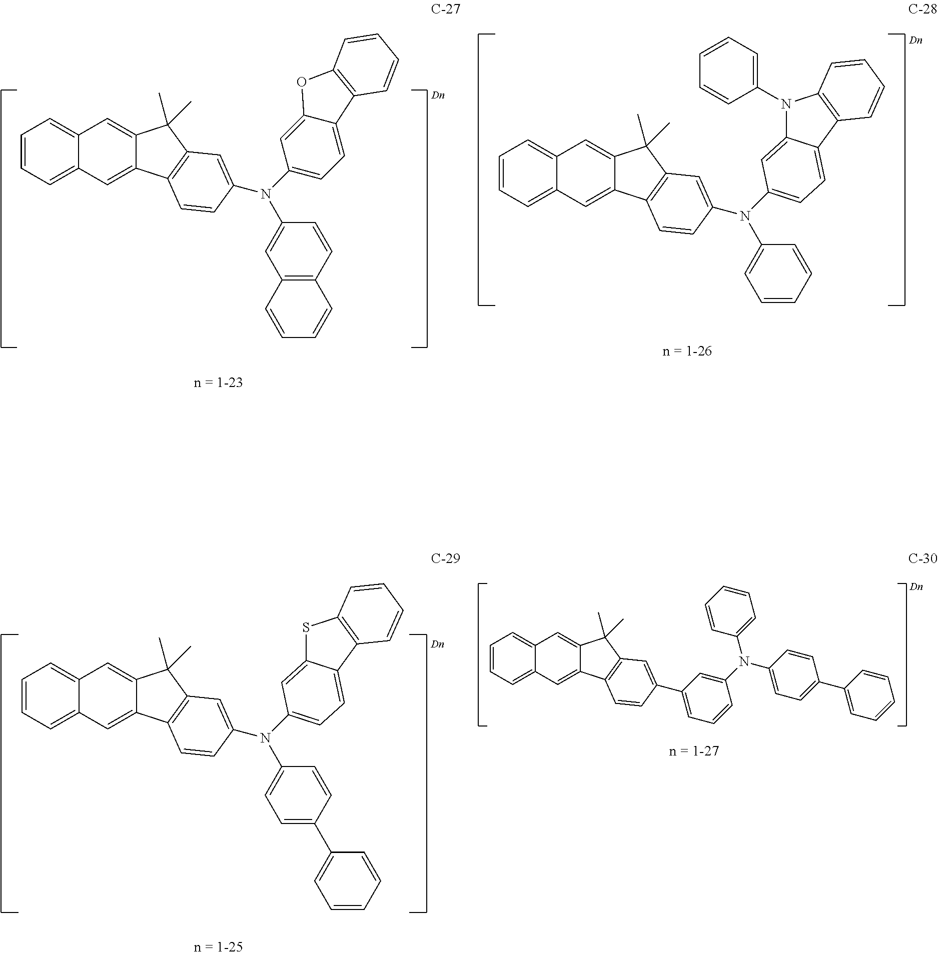

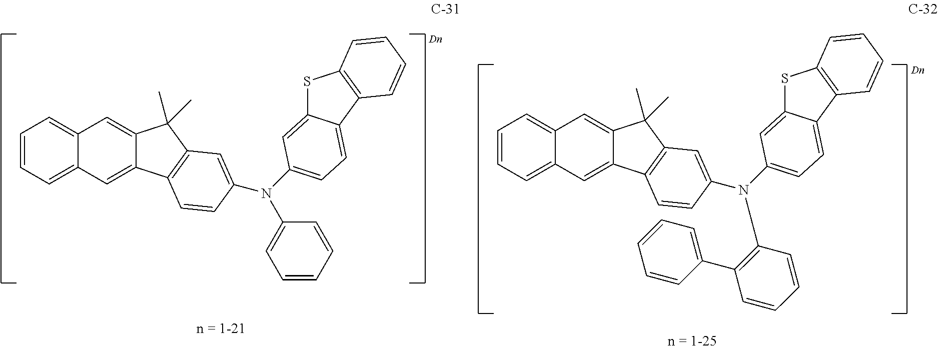

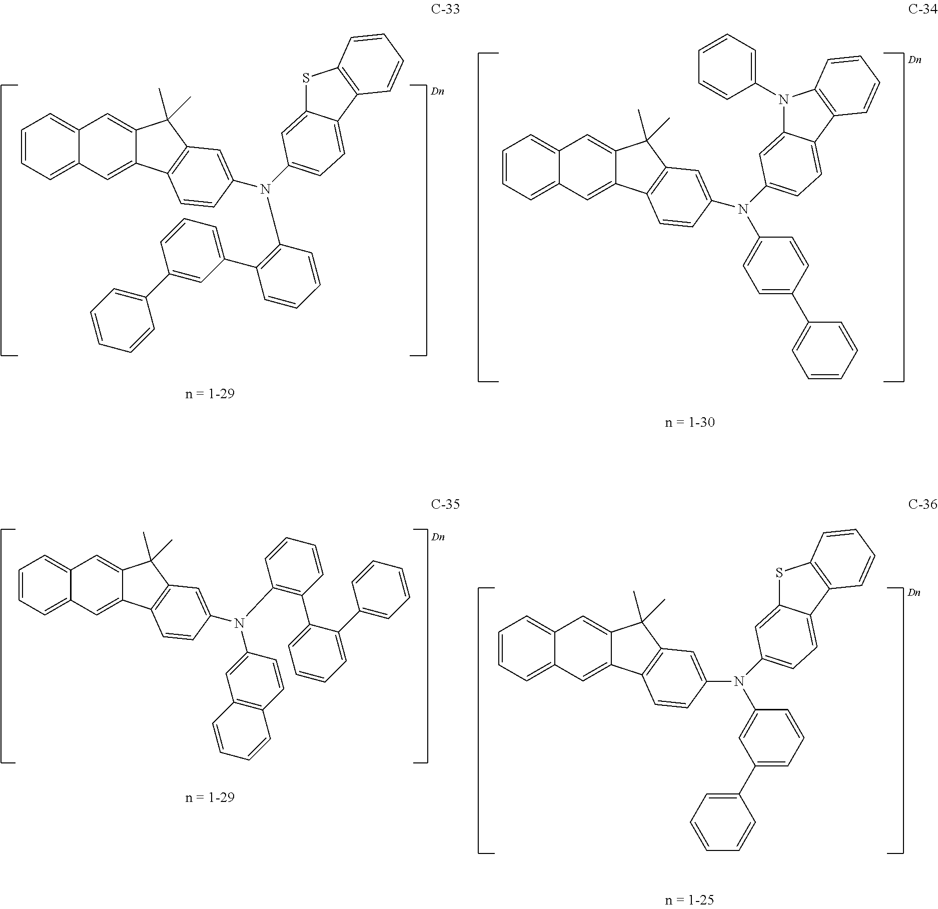

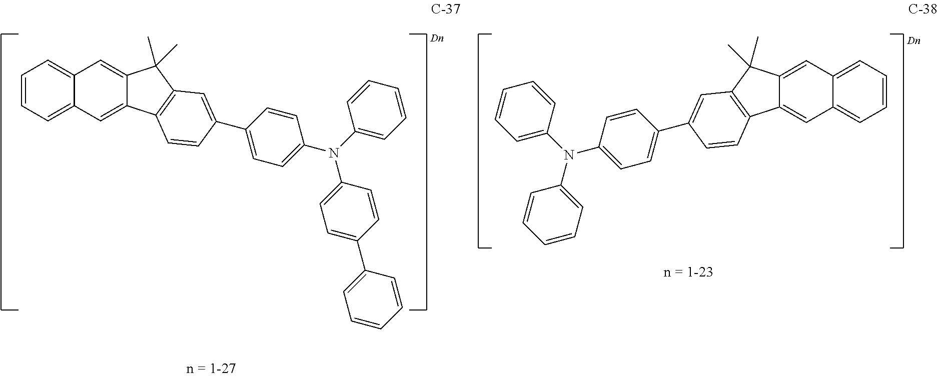

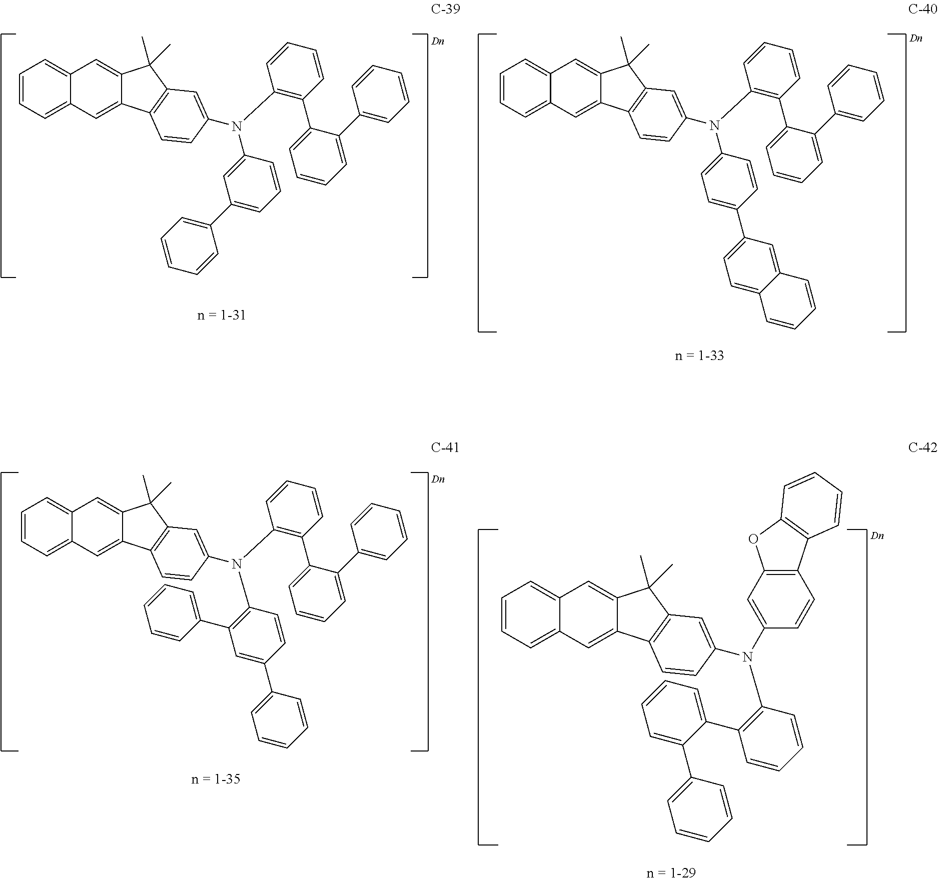

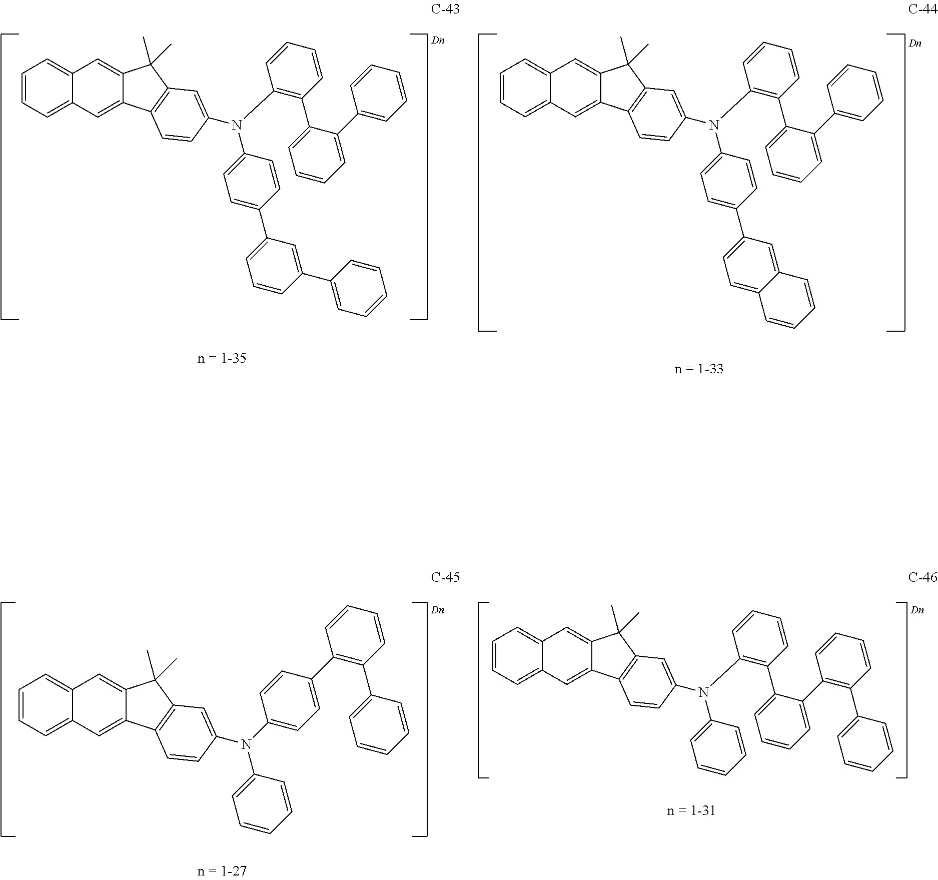

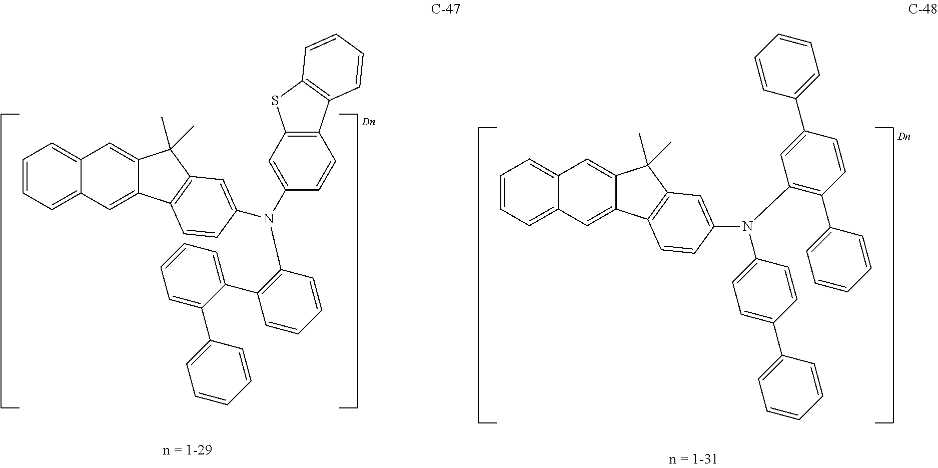

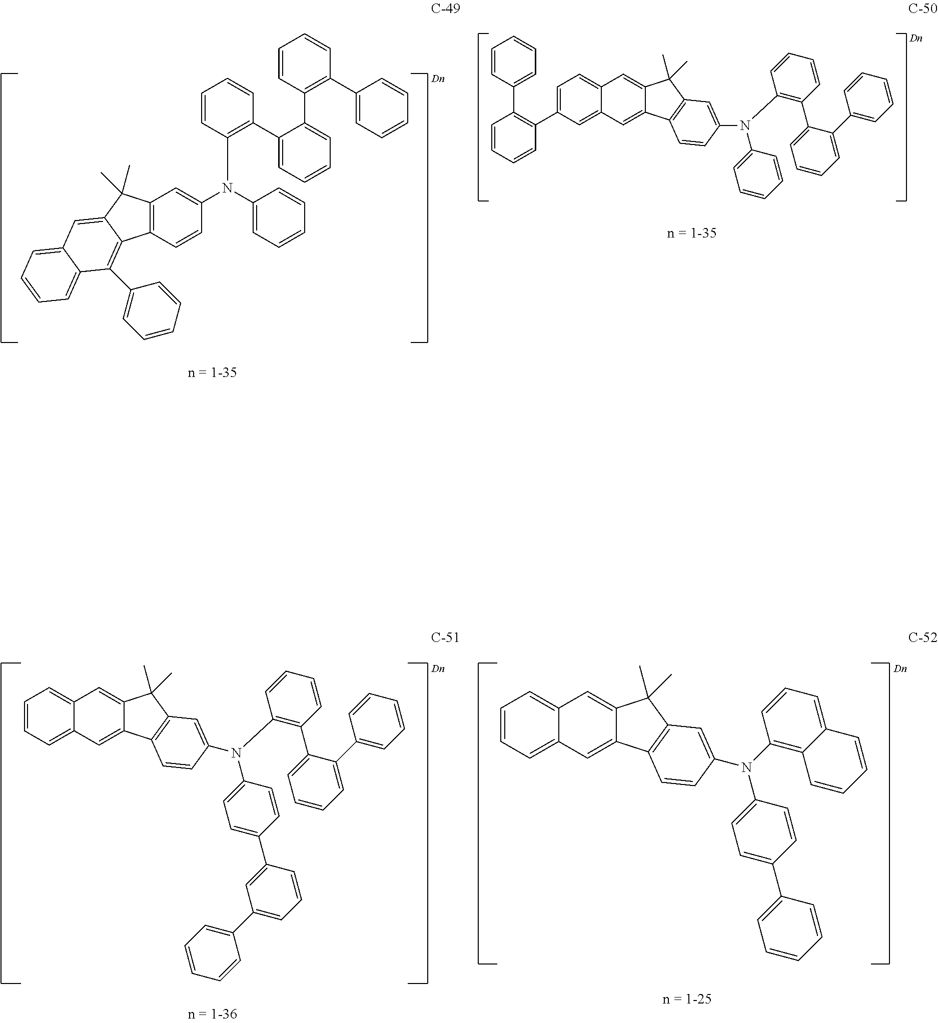

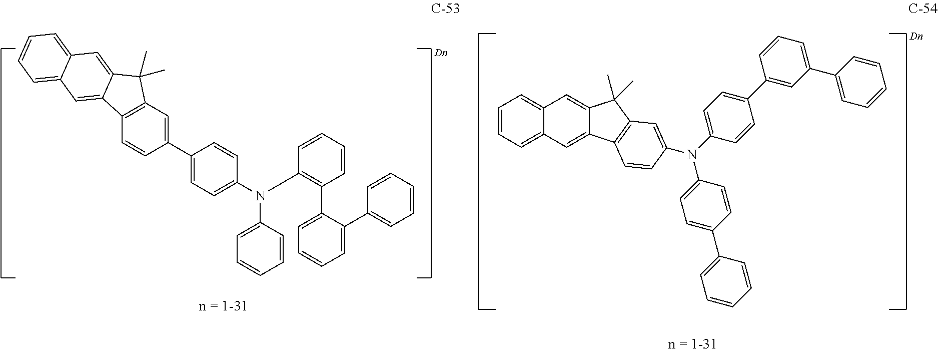

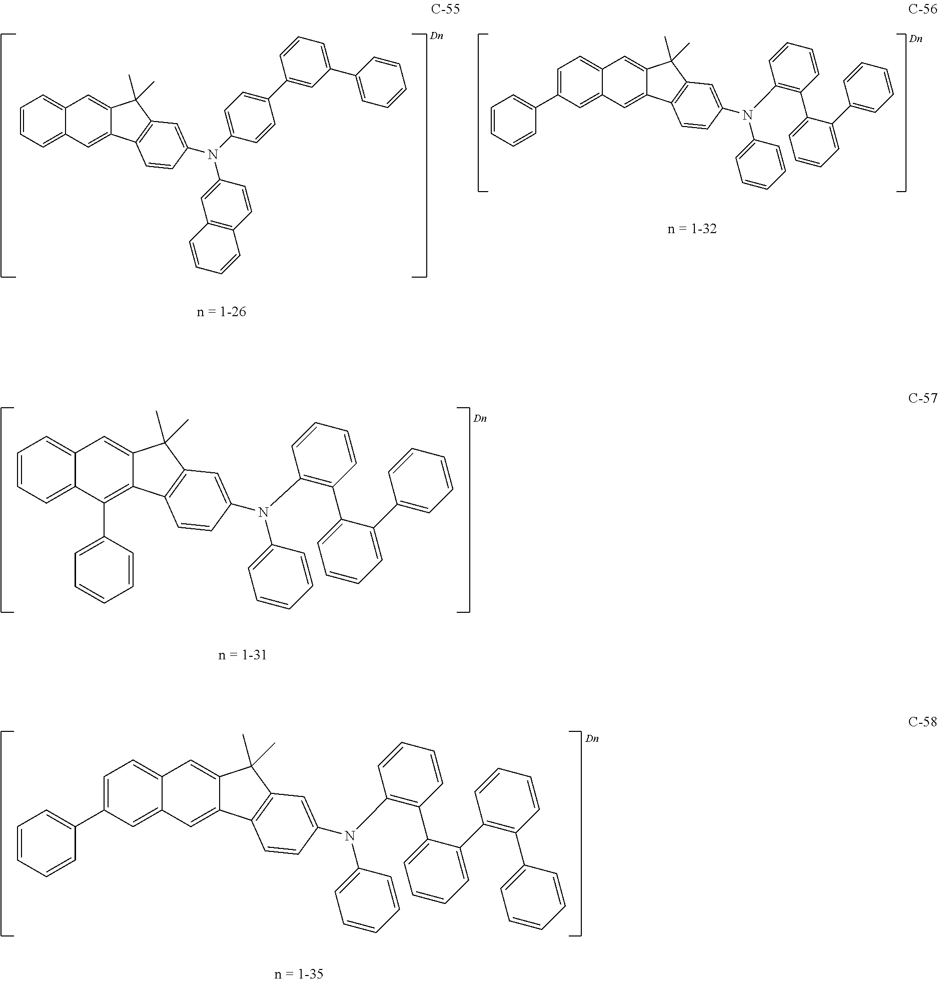

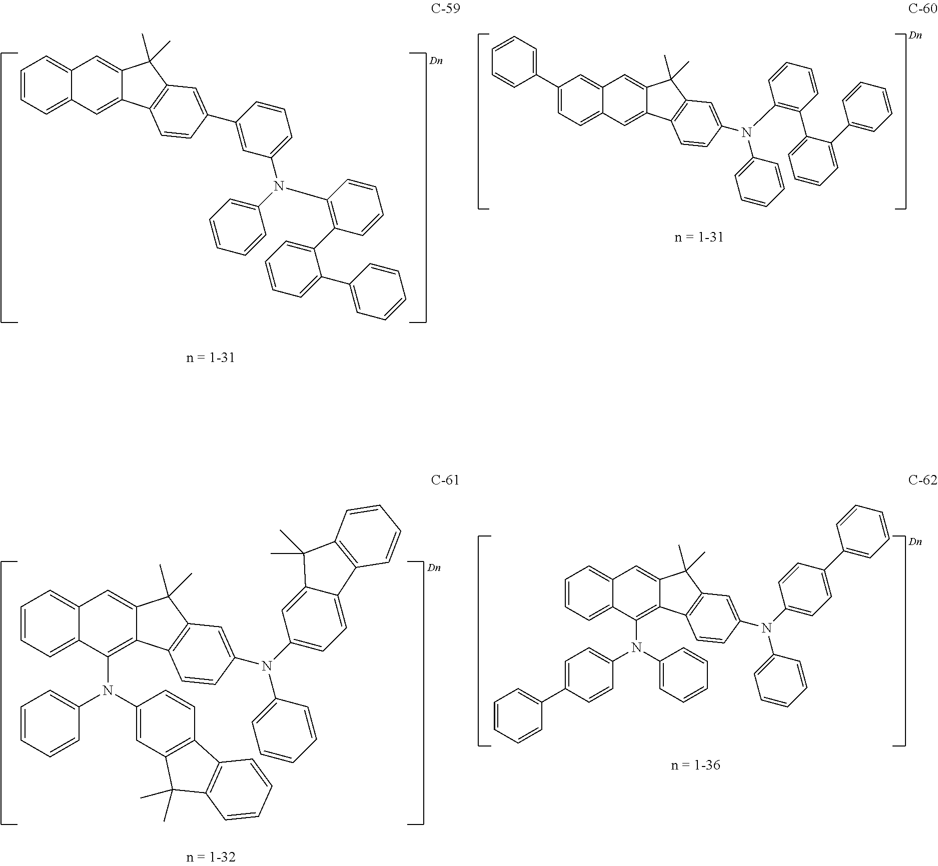

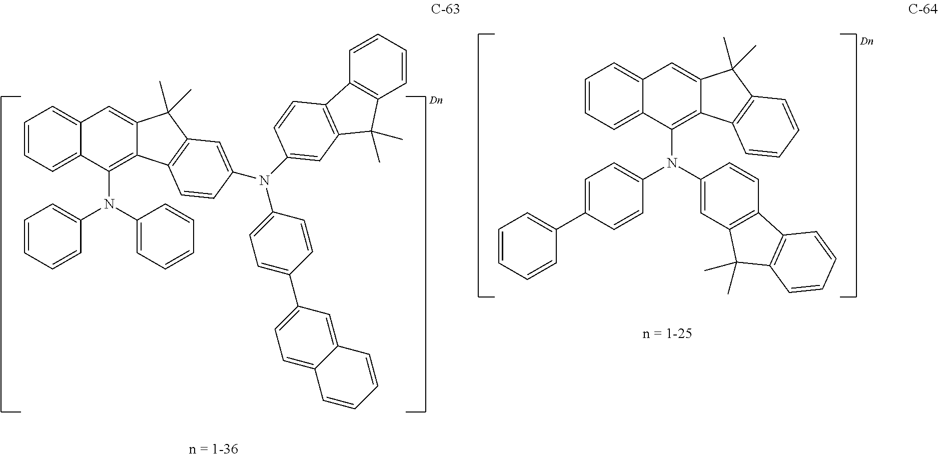

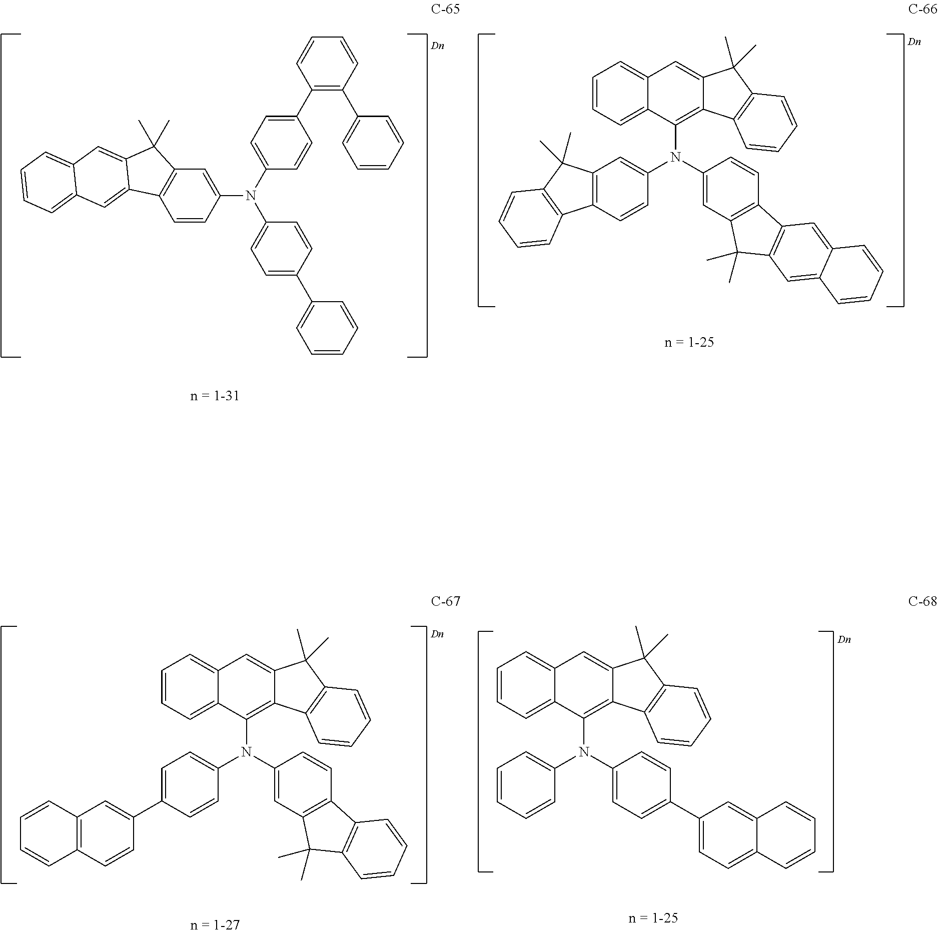

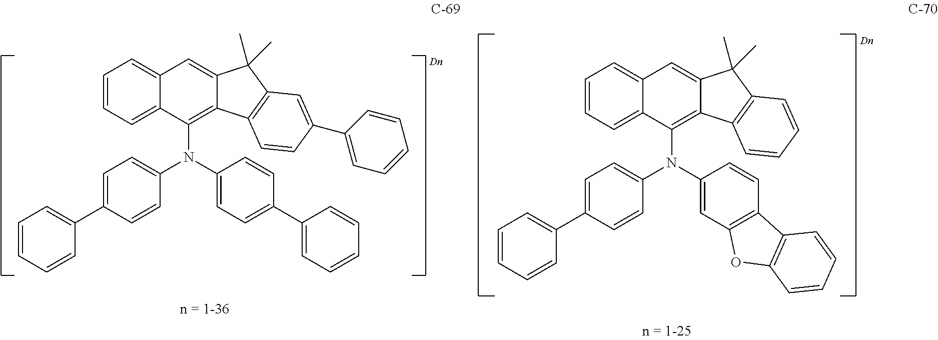

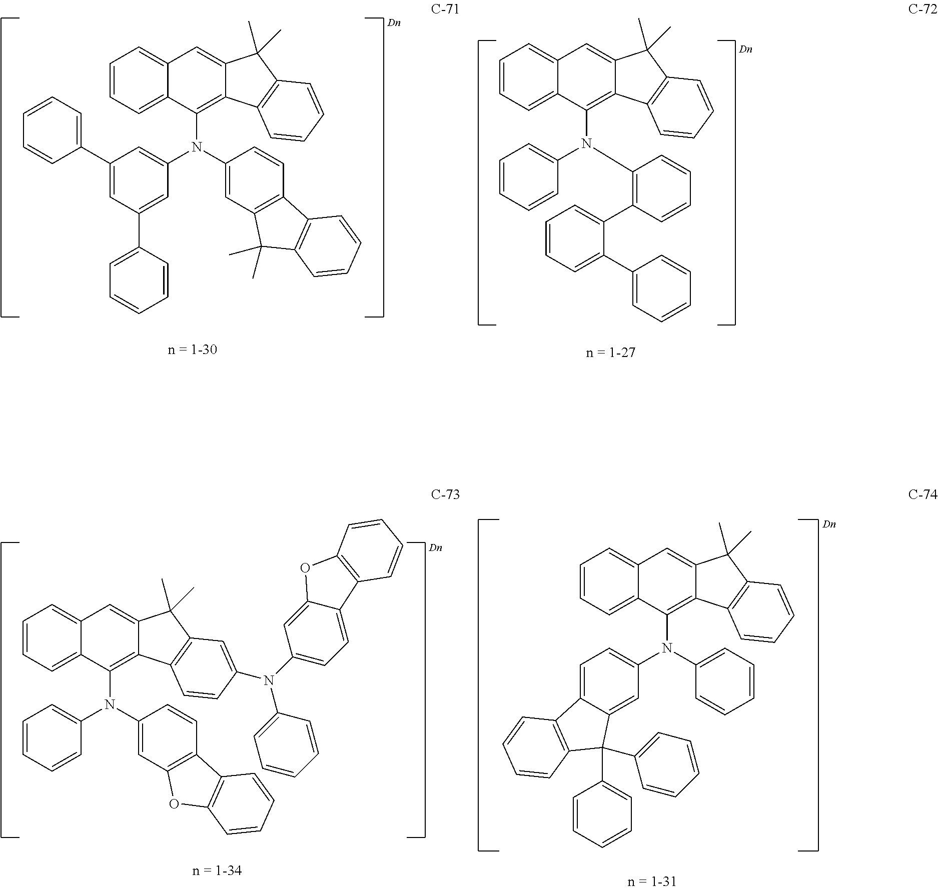

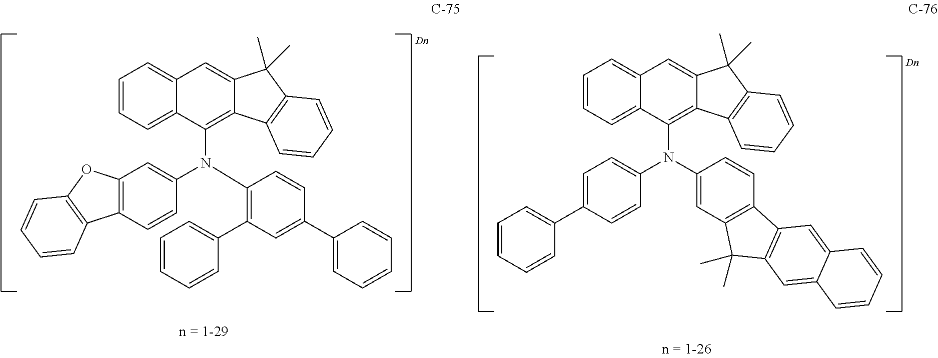

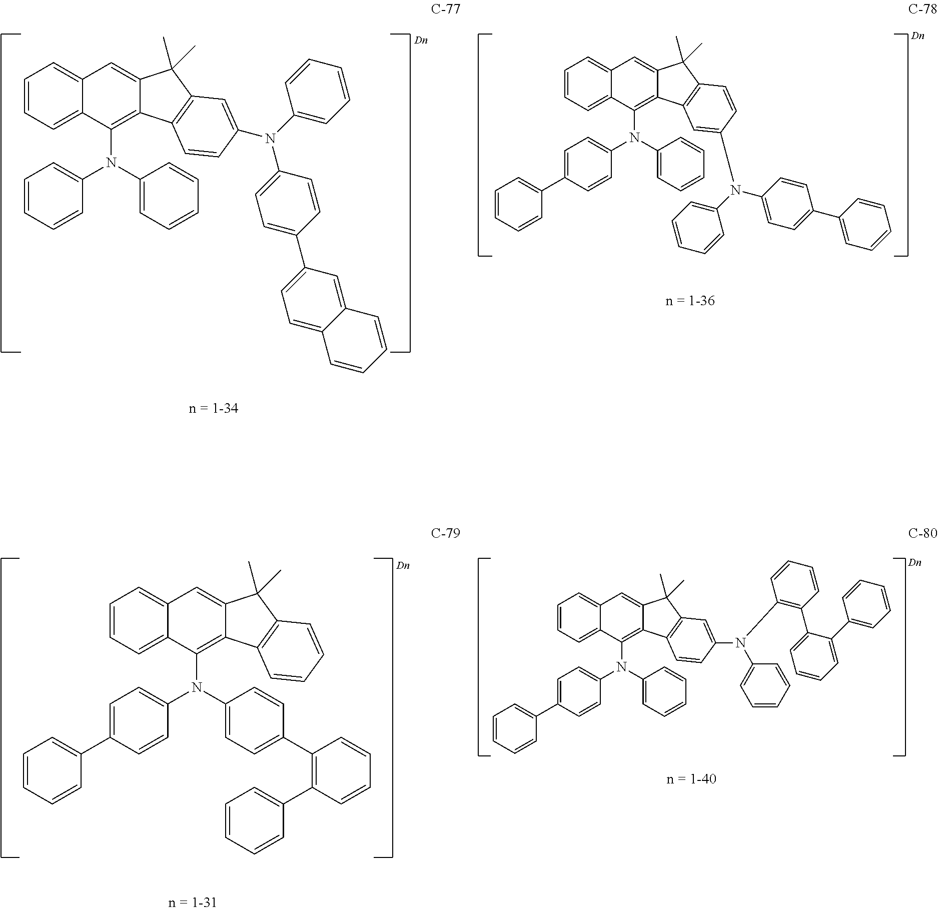

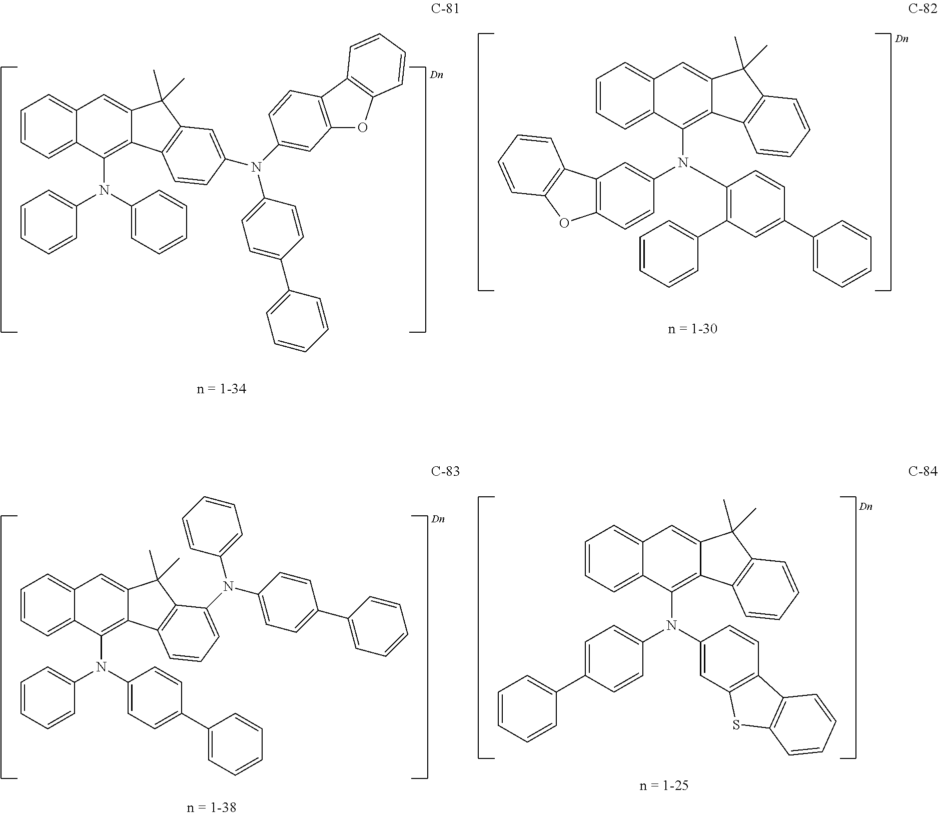

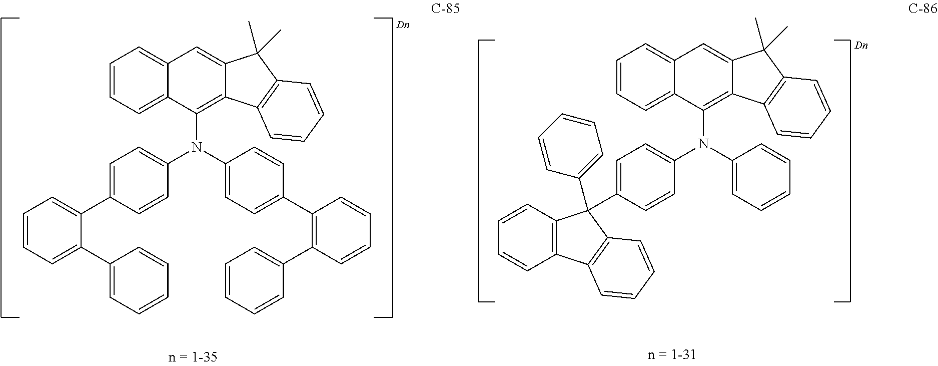

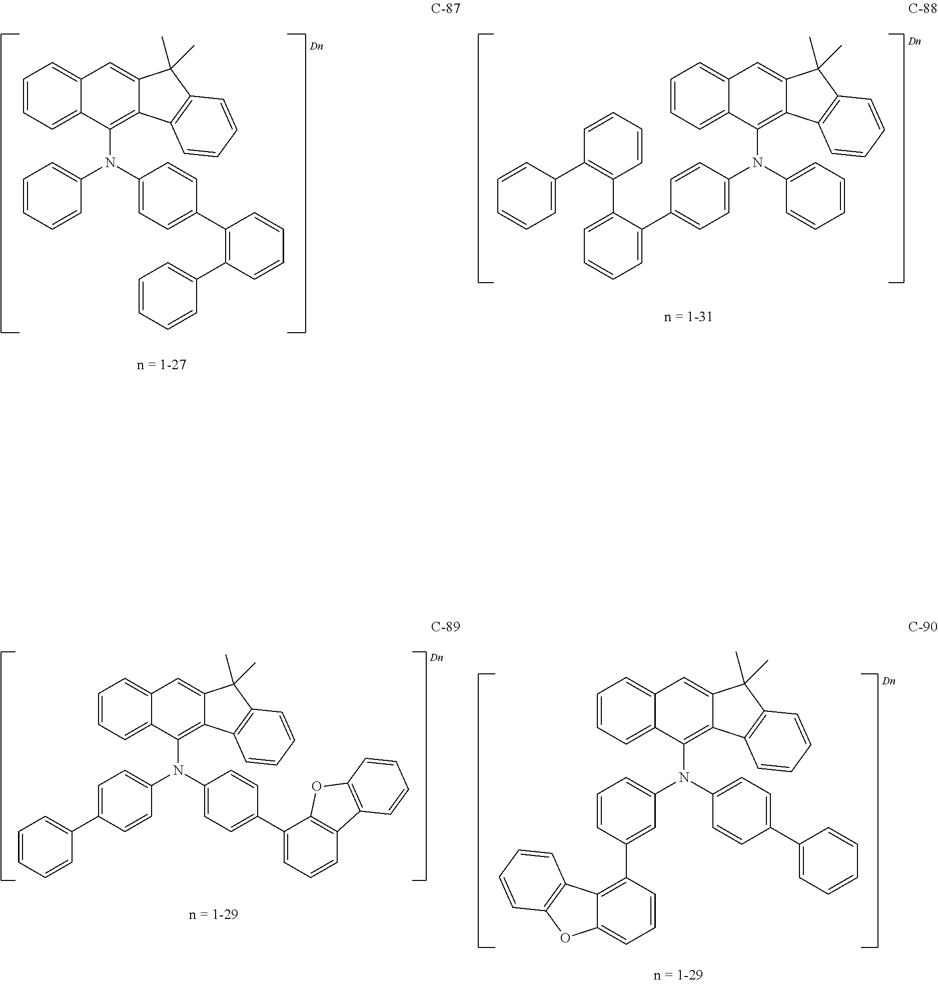

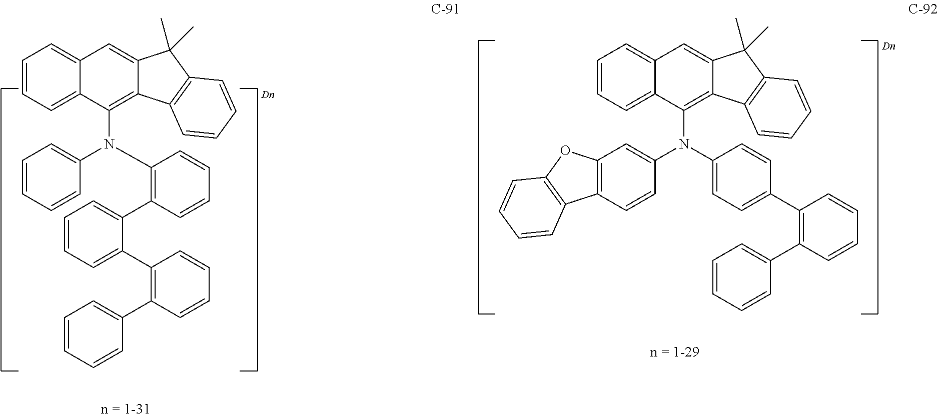

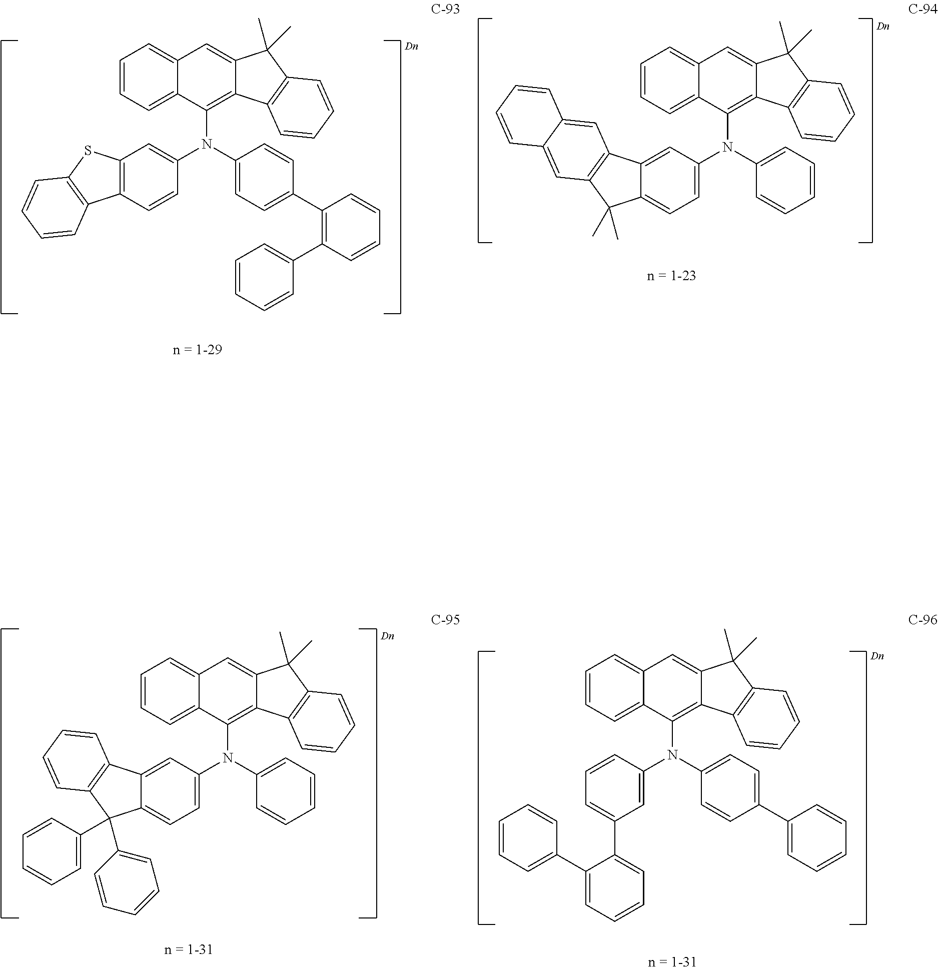

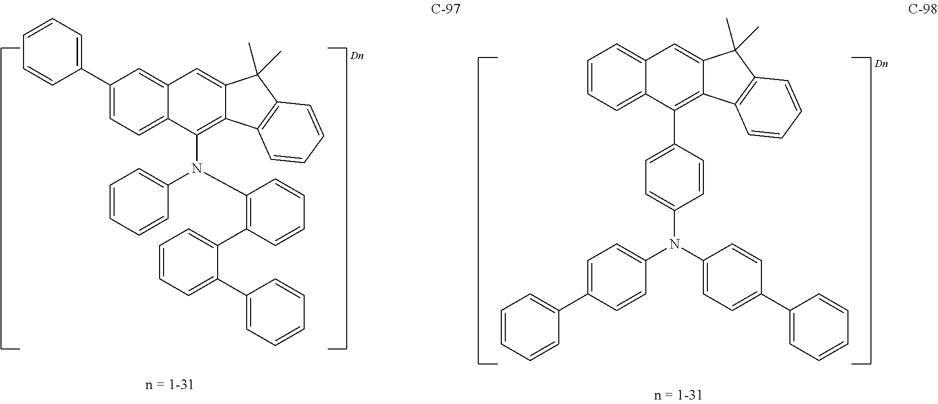

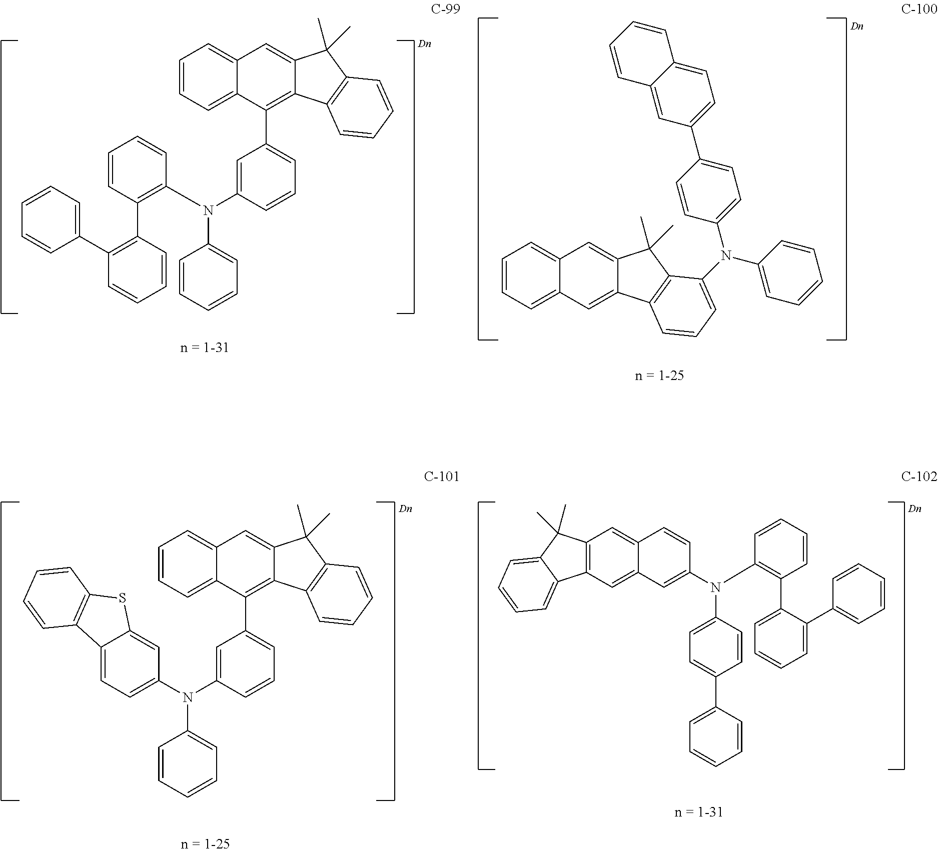

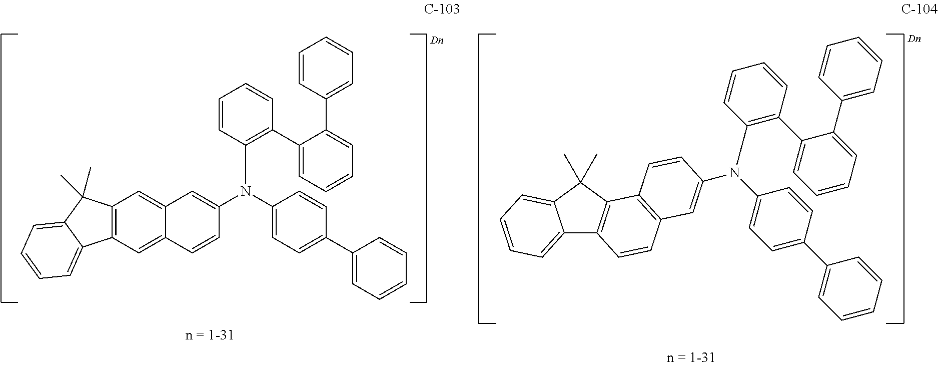

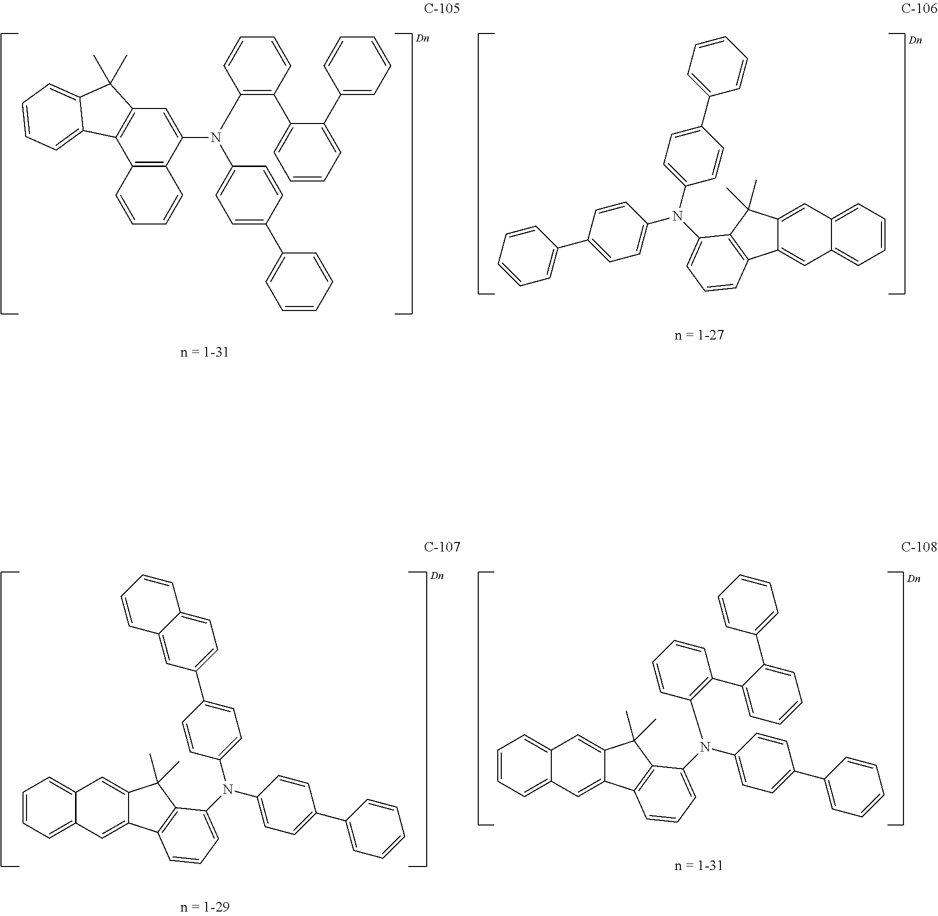

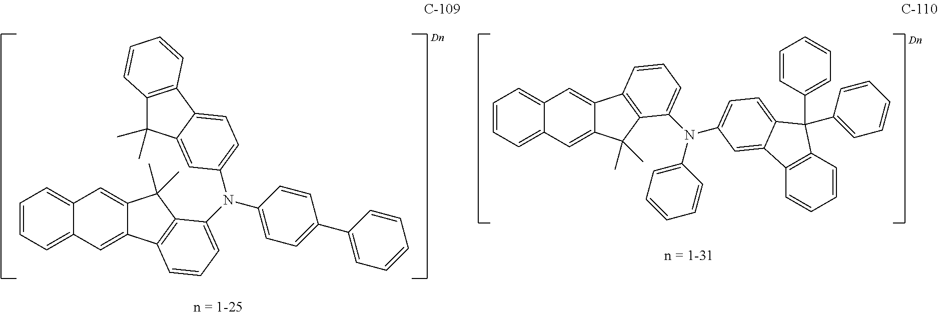

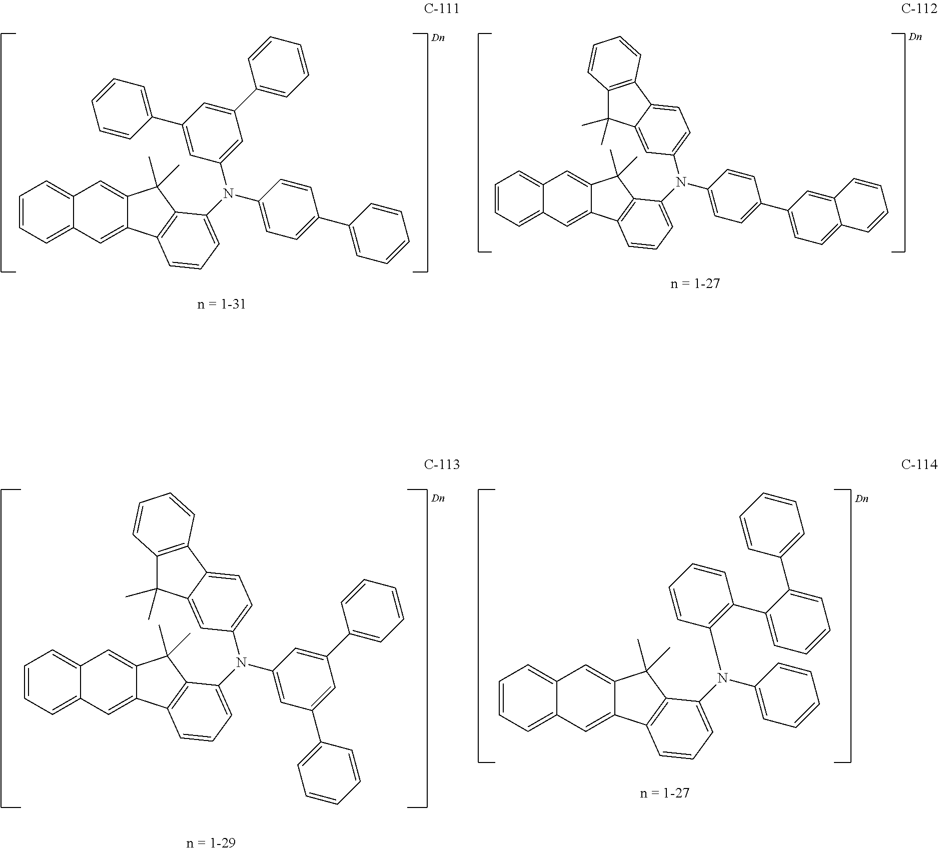

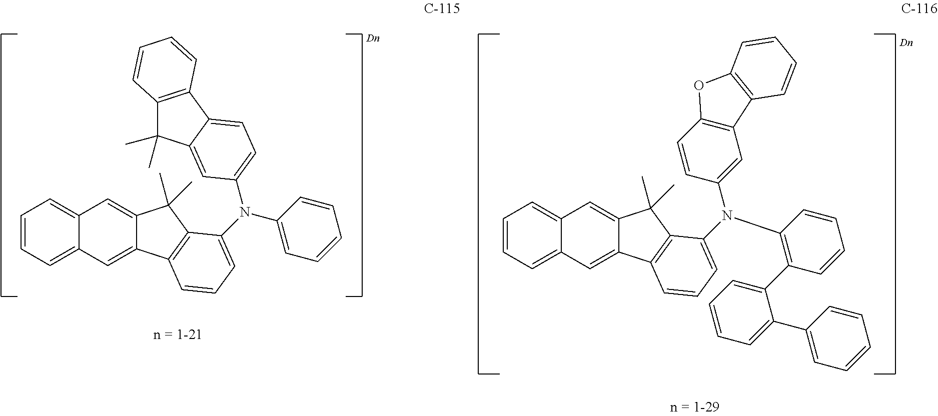

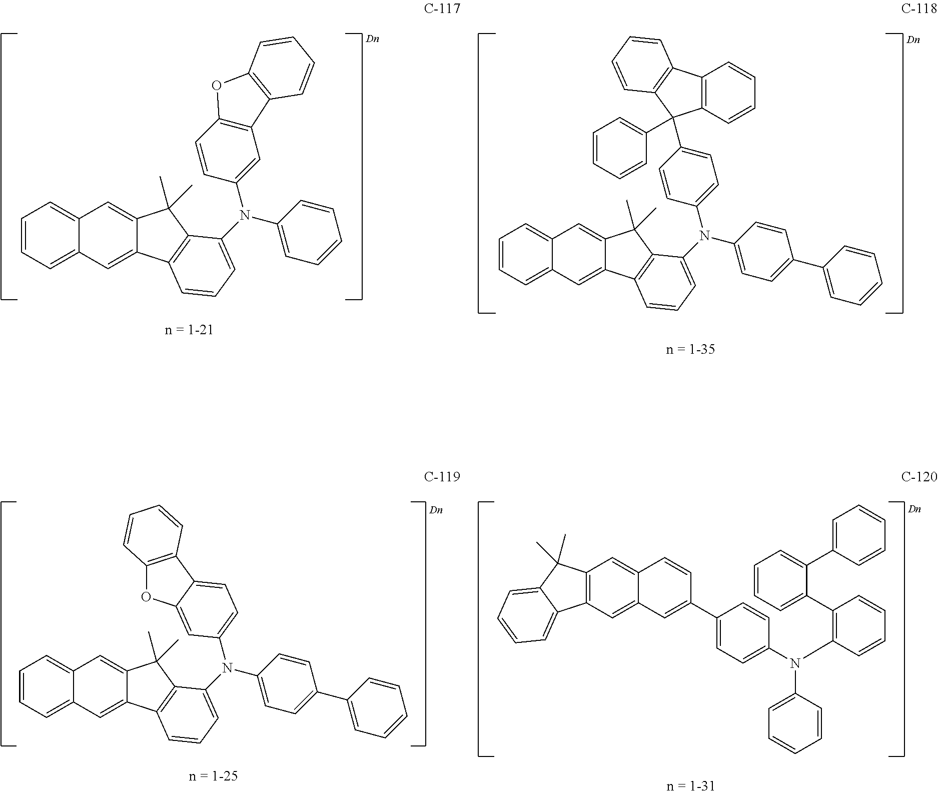

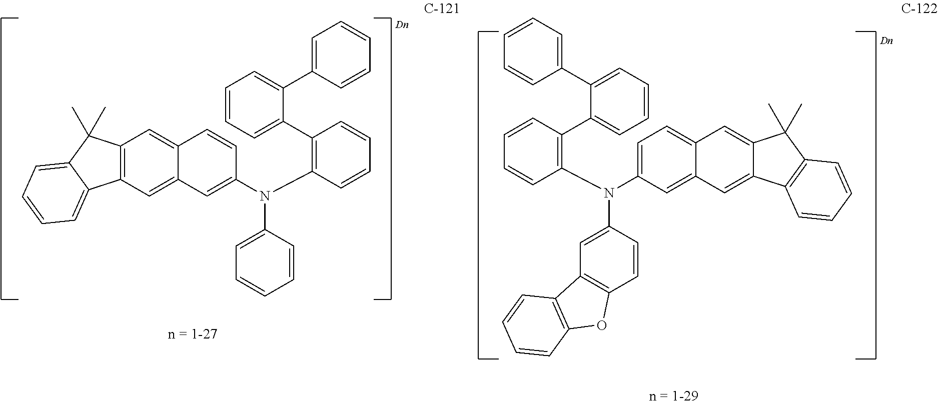

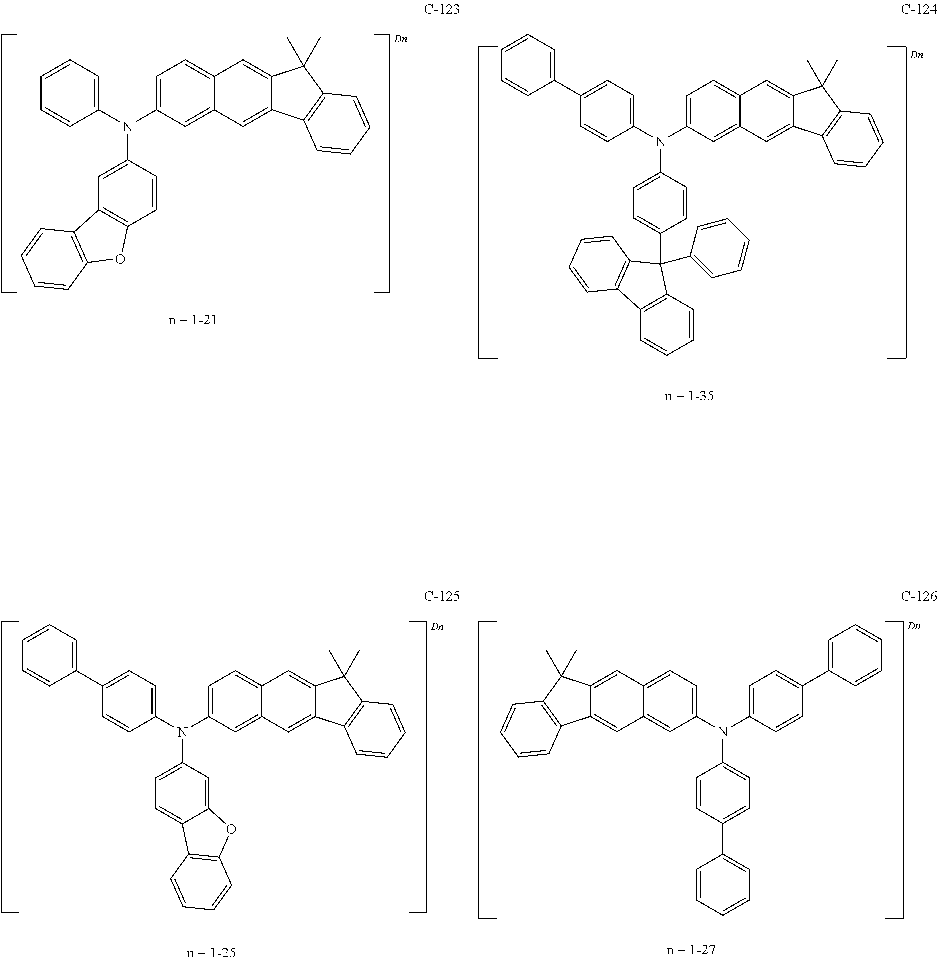

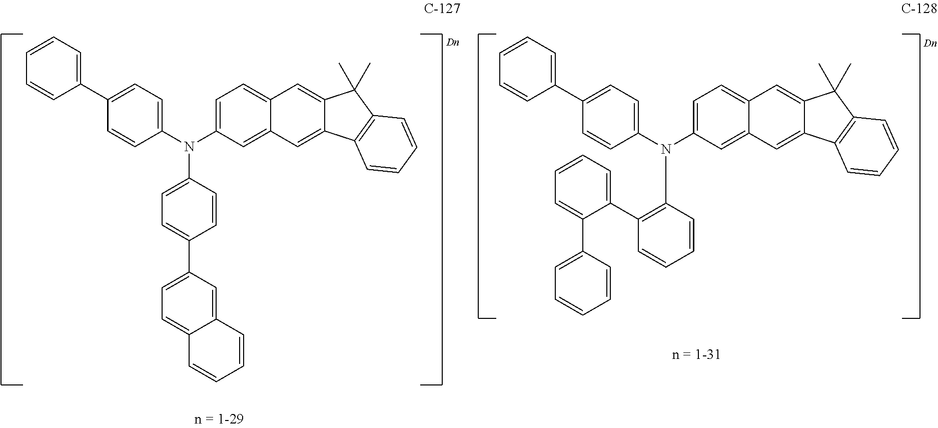

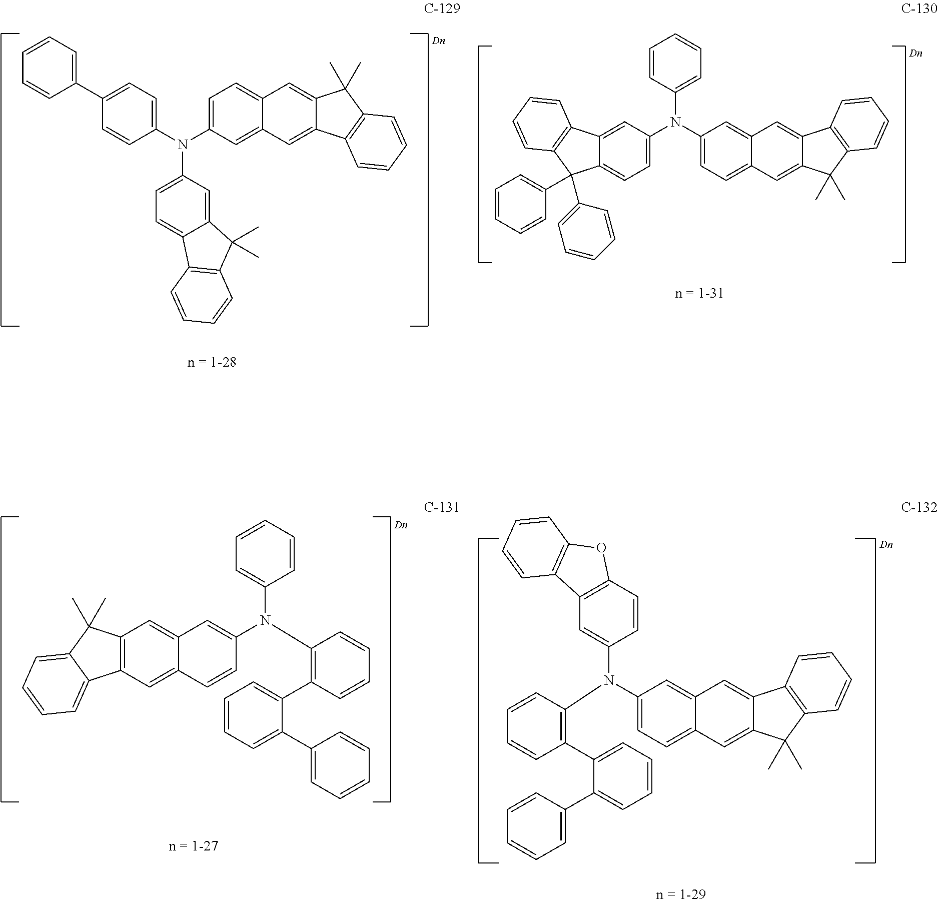

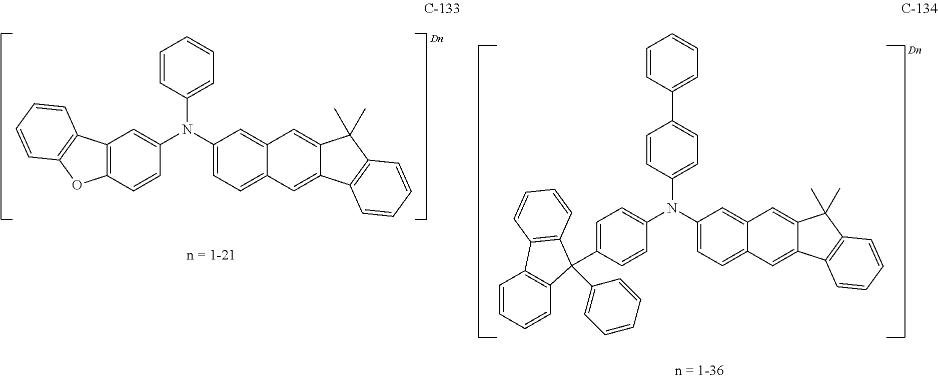

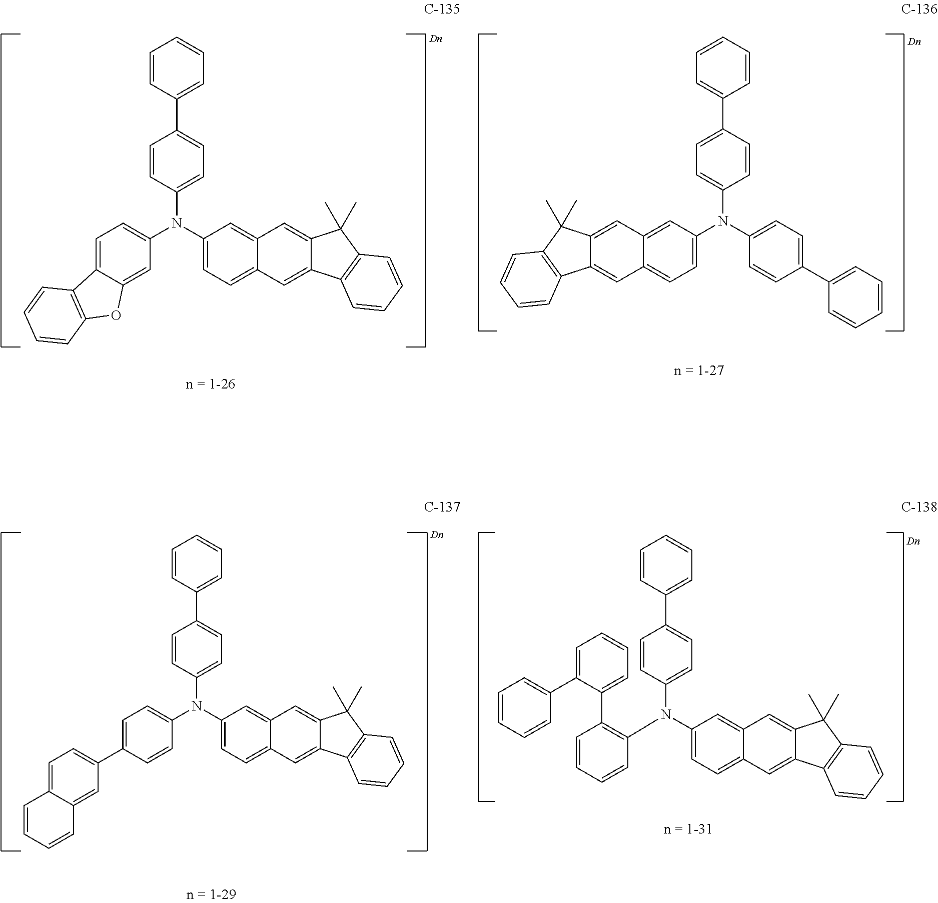

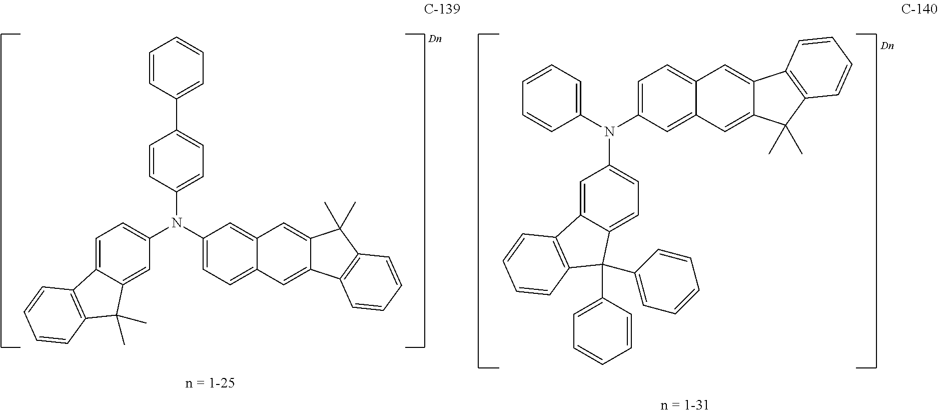

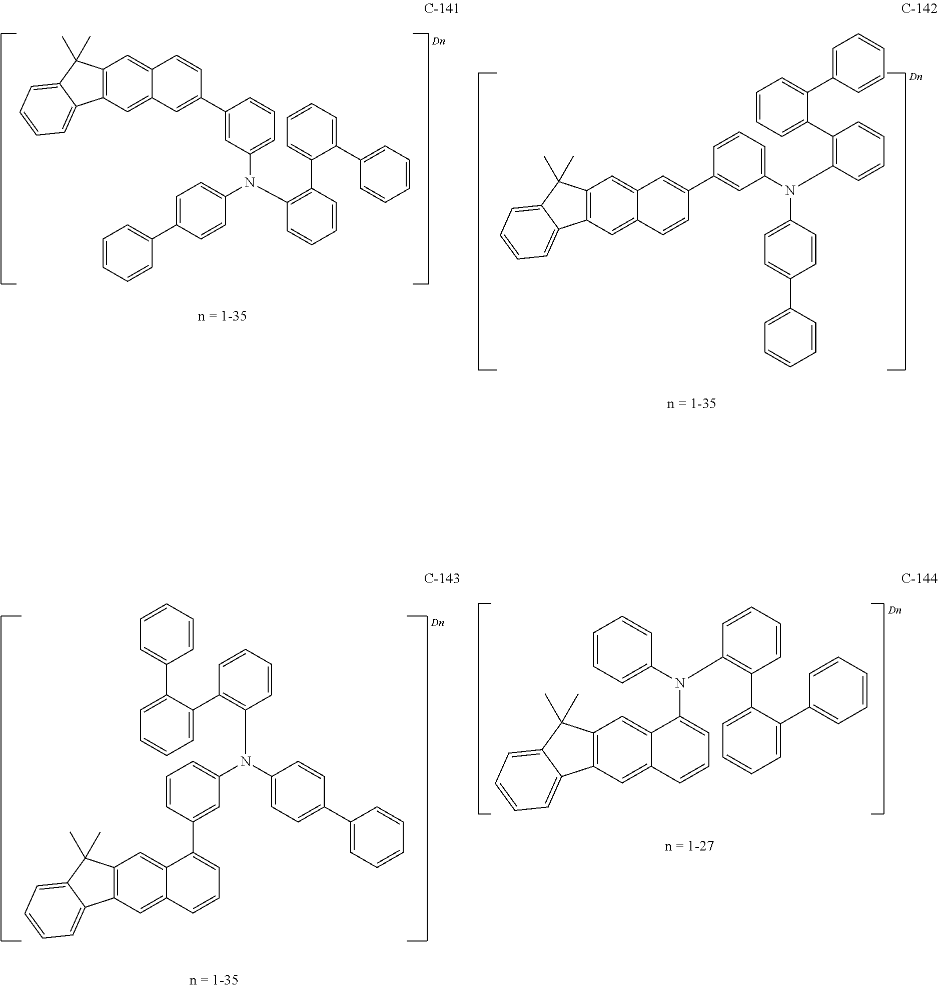

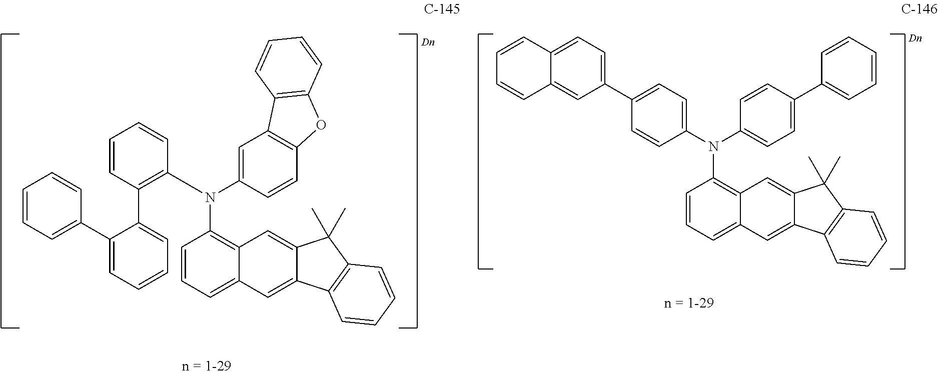

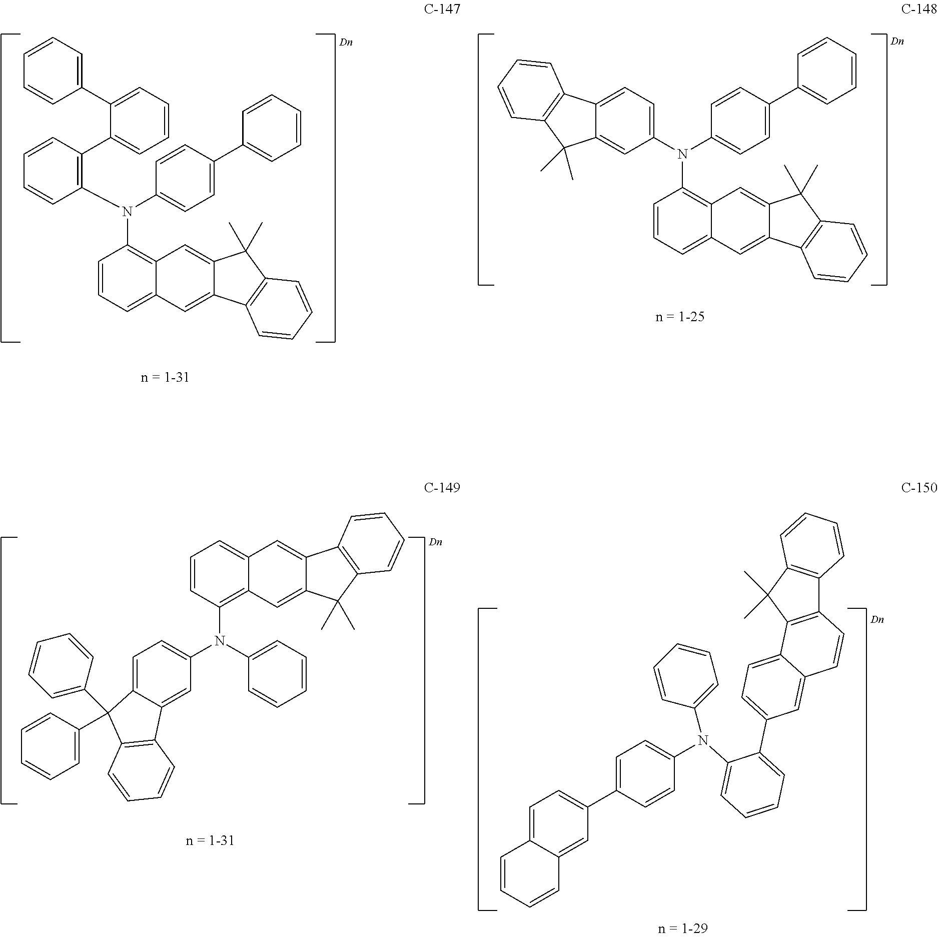

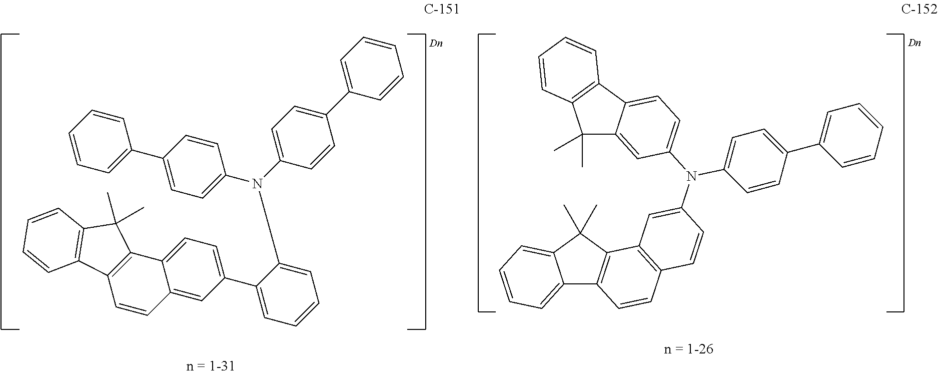

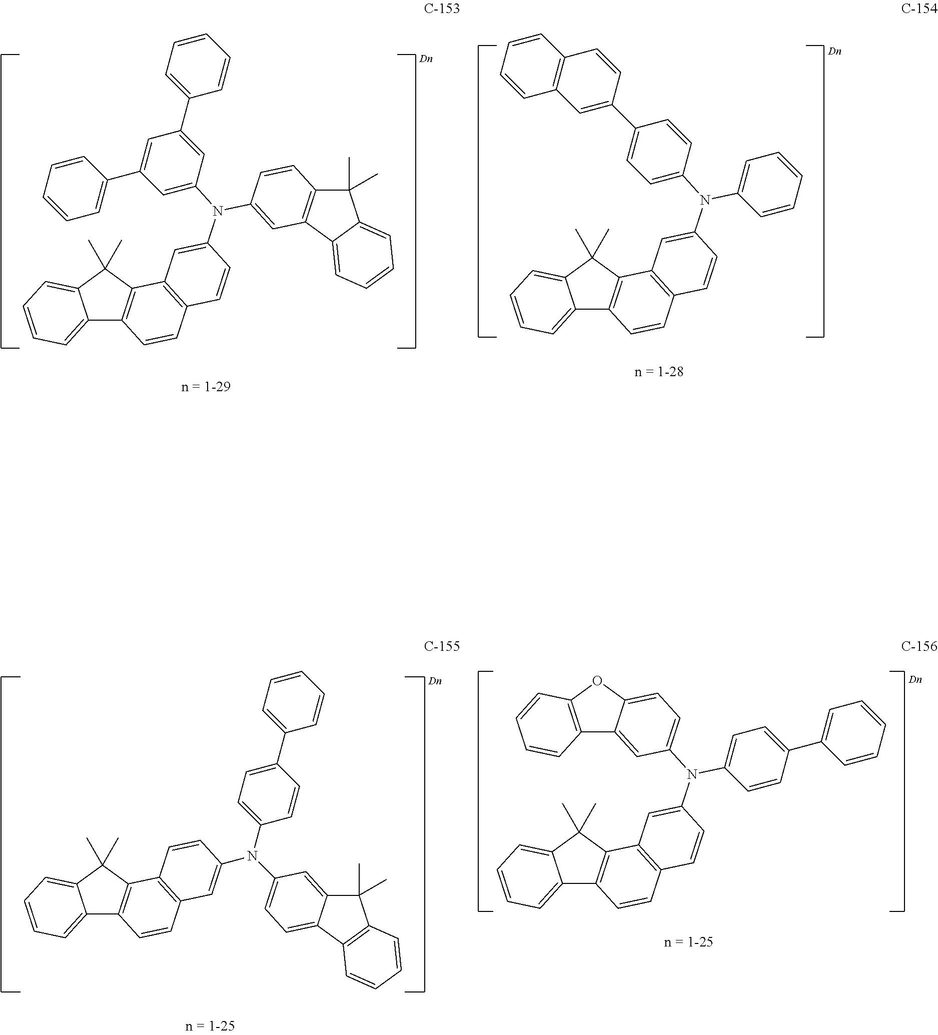

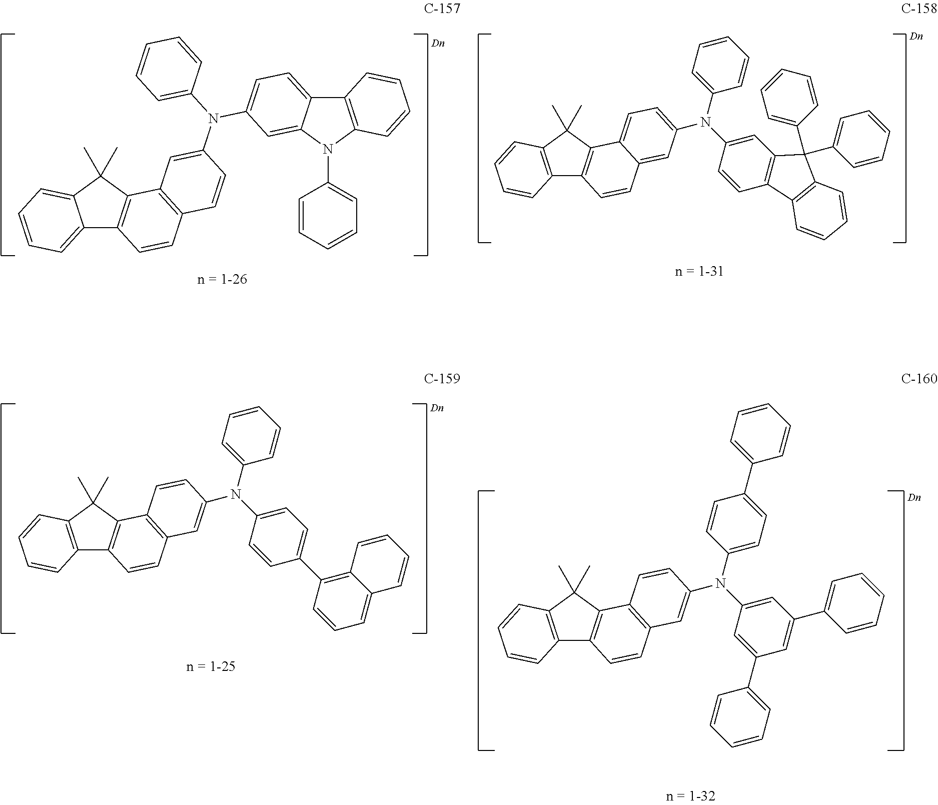

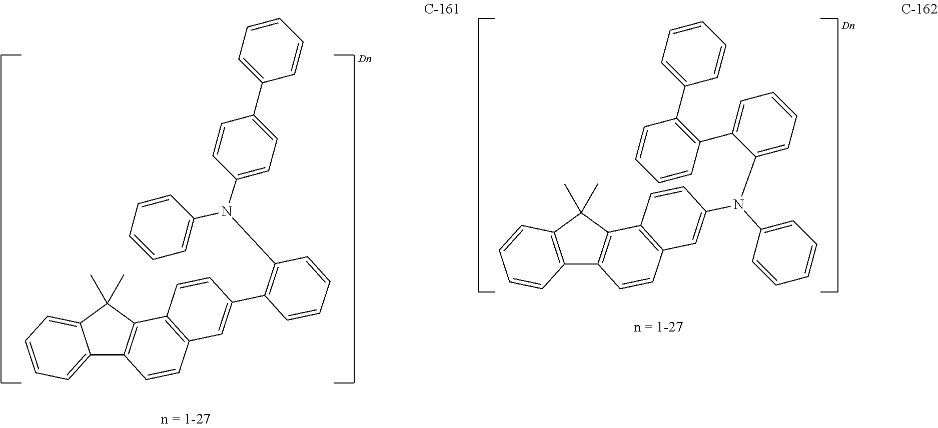

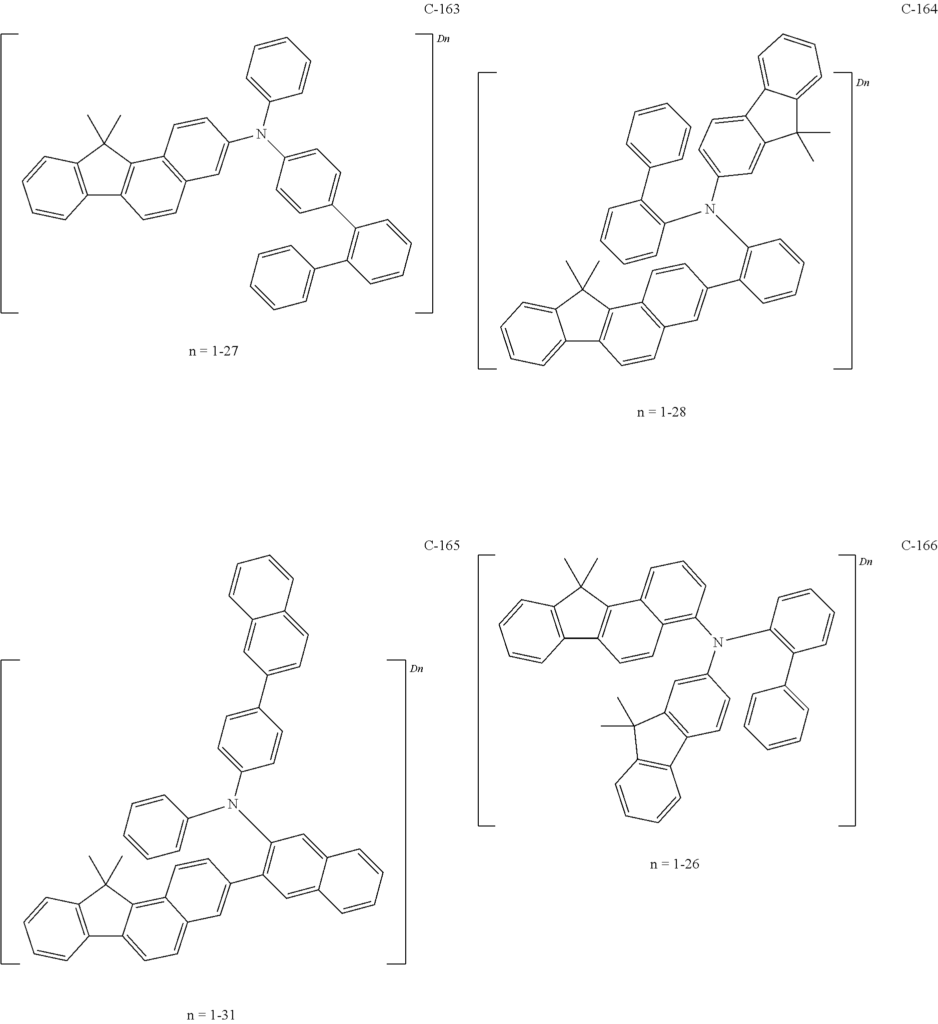

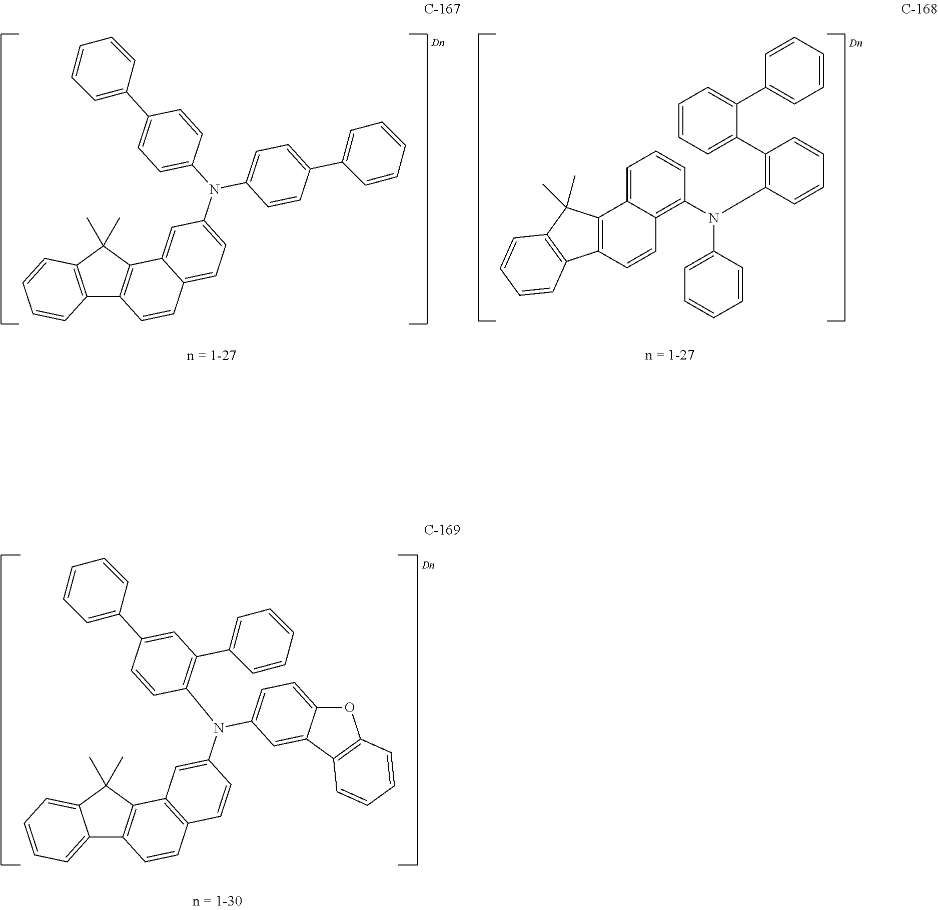

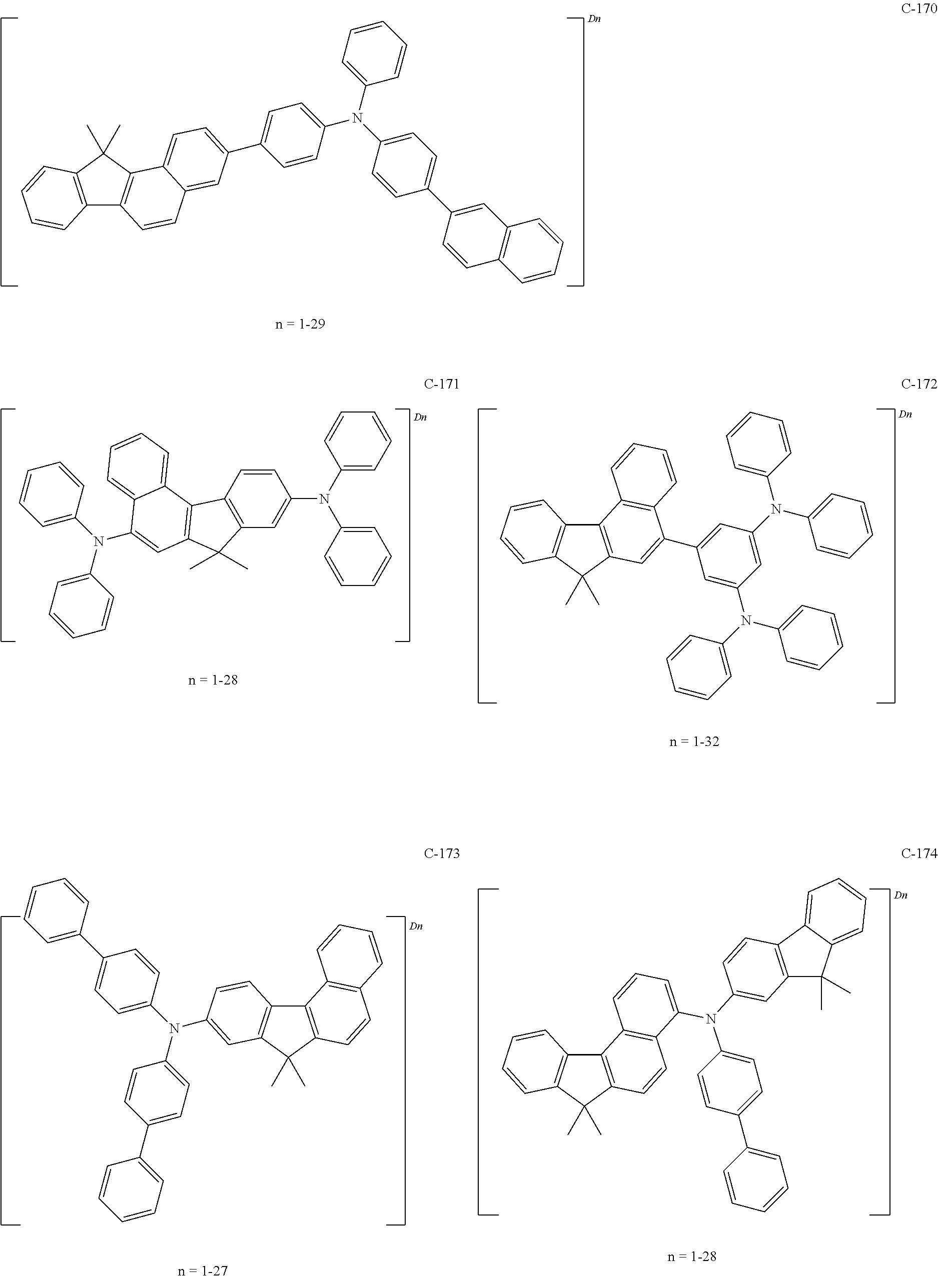

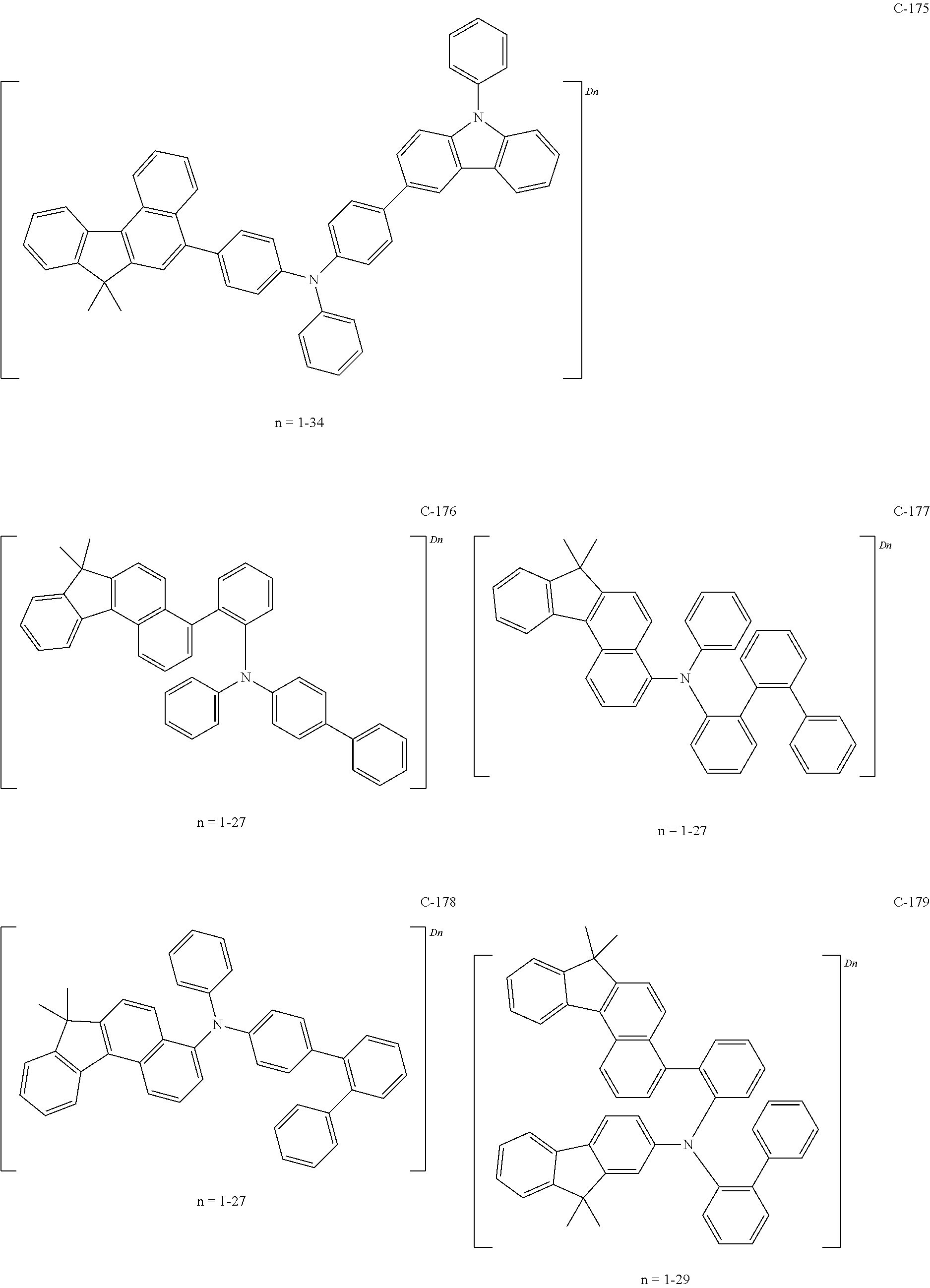

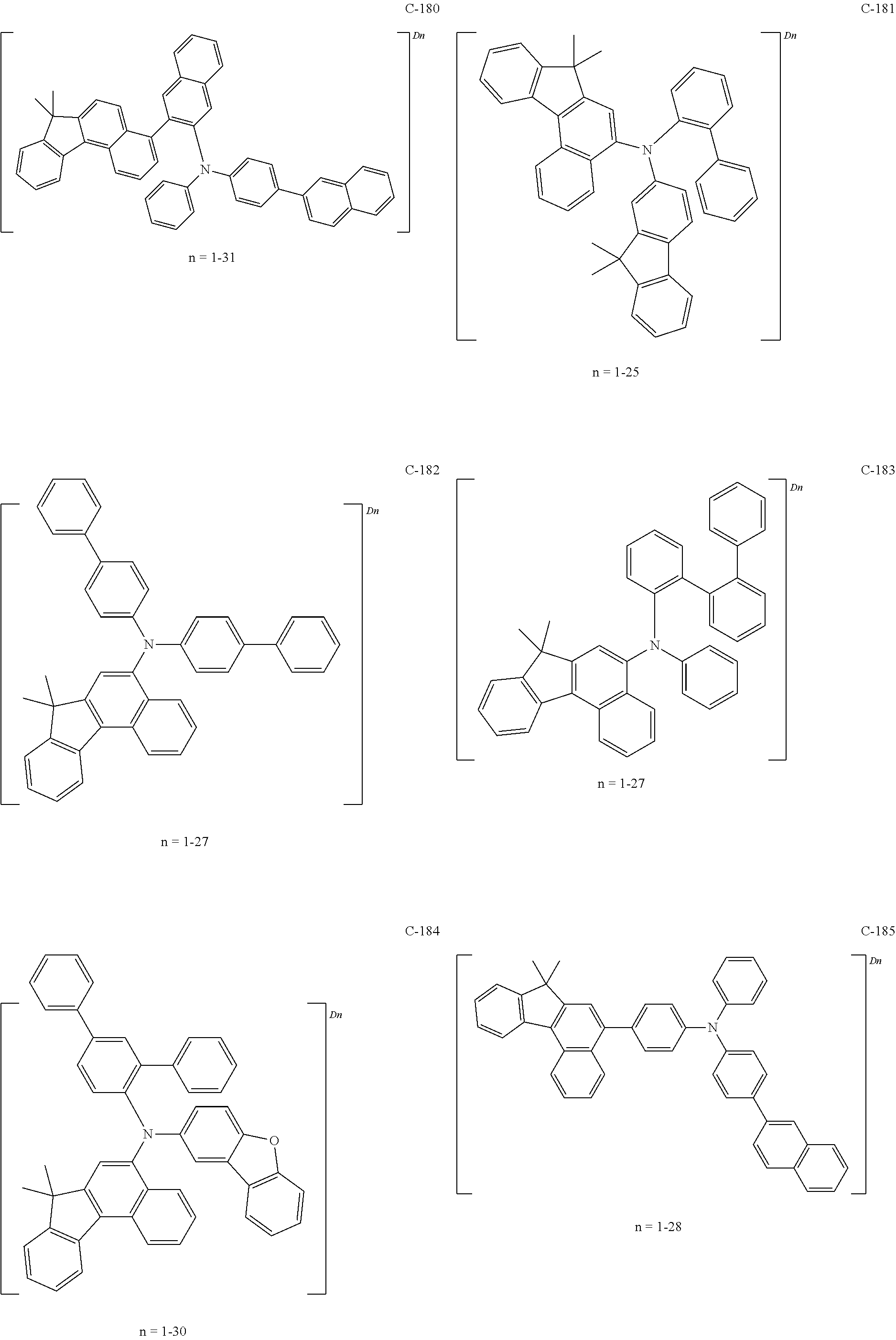

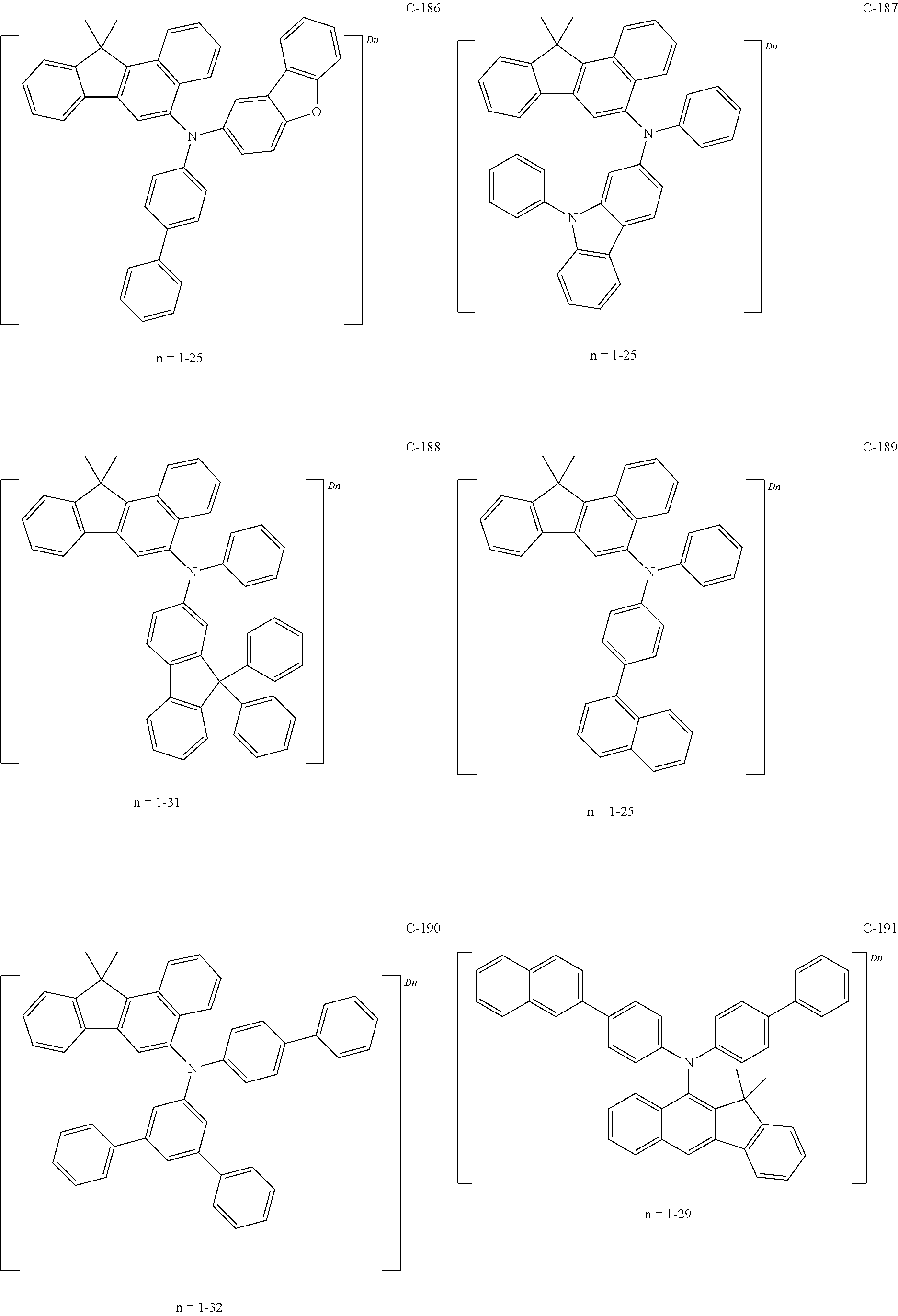

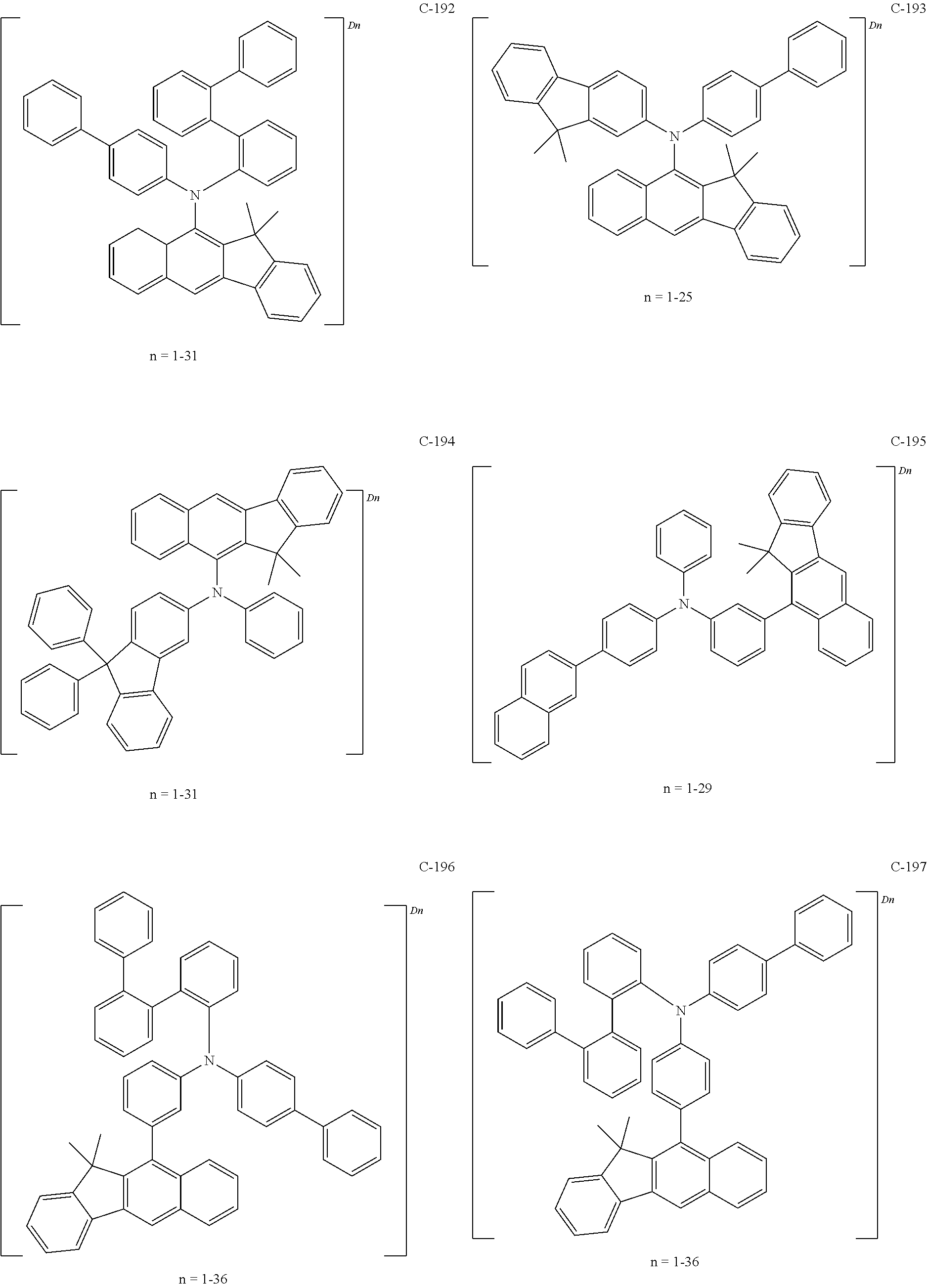

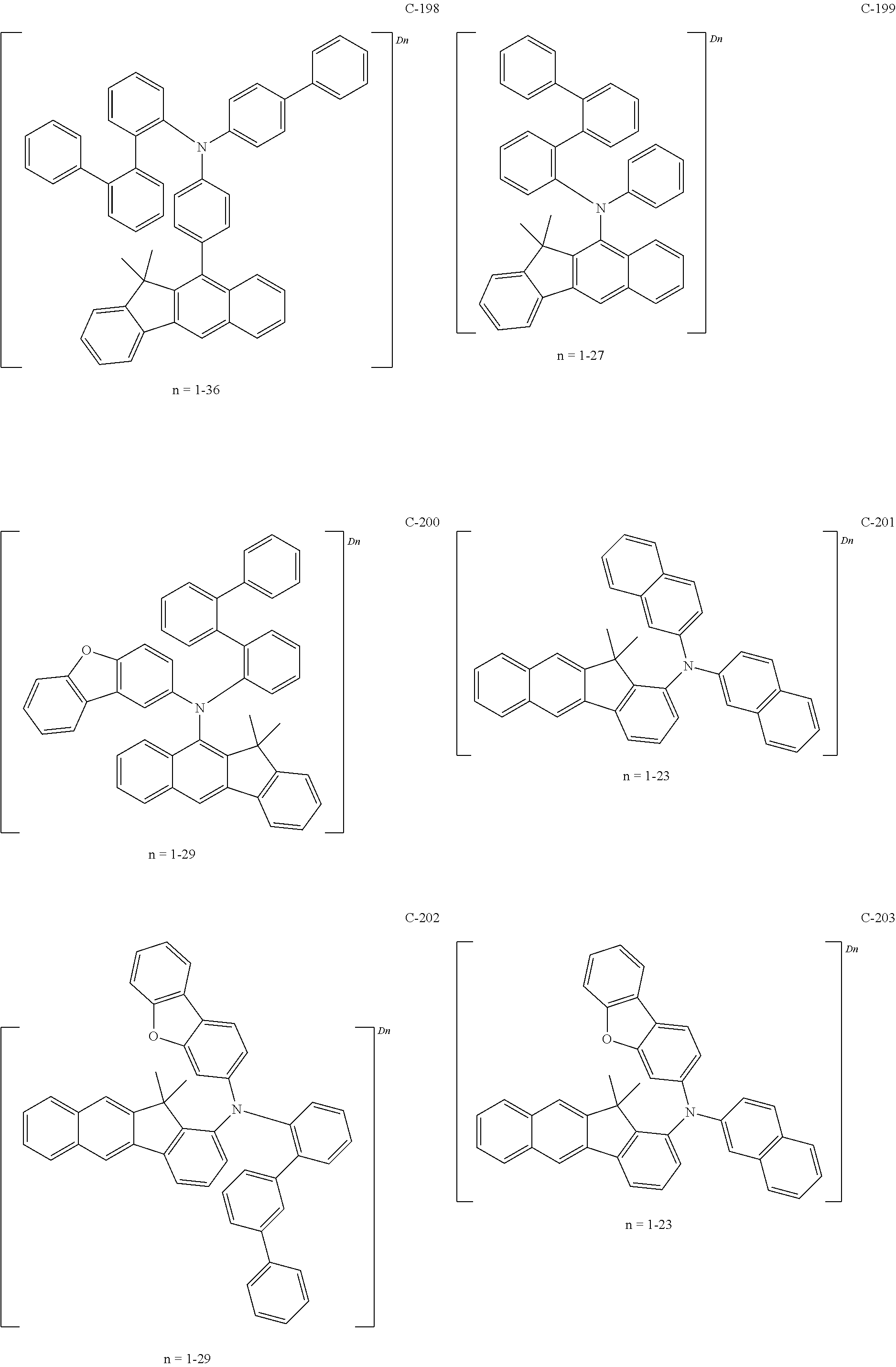

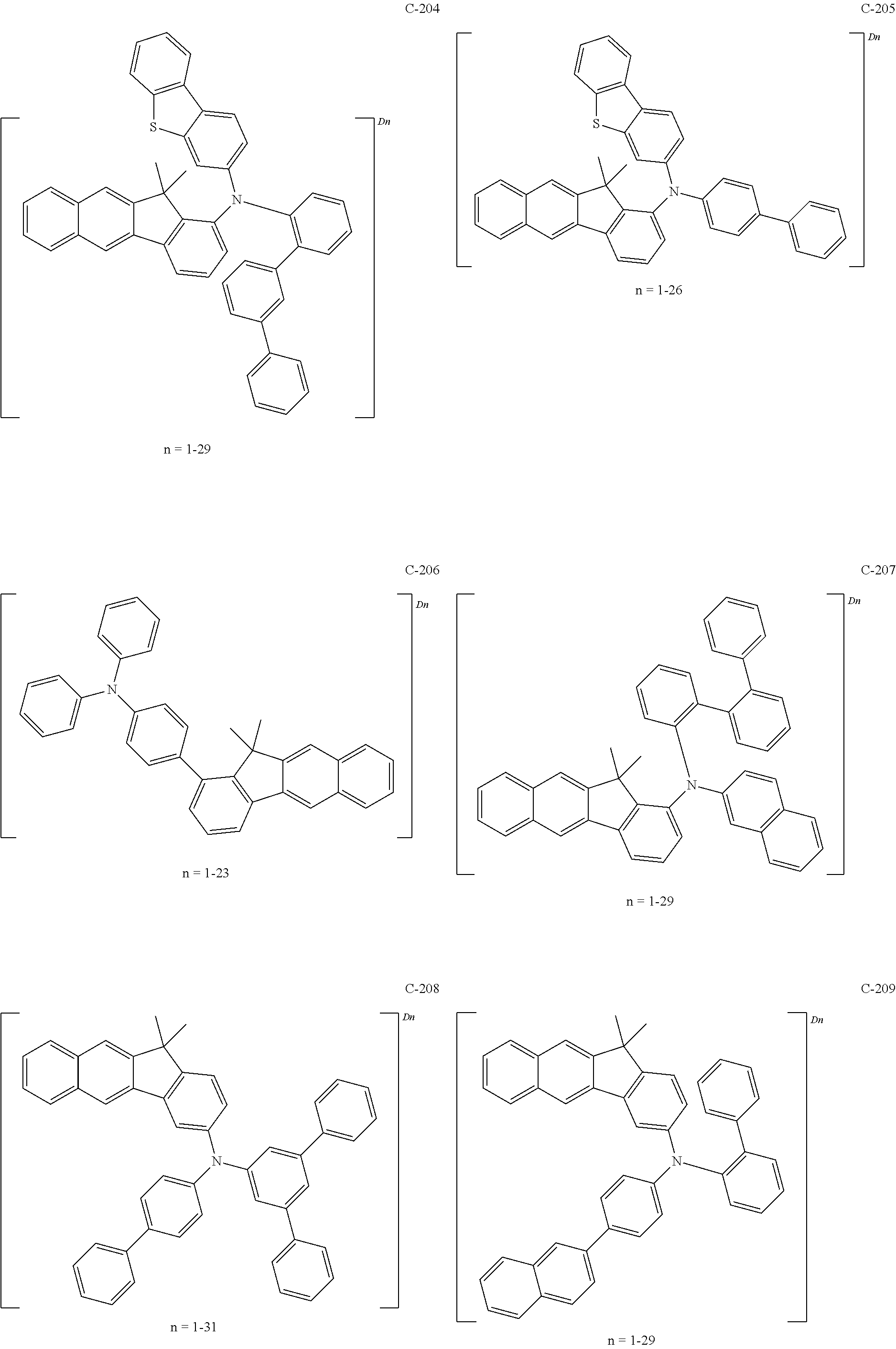

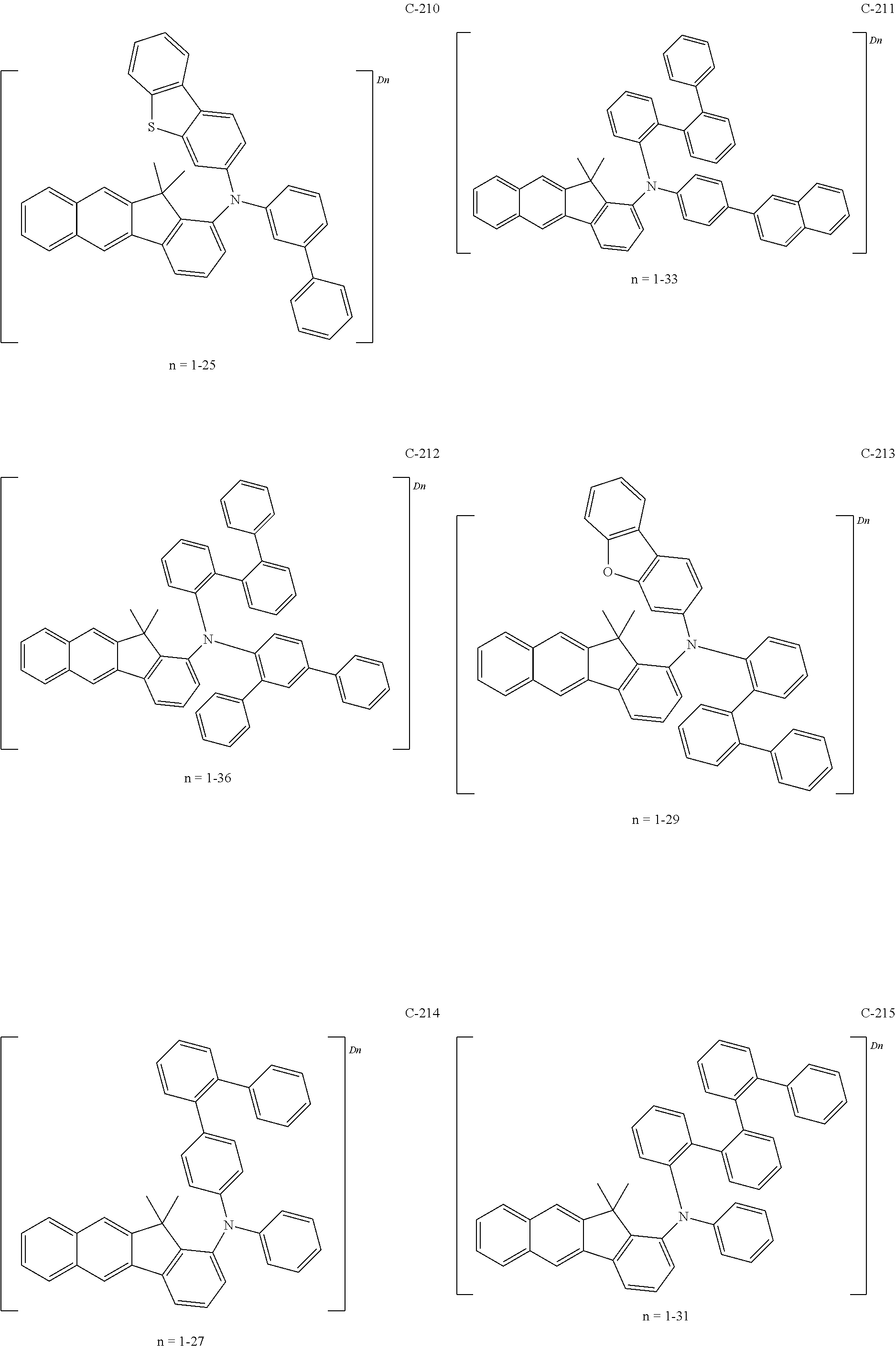

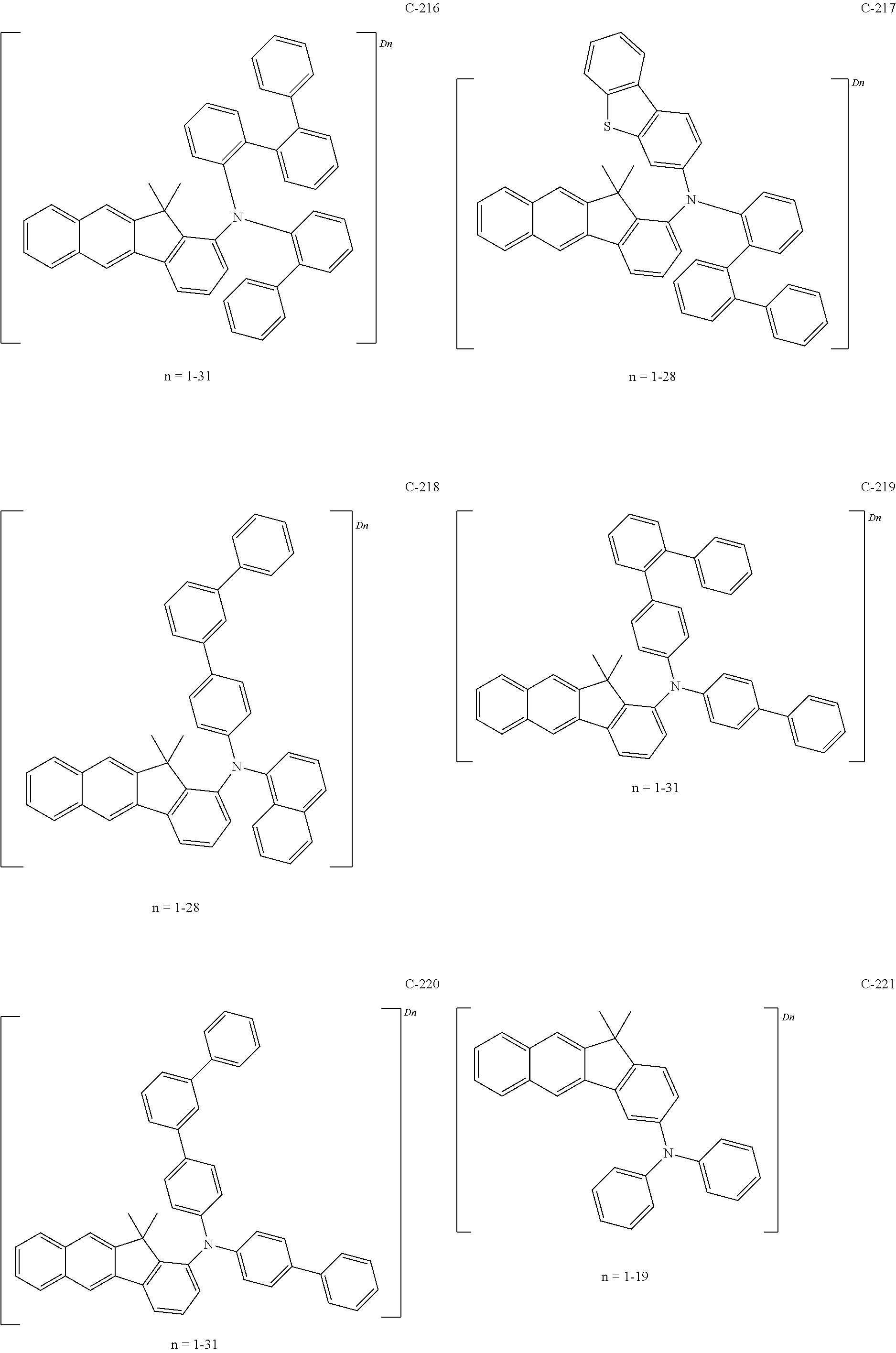

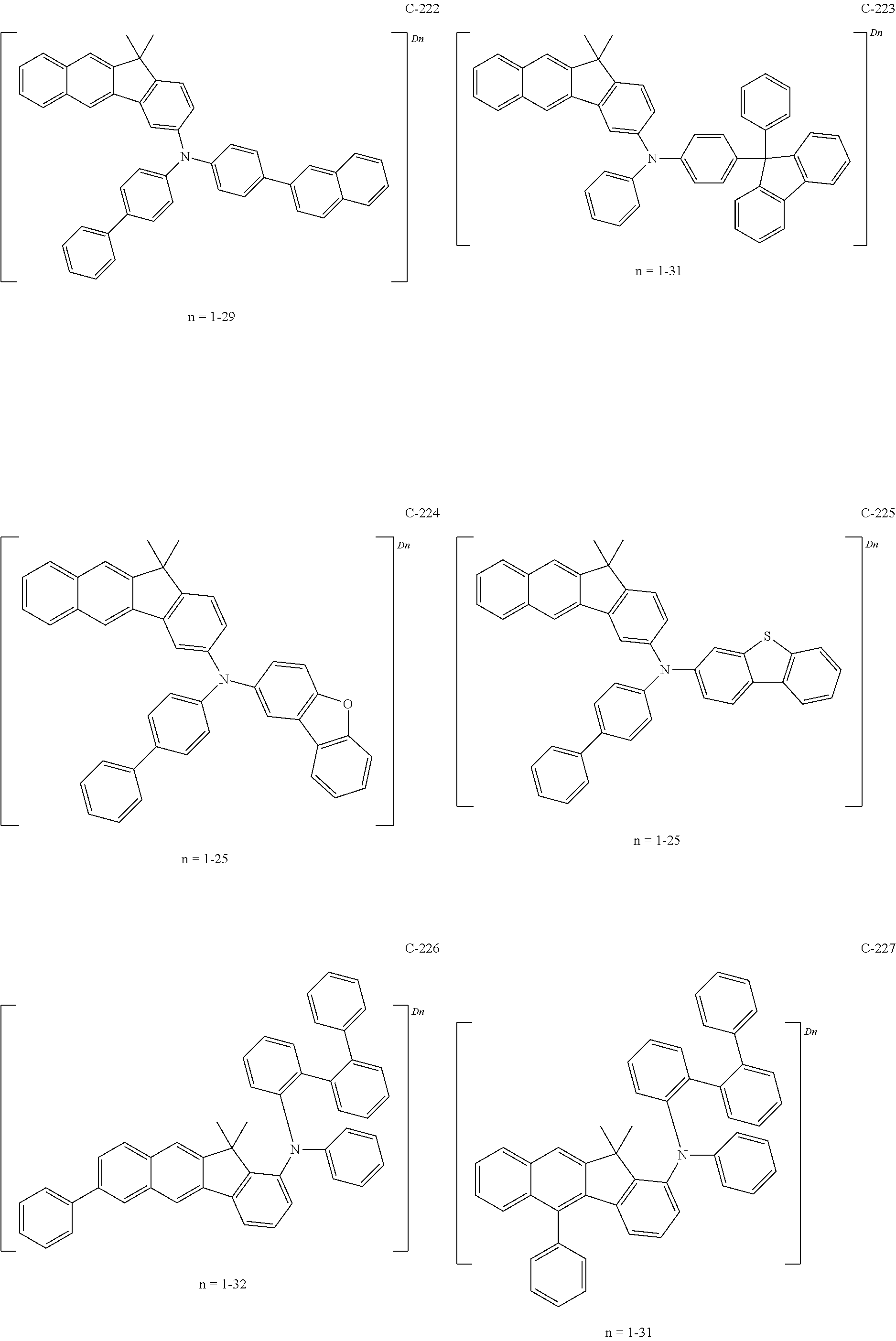

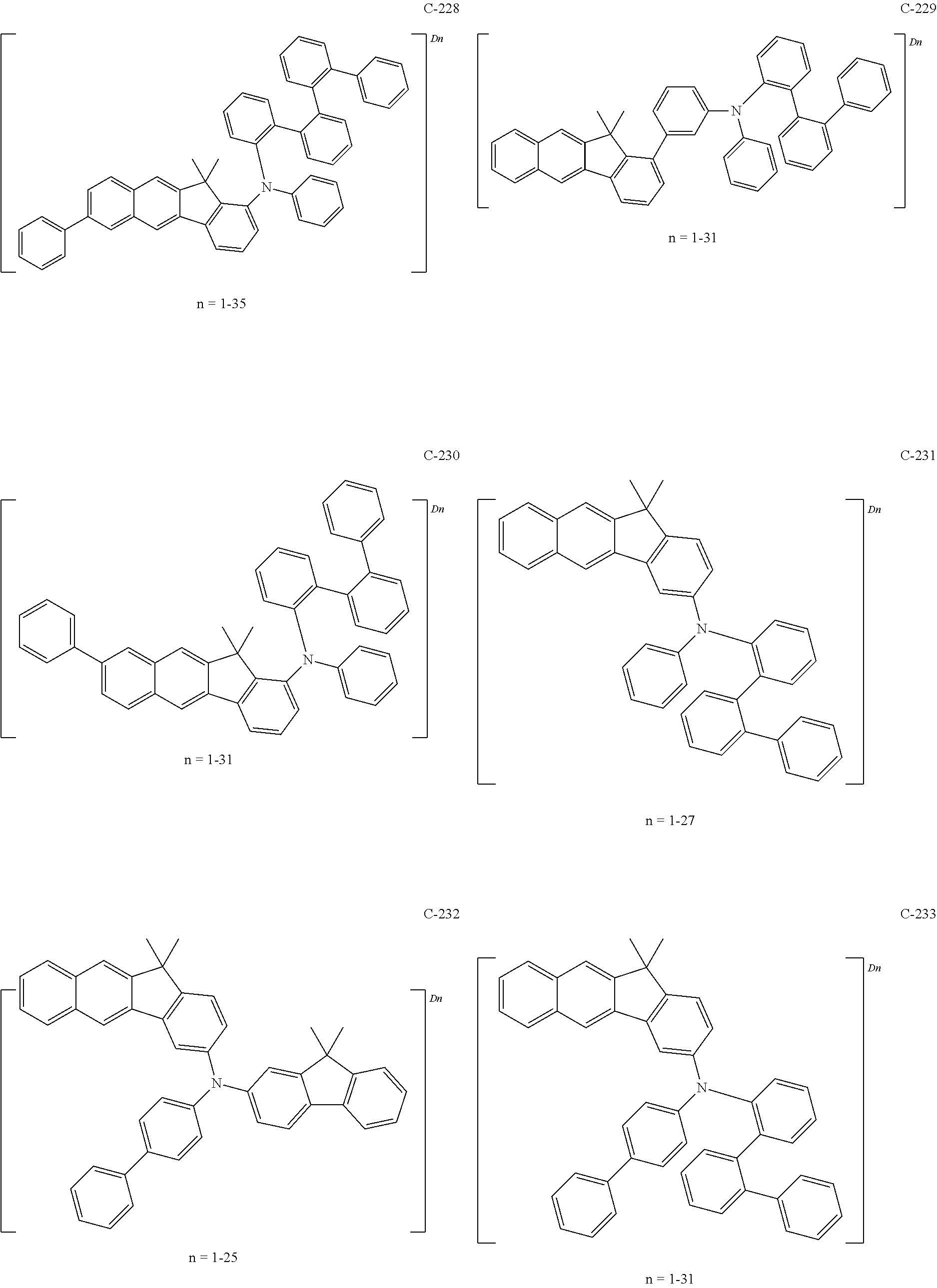

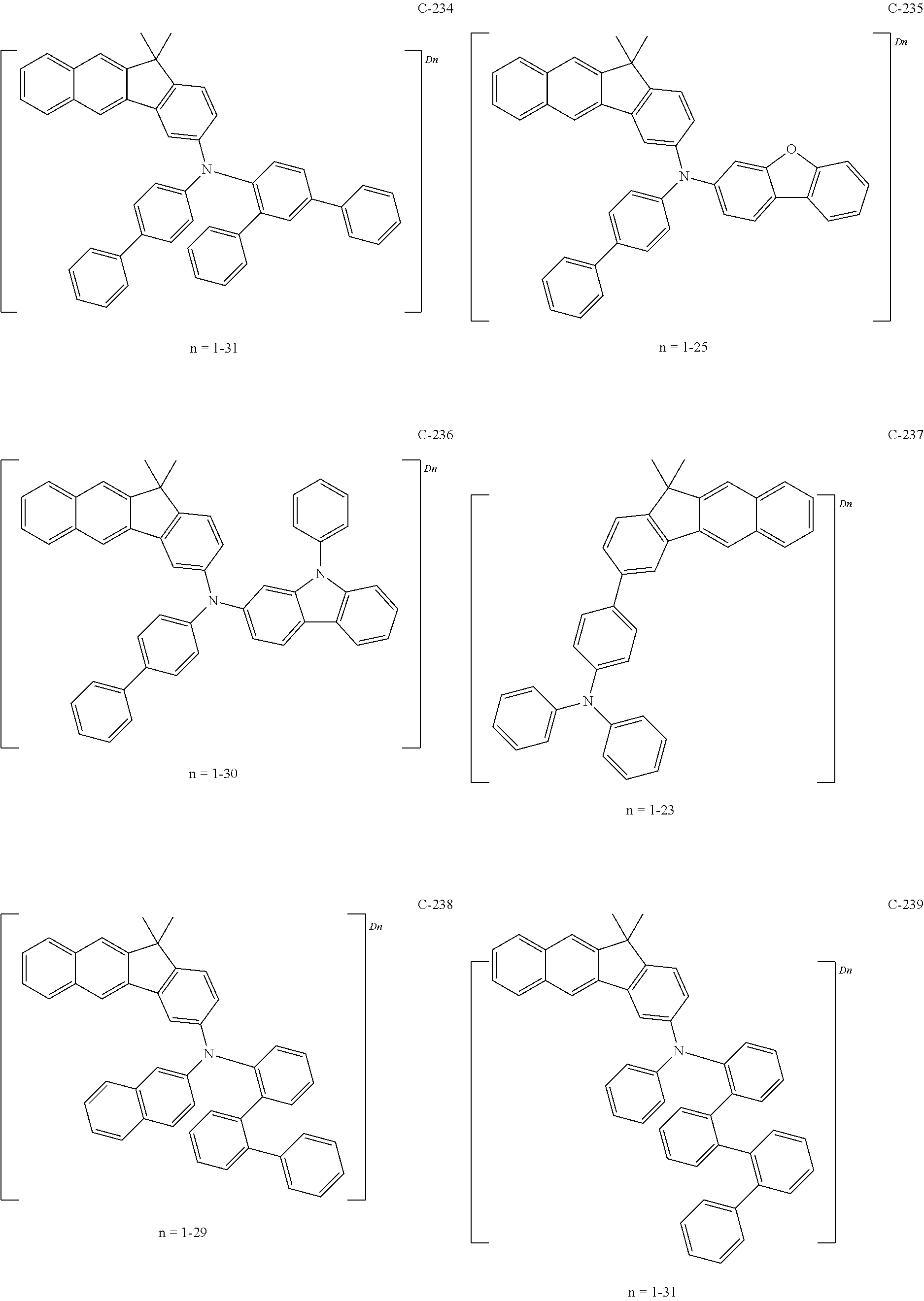

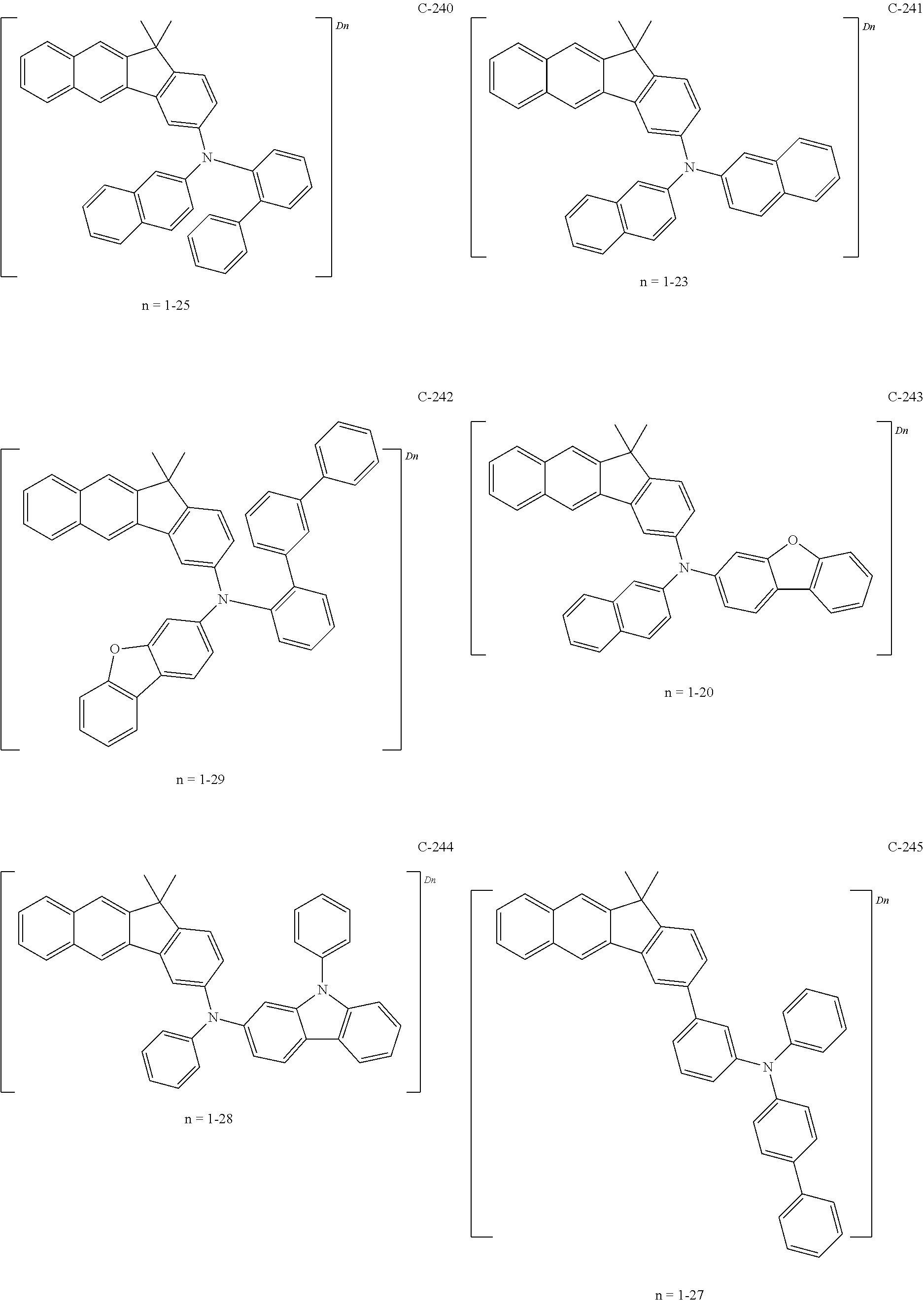

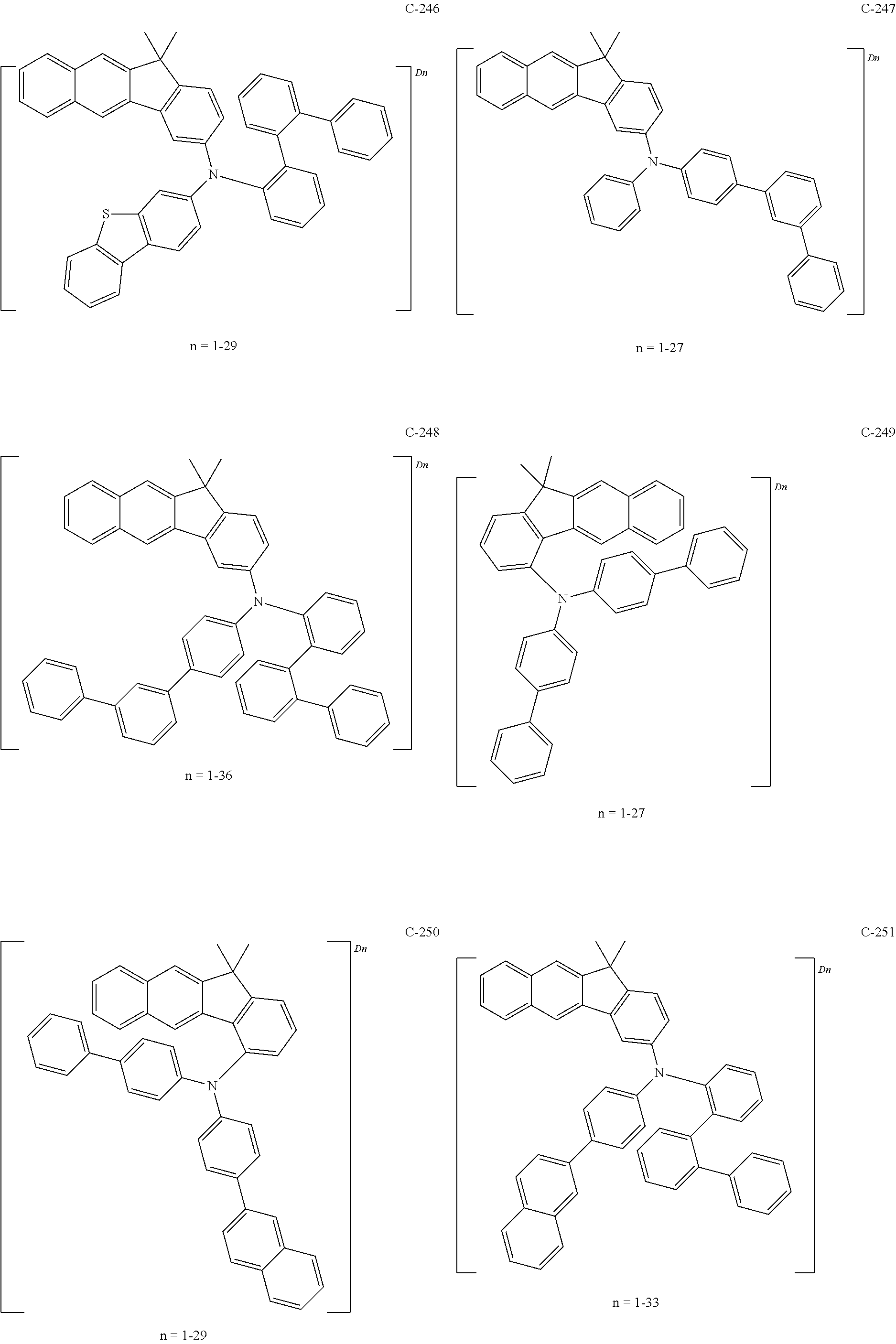

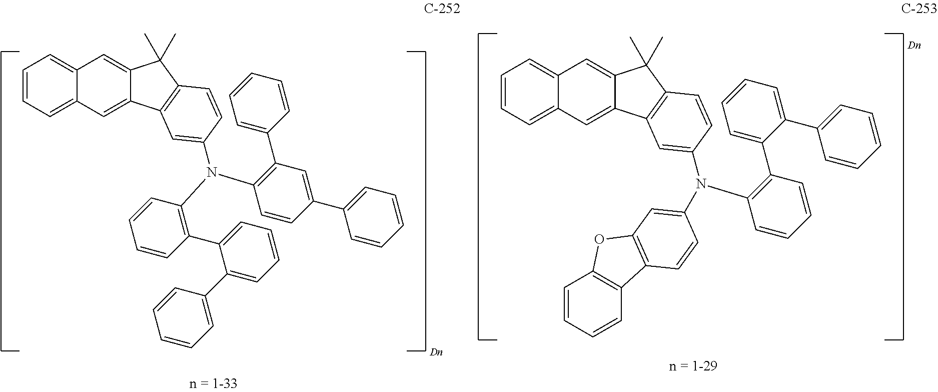

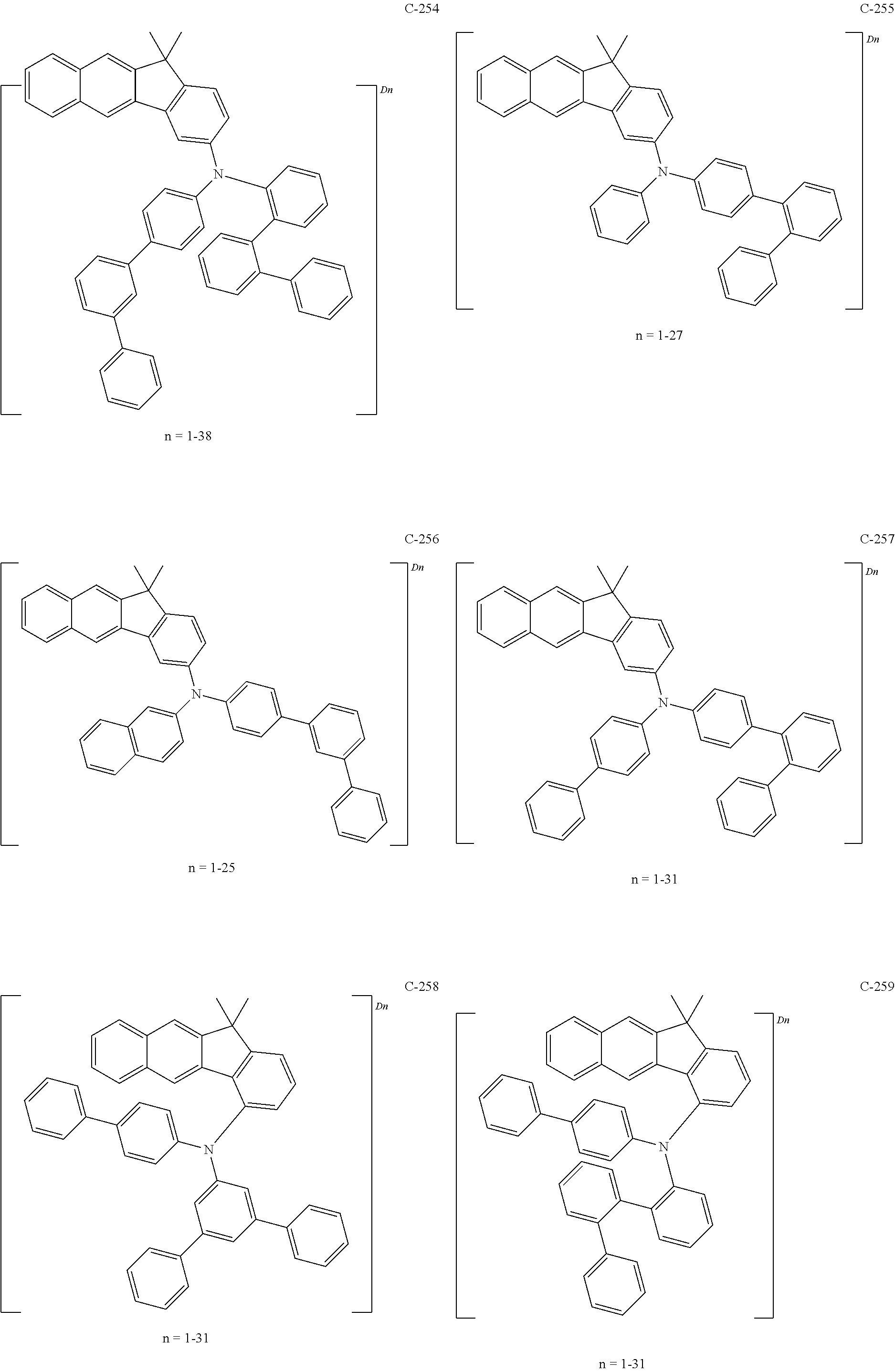

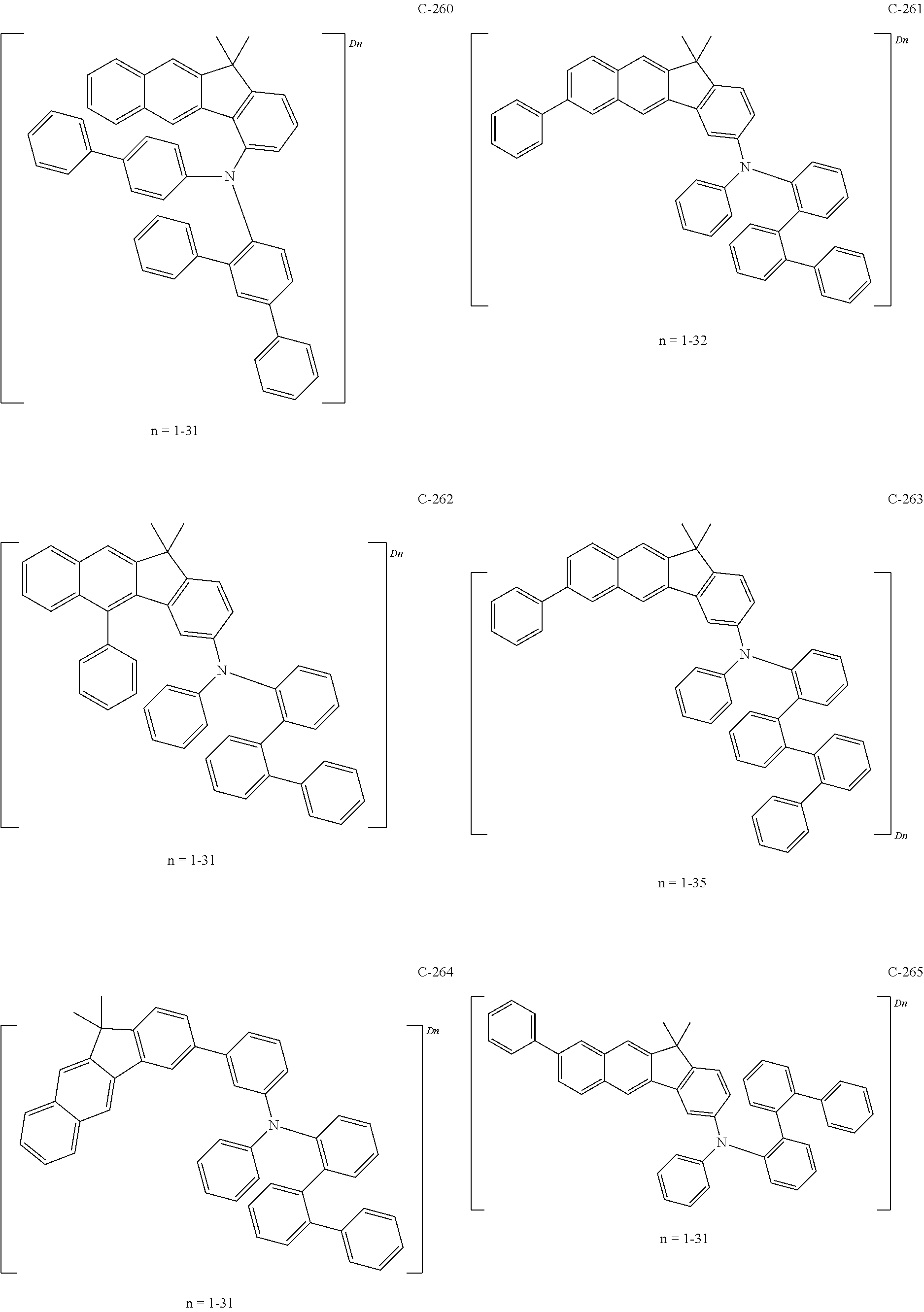

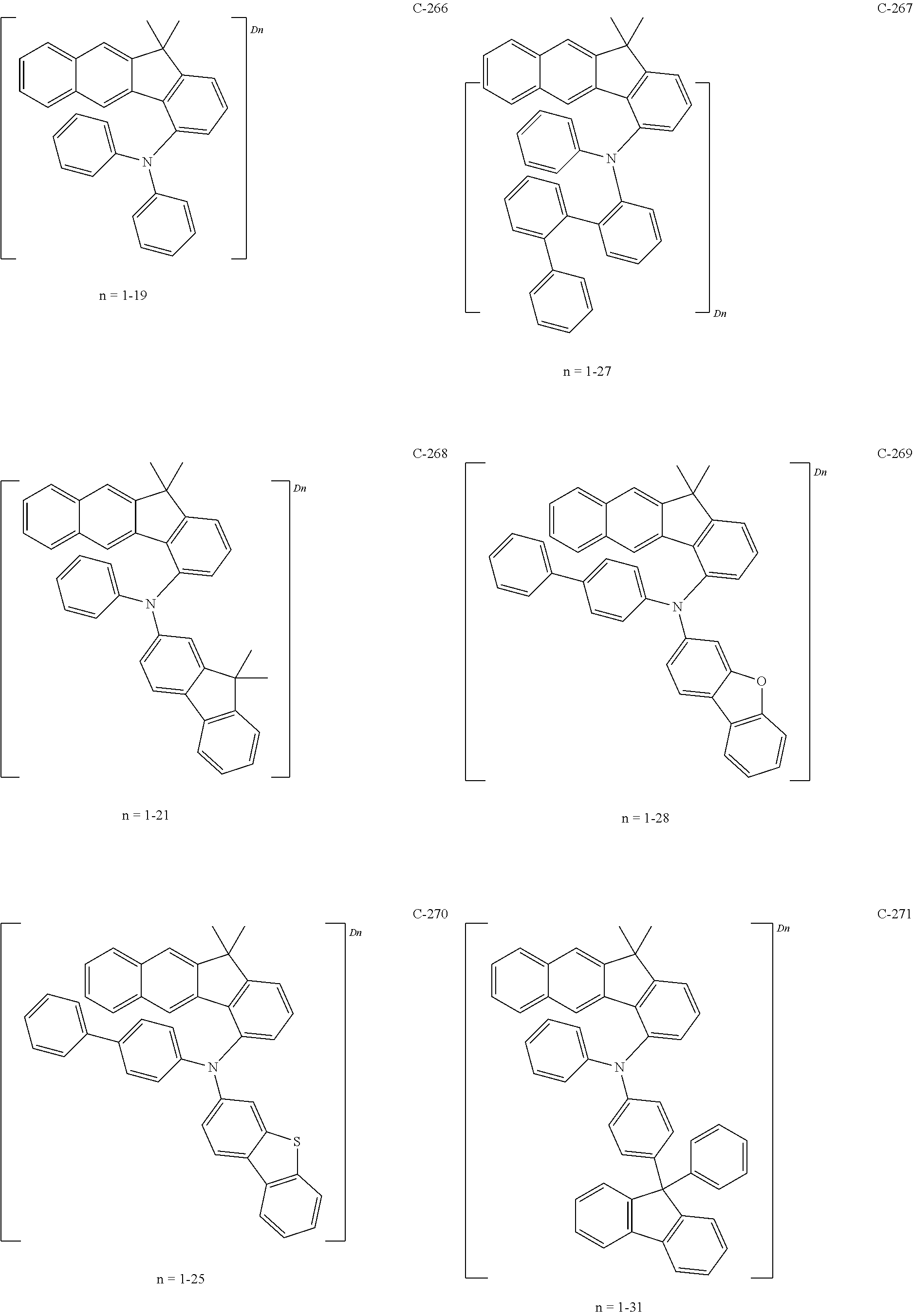

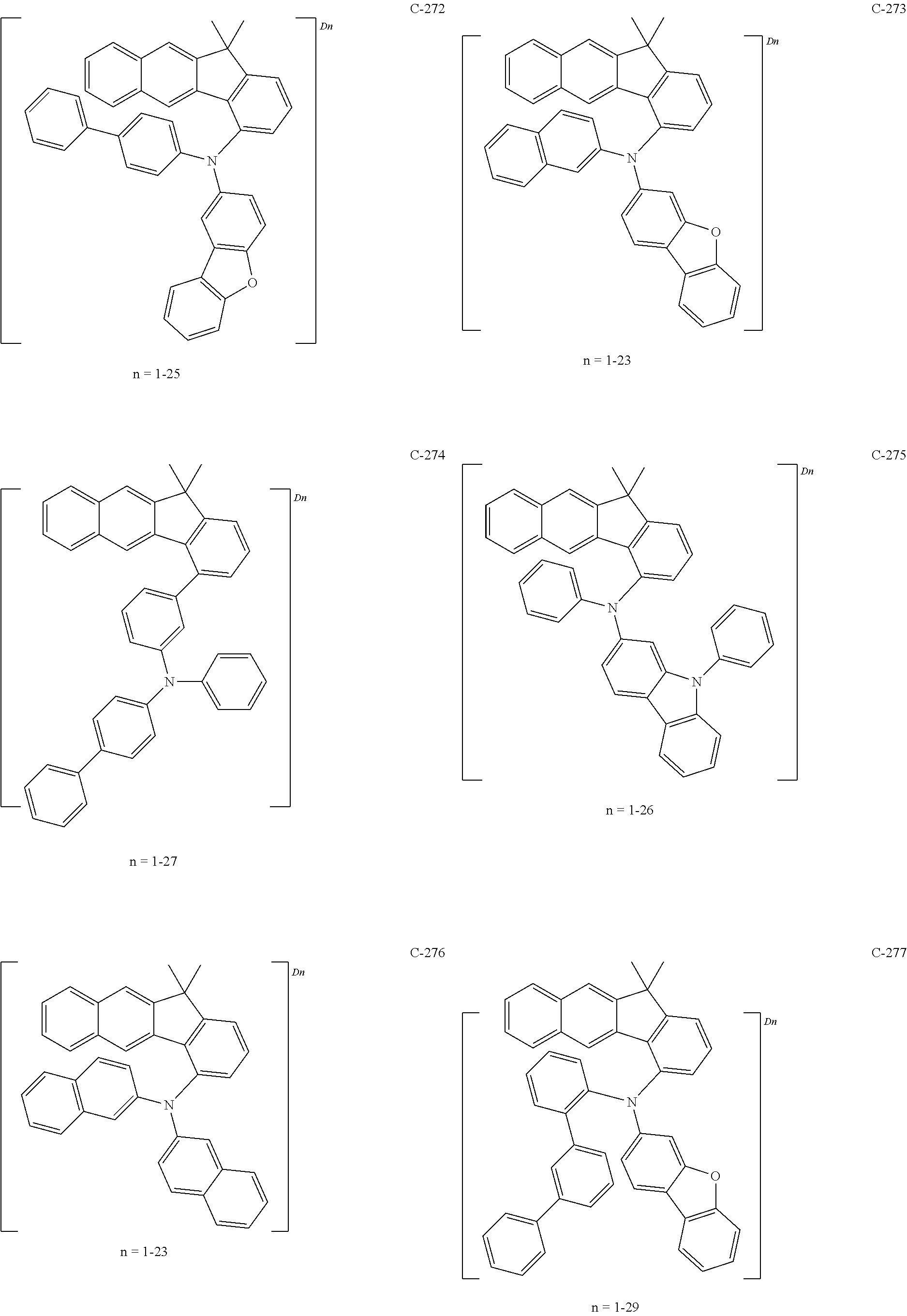

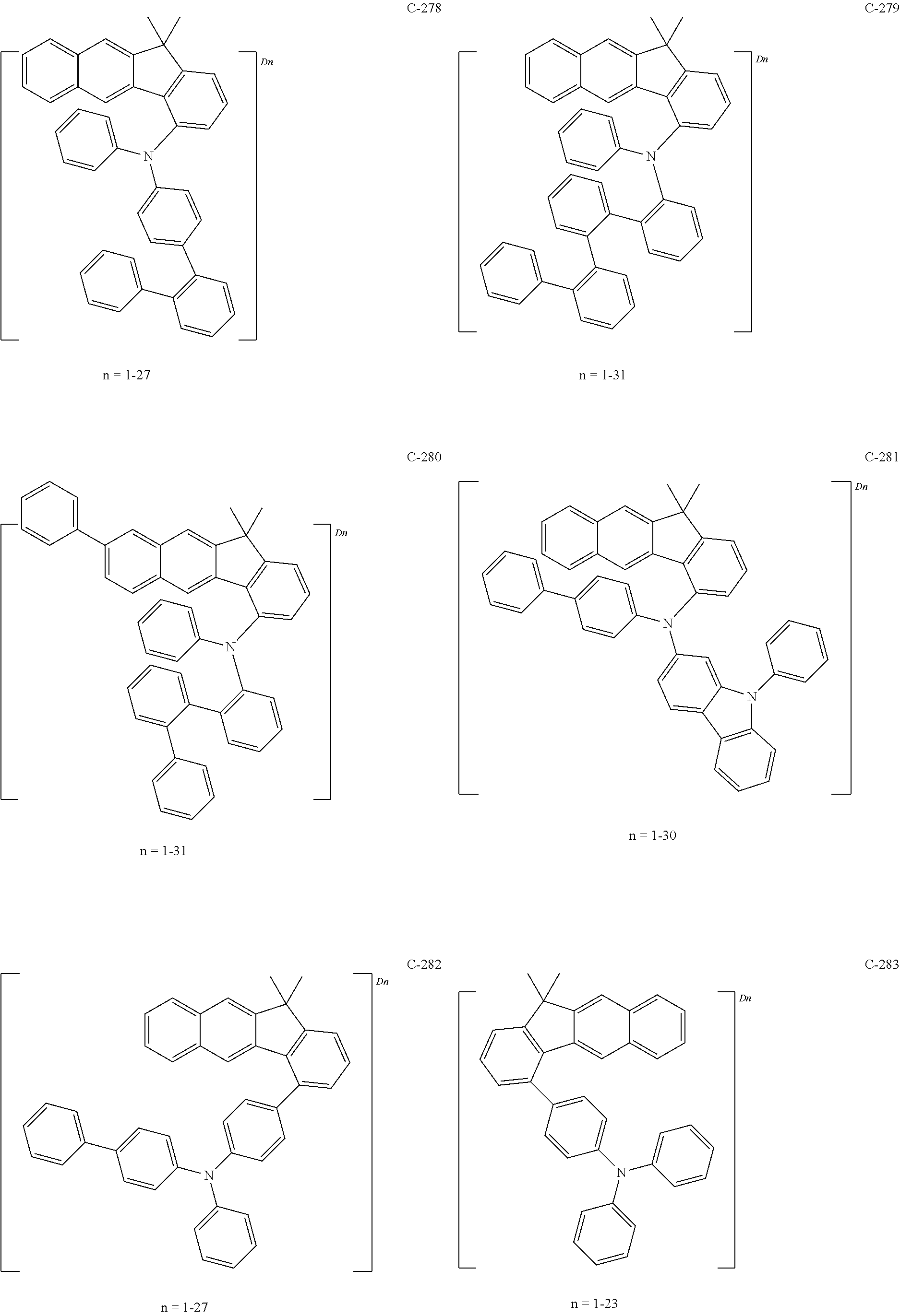

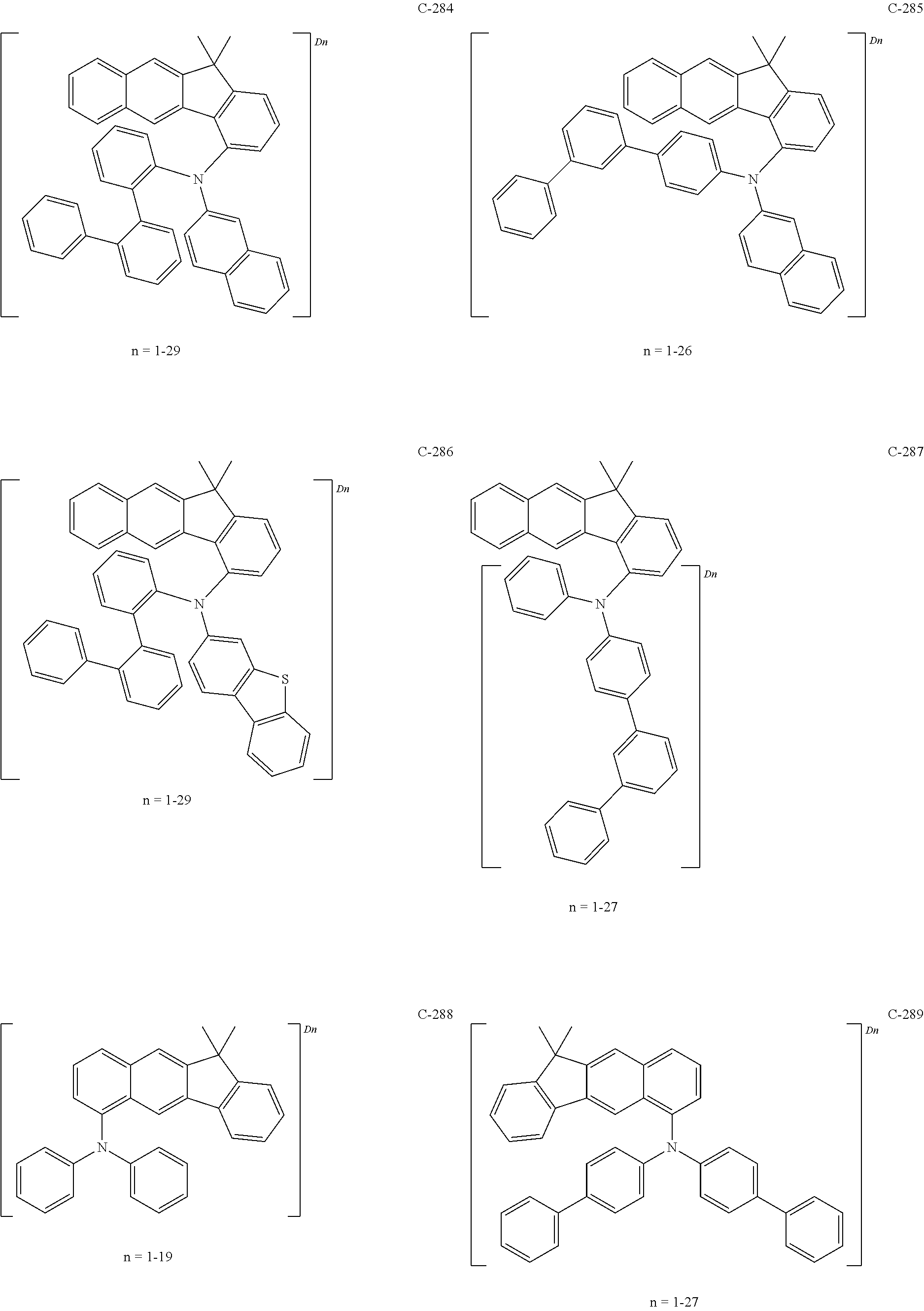

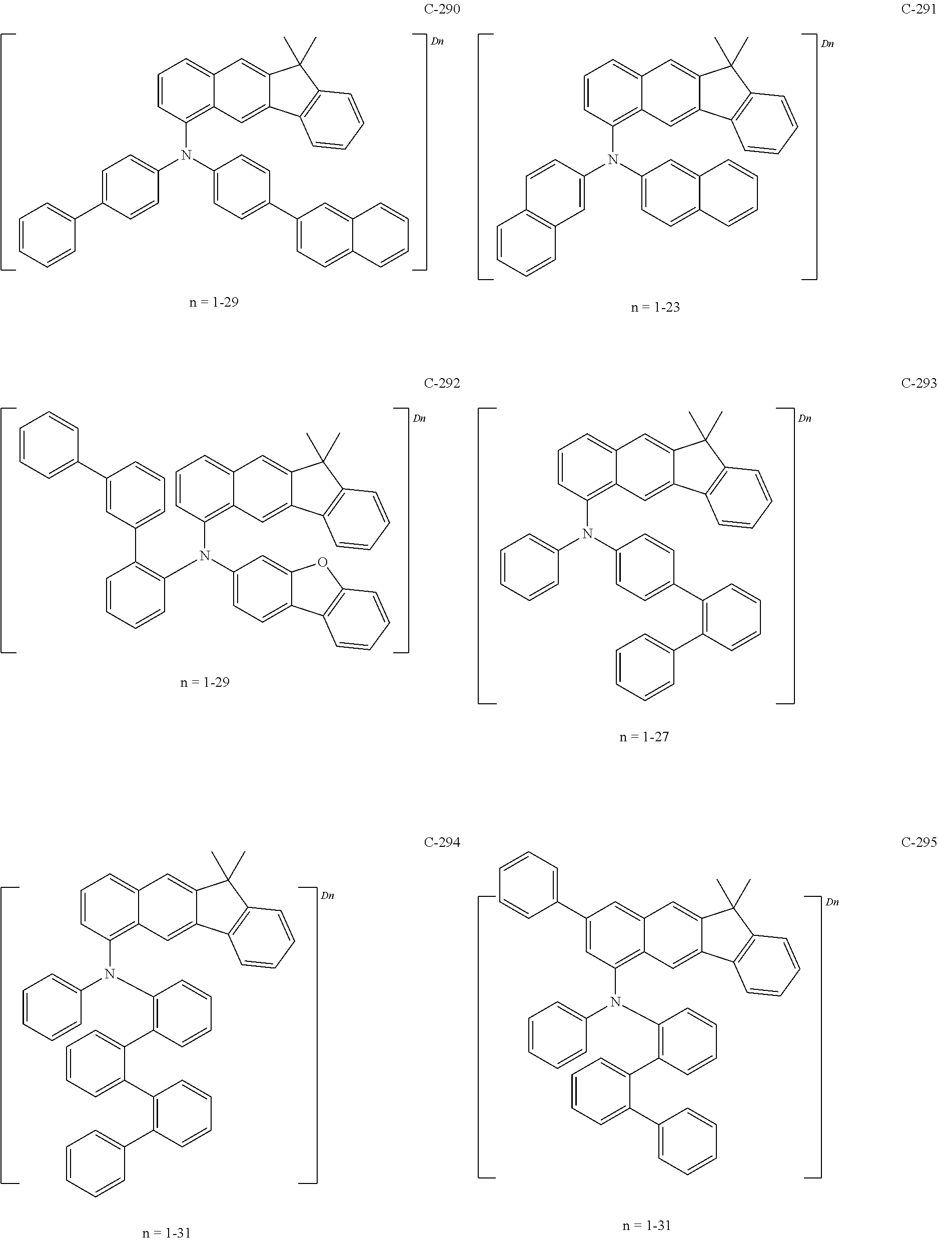

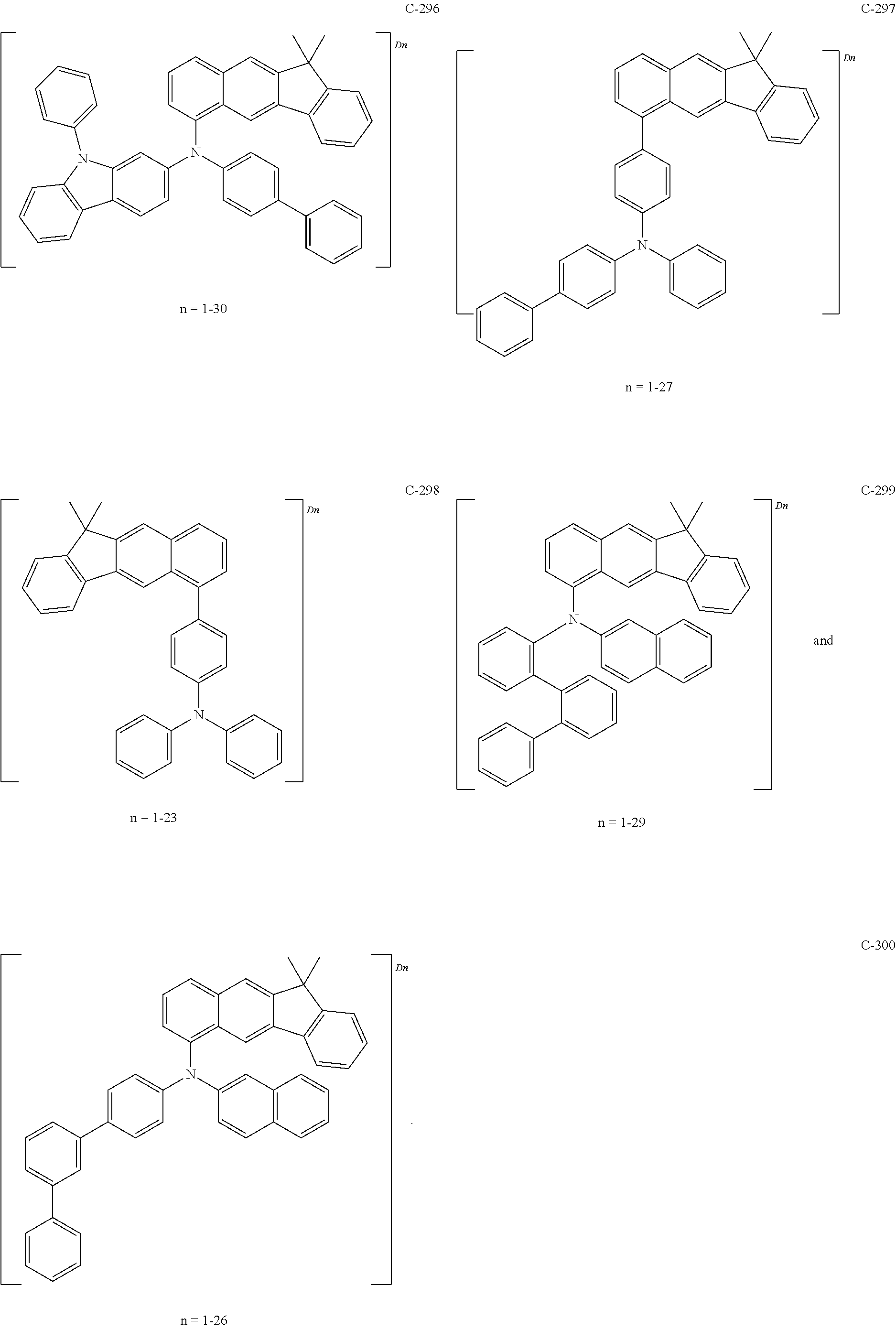

5. The organic electroluminescent compound according to claim 2, wherein the Formula 1-1 is represented by any one of the following formulas of 1-1-1 to 1-1-6. 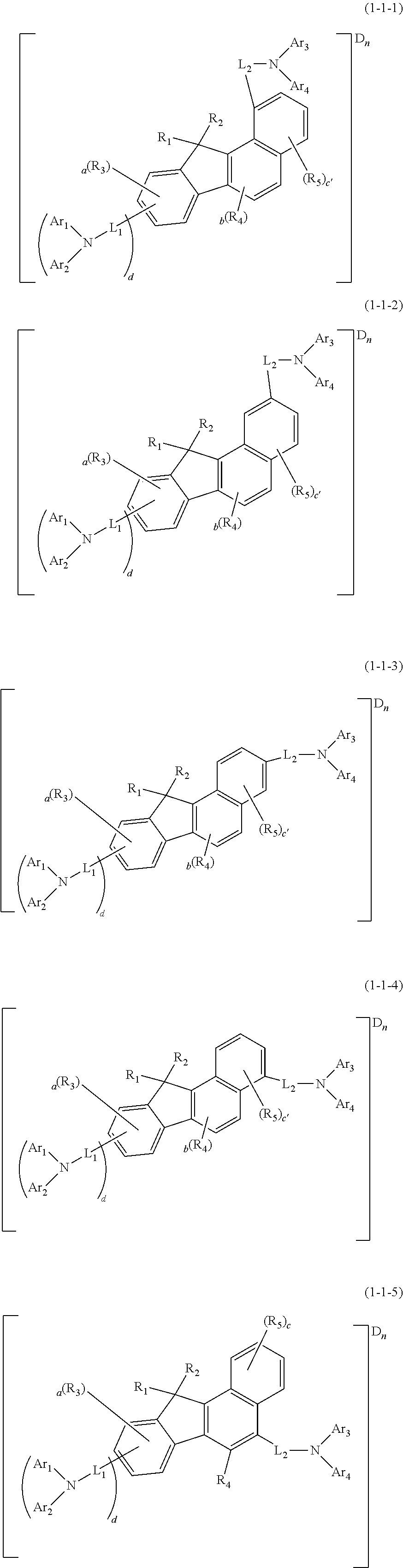 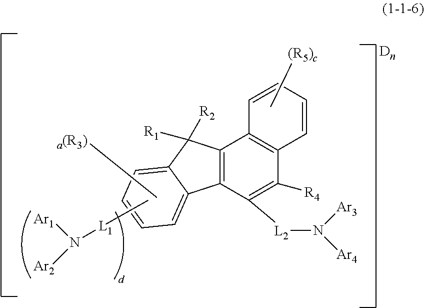
wherein
R₁ to R₅, L₁, L₂, Ar₁ to Ar₄, a to d, and Dn are as defined in claim 2; and
c′ represents an integer of 1 to 3.

6. The organic electroluminescent compound according to claim 2, wherein the Formula 1-2 is represented by any one of the following formulas of 1-2-1 to 1-2-6. 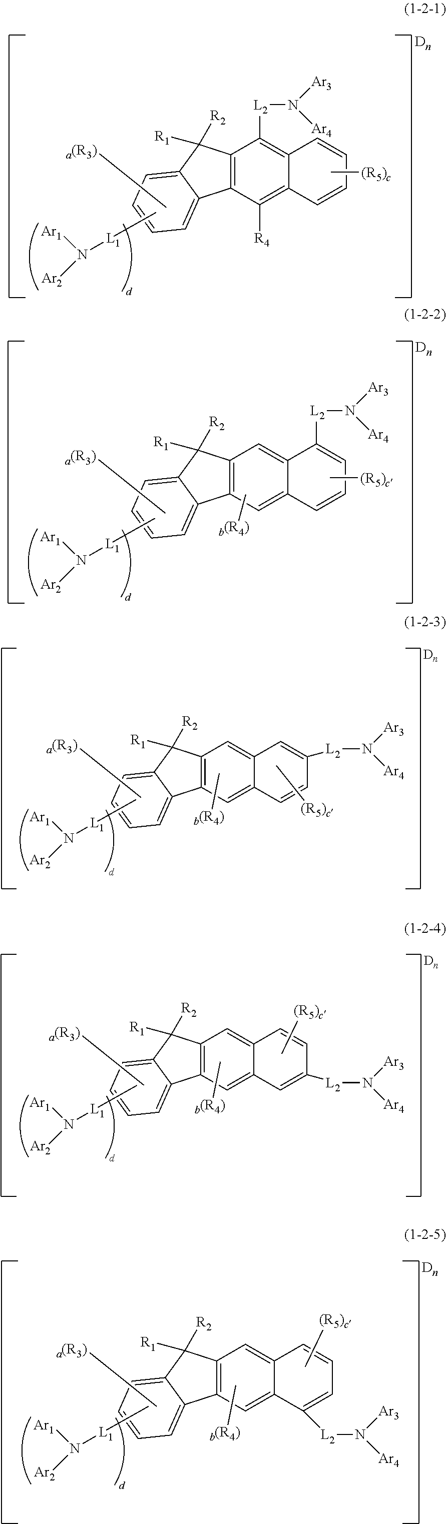 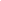
wherein,
R₁ to R₅, L₁, L₂, Ar₁ to Ar₄, a to d, and Dn are as defined in claim 2; and
c′ represents an integer of 1 to 3.

7. The organic electroluminescent compound according to claim 2, wherein the Formula 1-3 is represented by any one of the following formulas of 1-3-1 to 1-3-6. 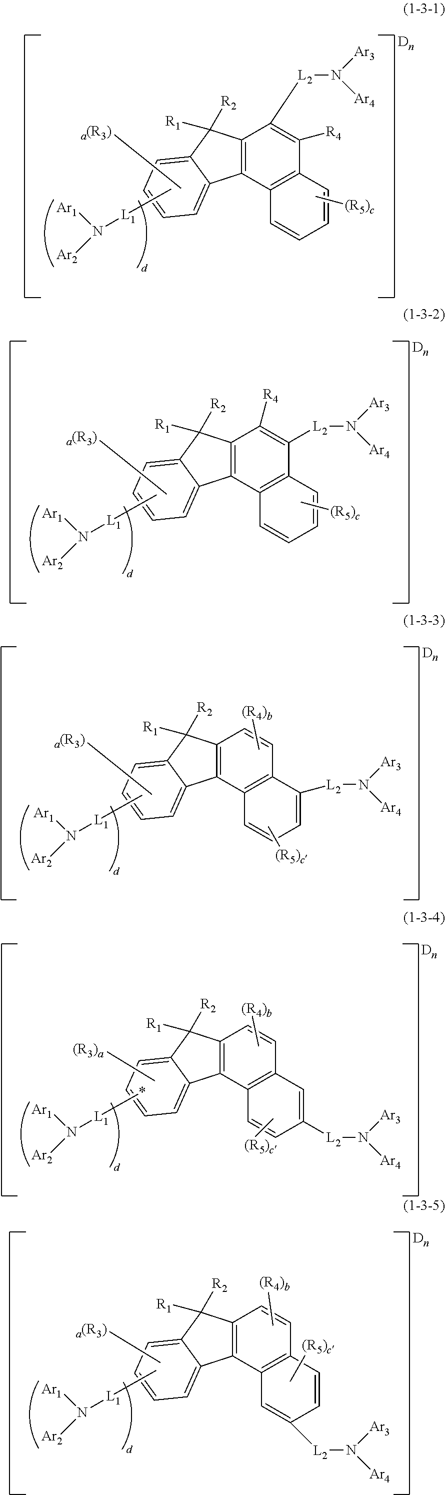 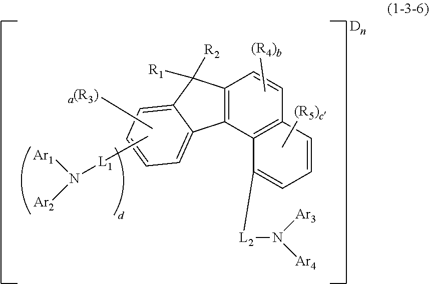
wherein,
R₁ to R₅, L₁, L₂, Ar₁ to Ar₄, a to d, and Dn are as defined in claim 2; and
c′ represents an integer of 1 to 3.

8. The organic electroluminescent compound according to claim 1, wherein the Formula 1 is represented by any one of the following formulas of 1-4 to 1-7. 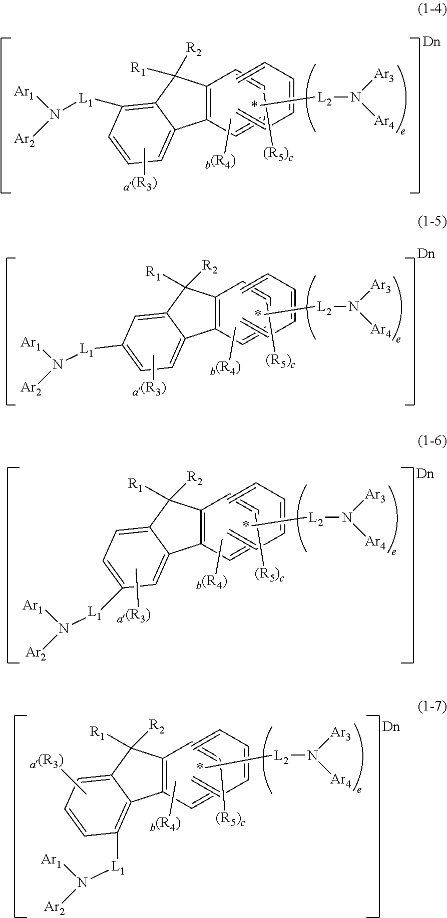
wherein,
R₁ to R₅, L₁, L₂, Ar₁ to Ar₄, b, c, e, and Dn are as defined in claim 1; and
a′ represents an integer of 1 to 3.

9. An organic electroluminescent material comprising an organic electroluminescent compound according to claim 1.

10. An organic electroluminescent device comprising an organic electroluminescent material according to claim 9.
